SEQUENCE LISTING (144 sequences)

SEQ ID NO: 1 - DNA, 64 bp, synthetic construct
  source 1..64: mol_type = other DNA
aggaaattccatttgaatacagcttctggagggagccgacaggagacgtggggagacggccacc

SEQ ID NO: 2 - DNA, 54 bp, synthetic construct
  source 1..54: mol_type = other DNA
aggtgcgacttaccgcgcaatctaacccagcagtctgcattgattaacgccacc

SEQ ID NO: 3 - DNA, 54 bp, synthetic construct
  source 1..54: mol_type = other DNA
aggcacttttattagtgttgtccagtcattacactcggataccttgttgccacc

SEQ ID NO: 4 - DNA, 54 bp, synthetic construct
  source 1..54: mol_type = other DNA
agaacggaacaacctgttgtcattggatcataccgtatcactgacctagccacc

SEQ ID NO: 5 - DNA, 53 bp, synthetic construct
  source 1..53: mol_type = other DNA
agaatatctcgctattctacacgtgatctcacgcagtgtatccctccgccacc

SEQ ID NO: 6 - DNA, 87 bp, synthetic construct
  source 1..87: mol_type = other DNA
agtgttccaagcttcgtctcaagttgagttgttttagattgctgagtttgatcacagaaaagggtaatttcggtccgcactgccacc

SEQ ID NO: 7 - DNA, 87 bp, synthetic construct
  source 1..87: mol_type = other DNA
aggggctatctaccttcaggccttgtcttgtagaacagttagtaaggacctactattcaagaactaactaaggcgcgtccagccacc

SEQ ID NO: 8 - DNA, 87 bp, synthetic construct
  source 1..87: mol_type = other DNA
agacgatcatagactaggacgtgtcgtcggtcattggaatataaaggaacttgccgtcatagtatctttaaggtcacgcgtgccacc

SEQ ID NO: 9 - DNA, 61 bp, synthetic construct
  source 1..61: mol_type = other DNA
agcgaaatttattaaaaaaacgttctgaatcggccaggagaggccgacaaaaaccgccacc

SEQ ID NO: 10 - DNA, 71 bp, synthetic construct
  source 1..71: mol_type = other DNA
aggcaaaatacatcatagtgcagcctatagataaatcggacaggatattatcggagacagaaaccgccacc

SEQ ID NO: 11 - DNA, 108 bp, synthetic construct
  source 1..108: mol_type = other DNA
agcaggcaagtacatccttcgtgtcctaggtgatagtaattggtatcgaggtatatcagaagacaggcaatttcatctggcggtgcacaccctaccagcaacgccacc

SEQ ID NO: 12 - DNA, 108 bp, synthetic construct
  source 1..108: mol_type = other DNA
agaatcaaatcatcttgacggttcagagtcgggcaacgagcagtttcccgcttgtgtcctatcacgttcgcagagggggcatcgaggatcttaactgcactcgccacc

SEQ ID NO: 13 - DNA, 39 bp, synthetic construct
  source 1..39: mol_type = other DNA
agactcttctggtccccacagactcagagagaacccacc

SEQ ID NO: 14 - DNA, 97 bp, synthetic construct
  source 1..97: mol_type = other DNA
agcgcccgctgcagccgccggctctgaatccttctggcttccgcctcagaggagttcttagcctgtcccgaaccgtaaccccggcgagcaggccacc

SEQ ID NO: 15 - DNA, 73 bp, synthetic construct
  source 1..73: mol_type = other DNA
agagagcgccggaccaccctctgtccgccccgaggagtttgccgcctgccggagcacctgcgcacagcccacc

SEQ ID NO: 16 - DNA, 101 bp, synthetic construct
  source 1..101: mol_type = other DNA
agcattccaaccttccagcctgcgacctgcggagaaaaaaaattacttattttcttgccccatacataccttgaggcgagcaaaaaaattaaattttaacc

SEQ ID NO: 17 - DNA, 80 bp, synthetic construct
  source 1..80: mol_type = other DNA
agcactctgcgcgcccgctcttctgctgctgtttgtctacttcctcctgcttccccgccgccgccgccgccatcgccacc

SEQ ID NO: 18 - DNA, 70 bp, synthetic construct
  source 1..70: mol_type = other DNA
aggggagatcttcgtcaccctttttcacttcctcagttttcgcggaccacttcaaggactaaaagccacc

SEQ ID NO: 19 - DNA, 58 bp, synthetic construct
  source 1..58: mol_type = other DNA
agctcctcccgaagtgctcctcttctacagctgttccgcagtcgccgccgccgccacc

SEQ ID NO: 20 - DNA, 56 bp, synthetic construct
  source 1..56: mol_type = other DNA
aggctcttccagactctgcgacctaactgactcttgggttcaaggctagggccacc

SEQ ID NO: 21 - DNA, 58 bp, synthetic construct
  source 1..58: mol_type = other DNA
agcgcagcctgctgtccgcctgccttttcgtctctagccgcgtgaagtcagcgccacc

SEQ ID NO: 22 - DNA, 52 bp, synthetic construct
  source 1..52: mol_type = other DNA
agctcaaagaaaaaccaaacgtaacaagggcgaattcgttggtaaagccacc

SEQ ID NO: 23 - DNA, 58 bp, synthetic construct
  source 1..58: mol_type = other DNA
agggtcccacagtctggtcctgcttcaccttcccctgacctgagtagtcgccgccacc

SEQ ID NO: 24 - DNA, 58 bp, synthetic construct
  source 1..58: mol_type = other DNA
agctcagtgagcaaccctaattaacctgattttttgctgataatcactctcagccacc

SEQ ID NO: 25 - DNA, 49 bp, synthetic construct
  source 1..49: mol_type = other DNA
agagtcccgagcctaaagcctcgcctgcctgcccagtgccagggccacc

SEQ ID NO: 26 - DNA, 59 bp, synthetic construct
  source 1..59: mol_type = other DNA
agctcagtcctggtcccccagtcccaagcctcacctgcctgcccagcgccagggccacc

SEQ ID NO: 27 - DNA, 110 bp, synthetic construct
  source 1..110: mol_type = other DNA
agaaaagaaagaagccagaacataccaccagccctttgagtatacaacataacaaacgtcattactttacaacttgattaggagacagtacagagatctgaagggccacc

SEQ ID NO: 28 - DNA, 83 bp, synthetic construct
  source 1..83: mol_type = other DNA
agagaacacgacacaacgaactttatattttaccacttccttgaatagttgcaggagaaataacaaggcattgaagagccacc

SEQ ID NO: 29 - DNA, 134 bp, synthetic construct
  source 1..134: mol_type = other DNA
aggagacgccatccacgctgttttgacctccatagaagacaccgggaccgatccagcctccgcggccgggaacggtgcattggaacgcggattccccgtgccaagagtgactcaccgtccttgacacggccacc

SEQ ID NO: 30 - DNA, 66 bp, synthetic construct
  source 1..66: mol_type = other DNA
agaacggtgcattggaacgcggattccccgtgccaagagtgactcaccgtccttgacacggccacc

SEQ ID NO: 31 - DNA, 51 bp, synthetic construct
  source 1..51: mol_type = other DNA
agggagacgccatccacgctgttttgacctccatagaagacaccgggaccg

SEQ ID NO: 32 - DNA, 22 bp, synthetic construct
  source 1..22: mol_type = other DNA
agataaacgctcaactttggcc

SEQ ID NO: 33 - DNA, 28 bp, synthetic construct
  source 1..28: mol_type = other DNA
agataaacgctcaactttggccgccacc

SEQ ID NO: 34 - DNA, 28 bp, synthetic construct
  source 1..28: mol_type = other DNA
agataaacgctcaactttggccgccaag

SEQ ID NO: 35 - DNA, 52 bp, synthetic construct
  source 1..52: mol_type = other DNA
agattccccgcctcctcctgtcccgcagtcggataaacgctcaactttggcc

SEQ ID NO: 36 - DNA, 45 bp, synthetic construct
  source 1..45: mol_type = other DNA
agggagaccatattgaagagacagagtgatatataaaactgctaa

SEQ ID NO: 37 - DNA, 51 bp, synthetic construct
  source 1..51: mol_type = other DNA
agggagaccatattgaagagacagagtgatatataaaactgctaagccacc

SEQ ID NO: 38 - DNA, 19 bp, synthetic construct
  source 1..19: mol_type = other DNA
agggagacaaaactgctaa

SEQ ID NO: 39 - DNA, 25 bp, synthetic construct
  source 1..25: mol_type = other DNA
agggagacaaaactgctaagccaag

SEQ ID NO: 40 - DNA, 15 bp, synthetic construct
  source 1..15: mol_type = other DNA
agggagactgccacc

SEQ ID NO: 41 - DNA, 15 bp, synthetic construct
  source 1..15: mol_type = other DNA
agggagactgccaag

SEQ ID NO: 42 - DNA, 47 bp, synthetic construct
  source 1..47: mol_type = other DNA
agggaagtgcagccccaggttcccgagcggcaggttgggtgcggacc

SEQ ID NO: 43 - DNA, 66 bp, synthetic construct
  source 1..66: mol_type = other DNA
agggaactcctccccatcctctccctctgtccctctgtccctctgaccctgcactgtcccagcacc

SEQ ID NO: 44 - DNA, 38 bp, synthetic construct
  source 1..38: mol_type = other DNA
agggactcccgggctggcagcagggccccagcggcacc

SEQ ID NO: 45 - DNA, 60 bp, synthetic construct
  source 1..60: mol_type = other DNA
agggaaatcctttctttcagctggagtgctcctcaggagccagccccacccttagaaaag

SEQ ID NO: 46 - DNA, 102 bp, synthetic construct
  source 1..102: mol_type = other DNA
agcactcgcgctgccatcactcttccgccgtcttcgccgccatcctcggcgcgactcgcttctttcggttctaccaggtagagtccgccgccatcctccacc

SEQ ID NO: 47 - DNA, 101 bp, synthetic construct
  source 1..101: mol_type = other DNA
agccggtggcggcaggatacagcggcttctgcgcgacttataagagctccttgtgcggcgccattttaagcctctcggtctgtggcagcagcgttggcccg

SEQ ID NO: 48 - DNA, 102 bp, synthetic construct
  source 1..102: mol_type = other DNA
agctacagaaacgaaagaaaaagtctgtataagccaaaggtgttcgggaagaaaataaccccattgccttgagtttgtaggtgccactactactctggaaaa

SEQ ID NO: 49 - DNA, 58 bp, synthetic construct
  source 1..58: mol_type = other DNA
agttgccgctgtcgtccgcagagccagttcctagcgcagagccgcgcccgccgccacc

SEQ ID NO: 50 - DNA, 133 bp, synthetic construct
  source 1..133: mol_type = other DNA
agcgcgttattgttctgccgggcggacacgtgacgcgaagcttaccgccgagaccgcgtccgccccgcgagcacagagcctcgcctttgccgatccgccgcccgtccacacccgccgccagctcaccgccacc

SEQ ID NO: 51 - DNA, 92 bp, synthetic construct
  source 1..92: mol_type = other DNA
agaccgccgagaccgcgtccgccccgcgagcacagagcctcgcctttgccgatccgccgcccgtccacacccgccgccagctcaccgccacc

SEQ ID NO: 52 - DNA, 119 bp, synthetic construct
  source 1..119: mol_type = other DNA
agtataaaacccggcggcgcaacgcgcagccactgtcgagtcgcgtccacccgcgagcacagcttctttgcagctccttcgttgccggtccacacccgccaccagttcgccccgccacc

SEQ ID NO: 53 - DNA, 160 bp, synthetic construct
  source 1..160: mol_type = other DNA
agcgcgttattgttctgccgggcggacacgtgacgcgaagctttataaaacccggcggcgcaacgcgcagccactgtcgagtcgcgtccacccgcgagcacagcttctttgcagctccttcgttgccggtccacacccgccaccagttcgccccgccacc

SEQ ID NO: 54 - DNA, 400 bp, synthetic construct
  source 1..400: mol_type = other DNA
agttgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaagggcgaattcgttggtaaagccacc

SEQ ID NO: 55 - DNA, 52 bp, synthetic construct
  source 1..52: mol_type = other DNA
agtcaacgtagttctaacagttttttaattagagagcagatctctggccacc

SEQ ID NO: 56 - DNA, 66 bp, synthetic construct
  source 1..66: mol_type = other DNA
agatctttgggtggtggagtgcaaaggaggcgacctgcaacagaggagtcccggtcaccagccacc

SEQ ID NO: 57 - DNA, 80 bp, synthetic construct
  source 1..80: mol_type = other DNA
agaaaccccagctgttggggccaggacacccagtgagcccatacttgctctttttgtcttcttcagactgcgccgccacc

SEQ ID NO: 58 - DNA, 52 bp, synthetic construct
  source 1..52: mol_type = other DNA
agcaaaaggaattcttctcgggaggcagtgctgggtcctttccaccgccacc

SEQ ID NO: 59 - DNA, 64 bp, synthetic construct
  source 1..64: mol_type = other DNA
aggaaattggatttggctgcagcttctggagggagccgacaggagacgtggggagacggccacc

SEQ ID NO: 60 - DNA, 64 bp, synthetic construct
  source 1..64: mol_type = other DNA
aggaaattccatttggctgcagcttctggagggagccgacaggagacgtggggagagtgccacc

SEQ ID NO: 61 - DNA, 64 bp, synthetic construct
  source 1..64: mol_type = other DNA
aggaaattccatttggctgcagcttaaagagggagccgacaggagacgtggggagacggccacc

SEQ ID NO: 62 - DNA, 64 bp, synthetic construct
  source 1..64: mol_type = other DNA
aggaaattccatttgaattcagcttctggagggagccgacaggagacgtggggagacggcatcc

SEQ ID NO: 63 - DNA, 54 bp, synthetic construct
  source 1..54: mol_type = other DNA
agggtctattcaaagactgcggacgcgatcaagtgctctgttccatctgccacc

SEQ ID NO: 64 - DNA, 87 bp, synthetic construct
  source 1..87: mol_type = other DNA
agtcggttgtctaatcaagatcatatcttgggaccgggctatacttcatcaaaaactgtccatatagtcgcggcggggaaagccacc

SEQ ID NO: 65 - DNA, 63 bp, synthetic construct
  source 1..63: mol_type = other DNA
agcgaacttacaaaaaaaagcatcttatcgaaaccgccgctcggagacgagcgcacagccacc

SEQ ID NO: 66 - DNA, 71 bp, synthetic construct
  source 1..71: mol_type = other DNA
agcgaaaaaaaattaaaatccagcttctgtaaagagccgacaggagaggtggcgagacaaaaaccgccacc

SEQ ID NO: 67 - DNA, 62 bp, synthetic construct
  source 1..62: mol_type = other DNA
aggtagtaaaaaagctgattgagttgttaggagaggtggcgagagtttaggggagggccacc

SEQ ID NO: 68 - DNA, 56 bp, synthetic construct
  source 1..56: mol_type = other DNA
aggcacggtaaccgaaagcgttcgaacaggggcgccaatacattaaaattgccacc

SEQ ID NO: 69 - DNA, 108 bp, synthetic construct
  source 1..108: mol_type = other DNA
agttagaaagagtactcataaacgataataaagtgaacctattaggcccgtcttgcggagttgaggagcttgcatctctgctgaggttaaaactaatatcttgccacc

SEQ ID NO: 70 - skipped

SEQ ID NO: 71 - DNA, 64 bp, synthetic construct
  source 1..64: mol_type = other DNA
aggaaattccatttggctgcagcttctggagggagccgacaggagacgtggggagacggccacc

SEQ ID NO: 72 - DNA, 94 bp, synthetic construct
  source 1..94: mol_type = other DNA
gctgccttctgcggggcttgccttctggccatgcccttcttctctcccttgcacctgtacctcttggtctttgaataaagcctgagtaggaagt

SEQ ID NO: 73 - DNA, 75 bp, synthetic construct
  source 1..75: mol_type = other DNA
aaaaaaaaaaaaaaaaaaaaaaaaaaaaaaaaaaaaaaaaaaaaaaaaaaaaaaaaaaaaaaaaaaaaaaaaaaa

SEQ ID NO: 74 - RNA, 64 nt, synthetic construct
  source 1..64: mol_type = other RNA
aggaaattccatttgaatacagcttctggagggagccgacaggagacgtggggagacggccacc

SEQ ID NO: 75 - RNA, 54 nt, synthetic construct
  source 1..54: mol_type = other RNA
aggtgcgacttaccgcgcaatctaacccagcagtctgcattgattaacgccacc

SEQ ID NO: 76 - RNA, 54 nt, synthetic construct
  source 1..54: mol_type = other RNA
aggcacttttattagtgttgtccagtcattacactcggataccttgttgccacc

SEQ ID NO: 77 - RNA, 54 nt, synthetic construct
  source 1..54: mol_type = other RNA
agaacggaacaacctgttgtcattggatcataccgtatcactgacctagccacc

SEQ ID NO: 78 - RNA, 53 nt, synthetic construct
  source 1..53: mol_type = other RNA
agaatatctcgctattctacacgtgatctcacgcagtgtatccctccgccacc

SEQ ID NO: 79 - RNA, 87 nt, synthetic construct
  source 1..87: mol_type = other RNA
agtgttccaagcttcgtctcaagttgagttgttttagattgctgagtttgatcacagaaaagggtaatttcggtccgcactgccacc

SEQ ID NO: 80 - RNA, 87 nt, synthetic construct
  source 1..87: mol_type = other RNA
aggggctatctaccttcaggccttgtcttgtagaacagttagtaaggacctactattcaagaactaactaaggcgcgtccagccacc

SEQ ID NO: 81 - RNA, 87 nt, synthetic construct
  source 1..87: mol_type = other RNA
agacgatcatagactaggacgtgtcgtcggtcattggaatataaaggaacttgccgtcatagtatctttaaggtcacgcgtgccacc

SEQ ID NO: 82 - RNA, 61 nt, synthetic construct
  source 1..61: mol_type = other RNA
agcgaaatttattaaaaaaacgttctgaatcggccaggagaggccgacaaaaaccgccacc

SEQ ID NO: 83 - RNA, 71 nt, synthetic construct
  source 1..71: mol_type = other RNA
aggcaaaatacatcatagtgcagcctatagataaatcggacaggatattatcggagacagaaaccgccacc

SEQ ID NO: 84 - RNA, 108 nt, synthetic construct
  source 1..108: mol_type = other RNA
agcaggcaagtacatccttcgtgtcctaggtgatagtaattggtatcgaggtatatcagaagacaggcaatttcatctggcggtgcacaccctaccagcaacgccacc

SEQ ID NO: 85 - RNA, 108 nt, synthetic construct
  source 1..108: mol_type = other RNA
agaatcaaatcatcttgacggttcagagtcgggcaacgagcagtttcccgcttgtgtcctatcacgttcgcagagggggcatcgaggatcttaactgcactcgccacc

SEQ ID NO: 86 - RNA, 39 nt, synthetic construct
  source 1..39: mol_type = other RNA
agactcttctggtccccacagactcagagagaacccacc

SEQ ID NO: 87 - RNA, 97 nt, synthetic construct
  source 1..97: mol_type = other RNA
agcgcccgctgcagccgccggctctgaatccttctggcttccgcctcagaggagttcttagcctgtcccgaaccgtaaccccggcgagcaggccacc

SEQ ID NO: 88 - RNA, 73 nt, synthetic construct
  source 1..73: mol_type = other RNA
agagagcgccggaccaccctctgtccgccccgaggagtttgccgcctgccggagcacctgcgcacagcccacc

SEQ ID NO: 89 - RNA, 101 nt, synthetic construct
  source 1..101: mol_type = other RNA
agcattccaaccttccagcctgcgacctgcggagaaaaaaaattacttattttcttgccccatacataccttgaggcgagcaaaaaaattaaattttaacc

SEQ ID NO: 90 - RNA, 80 nt, synthetic construct
  source 1..80: mol_type = other RNA
agcactctgcgcgcccgctcttctgctgctgtttgtctacttcctcctgcttccccgccgccgccgccgccatcgccacc

SEQ ID NO: 91 - RNA, 70 nt, synthetic construct
  source 1..70: mol_type = other RNA
aggggagatcttcgtcaccctttttcacttcctcagttttcgcggaccacttcaaggactaaaagccacc

SEQ ID NO: 92 - RNA, 58 nt, synthetic construct
  source 1..58: mol_type = other RNA
agctcctcccgaagtgctcctcttctacagctgttccgcagtcgccgccgccgccacc

SEQ ID NO: 93 - RNA, 56 nt, synthetic construct
  source 1..56: mol_type = other RNA
aggctcttccagactctgcgacctaactgactcttgggttcaaggctagggccacc

SEQ ID NO: 94 - RNA, 58 nt, synthetic construct
  source 1..58: mol_type = other RNA
agcgcagcctgctgtccgcctgccttttcgtctctagccgcgtgaagtcagcgccacc

SEQ ID NO: 95 - RNA, 52 nt, synthetic construct
  source 1..52: mol_type = other RNA
agctcaaagaaaaaccaaacgtaacaagggcgaattcgttggtaaagccacc

SEQ ID NO: 96 - RNA, 58 nt, synthetic construct
  source 1..58: mol_type = other RNA
agggtcccacagtctggtcctgcttcaccttcccctgacctgagtagtcgccgccacc

SEQ ID NO: 97 - RNA, 58 nt, synthetic construct
  source 1..58: mol_type = other RNA
agctcagtgagcaaccctaattaacctgattttttgctgataatcactctcagccacc

SEQ ID NO: 98 - RNA, 49 nt, synthetic construct
  source 1..49: mol_type = other RNA
agagtcccgagcctaaagcctcgcctgcctgcccagtgccagggccacc

SEQ ID NO: 99 - RNA, 59 nt, synthetic construct
  source 1..59: mol_type = other RNA
agctcagtcctggtcccccagtcccaagcctcacctgcctgcccagcgccagggccacc

SEQ ID NO: 100 - RNA, 110 nt, synthetic construct
  source 1..110: mol_type = other RNA
agaaaagaaagaagccagaacataccaccagccctttgagtatacaacataacaaacgtcattactttacaacttgattaggagacagtacagagatctgaagggccacc

SEQ ID NO: 101 - RNA, 83 nt, synthetic construct
  source 1..83: mol_type = other RNA
agagaacacgacacaacgaactttatattttaccacttccttgaatagttgcaggagaaataacaaggcattgaagagccacc

SEQ ID NO: 102 - RNA, 134 nt, synthetic construct
  source 1..134: mol_type = other RNA
aggagacgccatccacgctgttttgacctccatagaagacaccgggaccgatccagcctccgcggccgggaacggtgcattggaacgcggattccccgtgccaagagtgactcaccgtccttgacacggccacc

SEQ ID NO: 103 - RNA, 66 nt, synthetic construct
  source 1..66: mol_type = other RNA
agaacggtgcattggaacgcggattccccgtgccaagagtgactcaccgtccttgacacggccacc

SEQ ID NO: 104 - RNA, 51 nt, synthetic construct
  source 1..51: mol_type = other RNA
agggagacgccatccacgctgttttgacctccatagaagacaccgggaccg

SEQ ID NO: 105 - RNA, 22 nt, synthetic construct
  source 1..22: mol_type = other RNA
agataaacgctcaactttggcc

SEQ ID NO: 106 - RNA, 28 nt, synthetic construct
  source 1..28: mol_type = other RNA
agataaacgctcaactttggccgccacc

SEQ ID NO: 107 - RNA, 28 nt, synthetic construct
  source 1..28: mol_type = other RNA
agataaacgctcaactttggccgccaag

SEQ ID NO: 108 - RNA, 52 nt, synthetic construct
  source 1..52: mol_type = other RNA
agattccccgcctcctcctgtcccgcagtcggataaacgctcaactttggcc

SEQ ID NO: 109 - RNA, 45 nt, synthetic construct
  source 1..45: mol_type = other RNA
agggagaccatattgaagagacagagtgatatataaaactgctaa

SEQ ID NO: 110 - RNA, 51 nt, synthetic construct
  source 1..51: mol_type = other RNA
agggagaccatattgaagagacagagtgatatataaaactgctaagccacc

SEQ ID NO: 111 - RNA, 19 nt, synthetic construct
  source 1..19: mol_type = other RNA
agggagacaaaactgctaa

SEQ ID NO: 112 - RNA, 25 nt, synthetic construct
  source 1..25: mol_type = other RNA
agggagacaaaactgctaagccaag

SEQ ID NO: 113 - RNA, 15 nt, synthetic construct
  source 1..15: mol_type = other RNA
agggagactgccacc

SEQ ID NO: 114 - RNA, 15 nt, synthetic construct
  source 1..15: mol_type = other RNA
agggagactgccaag

SEQ ID NO: 115 - RNA, 47 nt, synthetic construct
  source 1..47: mol_type = other RNA
agggaagtgcagccccaggttcccgagcggcaggttgggtgcggacc

SEQ ID NO: 116 - RNA, 66 nt, synthetic construct
  source 1..66: mol_type = other RNA
agggaactcctccccatcctctccctctgtccctctgtccctctgaccctgcactgtcccagcacc

SEQ ID NO: 117 - RNA, 38 nt, synthetic construct
  source 1..38: mol_type = other RNA
agggactcccgggctggcagcagggccccagcggcacc

SEQ ID NO: 118 - RNA, 60 nt, synthetic construct
  source 1..60: mol_type = other RNA
agggaaatcctttctttcagctggagtgctcctcaggagccagccccacccttagaaaag

SEQ ID NO: 119 - RNA, 102 nt, synthetic construct
  source 1..102: mol_type = other RNA
agcactcgcgctgccatcactcttccgccgtcttcgccgccatcctcggcgcgactcgcttctttcggttctaccaggtagagtccgccgccatcctccacc

SEQ ID NO: 120 - RNA, 101 nt, synthetic construct
  source 1..101: mol_type = other RNA
agccggtggcggcaggatacagcggcttctgcgcgacttataagagctccttgtgcggcgccattttaagcctctcggtctgtggcagcagcgttggcccg

SEQ ID NO: 121 - RNA, 102 nt, synthetic construct
  source 1..102: mol_type = other RNA
agctacagaaacgaaagaaaaagtctgtataagccaaaggtgttcgggaagaaaataaccccattgccttgagtttgtaggtgccactactactctggaaaa

SEQ ID NO: 122 - RNA, 58 nt, synthetic construct
  source 1..58: mol_type = other RNA
agttgccgctgtcgtccgcagagccagttcctagcgcagagccgcgcccgccgccacc

SEQ ID NO: 123 - RNA, 133 nt, synthetic construct
  source 1..133: mol_type = other RNA
agcgcgttattgttctgccgggcggacacgtgacgcgaagcttaccgccgagaccgcgtccgccccgcgagcacagagcctcgcctttgccgatccgccgcccgtccacacccgccgccagctcaccgccacc

SEQ ID NO: 124 - RNA, 92 nt, synthetic construct
  source 1..92: mol_type = other RNA
agaccgccgagaccgcgtccgccccgcgagcacagagcctcgcctttgccgatccgccgcccgtccacacccgccgccagctcaccgccacc

SEQ ID NO: 125 - RNA, 119 nt, synthetic construct
  source 1..119: mol_type = other RNA
agtataaaacccggcggcgcaacgcgcagccactgtcgagtcgcgtccacccgcgagcacagcttctttgcagctccttcgttgccggtccacacccgccaccagttcgccccgccacc

SEQ ID NO: 126 - RNA, 160 nt, synthetic construct
  source 1..160: mol_type = other RNA
agcgcgttattgttctgccgggcggacacgtgacgcgaagctttataaaacccggcggcgcaacgcgcagccactgtcgagtcgcgtccacccgcgagcacagcttctttgcagctccttcgttgccggtccacacccgccaccagttcgccccgccacc

SEQ ID NO: 127 - RNA, 400 nt, synthetic construct
  source 1..400: mol_type = other RNA
agttgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggattaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaagggcgaattcgttggtaaagccacc

SEQ ID NO: 128 - RNA, 52 nt, synthetic construct
  source 1..52: mol_type = other RNA
agtcaacgtagttctaacagttttttaattagagagcagatctctggccacc

SEQ ID NO: 129 - RNA, 66 nt, synthetic construct
  source 1..66: mol_type = other RNA
agatctttgggtggtggagtgcaaaggaggcgacctgcaacagaggagtcccggtcaccagccacc

SEQ ID NO: 130 - RNA, 80 nt, synthetic construct
  source 1..80: mol_type = other RNA
agaaaccccagctgttggggccaggacacccagtgagcccatacttgctctttttgtcttcttcagactgcgccgccacc

SEQ ID NO: 131 - RNA, 52 nt, synthetic construct
  source 1..52: mol_type = other RNA
agcaaaaggaattcttctcgggaggcagtgctgggtcctttccaccgccacc

SEQ ID NO: 132 - RNA, 64 nt, synthetic construct
  source 1..64: mol_type = other RNA
aggaaattggatttggctgcagcttctggagggagccgacaggagacgtggggagacggccacc

SEQ ID NO: 133 - RNA, 64 nt, synthetic construct
  source 1..64: mol_type = other RNA
aggaaattccatttggctgcagcttctggagggagccgacaggagacgtggggagagtgccacc

SEQ ID NO: 134 - RNA, 64 nt, synthetic construct
  source 1..64: mol_type = other RNA
aggaaattccatttggctgcagcttaaagagggagccgacaggagacgtggggagacggccacc

SEQ ID NO: 135 - RNA, 64 nt, synthetic construct
  source 1..64: mol_type = other RNA
aggaaattccatttgaattcagcttctggagggagccgacaggagacgtggggagacggcatcc

SEQ ID NO: 136 - RNA, 54 nt, synthetic construct
  source 1..54: mol_type = other RNA
agggtctattcaaagactgcggacgcgatcaagtgctctgttccatctgccacc

SEQ ID NO: 137 - RNA, 87 nt, synthetic construct
  source 1..87: mol_type = other RNA
agtcggttgtctaatcaagatcatatcttgggaccgggctatacttcatcaaaaactgtccatatagtcgcggcggggaaagccacc

SEQ ID NO: 138 - RNA, 63 nt, synthetic construct
  source 1..63: mol_type = other RNA
agcgaacttacaaaaaaaagcatcttatcgaaaccgccgctcggagacgagcgcacagccacc

SEQ ID NO: 139 - RNA, 71 nt, synthetic construct
  source 1..71: mol_type = other RNA
agcgaaaaaaaattaaaatccagcttctgtaaagagccgacaggagaggtggcgagacaaaaaccgccacc

SEQ ID NO: 140 - RNA, 62 nt, synthetic construct
  source 1..62: mol_type = other RNA
aggtagtaaaaaagctgattgagttgttaggagaggtggcgagagtttaggggagggccacc

SEQ ID NO: 141 - RNA, 56 nt, synthetic construct
  source 1..56: mol_type = other RNA
aggcacggtaaccgaaagcgttcgaacaggggcgccaatacattaaaattgccacc

SEQ ID NO: 142 - RNA, 108 nt, synthetic construct
  source 1..108: mol_type = other RNA
agttagaaagagtactcataaacgataataaagtgaacctattaggcccgtcttgcggagttgaggagcttgcatctctgctgaggttaaaactaatatcttgccacc

SEQ ID NO: 143 - DNA, 1889 bp, synthetic construct
  source 1..1889: mol_type = other DNA
aggaaattccatttgaatacagcttctggagggagccgacaggagacgtggggagacggccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaaaagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataggctgccttctgcggggcttgccttctggccatgcccttcttctctcccttgcacctgtacctcttggtctttgaataaagcctgagtaggaagtaaaaaaaaaaaaaaaaaaaaaaaaaaaaaaaaaaaaaaaaaaaaaaaaaaaaaaaaaaaaaaaaaaaaaaaaaaa

SEQ ID NO: 144 - DNA, 1831 bp, synthetic construct
  source 1..1831: mol_type = other DNA
gccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaaaagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataggctgccttctgcggggcttgccttctggccatgcccttcttctctcccttgcacctgtacctcttggtctttgaataaagcctgagtaggaagtaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0366040 A1
(43) Pub. Date: Nov. 19, 2026

(54) ARTIFICIAL NUCLEIC ACID MOLECULE

(71) Applicant: STEMIRNA THERAPEUTICS CO., LTD., Pudong New Area, Shanghai (CN)

(72) Inventors: Jun ZHANG, Shanghai (CN); Taiyun WANG, Shanghai (CN); Hangwen LI, Shanghai (CN); Huifang DONG, Shanghai (CN)

(21) Appl. No.: 19/123,010

(22) PCT Filed: Nov. 11, 2022
(86) PCT No.: PCT/CN2022/131286

§ 371 Date: Apr. 21, 2025

(51) Int. Cl. C12N 15/86; C12N 15/67

(52) U.S. Cl. CPC C12N 15/86; C12N 15/67; C12N 2310/317; C12N 2310/531; C12N 2310/532; C12N 2800/10; C12N 2830/50; C12N 2840/105

(57) ABSTRACT

Provided is an artificial nucleic acid molecule, which comprises at least one open reading frame and at least one 5-untranslated region element, and has high translation efficiency.

### TECHNICAL FIELD

The invention belongs to the field of biomedicine. The present invention relates to artificial nucleic acid molecules comprising an open reading frame, a 5′-untranslated region element (5′-UTR element) and/or a 3′-untranslated region element (3′-UTR element). The invention also relates to a vector comprising a 5′-UTR element and/or a 3′-UTR element, a cell comprising the artificial nucleic acid molecule or the vector, and a lipid combination comprising the artificial nucleic acid molecule or the vector or pharmaceutical compositions, as well as kits comprising the artificial nucleic acid molecule, the vector, the lipid composition and/or the pharmaceutical composition, preferably in the fields of gene therapy and/or gene vaccination.

### BACKGROUND TECHNIQUE

For gene therapy and genetic vaccination, stable RNA is often required. Stable RNA enables the product encoded by the RNA sequence to accumulate in the body and maintains its structural and functional integrity during its storage, preparation and administration. Therefore, there is a need to provide stable RNA molecules for gene therapy or genetic vaccination that prevent them from undergoing early degradation or decay. As a solution for mRNA stabilization, naturally occurring eukaryotic mRNA molecules have been found to contain specific stabilizing elements. For example, its 5′-untranslated region (5′-UTR) and/or 3′-untranslated region (3′-UTR). Both 5′-UTR and 3′-UTR are typical premature mRNA elements.

An mRNA molecule carries a gene that codes for the corresponding protein. This gene is flanked by specific untranslated regions 5′ to the AUG start codon and 3′ to the stop codon. These regions are called 5′-untranslated regions and 3′-untranslated regions. The 5′-untranslated region, also known as the leader sequence, the 5′-UTR begins at position +1 (where transcription begins) and ends one nucleotide before the start codon. In prokaryotic mRNA, the 5′-UTR is usually short and contains a specific ribosome-binding site, called the Shine Dalgarno (SD) sequence. In eukaryotes, it usually contains elements that control gene expression, such as short uORFs, IRESs, etc. Similarly, 3′-UTR usually carries regulatory functions, such as polyadenylation signals (AAUAAA), AU-rich elements (AREs), seed regions of miRNAs, etc., of which part of the 3′-untranslated region is also polyadenine Sour tail.

In the prior art, many untranslated region sequences that are beneficial to improving the stability and expression level of mRNA have been designed and experimentally studied based on the untranslated regions of some naturally occurring proteins. However, the field still needs more untranslated region sequences that are more conducive to improving the stability and safety of gene therapy and gene vaccination.

### SUMMARY OF THIS INVENTION

In one aspect, the invention provides an artificial nucleic acid molecule comprising:
a. at least one open reading frame (ORF); and
b. at least one 5′-untranslated region element (5′-UTR element), the 5′-UTR element comprising a nucleic acid sequence selected from the following:
   (i) wherein the 5′-UTR element is identical to SEQ ID NO:71, comprising 1, 2, 3 or more mutations; or
   (ii) wherein the 5′-UTR element comprises SEQ ID NO: 2, 3, 4, 5, 6, 7, 8, 9, 10, 11 or 12; or
   (iii) a nucleic acid sequence wherein the 5′-UTR element comprises a 5′-UTR derived from a transcript of the following gene: alpha-1-globin, ARC, TUBB1, TUBB4B, ANXA5, A1CF, TUBA1C, TUBB3 and HCV, and are truncated; or the corresponding RNA sequence of the above nucleic acid sequence.

In one embodiment, the 5′-UTR element is compared to SEQ ID NO:71, which includes G16A, C17A and G19A.

In one embodiment, the 5′-UTR element comprises the nucleic acid sequence of SEQ ID NO:1.

In one embodiment, the nucleic acid sequence is derived from the 5′-UTR of the transcript of the following human genes: alpha-1-globin, ARC, TUBB1, TUBB4B and ANXA5; or derived from the transcript of the following mouse gene The nucleic acid sequence of the 5′-UTR: A1CF, TUBB1, TUBA1C, TUBB4B and TUBB3; or the nucleic acid sequence of the 5′-UTR derived from the transcript of the rat gene ARC; or the 5′-UTR derived from the transcript of the viral gene HCV′-UTR nucleic acid sequence.

In one embodiment, the 5′-UTR element exhibits a length of 3-500 nucleotides, preferably 5-150 nucleotides, more preferably 50-110 nucleotides.

In one embodiment, the 5′-UTR element comprises the nucleic acid sequence of SEQ ID NO: 13, 14, 15, 16, 17, 18, 19, 20, 21, 22, 23 or 24.

In one embodiment, the 5′-UTR element comprises a Kozak sequence.

In one embodiment, the Kozak sequence comprises the nucleic acid sequence of SEQ ID NO:70.

In one embodiment, the artificial nucleic acid molecule further comprises at least one 3′-untranslated region element (3′-UTR element).

In one embodiment, the 3′-UTR element comprises the nucleic acid sequence of SEQ ID NO:72.

In one embodiment, the artificial nucleic acid molecule further comprises a 5′ cap structure, a polycytidylic acid sequence, a polyadenylic acid sequence or a histone stem-loop.

In one embodiment, the artificial nucleic acid molecule further comprises a 5′ cap structure, a polycytidylic acid sequence, a polyadenylic acid sequence, and a histone stem-loop.

In one embodiment, the ORF is codon optimized.

In one embodiment, the artificial nucleic acid molecule is RNA, preferably mRNA.

In one embodiment, the 5′-UTR element comprises SEQ ID NO: 74, 75, 76, 77, 78, 79, 80, 81, 82, 83, 84, 85, 86, 87, 88, 89, nucleic acid sequence 90, 91, 92, 93, 94, 95, 96 or 97.

In yet another aspect, the present invention also provides a vector comprising the artificial nucleic acid molecule of the present invention.

In on the other hand. The present invention also provides a cell comprising the artificial nucleic acid molecule of the present invention or the vector of the present invention.

In another aspect, the present invention also provides a lipid composition of the present invention, which includes an artificial nucleic acid molecule of the present invention and a lipid encapsulating the artificial nucleic acid molecule, wherein the lipid encapsulating the artificial nucleic acid molecule contains cations. Lipids, phospholipids, steroids and polyethylene glycol modified lipids; the lipid composition also includes a cationic polymer, wherein the cationic polymer is associated with the artificial nucleic acid molecule into a complex, and is co-encapsulated in Lipid multimeric complexes are formed in lipids.

In one embodiment, the cationic lipid comprises a lipid compound of formula (I), (II), (III), (IV), or a pharmaceutically acceptable salt thereof, as defined herein. In a preferred embodiment, the cationic lipid is M5, MC3, ALC-0315, SM-102, SW-II-115, SW-II-121, SW-II-122, SW-II-134-3. SW-II-138-2, SW-II-139-2 or SW-II-140-2.

In one embodiment, the lipid composition contains 10-70 mol % cationic lipids, 10-70 mol % phospholipids, 10-70 mol % steroids, and 0.05-20 mol % polyethylene glycol modification of lipids.

In one embodiment, the lipid composition contains 35-50 mol % cationic lipids, 10-30 mol % phospholipids, 24-44 mol % steroids, and 1-1.5 mol % polyethylene glycol modification of lipids.

In a preferred embodiment, the lipid composition contains 35-50 mol % cationic lipids, 10-30 mol % DOPE, 24-44 mol % cholesterol and 1-1.5 mol % DMG-PEG.

In a preferred embodiment, the lipid composition contains 50 mol % cationic lipids, 10 mol % DOPE, 38.5 mol % cholesterol, and 1.5 mol % DMG-PEG.

In a preferred embodiment, the lipid composition contains 40 mol % cationic lipids, 15 mol % DOPE, 43.5 mol % cholesterol, and 1.5 mol % DMG-PEG.

In yet another aspect, the present invention also provides a pharmaceutical composition, which includes the artificial nucleic acid molecule of the present invention, the carrier of the present invention, the cell of the present invention or the lipid composition of the present invention, and a pharmaceutically acceptable carrier, excipients or diluents.

In another aspect, the present invention also provides the use of the artificial nucleic acid molecule of the present invention, the vector of the present invention, the cell of the present invention, the lipid composition of the present invention or the pharmaceutical composition of the present invention in the preparation of vaccines or drugs for gene therap.

In another aspect, the present invention also provides the use of the artificial nucleic acid molecule of the present invention, the vector of the present invention, the cell of the present invention, the lipid composition of the present invention or the pharmaceutical composition of the present invention in the preparation of drugs for treating or preventing diseases.

In yet another aspect, the present invention also provides a method for increasing the translation efficiency of an artificial nucleic acid molecule, preferably an mRNA molecule or a vector, the method comprising connecting an open reading frame and a 5′-UTR element as defined in the present invention.

In yet another aspect, the present invention also provides a kit, which contains the artificial nucleic acid molecule of the present invention, the vector of the present invention, the cell of the present invention, the lipid composition of the present invention or the pharmaceutical composition of the present invention.

In yet another aspect, the present invention also provides a method for producing artificial nucleic acid molecules, the method comprising:
a) Synthesize the artificial nucleic acid molecule of the present invention; or
b) Synthesize artificial nucleic acid molecules through the vector of the present invention.

### DESCRIPTION OF THE DRAWINGS

FIG. 1 shows the pUC57-Luc plasmid map.

FIG. 2 shows exemplary sequences of subject artificial nucleic acid molecules.

FIG. 3 shows the effects of different 5′-UTR elements on the expression of reporter gene luciferase.

The ordinate is the number of the corresponding 5′-UTR element contained in different artificial nucleic acid molecules. Compared with the tested artificial nucleic acid molecule, the artificial nucleic acid molecule that only contains the Kozak sequence before the start codon of the luciferase gene is the reference artificial nucleic acid molecule. Nucleic acid molecules (ctrl).

### DETAILED DESCRIPTION

### Definition

All patents, patent applications, scientific publications, manufacturer's instructions and instructions, etc. cited herein, whether supra or infra, are hereby incorporated by reference in their entirety. Nothing contained herein should be construed as an admission that this disclosure is not entitled to antedate such disclosure.

In the present invention, unless otherwise stated, scientific and technical terms used herein have the meanings commonly understood by those skilled in the art. Furthermore, the terms and laboratory procedures related to protein and nucleic acid chemistry, molecular biology, cell and tissue culture, microbiology, and immunology used in this article are terms and routine procedures widely used in the corresponding fields. Meanwhile, in order to better understand the present invention, definitions and explanations of relevant terms are provided below.

As used herein, “at least one” or “one or more” may mean 1, 2, 3, 4, 5, 6, 7, 8 or more.

As used herein, the expressions “comprises,” “comprising,” “containing,” and “having” are open-ended and mean the inclusion of recited elements, steps or components but not the exclusion of other unrecited elements, steps or components. The expression “consisting of” does not include any element, step or component not specified. The expression “consisting essentially of” means that the scope is limited to the specified elements, steps or components plus the optional presence of elements, steps or components that do not materially affect the basic and novel properties of the claimed subject matter. It will be understood that the expressions “consisting essentially of” and “consisting of” are encompassed within the meaning of the expression “including”.

As used herein, the conjunctive term “and/or” between multiple recited elements is to be understood to include both individual and combined options. In other words, “and/or” includes “and” and “or.” For example, A and/or B includes A, B, and A+B. A, B and/or C include A, B, C and any combination thereof, such as A+B, A+C, B+C and A+B+C. Further elements qualified by “and/or” are to be understood in a similar manner and include any one and any combination thereof.

Unless otherwise stated, any numerical value or range of values, such as a concentration or range of concentrations, is to be understood in all cases as modified by the term “about.” Therefore, numerical values generally include ±10% of the stated value. For example, a concentration of 1 mg/mL includes 0.9 mg/mL to 1.1 mg/mL. Likewise, the concentration range of 1% to 10% (w/v) includes 0.9% (w/v) to 11% (w/v). As used herein, the use of a numerical range expressly includes all possible subranges and all individual values within the range, including integers and fractions within the range, unless the context clearly indicates otherwise.

As used herein, “nucleotide” includes deoxyribonucleotides and ribonucleotides and their derivatives. As used herein, “ribonucleotide” is the constituent material of ribonucleic acid (RNA), which is composed of one molecule of base, one molecule of five-carbon sugar, and one molecule of phosphate, which refers to the sugar in β-D-ribofuranose (β-D-ribofuranosyl) A nucleotide with a hydroxyl group at the 2′ position. “Deoxyribonucleotide” is the constituent material of deoxyribonucleic acid (DNA). It is also composed of one base molecule, one five-carbon sugar molecule, and one phosphate molecule. It refers to the sugar in β-D-ribofuranose (β-D-ribofuranosyl) is a nucleotide in which the hydroxyl group at the 2′ position is replaced by hydrogen. It is the main chemical component of chromosomes. “Nucleotide” is usually referred to by a single letter that represents the base within it: “A(a)” refers to deoxyadenosine or adenylate containing adenine, and “C(c)” refers to deoxyadenosine containing cytosine. Cytidylic acid or cytidylic acid, “G(g)” refers to deoxyguanylic acid or guanylic acid containing guanine, “U(u)” refers to uridylic acid containing uracil, “T(t)” refers to Deoxythymidylate containing thymine.

As used herein, the terms “polynucleotide” and “nucleic acid” are used interchangeably to refer to a polymer of deoxyribonucleotides (DNA) or a polymer of ribonucleotides (ribonucleic acid, RNA). “Polynucleotide sequence,” “nucleic acid sequence,” and “nucleotide sequence” are used interchangeably to refer to the ordering of nucleotides in a polynucleotide. Those in the art should understand that the DNA coding strand (sense strand) and the RNA it codes for can be regarded as having the same nucleotide sequence, and the deoxythymidylate in the DNA coding strand sequence corresponds to the uridylic acid in the RNA sequence.

As used herein, “artificial nucleic acid molecules” may be understood to mean non-natural nucleic acid molecules. Such nucleic acid molecules may be non-natural due to their individual sequences (which do not occur in nature) and/or due to other modifications (e.g. structural modifications of nucleotides that do not occur in nature). Artificial nucleic acid molecules can be DNA molecules, RNA molecules, or hybrid molecules containing DNA and RNA parts. Typically, artificial nucleic acid molecules can be designed and/or produced by genetic engineering methods. In this case, the artificial nucleic acid molecule contains an artificial sequence that does not normally occur in nature and that differs from the wild-type sequence by at least one nucleotide. The term “wild-type sequence” is understood to mean a naturally occurring sequence.

As used herein, “modified” refers to non-natural. For example, the RNA can be modified RNA. That is, RNA may include one or more non-naturally occurring nucleobases, nucleosides, nucleotides, or linking groups. “Modified” groups may also be referred to herein as “altered” groups. Groups may be modified or altered chemically, structurally, or functionally. For example, a modified nucleobase may include one or more non-naturally occurring substitutions.

As used herein, the term “transfection” refers to the introduction of nucleic acid molecules, such as DNA or RNA (e.g. mRNA) molecules into a cell, preferably into a eukaryotic cell. Within the scope of the present invention, the term “transfection” includes any method known to the person skilled in the art for introducing nucleic acid molecules into cells, preferably into eukaryotic cells, such as into mammalian cells. Such methods include, for example, electroporation, e.g. cationic lipid and/or liposome based lipofection, calcium phosphate precipitation, nanoparticle-based transfection, virus-based transfection, or based on cationic polymers such as DEAE-Transfection of dextran or polyethylenimine), etc. Preferably, the method is lipofection.

As used herein, the term “expression” includes the transcription and/or translation of a nucleotide sequence. Thus, expression may involve the production of transcripts and/or polypeptides.

As used herein, the term “translation efficiency” relates to nucleic acid molecules (e.g. mRNA) containing an open reading frame (ORF). Translation efficiency is experimentally measurable. Translation efficiency is typically measured by determining the amount of protein translated by the ORE. For experimental measurement of translation efficiency, the ORF preferably encodes a reporter protein or any other protein that can be quantified. In the context of the present invention, translation efficiency applies in particular to nucleic acid molecules which, in addition to the ORF, comprise at least one 5′-UTR element, preferably as defined herein. It should be understood that in the present invention, high translation efficiency is generally provided by specific UTR elements (specific 5′-UTR elements). Although for the purpose of experimentally quantifying translation efficiency, the ORF suitably encodes a reporter protein or any other protein that can be quantified, the invention is not limited to such purposes; therefore, at least one 5′-UTR element of the invention (which provides a high Translation efficiency) can be included in nucleic acid molecules containing an ORF that does not encode a reporter protein.

Translation efficiency is a relative term derived by determining and comparing the translation efficiencies of multiple (eg, two or more) nucleic acid molecules, eg, experimentally to quantify the protein encoded by an ORE. One of the nucleic acid molecules may be referred to as a “reference nucleic acid molecule” or “reference construct” and the other as a “test nucleic acid molecule” or “test construct”, and the test nucleic acid molecule. It is an artificial nucleic acid molecule according to the present invention. For this purpose, the reference nucleic acid molecule and the test nucleic acid molecule share the same ORF (same nucleic acid sequence); and preferably, the nucleic acid sequence of the test nucleic acid molecule is the same as the nucleic acid sequence of the reference nucleic acid molecule, except that The UTR element being tested, that is, the 5′-UTR element; in other words, preferably, the test nucleic acid molecule and the reference nucleic acid molecule differ from each other only in that the 5′-UTR element has a different nucleic acid sequence; such that the 5′-UTR element becomes A unique structural feature that distinguishes the test nucleic acid molecule from the reference nucleic acid molecule.

As used herein, a “vector” is a vehicle used to introduce an exogenous polynucleotide into a host cell so that when the vector is transformed into an appropriate host cell, the exogenous polynucleotide is amplified or expressed. Vectors usually remain episomal, but can be designed to integrate the gene or part thereof into the chromosome of the genome. As used herein, the definition of vector encompasses plasmids, linearized plasmids, viral vectors, cosmids, phage vectors, phagemids, artificial chromosomes (e.g. yeast artificial chromosomes and mammalian artificial chromosomes), and the like. Viral vectors include, but are not limited to, retroviral vectors (including lentiviral vectors), adenoviral vectors, adeno-associated virus vectors, herpes virus vectors, poxvirus vectors, and baculovirus vectors.

As used herein, a “cell” is a cell used to receive, maintain, replicate and amplify a vector. Cells can also be used to express polypeptides encoded by vectors. When cells divide, the nucleic acid contained in the vector replicates, thereby amplifying the nucleic acid. Cells can be eukaryotic or prokaryotic. Suitable cells include, but are not limited to, CHO cells, various COS cells, HeLa cells, HEK cells such as HEK 293 cells.

As used herein, an “aliphatic” group is a non-aromatic group in which carbon atoms are linked into a chain, and may be saturated or unsaturated.

As used herein, the term “alkyl” refers to an optionally substituted straight or branched chain saturated hydrocarbon including one or more carbon atoms. The term “C₁-C₁₂ Alkyl” or “C₁₋₁₂ “Alkyl” refers to an optionally substituted straight or branched chain saturated hydrocarbon containing 1 to 12 carbon atoms.

As used herein, the term “alkenyl” refers to an optionally substituted straight or branched hydrocarbon chain including two or more carbon atoms and at least one double bond. The term “C₂-C₁₂ Alkenyl” or “C₂₋₁₂ “Alkenyl” refers to an optionally substituted straight or branched hydrocarbon containing 2 to 12 carbon atoms and at least one carbon-carbon double bond. Alkenyl may include one, two, three, four or more carbon-carbon double bond.

As used herein, the term “halogen” refers to fluorine, chlorine, bromine and iodine.

As used herein, the term “carbocycle” refers to a monocyclic or polycyclic non-aromatic system that includes one or more rings composed of carbon atoms. The term “C3-8 “Carbocycle” means a carbocycle including 3 to 8 carbon atoms. A carbocycle may include one or more carbon-carbon double or triple bonds. Examples of carbocycles include, but are not limited to, cyclopropyl, cyclopentyl, cyclopentyl, Hexyl, etc. As used herein, when a carbocycle is saturated (i.e., contains no unsaturated bonds), the corresponding cycloalkyl group is also referred to. Unless otherwise specified, the carbocycle described herein refers to unsubstituted. and substituted, i.e., optionally substituted carbocyclic rings.

As used herein, the term “heterocycle” refers to a monocyclic or polycyclic ring system that includes one or more rings and includes at least one heteroatom. Heteroatoms may be, for example, nitrogen, oxygen, phosphorus or sulfur atoms. Heterocycles may include one or more double or triple bonds and may be nonaromatic. Examples of heterocycles include, but are not limited to, imidazolidinyl, oxazolidinyl, thiazolidinyl, pyrazolidinyl, isoxazolidinyl, isothiazolidinyl, morpholinyl, pyrrolidinyl, tetrahydrofuranyl, and piperidine base. Heterocycles may contain, for example, 3-10 atoms (non-hydrogen), i.e., 3-10 membered heterocycles (e.g., 3, 4, 5, 6, 7, 8, 9, or 10 members), one or more of which are heterocycles. Atom (such as N, O, S or P). When the heterocycle is saturated (ie, contains no unsaturated bonds), the corresponding heterocycloalkyl group may also be referred to. Unless otherwise specifically stated, heterocycle as used herein refers to both unsubstituted and substituted heterocyclic groups, ie, optionally substituted heterocycles.

As used herein, the term “aryl” refers to an all-carbon monocyclic or fused polycyclic aromatic ring group having a conjugated pi electron system. For example, C6-C10 Alkylaryl groups may have 6 to 10 carbon atoms, such as 6, 7, 8, 9, 10 carbon atoms. Examples of aryl groups include, but are not limited to, phenyl, naphthyl, and the like.

As used herein, the term “heteroaryl” refers to a monocyclic or fused polycyclic ring system containing at least one ring atom selected from N, O, S, the remaining ring atoms being C, and having at least one aromatic ring. Heteroaryl groups may have 5 to 10 ring atoms (5-10 membered heteroaryl groups), including 5, 6, 7, 8, 9 or 10 membered heteroaryl groups, especially 5 or 6 membered heteroaryl groups. Examples of heteroaryl groups include, but are not limited to, pyrrolyl, furyl, thienyl, imidazolyl, oxazolyl, pyrazolyl, pyridyl, pyrimidinyl, pyrazinyl, quinolyl, isoquinolyl, tetrazole base, triazolyl, triazinyl, benzofuranyl, benzothienyl, indolyl, isoindolyl, etc.

As used herein, the term “interrupted by one or more groups” means that the one or more groups are present on the carbon chain and the remainder of the carbon chain is connected to both ends of the one or more groups.

Unless otherwise specified, the groups described herein (e.g., R1-R7 ay of them, such as alkyl, alkylene, alkenyl, aryl, amino, etc.) may be optionally substituted. Optional substituents may be selected from the following, but are not limited to: halogen atoms (such as chlorine, bromo, fluoro or iodine), carboxylic acids (such as —C(O)OH), alcohols (such as hydroxyl, —OH), ester (such as —C(O)OR or —OC(O)R), aldehyde (such as —C(O)H), carbonyl group (such as —C(O)R, or represented by C═O), acid halide (e.g. —C(O)X, where X is fluorine, chlorine, bromine or iodine). (e.g. —C(OR)₂R″″, where each OR is the same or different alkoxy and R″″ is alkyl or alkenyl), phosphate (e.g. P(O)₄³⁻), thiols (e.g. —SH), sulfoxides (e.g. —S(O)R), sulfinic acids (e.g. —S(O)OH), sulfonic acids (e.g. —S(O) 2OH), sulfide (e.g. —C(S)H), sulfate (e.g. S(O)₄²⁻), sulfonyl group (e.g. —S(O)₂—), amides (e.g. —C(O)NR₂ or —N(R)C(O)R), azido (such as —N₃), nitro (for example —NO₂), cyano group (such as —CN), isocyanate group (such as —NC), acyloxy group (such as —OC(O)R), amino group (such as —NR₂, NRH or —NH₂), carbamoyl (e.g. —OC(O)NR₂, —OC(O)NRH or —OC(O)NH₂), sulfonamide (e.g. —S(O)₂NR₂, —S(O)₂NRH, —S(O)₂NH₂, —N(R)S(O)₂R, —N(H)S(O)₂R, —N(R)S(O)₂H, —N(H)S(O)₂H), C1-C12 Alkyl, C2-C12 Alkenyl, C6-C10 Aryl, 5-10 membered heteroaryl or 3-10 membered heterocycle. In any of the foregoing, R can each independently be a substituent as defined herein, such as alkyl, alkoxy, alkylene, halogen, carbocycle, heterocycle, aryl, heteroaryl, alkenyl. In some embodiments, the substituents themselves may be further substituted by, for example, one, two, three, four, five or six substituents as defined herein. For example, an alkyl group may be further substituted with one, two, three, four, five or six substituents as described herein.

As used herein, the term “compound” is intended to include isotopic compounds of the depicted structures. “Isotopes” are atoms that have the same atomic number but different mass numbers due to the number of neutrons in the nucleus, such as deuterium isotopes. For example, isotopes of hydrogen include tritium and deuterium. Additionally, the compounds, salts or complexes of the present invention can be prepared in combination with solvents or water molecules to form solvates and hydrates by conventional methods.

The term “optionally” or “optionally” (e.g., substituted optionally) means that the subsequently described event may or may not occur, and that the description includes instances where the event or circumstance occurs as well as where the event or circumstance occurs. Instances where the situation does not occur. For example, “optionally substituted alkyl” means that the alkyl group may or may not be substituted, and this description includes both substituted and unsubstituted alkyl radicals.

It will be understood that when chemical groups are written in a specific order, the reverse order is also contemplated unless otherwise stated. For example in M₁ defined as the general formula —C(O)NH—, —(R)i-(M1)ₖ—(R)ₘ— (i.e. —(R)ᵢ—C(O)—NH—(R)m-), unless otherwise stated, also covers M1 Compounds that are —NHC(O)— (i.e., —(R)ᵢ—NHC(O)—(R)m-).

As used herein, the term “contacting” refers to establishing a physical connection between two or more entities. For example, contacting a mammalian cell with a lipid composition means causing the mammalian cell and the lipid nanoparticle to share a physical connection. Methods of bringing cells into contact with external entities in vivo and ex vivo are well known in the biological field. For example, contacting the lipid composition with mammalian cells within the body of a mammal can be performed by different routes of administration (eg, intravenous, intramuscular, intradermal, and subcutaneous) and can involve different amounts of the lipid composition. Additionally, the lipid composition can contact more than one mammalian cell.

As used herein, the term “delivery” refers to providing an entity to a target. For example, delivering an artificial nucleic acid molecule to a subject may involve administering a lipid composition including the artificial nucleic acid molecule to the subject.

As used herein, a “lipid component” is a component of a composition that includes one or more lipids. For example, the lipid component may include one or more cationic lipids, pegylated lipids, structural lipids, or helper lipids.

The phrase “pharmaceutically acceptable” is used herein to mean, within the scope of reasonable medical judgment, suitable for use in contact with human and animal tissue without undue toxicity, irritation, allergic reaction, or other problems or complications, and with reasonable compounds, salts, materials, compositions and/or dosage forms that have a benefit/risk ratio.

As used herein, “pharmaceutically acceptable salts” refer to derivatives of the disclosed compounds in which the parent compound is converted into its salt form by converting an existing acid or base moiety (e.g., by combining the free basic group with a suitable organic acid reaction).

Examples of pharmaceutically acceptable salts include, but are not limited to, inorganic or organic acid salts of basic residues such as amines; alkali metal or organic salts of acidic residues such as carboxylic acids, and the like. Representative acid addition salts include, but are not limited to, acetate, adipate, alginate, ascorbate, aspartate, benzenesulfonate, benzoate, bisulfate, borate, Butyrate, camphorate, camphorsulfonate, citrate, cyclopentane propionate, digluconate, lauryl sulfate, ethane sulfonate, fumarate, Glucoheptonate, glycerophosphate, hemisulfate, enanthate, caproate, hydrobromide, hydrochloride, hydroiodide, 2-hydroxy-ethanesulfonate, lactocuronate lactate, laurate, lauryl sulfate, malate, maleate, malonate, methane sulfonate, 2-naphthalene sulfonate, nicotinate, nitrate, Oleate, oxalate, palmitate, pamoate, pectate, persulfate, 3-phenylpropionate, phosphate, picrate, pivalate, propionate, stearate, succinate, sulfate, tartrate, thiocyanate, tosylate, undecanoate, valerate, etc. Representative alkali metal or alkaline earth metal salts include, but are not limited to, sodium, lithium, potassium, calcium, magnesium salts, etc.; and non-toxic ammonium, quaternary ammonium, and amine cations, including, but are not limited to, ammonium, tetramethylammonium, tetraethylammonium, methylamine, dimethylamine, trimethylamine, triethylamine, ethylamine, etc. Pharmaceutically acceptable salts of the present invention include, for example, conventional nontoxic salts of the parent compounds formed from nontoxic inorganic or organic acids. Pharmaceutically acceptable salts of the present invention can be synthesized by conventional chemical methods from parent compounds containing basic or acidic moieties. In general, these salts can be prepared by reacting the free acid or base form of these compounds with a stoichiometric amount of the appropriate base or acid in water or in an organic solvent, or in a mixture of the two; generally preferred Non-aqueous media such as diethyl ether, ethyl acetate, ethanol, isopropyl alcohol or acetonitrile.

As used herein, “gene therapy” is understood to mean the treatment of a patient's body or an isolated component of a patient's body, such as isolated tissue/cells, by nucleic acids encoding peptides or proteins. It may typically comprise at least one of the following steps: a) administering a nucleic acid (preferably an artificial nucleic acid molecule as defined herein) directly to the patient by any route of administration or in vitro to the patient's isolated cells/tissues, which results in in vivo/ex vivo transfection of the patient's cells in vivo or in vitro; b) transcription and/or translation of the introduced nucleic acid molecules; and optionally c) if the nucleic acid is not administered directly to the patient, then the isolated, transfected cells are re-administered to the patient.

As used herein, “genetic vaccination” may typically be understood as vaccination by administration of nucleic acid molecules encoding antigens or immunogens or fragments thereof. The nucleic acid molecules can be administered to the subject's body or to isolated cells of the subject. When certain cells of the body are transfected or when isolated cells are transfected, the antigen or immunogen can be expressed by those cells and subsequently presented to the immune system, eliciting an adaptive (i.e., antigen-specific) immune response. Thus, genetic vaccination typically involves at least one of the following steps: a) administering a nucleic acid (preferably an artificial nucleic acid molecule as defined herein) to a subject (preferably a patient), or administering to an isolated form of a subject (preferably a patient) cells, which typically result in in vivo or in vitro transfection of the subject's cells; b) transcription and/or translation of the introduced nucleic acid molecule; and optionally c) if the nucleic acid is not administered directly to the patient, the isolated, transfected cells and then administered to a subject (preferably a patient).

As used herein, “vaccine” refers to a composition containing an active ingredient (eg, an artificial nucleic acid molecule of the invention) that is capable of eliciting an immune response in a subject upon vaccination. In specific embodiments, it induces an immune response that provides immune protection and is sufficient to prevent and/or alleviate at least one symptom associated with infection by a pathogen or disease.

As used herein, the term “treatment” means to partially or completely alleviate, ameliorate, ameliorate, alleviate, delay the onset of, inhibit the progression of, or reduce the severity of one or more symptoms or characteristics of a particular infection, disease, disorder or condition. degree or reduce its occurrence. “Prevention” means guarding against an underlying disease or preventing the worsening of symptoms or progression of a disease.

The term “prophylactically or therapeutically effective amount” refers to an agent (e.g., nucleic acid, drug, composition, therapeutic agent, diagnostic agent, preventive agent, etc.). The prophylactically or therapeutically effective amount is affected by factors including but not limited to the following factors: the speed and severity of the development of the disease or symptoms, the age, gender, weight and physiological condition of the subject, the duration of treatment and the specific route of administration. A prophylactically or therapeutically effective amount may be administered in one or more doses. Prophylactically or therapeutically effective amounts can be achieved by continuous or intermittent administration.

### Artificial Nucleic Acid Molecules

This article provides an artificial nucleic acid molecule, which contains:
a. At least one open reading frame (ORF); and
b. At least one 5′-untranslated region element (5′-UTR element).

As used herein, an “open reading frame (ORF)” is a sequence of triplets of nucleotides that can be translated into a peptide or protein. The open reading frame preferably contains an initiation codon at the end of its 5′-untranslated region element, a combination of three consecutive nucleotides that usually encodes the amino acid methionine (ATG), and usually presents multiple 3 nucleotides the immediate region of acid length. The open reading frame of the present invention is preferably a nucleotide sequence consisting of a number of nucleotides divisible by three, which starts with a start codon (e.g. ATG) and which preferably ends with a stop codon (eg TAA, TGA, or TAG) terminated. The open reading frame may be isolated or it may be integrated into a longer nucleic acid sequence, such as a vector or mRNA. The open reading frame can also be called the “protein coding region”.

Untranslated regions (UTRs) can have characteristics that provide regulatory effects, such as increases or decreases in stability, localization, and/or translation efficiency. A polynucleotide comprising a UTR can be administered to a cell, tissue, or organism, and one or more regulatory characteristics can be measured using conventional methods.

As used herein, the term “5′-untranslated region element (5′-UTR element)” refers to that portion of an artificial nucleic acid molecule that is located at the 5′ end (i.e., “upstream”) of the open reading frame and that is not translated as protein. Typically, the 5′-UTR initiates at the transcription start site and terminates one nucleotide before the start codon of the open reading frame. 5′-UTR plays a key role in regulating gene expression. The DNA sequence of the 5′-UTR can contain many cis-regulatory elements that can interact with the transcription machinery to regulate the abundance of messenger RNA (mRNA). In addition, the transcribed 5′-UTR is composed of a variety of RNA-based regulatory elements, including 5′ cap structure, secondary structure, RNA-binding protein motifs, upstream open reading frame (uORF), internal ribosome entry site, terminus Oligopyrimidine (TOP) tracts and G-quadruplexes. These elements can alter the efficiency of mRNA translation; some can also affect mRNA transcript levels through changes in stability or degradation (see, e.g., Lim, Y., et al. Multiplexed functional genomic analysis of 5′untranslated region mutations across the spectrum of prostate cancer. Nat Commun 12, 4217(2021)).

In one embodiment, the artificial nucleic acid molecule comprises:
a. At least one open reading frame (ORF); and
b. At least one 5′-untranslated region element (5′-UTR element) comprising 1, 2, 3 or more mutations compared to SEQ ID NO:71.

In one embodiment, the 5′-UTR element compared to SEQ ID NO: 71 includes one or more selected from the group consisting of mutations: C9G, C10G, G16A, C17A, G19A, G19T, C26G, T27A, G28C, C57G, G58T, C61A and A62C.

In one embodiment, the 5′-UTR element is compared to SEQ ID NO:71, which includes C9G and C10G.

In one embodiment, the 5′-UTR element comprises the nucleic acid sequence of SEQ ID NO:59.

In one embodiment, the 5′-UTR element is compared to SEQ ID NO:71, which includes C57G and G58T.

In one embodiment, the 5′-UTR element comprises the nucleic acid sequence of SEQ ID NO:60.

In one embodiment, the 5′-UTR element is compared to SEQ ID NO:71, which includes C26G, T27A, and G28C.

In one embodiment, the 5′-UTR element comprises the nucleic acid sequence of SEQ ID NO:61.

In one embodiment, the 5′-UTR element is compared to SEQ ID NO:71, which includes G16A, C17A, G19T, C61A, and A62C.

In one embodiment, the 5′-UTR element comprises the nucleic acid sequence of SEQ ID NO:62.

In a preferred embodiment, the 5′-UTR element is compared to SEQ ID NO:71, which includes G16A, C17A and G19A.

In a preferred embodiment, the 5′-UTR element comprises the nucleic acid sequence of SEQ ID NO:1.

In one embodiment, the 5′-UTR element comprises the nucleic acid sequence of SEQ ID NO: 1, 59, 60, 61 or 62.

In one embodiment, the artificial nucleic acid molecule comprises
a. At least one open reading frame (ORF); and
b. At least one 5′-untranslated region element (5′-UTR element), the 5′-UTR element includes SEQ ID NO: 2, 3, 4, 5, 6, 7, 8, 9, 10, 11, 12, 63, 64, 65, 66, 67, 68 or 69 nucleic acid sequences.

In a preferred embodiment, the 5′-UTR element comprises the nucleic acid sequence of SEQ ID NO: 2, 3, 4, 5, 6, 7, 8, 9, 10, 11 or 12.

In one embodiment, the artificial nucleic acid molecule comprises
a. At least one open reading frame (ORF); and
b. At least one 5′-untranslated region element (5′-UTR element) comprising a nucleic acid sequence derived from the 5′-UTR of the transcript of the following gene: alpha-1-globin, ARC, TUBB1, TUBB4B, ANXA5, A1CF, TUBA1C, TUBB3, HCV, CAMK2A, MAP2, HHV-5, beta globin, AGXT, C3, CYP2E1, FGA, EEF2, CTNNB1, NEB, TUBB6, ACTB, DEN2, TNNC2, MB and MYL2, and is truncated.

The term “nucleic acid sequence derived from the 5′-UTR of a transcript of a gene” refers to a nucleic acid sequence based on the 5′-UTR sequence of a transcript of a gene or a fragment or part thereof (preferably a naturally occurring gene or a fragment or part thereof). “Nucleic acid sequence derived from the 5′-UTR of the transcript of the gene” includes a sequence corresponding to the entire 5′-UTR sequence, that is, the full-length 5′-UTR sequence of the transcript of the gene, and the sequence corresponding to the transcript of the gene Sequence of the fragment of 5′-UTR sequence. Preferably, the fragment of the 5′-UTR of the transcript of the gene includes a stretch of continuous nucleotides corresponding to a stretch of continuous nucleotides in the full-length 5′-UTR of the transcript of the gene, which represents the entire length of the transcript of the gene at least 5%, at least 10%, at least 20%, at least 30%, at least 40%, at least 50%, at least 60%, at least 70%, at least 80%, or at least 90% of the long 5′-UTR. In this context, preferably the fragment retains a regulatory function on the translation of the ORF linked to the 5′-UTR or a fragment thereof.

The term “truncated” refers to a fragment or portion of a nucleic acid sequence based on the 5′-UTR sequence of a transcript of a gene. Preferably, the truncated 5′-UTR sequence comprises a stretch of contiguous nucleotides corresponding to a stretch of contiguous nucleotides in the full-length 5′-UTR of the transcript of the gene, which represents the full-length 5′ of the transcripts of the gene at least 5%, at least 10%, at least 20%, at least 30%, at least 40%, at least 50%1, at least 60%, at least 70%, at least 80% or at least 90% of the UTR.

In one embodiment, the nucleic acid sequence is derived from the nucleic acid sequence of the 5′-UTR of the transcript of the following human gene: alpha-1-globin, ARC, TUBB1, TUBB4B, ANXA5, CAMK2A, MAP2, AGXT, C3, CYP2E1, FGA, EEF2, CTNNB1, NEB, TUBB6, ACTB, TNNC2, MB and MYL2.

In one embodiment, the nucleic acid sequence is derived from the nucleic acid sequence of the 5′-UTR of the transcript of the following mouse genes: A1CF, TUBB1, TUBA1C, TUBB4B, TUBB3, and ACTB.

In one embodiment, the nucleic acid sequence is derived from the nucleic acid sequence of the 5′-UTR of the transcript of the following rat genes: ARC, CAMK2A and MAP2.

In one embodiment, the nucleic acid sequence is derived from the nucleic acid sequence of the 5′-UTR of the transcripts of the following viral genes: HCV, HHV-5 and DEN2.

In one embodiment, the nucleic acid sequence is derived from the nucleic acid sequence of the 5′-UTR of the transcript of the Xenopus gene beta globin.

In one embodiment, the 5′-UTR element exhibits at least about 3 nucleotides, preferably at least about 5 nucleotides, more preferably at least about 10, 15, 20, 25 or 30 nucleotides, or even a length of at least about 50 nucleotides is more preferred, and a length of at least about 70 nucleotides is most preferred. In preferred embodiments, the 5′-UTR element exhibits a length of 3 to about 500 nucleotides, preferably 5 to about 150 nucleotides, more preferably 50 to 110 nucleotides.

In one embodiment, the 5′-UTR element comprises SEQ ID NO: 13, 14, 15, 16, 17, 18, 19, 20, 21, 22, 23, 24, 25, 26, 27, 28, 29, 30, 31, 32, 33, 42, 43, 44, 45, 46, 47, 48, 49, 50, 51, 52, 53, 54, 55, 56, 57 or 58 nucleic acid sequence.

In a preferred embodiment, the artificial nucleic acid molecule comprises
a. at least one open reading frame (ORF); and
b. at least one 5′-untranslated region element (5′-UTR element) comprising a nucleic acid sequence derived from the 5′-UTR of the transcript of the following gene: alpha-1-globin, ARC, TUBB1, TUBB4B, ANXA5, A1CF, TUBA1C, TUBB3, and HCV, and are truncated.

In a preferred embodiment, the nucleic acid sequence is derived from the nucleic acid sequence of the 5′-UTR of the transcript of the following human genes: alpha-1-globin, ARC, TUBB1, TUBB4B and ANXA5.

In a preferred embodiment, the nucleic acid sequence is derived from the nucleic acid sequence of the 5′-UTR of the transcript of the following mouse genes: A1CF, TUBB1, TUBA1C, TUBB4B and TUBB3.

In a preferred embodiment, the nucleic acid sequence is derived from the nucleic acid sequence of the 5′-UTR of the transcript of the rat gene ARC.

In a preferred embodiment, the nucleic acid sequence is derived from the nucleic acid sequence of the 5′-UTR of the transcript of the viral gene HCV.

In a preferred embodiment, the nucleic acid sequence is derived from the nucleic acid sequence of the 5′-UTR of the transcript of the following human genes: alpha-1-globin, ARC, TUBB1, TUBB4B and ANXA5; or derived from the following mouse genes The nucleic acid sequence of the 5′-UTR of the transcript: A1CF, TUBB1, TUBA1C, TUBB4B and TUBB3; or the nucleic acid sequence of the 5′-UTR of the transcript derived from the rat gene ARC; or the transcript derived from the viral gene HCV. The nucleic acid sequence of the 5′-UTR.

In a preferred embodiment, the 5′-UTR element exhibits a length of 3-500 nucleotides, preferably 5-150 nucleotides, more preferably 50-110 nucleotides.

In one embodiment, the 5′-UTR element comprises the nucleic acid sequence of SEQ ID NO: 13, 14, 15, 16, 17, 18, 19, 20, 21, 22, 23 or 24.

In one embodiment, the artificial nucleic acid molecule comprises
a. At least one open reading frame (ORF); and
b. At least one 5′-untranslated region element (5′-UTR element) comprising SEQ ID NO: 34, 35, 36, 37, 38, 39, 40 or 41.

In one embodiment, the artificial nucleic acid molecule comprises
a. At least one open reading frame (ORF); and
b. At least one 5′-untranslated region element (5′-UTR element), the 5′-UTR element comprising a nucleic acid sequence selected from the following:
(i) wherein the 5′-UTR element contains 1, 2, 3 or more mutations compared to SEQ ID NO: 71; or
(ii) wherein the 5′-UTR element comprises SEQ ID NO: 2, 3, 4, 5, 6, 7, 8, 9, 10, 11, 12, 63, 64, 65, 66, 67, 68 or nucleic acid sequence of 69;
or
(iii) wherein the 5′-UTR element comprises a nucleic acid sequence derived from the 5′-UTR of a transcript of the following gene: alpha-1-globin, ARC, TUBB1, TUBB4B, ANXA5, A1CF, TUBA1C, TUBB3, HCV, CAMK2A, MAP2, HHV-5, beta globin, AGXT, C3, CYP2E1, FGA, EEF2, CTNNB1, NEB, TUBB6, ACTB, DEN2, TNNC2, MB, and MYL2, and are truncated;
or
(iv) wherein the 5′-UTR element comprises the nucleic acid sequence of SEQ ID NO: 34, 35, 36, 37, 38, 39, 40 or 41.

In one embodiment, the artificial nucleic acid molecule comprises
a. At least one open reading frame (ORF); and
b. At least one 5′-untranslated region element (5′-UTR element), the 5′-UTR element comprising a nucleic acid sequence selected from the following: SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69.

The specific nucleic acid sequence of the 5′-UTR element is shown in Table 1.

TABLE 1
Nucleic acid sequences of 5′-UTR elements

| numbers | Description Includes | SEQ ID NO: |
|---|---|---|
| U5061 | G16A, C17A, and G19A | 1. |
| | compared with SEQ ID NO: 71 | |
| U5065 | artificial nucleic acid | 2. |
| U5066 | artificial nucleic acid | 3. |
| U5067 | artificial nucleic acid | 4. |
| U5069 | artificial nucleic acid | 5. |
| U5071 | artificial nucleic acid | 6. |
| U5072 | artificial nucleic acid | 7. |
| U5073 | artificial nucleic acid | 8. |
| U5076 | artificial nucleic acid | 9. |
| U5079 | artificial nucleic acid | 10. |
| U5080 | artificial nucleic acid | 11. |
| U5082 | artificial nucleic acid | 12. |
| U5011 | human genes alpha-1-globin, | 13. |
| U5012 | Big mouseARC | 14. |
| U5013 | human genes ARC | 15. |
| U5038 | human genes TUBB1 | 16. |
| U5041 | human genes TUBB4B | 17. |
| U5043 | Mouse TUBA1C | 18. |
| U5044 | Mouse TUBB4B | 19. |
| U5045 | Mouse TUBB1 | 20. |
| U5046 | Mouse TUBB3 | 21. |
| U5052 | HCV | 22. |
| U5058 | human genesANXA5 | 23. |
| U5059 | Mouse A1CF | 24. |
| U5014 | Big Mouse CAMK2A | 25. |
| U5015 | human genes CAMK2A | 26. |
| U5016 | Big Mouse MAP2 | 27. |
| U5017 | human genes MAP2 | 28. |
| U5018 | HHV-5 | 29. |
| U5019 | HHV-5 | 30. |
| U5020 | HHV-5 | 31. |
| U5021 | Beta globin | 32. |
| U5022 | Beta globin | 33. |
| U5023 | artificial nucleic acid | 34. |
| U5024 | artificial nucleic acid | 35. |
| U5025 | artificial nucleic acid | 36. |
| U5026 | artificial nucleic acid | 37. |
| U5027 | artificial nucleic acid | 38. |
| U5028 | artificial nucleic acid | 39. |
| U5029 | artificial nucleic acid | 40. |
| U5030 | artificial nucleic acid | 41. |
| U5031 | human genesAGXT | 42. |
| U5032 | human genesFGA | 43. |
| U5033 | human genesEEF2 | 44. |
| U5034 | human genesCTNNB1 | 45. |
| U5036 | human genesNEB | 46. |
| U5036 | human genesTUBB6 | 47. |
| U5040 | human genesACTB | 48. |
| U5042 | human genesACTBV | 49. |
| U5047 | human genesACTINB | 50. |
| U5048 | human genesACTINB | 51. |
| U5049 | Mouse ACTINB | 52. |
| U5050 | Mouse ACTINB | 53. |
| U5051 | hcv | 54. |
| U5054 | DEN2 | 55. |
| U5055 | HumanTNNC2 | 56. |
| U5056 | humanMB | 57. |
| U5057 | HumanMYL2 | 58. |
| U5060 | Includes C9G, C10G | 59. |
| | compare with SEQ ID NO: 71 | |
| U5062 | Includes C57G, G58T | 60. |
| | compare with SEQ ID NO: 71 | |
| U5063 | Includes C26G, T27A, G28C | 61. |
| | compare with SEQ ID NO: 71 | |
| U5064 | Includes | 62. |
| | G16A, C17A, G19T, C16A | |
| | compare with SEQ ID NO: 71 | |
| U5068 | artificial nucleic acid | 63. |
| U5070 | artificial nucleic acid | 64. |
| U5074 | artificial nucleic acid | 65. |
| U5075 | artificial nucleic acid | 66. |
| U5077 | artificial nucleic acid | 67. |
| U5078 | artificial nucleic acid | 68. |
| U5081 | artificial nucleic acid | 69. |

In a preferred embodiment, the artificial nucleic acid molecule comprises
a. At least one open reading frame (ORF); and
b. At least one 5′-untranslated region element (5′-UTR element), the 5′-UTR element comprising a nucleic acid sequence selected from the following:
(i) wherein the 5′-UTR element contains 1, 2, 3 or more mutations compared to SEQ ID NO: 71; or
(ii) wherein the 5′-UTR element comprises the nucleic acid sequence of SEQ ID NO: 2, 3, 4, 5, 6, 7, 8, 9, 10, 11 or 12; or
(iii) wherein said 5′-UTR element comprises a nucleic acid sequence derived from a 5′-UTR of a transcript of the following genes: alpha-1-globin, ARC, TUBB1, TUBB4B, ANXA5, A1CF, TUBA1C, TUBB3 and HCV, and is truncated.

In a particularly preferred embodiment, the artificial nucleic acid molecule comprises
a. At least one open reading frame (ORF); and
b. At least one 5′-untranslated region element (5′UTR element), the 5′-UTR element comprising a nucleic acid sequence selected from the following:
(i) wherein the 5′-UTR element contains 1, 2, 3 or more mutations compared to SEQ ID NO: 71; or
(ii) wherein the 5′-UTR element comprises the nucleic acid sequence of SEQ ID NO: 2, 3, 4, 5, 6, 7, 8, 9, 10, 11 or 12;
or
(iii) wherein said 5′-UTR element comprises a nucleic acid sequence derived from a 5′-UTR of a transcript of the following genes: alpha-1-globin, ARC, TUBB1, TUBB4B, ANXA5, A1CF, TUBA1C, TUBB3 and HCV, and is truncated;
or the corresponding RNA sequence of the above nucleic acid sequence.

In a specific embodiment, the artificial nucleic acid molecule comprises
a. At least one open reading frame (ORF); and
b. At least one 5′-untranslated region element (5′-UTR element), the 5′-UTR element comprising a nucleic acid sequence selected from the following:

### SEQ ID NO: 1, 2, 3, 4, 5, 6, 7, 8, 9, 10, 11, 12, 13, 14, 15, 16, 17, 18, 19, 20, 21, 22, 23 or 24.

The term “Kozak sequence” (also known as “Kozak consensus sequence”) refers to a translation initiation enhancer element used to enhance expression of a gene or open reading frame, and which in eukaryotes is located in the 5′-UTR. The 5′-UTR elements as defined herein may comprise the Kozak consensus sequence, or derivatives or modifications thereof. In a preferred embodiment, the Kozak sequence comprises the nucleic acid sequence of SEQ ID NO:70.

As used herein, the term “3′-UTR” refers to the portion of an artificial nucleic acid molecule that is 3′ (i.e., “downstream”) of the open reading frame and that is not translated into a protein. Typically, the 3′-UTR is a portion of the mRNA between the protein coding region (open reading frame (ORF) or coding sequence (CDS)) and the poly(A) sequence. In the context of the present invention, the term 3′-UTR may also include elements which are not encoded in the template from which the RNA is transcribed, but which are added post-transcriptionally during maturation, for example polyadenylation sequences. The 3′-UTR of mRNA is not translated into an amino acid sequence. 3′-UTR plays an important role in the regulation of biological complexity. It can regulate the localization and expression of mRNA, can regulate the translation of mRNA, and can also regulate protein-protein interactions (see, for example, Mayr C. What Are 3′UTRs Doing Cold Spring Harb Perspect Biol. 2019 Oct. 1; 11(10):a034728).

In a preferred embodiment, the artificial nucleic acid molecule further comprises at least one 3′-untranslated region element (3′-UTR element).

In a preferred embodiment, the 3′-untranslated region element (3′-UTR element) comprises the nucleic acid sequence of SEQ ID NO:72.

As used herein, the term “poly(A) sequence” or “poly(A) tail” refers to a nucleotide sequence containing contiguous or discontinuous adenosine nucleotides. The poly(A) sequence is usually located at the 3′ end of the RNA, such as the 3′ end (downstream) of the 3′-UTR. In some embodiments, the poly(A) sequence contains no nucleotides other than adenylate at its 3′ end. The poly(A) sequence can be transcribed by DNA-dependent RNA polymerase according to the coding sequence of the DNA template during the preparation of IVT-RNA, or linked to the IVT by a DNA-independent RNA polymerase (poly(A) polymerase)—The free 3′ end of the RNA, for example the 3′ end of the 3′-UTR. In one embodiment, the artificial nucleic acid molecule further comprises a poly(A) sequence.

As used herein, the term “polycytidylic acid sequence” refers to a nucleotide sequence containing contiguous or discontinuous cytidylic acids. The polycytidylic acid sequence is usually located at the 3′ end of the RNA, such as the 3′ end (downstream) of the 3′-UTR. In some embodiments, the polycytidylic acid sequence contains no nucleotides other than cytidylic acid at its 3′ end. In one embodiment, the artificial nucleic acid molecule further comprises a polycytidylic acid sequence.

As used herein, the term “5′ cap” generally refers to an N7-methylguanosine structure (also known as “m7G cap”, “m7Gppp-”) linked to the 5′ end of an mRNA via a 5′ to 5′ triphosphate bond.

The 5′ cap can be co-transcriptionally added to the RNA during in vitro transcription (eg using the anti-reverse cap analog “ARCA”), or can be ligated to the RNA post-transcriptionally using a capping enzyme. In one embodiment, the artificial nucleic acid molecule further comprises a 5′ cap structure.

As used herein, a “stem-loop” (whether it is a histone stem-loop or not) may generally be present in single-stranded DNA, or more commonly in RNA. This structure is also called a hairpin or hairpin loop, and usually consists of a stem and a (terminal) loop within a continuous sequence, where the stem consists of two adjacent lines separated by a short sequence that acts to some extent as a spacer A complete or partial reverse complementary sequence is formed, and the short sequence as a spacer becomes a loop of the stem-loop structure. These two adjacent sequences that are fully or partially reverse complementary can be defined, for example, as stem-loop elements stem1 and stem2. When these two adjacent fully or partially reverse complementary sequences, for example, stem-loop elements Stem 1 and Stem 2, form base pairs with each other, a double-stranded nucleic acid sequence fragment is formed, which contains at its ends a sequence consisting of two consecutively located. The short sequence on the stem-loop element between stem1 and stem2 forms an unpaired loop, thus forming a stem-loop.

As used herein, a “histone stem-loop” is derived from a histone gene (e.g., a gene derived from histone families H1, H2A, H2B, H3, H4) and consists of two adjacent, fully or partially reverse complementary Intramolecular base pairing of sequences, thereby forming stem-loops. In one embodiment, the artificial nucleic acid molecule further comprises a histone stem loop.

In one embodiment, the artificial nucleic acid molecule further comprises a 5′ cap structure, a polycytidylic acid sequence, a polyadenylic acid sequence, or a histone stem-loop.

In one embodiment, the artificial nucleic acid molecule further comprises a 5′ cap structure and a polycytidylic acid sequence.

In one embodiment, the artificial nucleic acid molecule further comprises a 5′ cap structure and a poly(A) sequence.

In one embodiment, the artificial nucleic acid molecule further comprises a 5′ cap structure, a polycytidylic acid sequence and a histone stem loop.

In one embodiment, the artificial nucleic acid molecule further comprises a 5′ cap structure, a poly(A) sequence, and a histone stem-loop.

In one embodiment, the artificial nucleic acid molecule further comprises a 5′ cap structure, a polycytidylic acid sequence, a polyadenylic acid sequence, and a histone stem-loop.

In a preferred embodiment, the artificial nucleic acid molecule comprises a poly(A) sequence comprising the nucleic acid sequence of SEQ ID NO:73.

Artificial nucleic acid molecules as defined herein may be prepared using any method known in the art, including synthetic methods, e.g., solid phase synthesis, and in vitro methods, e.g., in vitro transcription reactions, or in vivo reactions, e.g., in vivo propagation of DNA plasmids in bacteria. The artificial nucleic acid molecules of the invention can be codon-optimized for the host cells used for expression.

In a preferred embodiment, the open reading frame (ORF) is codon optimized.

In one embodiment, the artificial nucleic acid molecule is RNA.

In a preferred embodiment, the artificial nucleic acid molecule is mRNA.

In one embodiment, the 5′-UTR element comprises SEQ ID NO: 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, The nucleic acid sequence of 140, 141 or 142.

In one embodiment, the 5′-UTR element comprises SEQ ID NO: 74, 75, 76, 77, 78, 79, 80, 81, 82, 83, 84, 85, 86, 87, 88, 89, Nucleic acid sequence 90, 91, 92, 93, 94, 95, 96 or 97.

### Modified Nucleotides

In some ways, the mRNA includes modified nucleotides, wherein the modified nucleotides are selected from one or several of the following nucleotides: 2-aminoadenosine, 2-thiothymidine, inosine, Pyrrolopyrimidine, 3-methyladenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxadenosine, 8-oxoguanosine, 0(6)-methylguanine, pseudouridine, N-1-methyl-pseudouridine, 2-thio Uridine and 2-thiocytidine; methylated bases; inserted bases; 2′-fluoribose, ribose, 2′-deoxyribose, arabinose, and hexose; phosphorothioate and 5′-N-Phosphoramidite bonds. And modified with the modified nucleotides described in PCT/CN2020/074825 and PCT/CN2020/106696.

### Vectors and Host Cells

In yet another aspect, the invention also provides an expression vector comprising the artificial nucleic acid molecule of the invention. The expression vector may further contain additional nucleic acid sequences, such as regulatory sequences and antibiotic resistance genes. The artificial nucleic acid molecules of the invention may be present in one or more expression vectors.

In one embodiment, the vector is a DNA vector.

In one embodiment, the vector is a plasmid vector or a viral vector.

In a preferred embodiment, the vector is a plasmid vector. For example, pUC57 plasmid vector.

In one embodiment, the carrier is a circular molecule.

In one embodiment, the artificial nucleic acid molecules of the invention are prepared as recombinant nucleic acids. Recombinant nucleic acids can be prepared using techniques well known in the art, such as chemical synthesis, DNA recombinant technology (such as polymerase chain reaction (PCR) technology), and the like.

In yet another aspect, the invention also provides a method for producing the artificial nucleic acid molecule of the invention, the method comprising:
a) Synthesize the artificial nucleic acid molecule of the present invention; or
b) Synthesize artificial nucleic acid molecules through the vector of the present invention.

In yet another aspect, the present invention also provides a method for increasing the translation efficiency of an artificial nucleic acid molecule, preferably an mRNA molecule or a vector, the method comprising connecting an open reading frame and a 5′-UTR element as defined in the present invention.

The invention also provides a cell comprising the artificial nucleic acid molecule or vector of the invention. Various methods known in the art can be used to introduce the artificial nucleic acid molecules or vectors of the present invention into appropriate cells. Such methods include, but are not limited to, lipofection, electroporation, viral transduction, calcium phosphate transfection, etc.

In a preferred embodiment, cells are used to express the peptide or protein encoded by the ORF in the artificial nucleic acid molecule of the present invention. Examples of cells include, but are not limited to, prokaryotic cells (eg bacteria, eg E. coli) and eukaryotic cells (eg yeast, insect cells, mammalian cells). Suitable mammalian host cells include, but are not limited to, human cervical cancer cells (HeLa cells), human embryonic kidney cells (HEK cells, such as HEK 293 cells), Chinese hamster ovary (CHO) cells, and other mammalian cells.

In one embodiment, the cell is a mammalian cell.

In a preferred embodiment, the cells are cells isolated from a human subject.

### Livid Composition

In yet another aspect, the invention also provides a lipid composition. The lipid composition is a lipid delivery carrier, and the lipid can encapsulate the artificial nucleic acid molecules of the present invention to form nanoparticles, thereby delivering them to the living body.

As used herein, the term “lipid” refers to an organic compound that contains a hydrophobic portion and, optionally, a hydrophilic portion. Lipids are generally poorly soluble in water but soluble in many organic solvents. Generally, amphipathic lipids containing hydrophobic and hydrophilic parts can be organized into lipid bilayer structures in an aqueous environment, for example in the form of vesicles. Lipids may include, but are not limited to: fatty acids, glycerides, phospholipids, sphingolipids, glycolipids, steroids, cholesterol esters, etc.

As used herein, “lipid nanoparticle” or “LNP” refers to a lipid vesicle with a uniform lipid core, which is a particle formed from lipids whose components undergo intermolecular interactions to form nanostructures entity. Nucleic acids (such as mRNA) are encapsulated in lipids.

A particularly preferred lipid composition may be, for example, a lipid polyplex (LPP) as described herein. Methods for preparing such compositions can be as described herein. LPP is a particle with a core-shell structure, in which nucleic acids are contained in a multimeric complex, and the multimeric complex itself is encapsulated in a biocompatible lipid bilayer shell to constitute the lipid nanoparticles of the present invention. In some embodiments, the lipid composition of the invention is a lipid polyplex (LPP). In some embodiments, the lipid compositions of the present invention are lipid polyplexes (LPPs) containing artificial nucleic acid molecules.

In some embodiments, the lipid encapsulating the artificial nucleic acid molecule of the present invention is selected from one or more of the following lipids: cationic lipids, phospholipids, steroids and/or polyethylene glycol modified lipids. In a preferred embodiment, the cationic lipid is an ionizable cationic lipid.

The lipid composition of the present invention includes the artificial nucleic acid molecule of the present invention and a lipid encapsulating the artificial nucleic acid molecule. The lipids encapsulating artificial nucleic acid molecules include cationic lipids, phospholipids, steroids and polyethylene glycol modified lipids.

In one embodiment, the lipid composition comprises a cationic lipid, wherein the cationic lipid comprises DOTMA, DOTAP, DDAB, DOSPA, DODAC, DODAP, DC-Chol, DMRIE, DMOBA, DLinDMA, DLenDMA, CLinDMA, DMORIE, DLDMA, DMDMA, DOGS, N₄-cholesteryl-spermine, DLin-KC2-DMA, DLin-MC3-DMA, compounds of formula (I), (II), (III) or (IV) as described herein, or combinations thereof. In a preferred embodiment, the cationic lipids include M5, MC3, ALC-0315, SM-102. In a preferred embodiment, the cationic lipids include SW-II-115, SW-II-121, SW-II-122, SW-II-134-3, SW-II-138-2, SW-II-139-2 or SW-II-140-2. In a preferred embodiment, the cationic lipids include M5, MC3, ALC-0315, SM-102, SW-II-115, SW-II-121, SW-II-122, SW-II-134-3, SW-II-138-2, SW-II-139-2 or SW-II-140-2.

In one embodiment, the lipid composition includes phospholipids and/or steroids. In one embodiment, the lipid composition comprises a phospholipid as described herein, wherein the phospholipid comprises 1,2-dilinoleoyl-sn-glycero-3-phosphocholine (DLPC), 1,2-dimyriste Acyl-sn-glycero-phosphocholine (DMPC), 1,2-dioleoyl-sn-glycero-3-phosphocholine (DOPC), 1,2-dipalmitoyl-sn-glycero-3-phosphocholine base (DPPC), 1,2-distearoyl-sn-glycero-3-phosphocholine (DSPC), 1,2-diundecanoyl-sn-glycero-phosphocholine (DUPC), 1-Palmitoyl-2-oleoyl-sn-glycero-3-phosphocholine (POPC), 1,2-di-O-octadecenyl-sn-glycero-3-phosphocholine (18:0Diether PC), 1-oleoyl-2-cholesteryl hemisuccinyl-sn-glycero-3-phosphocholine (OChemsPC), 1-hexadecyl-sn-glycero-3-phosphocholine (C16Lyso PC), 1, 2-Dilinolenoyl-sn-glycero-3-phosphocholine, 1,2-Diarachidonoyl-sn-glycero-3-phosphocholine, 1,2-bisdocosahexaenoyl-sn-Glyceryl-3-phosphocholine, 1,2-dioleoyl-sn-glycero-3-phosphoethanolamine (DOPE), 1,2-diphytanoyl-sn-glycero-3-phosphoethanolamine (ME 16.0PE), 1,2-distearoyl-sn-glycerol-3-phosphoethanolamine, 1,2-dilinoleoyl-sn-glycerol-3-phosphoethanolamine, 1,2-dilinolenoyl-sn-glycerol-3-Phosphoethanolamine, 1,2-diarachidonoyl-sn-glycero-3-phosphoethanolamine, 1,2-bidocosahexaenoyl-sn-glycero-3-phosphoethanolamine, 1,2-Dioleoyl-sn-glycerol-3-phosphate-rac-(1-glycerol) sodium salt (DOPG), dipalmitoyl phosphatidylglycerol (DPPG), palmitoyloleoyl phosphatidylethanolamine (POPE), distearoyl-Phosphatidyl-ethanolamine (DSPE), dipalmitoyl phosphatidylethanolamine (DPPE), dimyristoyl phosphoeth anolamine (DMPE), 1-stearoyl-2-oleoyl-stearoylethanolamine (SOPE), 1-Stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, Lysophosphatidylcholine, lysophosphatidylethanolamine (LPE) or combinations thereof. In one embodiment, the lipid composition comprises a steroid as described herein, wherein the steroid comprises cholesterol, coprosterol, sitosterol, ergosterol, campest Erol, stigmasterol, brassica sterol, tomatine, unsolid acid, α-tocopherol and its derivatives. In one embodiment, the lipid composition includes a phospholipid and a steroid as described herein. In one embodiment, the lipid composition includes DOPE. In one embodiment, the lipid composition comprises DSPC. In one embodiment, the lipid composition includes cholesterol. In one embodiment, the lipid composition includes DOPE and cholesterol. In one embodiment, the lipid composition includes DSPC and cholesterol.

In one embodiment, the lipid composition includes cationic lipids M5, MC3, ALC-0315, SM-102, SW-II-115, SW-II-121, SW-II-122, SW-II-134-3. SW-II-138-2, SW-II-139-2 or SW-II-140-2, phospholipid DOPE and cholesterol. In one embodiment, the lipid composition includes cationic lipids M5, MC3, ALC-0315, SM-102, SW-II-115, SW-II-121, SW-II-122, SW-II-134-3. SW-II-138-2, SW-II-139-2 or SW-II-140-2, phospholipid DSPC and cholesterol.

In some embodiments, the lipid encapsulating the artificial nucleic acid molecule of the invention further comprises polyethylene glycol modified lipid. In one embodiment, the polyethylene glycol modified lipids comprise DMG-PEG (eg, DMG-PEG 2000), DOG-PEG, and DSPE-PEG, or combinations thereof. In one embodiment, the polyethylene glycol modified lipid is DSPE-PEG. In one embodiment, the polyethylene glycol modified lipid is DMG-PEG (eg, DMG-PEG 2000).

In one embodiment, the lipid composition includes cationic lipids, DOPE, cholesterol, and DSPE-PEG.

In one embodiment, the lipid composition includes cationic lipids, DSPC, cholesterol, and DSPE-PEG. In one embodiment, the lipid composition includes cationic lipids, DSPC, cholesterol, and DMG-PEG.

In a preferred embodiment, the lipid composition contains cationic lipids, DOPE, cholesterol and DMG-PEG.

In a preferred embodiment, the lipid composition includes cationic lipids M5, MC3, ALC-0315, SM-102, SW-II-115, SW-II-121, SW-II-122, SW-II-134-3, SW-II-138-2, SW-II-139-2 or SW-II-140-2, DOPE, cholesterol and DMG-PEG.

In some embodiments, the lipid composition of the present invention further comprises a cationic polymer associated with the artificial nucleic acid molecule into a complex and co-encapsulated in the lipid.

In one embodiment, the cationic polymer includes poly-L-lysine, protamine, polyethylenimine (PEI), or combinations thereof. In one embodiment, the cationic polymer is protamine. In one embodiment, the cationic polymer is polyethyleneimine.

In one embodiment, the amount of lipid in the lipid composition is calculated as mole percent (mol %), which is determined based on the total moles of lipids in the composition.

In one embodiment, the amount of cationic lipids in the lipid composition is from about 10 to about 70 mole percent. In some embodiments, the amount of cationic lipids in the lipid composition is about 20 to about 60 mol %, about 30 to about 50 mol %, about 35 to about 50 mol %, about 35 to about 45 mol %, about 38 to about 45 mol %, about 40 to about 45 mol %, about 40 to about 50 mol %, or about 45 to about 50 mol %.

In one embodiment, the number of phospholipids in the lipid composition is from about 10 to about 70 mole percent. In one embodiment, the number of phospholipids in the lipid composition is about 20 to about 60 mol %, about 30 to about 50 mol %, about 10 to about 30 mol %, about 10 to about 20 mol %, or about 10-About 15 mol %.

In one embodiment, the amount of cholesterol in the lipid composition is from about 10 to about 70 mole percent. In one embodiment, the amount of cholesterol in the lipid composition is from about 20 to about 60 mol %, from about 24 to about 44 mol %, from about 30 to about 50 mol %, from about 35 to about 40 mol %, from about 35 to about 35 mol %. 45 mol %, about 40 to about 45 mol %, or about 45 to about 50 mol %.

In one embodiment, the amount of polyethylene glycol modified lipid in the lipid composition is from about 0.05 to about 20 mole percent. In one embodiment, the amount of polyethylene glycol modified lipid in the lipid composition is about 0.5 to about 15 mol %, about 1 to about 10 mol %, about 5 to about 15 mol %, about 1 to about 5 mol %, about 1 to about 1.5 mol %, about 1.5 to about 3 mol %, or about 2 to 5 mol %.

In one embodiment, the lipid composition contains 10-70 mol % cationic lipids, 10-70 mol % phospholipids, 10-70 mol % steroids, and 0.05-20 mol % polyethylene glycol modified lipids quality.

In a preferred embodiment, the lipid composition contains 35-50 mol % cationic lipids, 10-30 mol % phospholipids, 24-44 mol % steroids and 1-1.5 mol % polyethylene glycol modified Lipids.

In one embodiment, the LPP comprises an artificial nucleic acid molecule of the invention and a lipid encapsulating the artificial nucleic acid molecule, wherein the lipid encapsulating the artificial nucleic acid molecule comprises cationic lipids, phospholipids, steroids and polyethylene glycol. Modified lipid; the LPP further includes a cationic polymer, wherein the cationic polymer is associated with the artificial nucleic acid molecule into a complex. In one embodiment, the lipid composition of the present invention includes an artificial nucleic acid molecule of the present invention and a lipid encapsulating the artificial nucleic acid molecule, wherein the lipid encapsulating the artificial nucleic acid molecule includes a cationic lipid, Phospholipids, steroids and polyethylene glycol modified lipids; the lipid composition also includes a cationic polymer, wherein the cationic polymer is associated with the artificial nucleic acid molecule into a complex and is co-encapsulated in the lipid. Lipid multimeric complexes are formed in the plasma. In one embodiment, the phospholipid is selected from 1,2-dioleoyl-sn-glycero-3-phosphoethanolamine (DOPE), distearoylphosphatidylcholine (DSPC), or a combination thereof. In one embodiment, the steroid is cholesterol. In one embodiment, the cationic polymer is protamine. In one embodiment, the polyethylene glycol-modified lipid is selected from the group consisting of 1,2-dimyristoyl-rac-glycerol-3-methoxypolyethylene glycol (DMG-PEG), 1,2-Distearoyl-sn-glycerol-3-phosphoethanolamine-poly (ethylene glycol) (DSPE-PEG) or combinations thereof. In one embodiment, the cationic lipid is selected from M5, MC3, ALC-0315, SM-102, SW-II-115, SW-II-121, SW-II-122, SW-II-134-3, SW-II-138-2, SW-II-139-2 or SW-II-140-2.

In one embodiment, the lipids of the encapsulated complex comprise 50 mol % of M5, MC3, ALC-0315, SM-102, SW-II-115, SW-II-121, SW-II-122, SW-II-134-3, SW-II-138-2, SW-II-139-2 or SW-II-140-2, 10 mol % DOPE, 38.5 mol % cholesterol and 1.5 mol % DMG-PEG. In one embodiment, the lipids of the encapsulated complex comprise 40 mol % of M5, MC3, ALC-0315, SM-102, SW-II-115, SW-II-121, SW-II-122, SW-II-134-3, SW-II-138-2, SW-II-139-2 or SW-II-140-2, 15 mol % DOPE, 43.5 mol % cholesterol and 1.5 mol % DMG-PEG.

### Cationic Lipids

Cationic lipids are lipids that can carry a net positive charge at a specified pH. Lipids with a net positive charge can associate with nucleic acids through electrostatic interactions. Examples of cationic lipids include, but are not limited to, 1,2-di-O-octadecenyl-3-trimethylammonium-propane (DOTMA), 1,2-Dioleoyl-3-trimethylammonium-propane (1,2-dioleoyl-3-trimethylammonium-propane, DOTAP), Didecyldimethylammonium bromide (DDAB), 2, 3-Dioleoyloxy-N-[2(sperminecarboxamide)ethyl]-N,N-dimethyl-1-propylamine trifluoroacetate (2,3-dioleoyloxy-N-[2 (spermine carboxamide)ethyl]-N,N-dimethyl-1-propanamium trifluoroacetate (DOSPA), dioctadecyldimethyl ammonium chloride (DODAC), 1,2-dioleoyl-3-Dimethylammonium-propane (1,2-dioleoyl-3-dimethylammonium-propane, DODAP), 3-(N—(N′,N′-dimethylaminoethane)-carbamoyl)cholesterol (3-(N—(N′,N′-dimethylaminoethane)-carbamoyl)cholesterol, DC-Chol), 2,3-di(tetradecyloxy)propyl-(2-hydroxyethyl)-dimethylamine Onium (2,3-di(tetradecoxy)propyl-(2-hydroxyethyl)-dimethylazanium, DMRIE), N,N-dimethyl-3,4-dioleyloxybenzylamine (N,N-dimethyl-3,4-dioleyloxybenzylamine (DMOBA), 1,2-dilinoleyloxy-N,N-dimethylaminopropane (1,2-dilinoleyloxy-N,N-dimethylaminopropane, DLinDMA), 1,2-dilinDMA Olyloxy-N,N-dimethylaminopropane (1,2-dilinolenyloxy-N,N-dimethylaminopropane, DLenDMA), 3-dimethylamino-2-(cholester-5-ene-3-O-Oxybutane-4-oxy)-1-(cis,cis-9,12-octadecadienyloxy)propane(3-dimethylamino-2-(cholest-5-en-3-beta-oxybutan-4-oxy)-1-(cis,cis-9,12-oc-tadecadienoxy)propane, CLinDMA), N-(2-aminoethyl)-N,N-dimethyl-2,3-Bis(tetradecyloxy)propane-1-aminium bromide (N-(2-aminoethyl)-N,N-dimethyl-2,3-bis(tetradecyloxy)propan-1-aminium bromide, DMORIE), N,N-dimethyl-2,3-bis(dodecyloxy)propan-1-amine (N,N-dimethyl-2,3-bis(dodecyloxy)propan-1-amine, DLDMA), N,N-dimethyl-2,3-bis(tetradecyloxy)propan-1-amine (N,N-dimethyl-2,3-bis(tetradecyloxy)propan-1-amine, DMDMA), dioctadecylamidoglycyl spermine (DOGS), N4-cholesteryl-spermine, 2,2-dilinoleyl-4-(2-Dimethylaminoethyl)-[1,3]-dioxolane (2,2-dilinoleyl-4-(2-dimethylaminoethyl)-[1,3]-dioxolane, DLin-KC2-DMA), tri Heptatriaconta-6,9,28,31-tetraen-19-yl-4-(dimethylamino)butyrate (heptatriaconta-6,9,28,31-tetraen-19-yl-4-(dimethylamino)butanoate, DLin-MC3-DMA), a compound of formula (I), (II), (III) or (IV) as described herein, or a combination thereof.

In some embodiments, the cationic lipid is preferably an ionizable cationic lipid. Ionizable cationic lipids have a net positive charge at, for example, acidic pH and are neutral at higher pH (eg, physiological pH). Examples of ionizable cationic lipids include, but are not limited to: dioctadecylmidoglycyl spermine (DOGS), N4-cholesteryl-spermine, 2,2-Dilinoleyl-4-(2-dimethylaminoethyl)-[1,3]-dioxolane(2,2-dilinoleyl-4-(2-dimethylaminoethyl)-[1,3]-dioxolane, DLin-KC2-DMA), heptatriaconta-6,9,28,31-tetraen-19-yl-4-(dimethylamino)butyrate (heptatriaconta-6,9,28, 31-tetraen-19-yl-4-(dimethylamino)butanoate, DLin-MC3-DMA), a compound of formula (I), (II), (III) or (IV) as described herein, or a combination thereof.

In one embodiment, the cationic lipid comprises a compound of formula (I) or a pharmaceutically acceptable salt thereof:

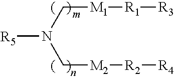

wherein
R1 and R2 each independently selected from key, C1-C12 Alkyl and C2-C12 Alkenyl;
R3 and R4 each is independently selected from C1-C12 Alkyl, C2-C12 Alkenyl, C6-C10 Aryl and 5-10 membered heteroaryl; and R3 and R4 ach independently selects t R6 Instead, t is an integer selected from 1-5;
R6 each is independently selected from C1-C12 Alkyl and C2-C12 Alkenyl; M1 and M2 each independently selected from bond, H, —O—, —S—, —C(O)—, —OC(O)—, —C(O)O—, —OC(O)O—, —SC(S)—, —C(S)S—, 3-10 membered heterocycle, —NR7-, or
R5 with M1 and M2 one of them together with the connected N atom forms a 3-10 membered heterocyclic ring, and the corresponding R1/R3 or
R2/R4 is not present, the heterocycle is optionally replaced by R7 replace;
R5 selected from C3-8 Carbocyclic ring, —C1-12 Alkylene-Q, Q is selected from H, —OR7, —SR7, —OC(O)R7, —C(O)OR7, —N(R7)C(O)R7, —N(R7)S(O)2 R7, —N(R7)C(S)R7, —N(R7)2, cyano group, C3-8 Carbocyclic ring, 3-10 membered heterocyclic ring, C6-C10 Aryl, each of the above groups is optionally substituted by one or more C1-C12 Alkyl, C2-C12 Alkenyl, C1-C12 Alkoxy, C6-C10 Aryl, 5-10 membered heteroaryl, 3-10 membered heterocycle, halogen, hydroxyl, oxo (═O) substitution;
m and n are each independently an integer selected from 0-12;
the alkyl, alkenyl and alkylene groups are each optionally and independently interrupted by one or more groups selected from: —O—, —S—, —NR7-, —C(O)—, —OC(O)—, —C(O)O—, —SC(S)—, —C(S)S—, C3-8 carbocyclic ring, and each of the alkyl, alkenyl and alkylene groups is optionally replaced by one or more R7 replace;
R7 each is independently selected from H, C1-C12 Alkyl, C2-C12 Alkenyl, C1-C12 Alkoxy, carboxylic acid, sulfinic acid, sulfonic acid, sulfonyl, nitro, cyano, amino, carbamoyl, sulfonamide, C6-C10 Aryl, 5-10 membered heteroaryl, 3-10 membered heterocycle, halogen, C3-8 Carbocyclic ring, each of the above groups is optionally replaced by one or more C1-C12 Alkyl, C2-C12 Alkenyl, C1-C12 Alkoxy, C6-C10 Aryl, 5-10 membered heteroaryl, 3-10 membered heterocycle, halogen, hydroxyl, oxo (═O) substitution.

In one embodiment, R1 and R2 each is independently selected from C1-C12 Alkyl and C2-C12 Alkenyl, such as C1-C12 alkyl. In yet another embodiment, R1 and R2 One is a key and the other is independently selected from C1-C12 Alkyl and C2-C12 Alkenyl, such as C1-C12 alkyl.

In one embodiment, R3 and R4 each is independently selected from C1-C12 Alkyl, C2-C12 Alkenyl, C6-C10 Aryl and 5-10 membered heteroaryl.

In yet another embodiment, R3 and R4 each is independently selected from C1-C12 Alkyl and C2-C12 alkenyl.

R3 and R4 Can be independently selected by t R6 instead, t is 1, 2, 3, 4, 5. In one embodiment, R6 each is independently selected from C1-C12 alkyl.

In yet another embodiment, R3 and R4 At least one of them is C6-C10 Aryl or 5-10 membered heteroaryl, such as C6-C10 Aryl.

In one embodiment, R5 Selected from C3-8 Carbocyclic ring, —C1-12 Alkylene-Q. Q can be selected from H, —OR7, —SR7, —OC(O)R7, —C(O)OR7, —N(R7)C(O)R7, —N(R7)S(O)2R7, —N(R7)C(S)R7, —N(R7) 2, cyano group, C3-8 Carbocyclic ring, 3-10 membered heterocyclic ring, C6-C10 Aryl. The above groups, including groups encompassing the options of Q, may each be optionally replaced by one or more C, where appropriate. 1-C12 Alkyl, C2-C12 Alkenyl, C1-C12 Alkoxy, C6-C10 Aryl, 5-10 membered heteroaryl, 3-10 membered heterocycle, halogen, hydroxyl, oxo (═O) substitution.

In yet another embodiment, R5 Selected from C3-8 Carbocyclic ring, —C1-12 Alkylene-Q, Q is selected from H, —OR7, —SR7, —OC(O)R7, —C(O)OR7, —N(R7)C(O)R7, —N(R7)S(O)2R7, —N(R7)C(S)R7, —N(R7)2, cyano group, C3-8 Carbocyclic ring, 3-10 membered heterocyclic ring, C6-C10 Aryl. The above groups, including groups encompassing the options of Q, may each be optionally replaced by one or more C, where appropriate. 1-C12 Alkyl, C2-C12 Alkenyl, C1-C12 Alkoxy, C6-C10 Aryl, 5-10 membered heteroaryl, 3-10 membered heterocycle, halogen, hydroxyl, oxo (═O) substitution.

In compounds of formula (I), R7 Can be selected independently from H, C1-C12 Alkyl, C2-C12 Alkenyl, C1-C12 Alkoxy, carboxylic acid, sulfinic acid, sulfonic acid, sulfonyl, nitro, cyano, amino, carbamoyl, sulfonamide, C6-C10 Aryl, 5-10 membered heteroaryl, 3-10 membered heterocycle, halogen, C3-8 Carbocyclic ring, preferably selected from H, C1-C12 Alkyl, C2-C12 Alkenyl, C1-C12 Alkoxy, carboxylic acid, sulfinic acid, sulfonic acid, sulfonyl, nitro, cyano, amino, carbamoyl, sulfonamide, C6-C10 Aryl and 5-10 membered heteroaryl. The above groups (when appropriate, e.g. H, C1-C12 Alkyl, C2-C12 Alkenyl, C1-C12 Alkoxy, carboxylic acid, sulfinic acid, sulfonic acid, sulfonyl, amino, carbamoyl, sulfonamide, C6-C10 Aryl, 5-10 membered heteroaryl, 3-10 membered heterocycle, C3-8 carbocyclic rings) are each optionally replaced by one or more C1-C12 Alkyl, C2-C12 Alkenyl, C1-C12 Alkoxy, C6-C10 Aryl, 5-10 membered heteroaryl, 3-10 membered heterocycle, halogen, hydroxyl, oxo (═O) substitution.

In one embodiment, each group in the above description, such as C3-8 Carbocyclic ring, —C1-12 Alkylene -Q, including —OR covering Q options OR 7, —SR7, —OC(O)R7, —C(O)OR7, —N(R7)C(O)R7, —N(R7)S(O) 2 R7, —N(R7)C(S)R7, —N(R7) 2, C3-8 Carbocyclic ring, 3-10 membered heterocyclic ring, C6-C10 Aryl, C1-C12 Alkyl, C2-C12 Alkenyl, C1-C12 Alkoxy, carboxylic acid, sulfinic acid, sulfonic acid, sulfonyl, amino, carbamoyl, sulfonamide, C6-C10 Aryl, 5-10 membered heteroaryl, 3-10 membered heterocycle, halogen, C3-8 Carbocycles, etc. may each be optionally replaced by one or more C1-C12 Alkyl, C2-C12 Alkenyl, C1-C12 Alkoxy, C6-C10 Aryl, 5-10 membered heteroaryl, 3-10 membered heterocycle, halogen, hydroxyl, oxo (═O) substitution.

In one embodiment, in the compound of formula (I) (e.g. R1-R7 (mentioned in) alkyl, alkenyl and alkylene groups may each optionally be interrupted independently by one or more groups selected from: —O—, —S—, —NR7-, —C(O)—, —OC(O)—, —C(O)O—, —SC(S)—, —C(S)S—, C3-8 carbocyclic ring, and each of the alkyl, alkenyl and alkylene groups is optionally replaced by one or more R7 replace.

That is, the chains (linear or branched) of the alkyl, alkenyl and alkylene groups may each optionally contain one or more groups selected from the following: —O—, —S—, —NR7-, —C(O)—, —OC(O)—, —C(O)O—, —SC(S)—, —C(S)S—, C3-8 Carbon ring.

R7 each is independently selected from H, C1-C12 Alkyl, C2-C12 Alkenyl, C1-C12 Alkoxy, carboxylic acid, sulfinic acid, sulfonic acid, sulfonyl, nitro, cyano, amino, carbamoyl, sulfonamide, C6-C10 Aryl, 5-10 membered heteroaryl, 3-10 membered heterocycle, halogen, C3-8 Carbocycle; preferably, R7 Independently selected from H, C1-C12 Alkyl, C2-C12 Alkenyl, C1-C12 Alkoxy, carboxylic acid, sulfinic acid, sulfonic acid, sulfonyl, nitro, cyano, amino, carbamoyl, sulfonamide, C6-C10 Aryl and 5-10 membered heteroaryl. The above groups (when appropriate, e.g. H, C1-C12 Alkyl, C2-C12 Alkenyl, C1-C12 Alkoxy, carboxylic acid, sulfinic acid, sulfonic acid, sulfonyl, amino, carbamoyl, sulfonamide, C6-C10 Aryl, 5-10 membered heteroaryl, 3-10 membered heterocycle, C3-8 carbocyclic rings) are each optionally replaced by one or more C1-C12 Alkyl, C2-C12 Alkenyl, C1-C12 Alkoxy, C6-C10 Aryl, 5-10 membered heteroaryl, 3-10 membered heterocycle, halogen, hydroxyl, oxo (═O) substitution.

In the compound of formula (I), m and n can each be independently an integer selected from 0-12, for example, 0, 1, 2, 3, 4, 5, 6, 7, 8, 9, 10, 11, 12. When taken as 0, it means that the corresponding group does not exist.

In one embodiment, M1 or M2 is a key, the corresponding m or n is not 0, M1 or M2 The first carbon chain and the corresponding R1 or R2 connect.

In one embodiment, m or n is 0, corresponding to M1 or M2 Not a bond, N atom is directly connected to M1 or M2 connected.

In one embodiment M1 or M2 is a bond, the corresponding m or n is 0, and the N atom is directly connected to the corresponding R1 or R2 connected.

In one embodiment, M1 and M2 each is independently selected from —C(O)—, —OC(O)—, —C(O)O— and —OC(O)O—. In yet another embodiment, M1 and M2 each independently selected from —NR7-, R7 As mentioned above.

In another embodiment, R5 with M1 and M2 One of them together with the connected N atom forms a 3-10 membered heterocyclic ring, and the corresponding R1/R3 or R2/R4 is not present, the heterocycle is optionally replaced by R7 Replacement, R7 As mentioned above.

In one embodiment, R5 selected from —C1-12 Alkylene-Q, Q is selected from H, —OR7, —OC(O)R7, —C(O)OR7, —N(R7)C(O)R7, —N(R7)2, cyano group, R7 As mentioned above.

In a preferred embodiment, R1 and R2 each is independently selected from C1-C12 Alkyl and C2-C12 Alkenyl; where R3 and R4 each is independently selected from C1-C12 Alkyl and C2-C12 Alkenyl; and R3 and R4 each independently selects t R6 Replace, t is an integer selected from 1-5; R6 each is independently selected from C1-C12 Alkyl and C2-C12 alkenyl.

M1 and M2 each is independently selected from —OC(O)—, —C(O)O—, —OC(O)O—, —SC(S)— and —C(S)S—; R5 selected from —C1-12 Alkylene-Q, Q is selected from —OR7 and —SR7, R7 Independently selected from H, C1-C12 Alkyl, C2-C12 Alkenyl, C1-C12 Alkoxy, carboxylic acid, sulfinic acid, sulfonic acid, sulfonyl, nitro, cyano, amino, carbamoyl, sulfonamide, C6-C10 Aryl and 5-10 membered heteroaryl;
m and n are each independently an integer selected from 1-12.

In a preferred embodiment, the cationic lipid comprises a lipid compound having the structure shown below or a pharmaceutically acceptable salt thereof:

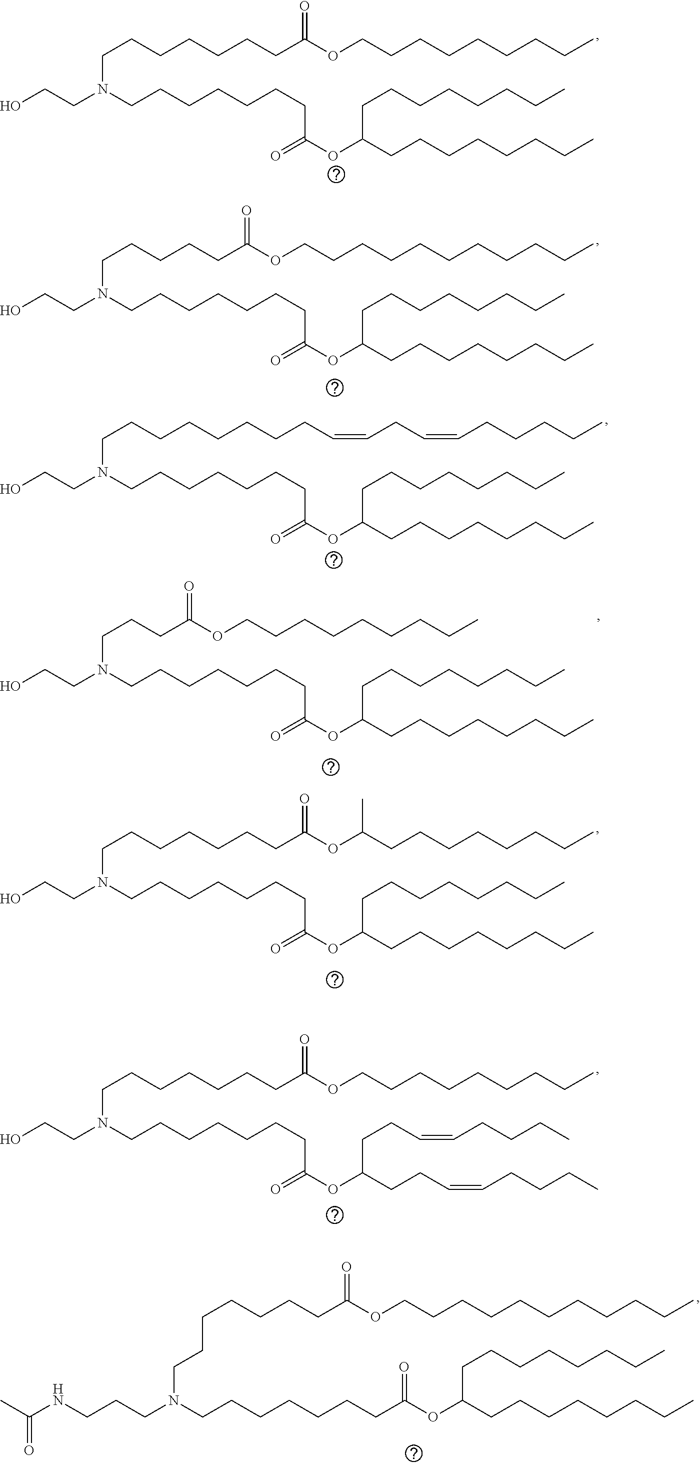

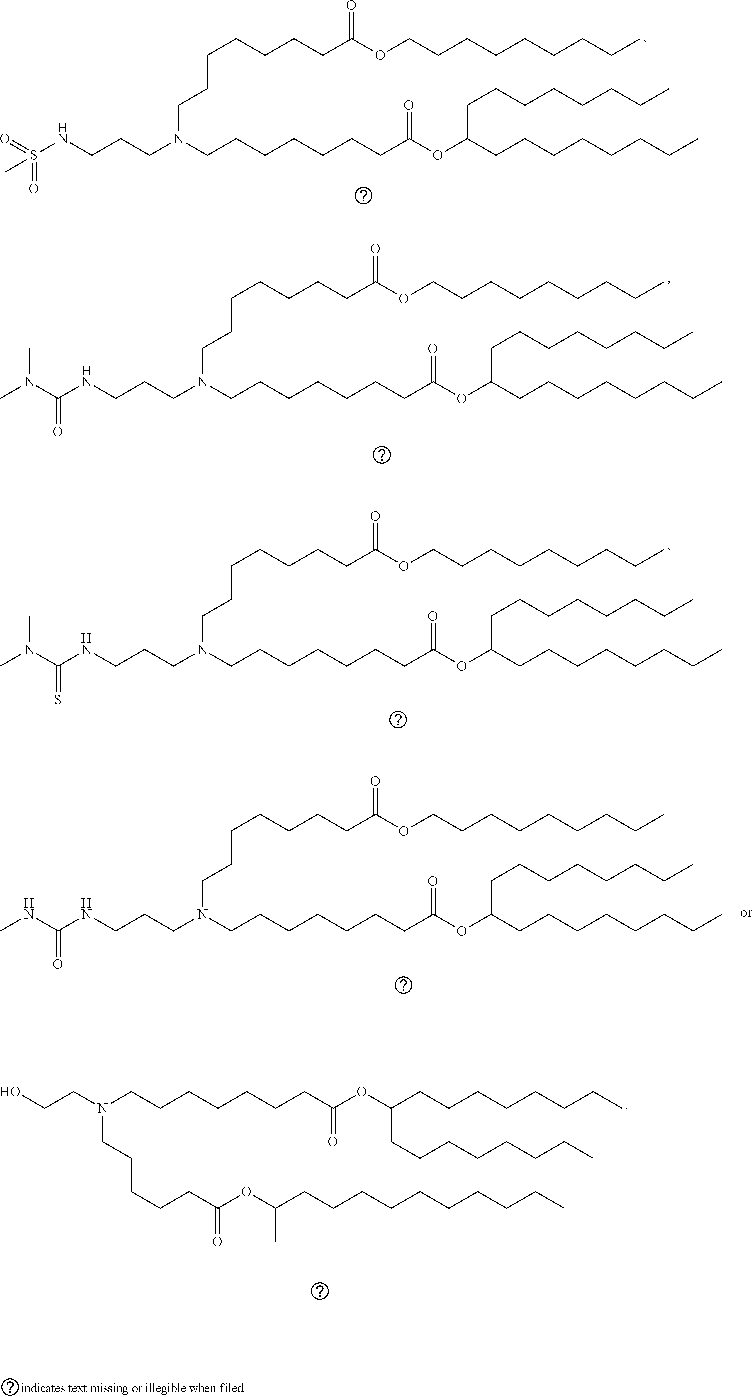

In a preferred embodiment, the cationic lipid contains M5 or SM-102.

In a preferred embodiment, the cationic lipid comprises a lipid compound having the structure shown below or a pharmaceutically acceptable salt thereof:

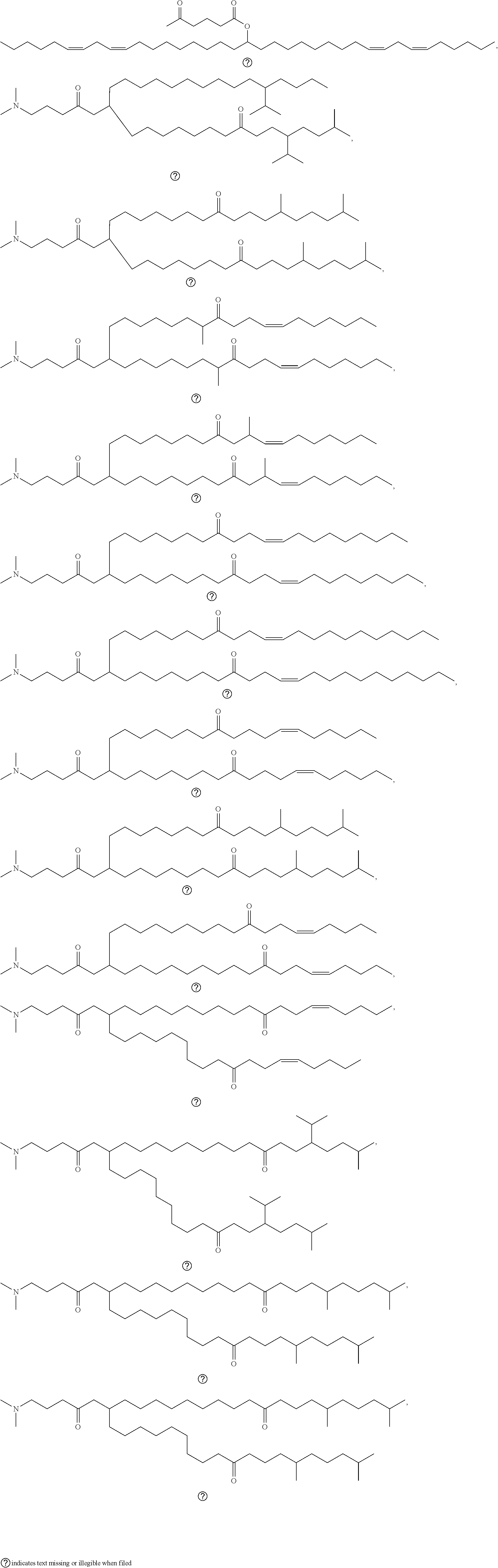

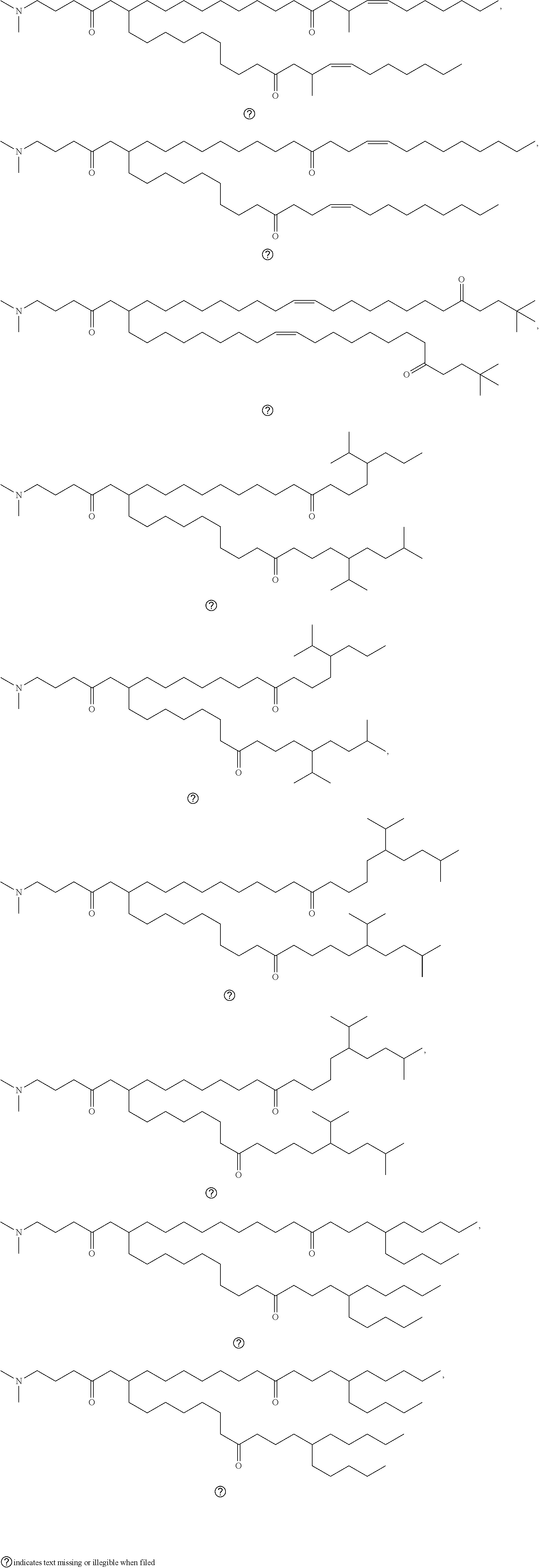

In a preferred embodiment, the cationic lipid comprises MC3.

In a preferred embodiment, the cationic lipid comprises a lipid compound having the structure shown below or a pharmaceutically acceptable salt thereof:

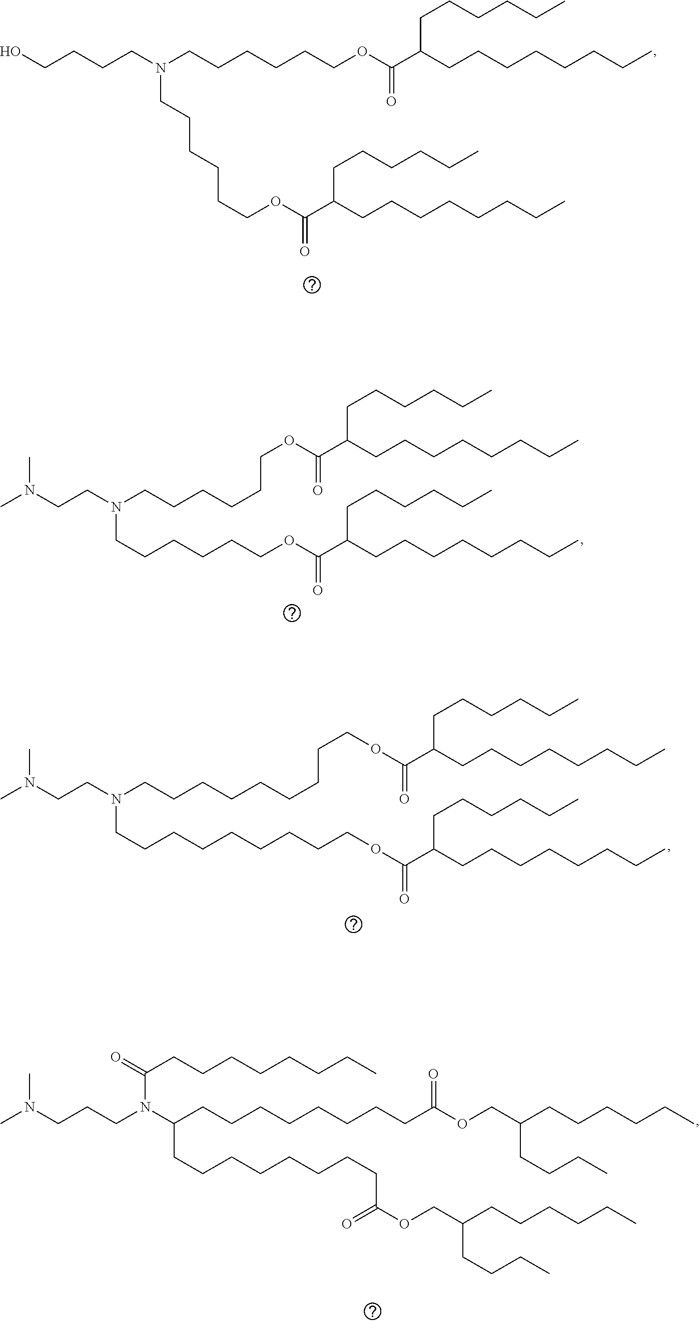

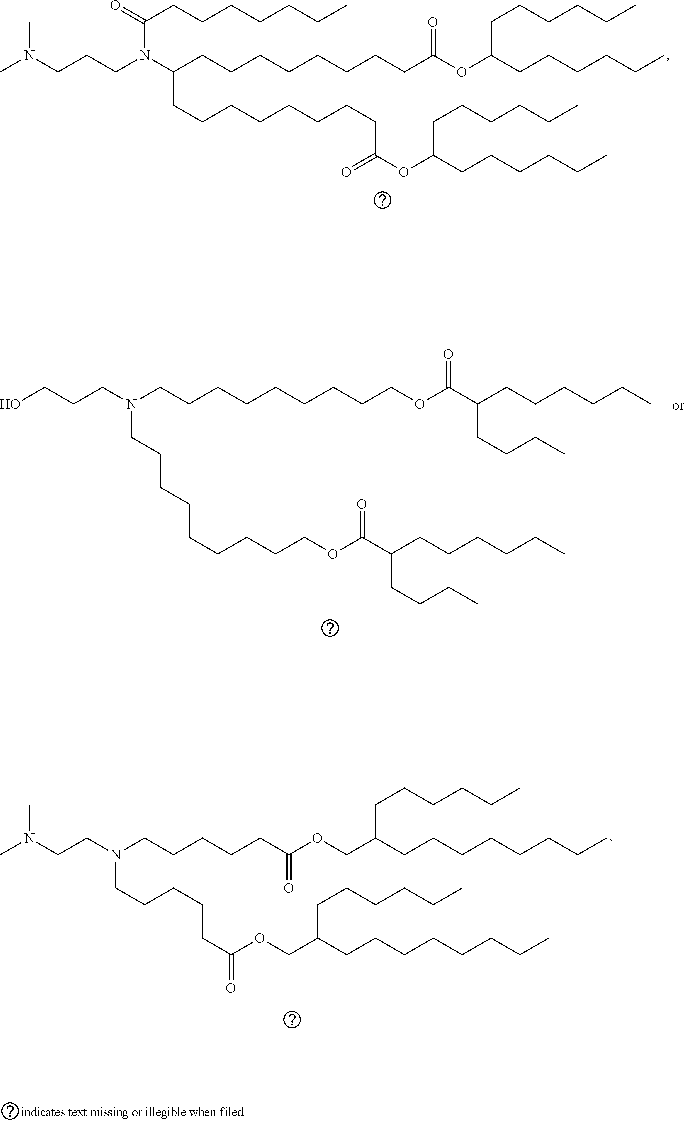

In a preferred embodiment, the cationic lipid contains ALC-0315.

In one embodiment, the cationic lipid comprises a compound of formula (I) or a pharmaceutically acceptable salt thereof:

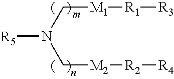

R1 and R2 each is independently selected from C1-C12 Alkyl and C2-C12 Alkenyl;
R3 and R4 each is independently selected from C1-C12 Alkyl, C2-C12 Alkenyl, C6-C10 Aryl and 5-10 membered heteroaryl; The condition is R3 and R4 At least one of them is C6-C10 Aryl or 5-10 membered heteroaryl, and R3 and R4 each independently selects t R6 Replace, t is an integer selected from 1-5; R6 each is independently selected from C1-C12 Alkyl and C2-C12 Alkenyl; M1 and M2 each is independently selected from —OC(O)—, —C(O)O—, —OC(O)O—, —SC(S)— and —C(S)S—;
R5 selected from —C1-12 Alkylene-Q, Q is selected from —OR7 and —SR7, R7 Independently selected from H, C1-C12 Alkyl, C2-C12 Alkenyl, C1-C12 Alkoxy, carboxylic acid, sulfinic acid, sulfonic acid, sulfonyl, nitro, cyano, amino, carbamoyl, sulfonamide, C6-C10 Aryl and 5-10 membered heteroaryl;
m and n are each independently an integer selected from 1-12.

In one embodiment, R2 Selected from C1-C12 alkyl. In another embodiment, R2 Selected from C1-C6 alkyl.

In one embodiment, R3 and R4, one is C6-C10 Aryl or 5-10 membered heteroaryl, the other is C1-C12 Alkyl or C₂-C12 alkenyl.

In a specific embodiment, R3 and R4 each is independently selected from C1-C12 Alkyl and phenyl, provided R3 and R4 At least one of them is phenyl. In another embodiment, R3 and R4 One is phenyl and the other is C1-C12 alkyl.

In yet another embodiment, R3 and R4 each independently by t R6 Instead, t is an integer selected from 1-5; for example, 1, 2, 3, 4 or 5. Preferably, t is an integer from 1 to 3, such as 1, 2 or 3, especially 1 or 2.

In one embodiment, R6 each is independently selected from C1-C12 Alkyl, such as C1-C10 alkyl.

In one embodiment, t is 1 and R6 Substituted on the benzene ring relative to R1 or R2 of metaposition or counterposition.

In another embodiment, t is 2, R6 Substituted on the benzene ring relative to R1 or R2 Metaposition and counterposition.

In one embodiment, R4 Replaced by R2 The 1st or last digit. The 1 bit refers to R2 Medium and M2 The position of the directly connected C atom. The last bit refers to R2 Medium and M2 The position of the farthest C atom. In a specific embodiment, R4 Selected from C1-C12 Alkyl, R3 is phenyl.

In one embodiment, R3 Replaced by R1 The 1st or last digit. The 1 bit refers to R1 Medium and M1 The position of the C atom directly connected to. The last bit refers to R1 Medium and M1 The position of the farthest C atom. In a specific embodiment, R3 Selected from C1-C12 Alkyl, R4 is phenyl.

In one embodiment, M1 and M2 each is independently selected from —OC(O)—, —C(O)O— and —OC(O)O—.

In one embodiment, R5 selected from —C1-5 Alkylene-Q, e.g. C1, C2, C3, C4 or C5 Alkylene-Q. In an exemplary embodiment, R5 selected from —C1-3 Alkylene-Q, e.g. C1, C2 or C3 Alkylene-Q.

In another embodiment, Q is selected from —OH and —SH, especially —OH.

In some embodiments, m and n are each independently an integer selected from 2-9, such as 2, 3, 4, 5, 6, 7, 8, or 9. Preferably, m and n are each independently an integer selected from 2-7, such as 2, 3, 4, 5, 6 or 7. More preferably, m and n are each independently an integer selected from 5-7, such as 5, 6 or 7.

In certain embodiments, compounds of Formula (I) include compounds of Formula (II):

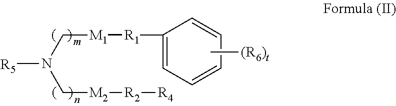

or a pharmaceutically acceptable salt thereof, wherein each group is as defined herein.

In one embodiment,
R1 Selected from C1-C6 alkyl;
R2 Selected from C1-C10 alkyl;
R4 Selected from C1-C10 alkyl; M1 and M2 each is independently selected from —OC(O)—, —C(O)O— and —OC(O)O—;
R5 selected from —C1-5 Alkylene-Q, Q is selected from —OR7 and —SR7, R7 Independently selected from H, C1-C12 Alkyl and C2-C12 Alkenyl;
R6 each is independently selected from C1-C12 Alkyl and C2-C12 Alkenyl, especially C1-C12 alkyl;
m and n are each independently an integer selected from 2-9, such as 2, 3, 4, 5, 6, 7, 8 or 9;
t is an integer selected from 1-3.

In one embodiment, R5 selected from —C1-3 Alkylene -Q, Q is selected from —OH and —SH, especially —OH.

In one embodiment, m and n are each independently an integer selected from 2-7, such as 2, 3, 4, 5, 6 or 7.

In some embodiments, t is 1 or 2.

In one embodiment, R4 Replaced by R2 The 1st or last digit. The 1 bit refers to R2 Medium and M2 The position of the directly connected C atom. The last bit refers to R2 Medium and M2 The position of the farthest C atom.

In one embodiment, t is 1 and R6 Substituted on the benzene ring relative to R1 of metaposition or counterposition.

In another embodiment, t is 2, R6 Substituted on the benzene ring relative to R1 Metaposition and counterposition.

In certain embodiments, compounds of Formula (I) include compounds of Formula (III):

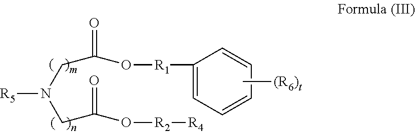

or a pharmaceutically acceptable salt thereof, wherein each group is as defined herein.

In one embodiment,
R1 Selected from C1-C6 alkyl;
R2 Selected from C1-C10 alkyl;
R4 Selected from C1-C10 alkyl;
R5 selected from —C1-3 Alkylene -Q, Q is selected from —OH and —SH, especially —OH;
t is 1 or 2;
R6 Selected from C1-C12 Alkyl and C2-C12 Alkenyl, especially C1-C12 alkyl;
m and n are each independently an integer selected from 2-7, such as 2, 3, 4, 5, 6 or 7.

In one embodiment, R4 Replaced by R2 The 1st or last digit. The 1 bit refers to

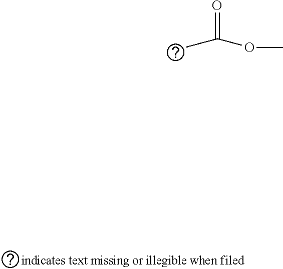

in R2 the position of some directly connected C atoms. The last bit refers to

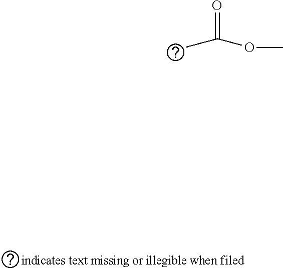

in R2 The position of the most distant C atom.

In one embodiment, t is 1 and R6 Substituted on the benzene ring relative to R1 of metaposition or counterposition.

In another embodiment, t is 2, R6 Substituted on the benzene ring relative to R1 Metaposition and counterposition.

In certain embodiments, compounds of Formula (I) include compounds of Formula (IV):

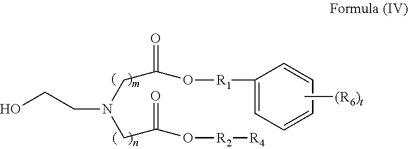

or a pharmaceutically acceptable salt thereof, wherein each group is as defined herein.

In one embodiment,
R1 Selected from C1-C6 alkyl;
R2 Selected from C1-C10 alkyl;
R4 Selected from C1-C10 alkyl;
t is 1 or 2;
R6 each is independently selected from C1-C12 Alkyl and C2-C12 Alkenyl, especially C1-C12 alkyl;
m and n are each independently an integer selected from 2-7, such as 2, 3, 4, 5, 6 or 7.

In one embodiment, R4 Replaced by R2 The 1st or last digit. The 1 bit refers to

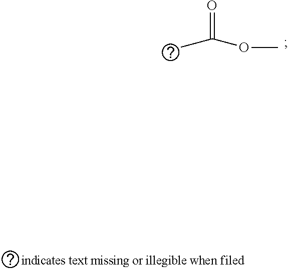

in R2 Position of some directly connected C atoms. The last bit refers to

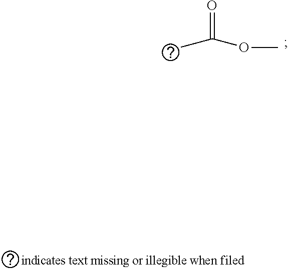

R2 The position of the most distant C atom.

In one embodiment, t is 1 and R6 Substituted on the benzene ring relative to R1 of metaposition or counterposition.

In another embodiment, t is 2, R6 Substituted on the benzene ring relative to R1 Metaposition and counterposition.

In a specific embodiment, the substituents in the lipid compounds of the invention (e.g., R1-R7) does not contain an alkenyl group.

In a preferred embodiment, the cationic lipid comprises a lipid compound having the structure shown below or a pharmaceutically acceptable salt thereof:

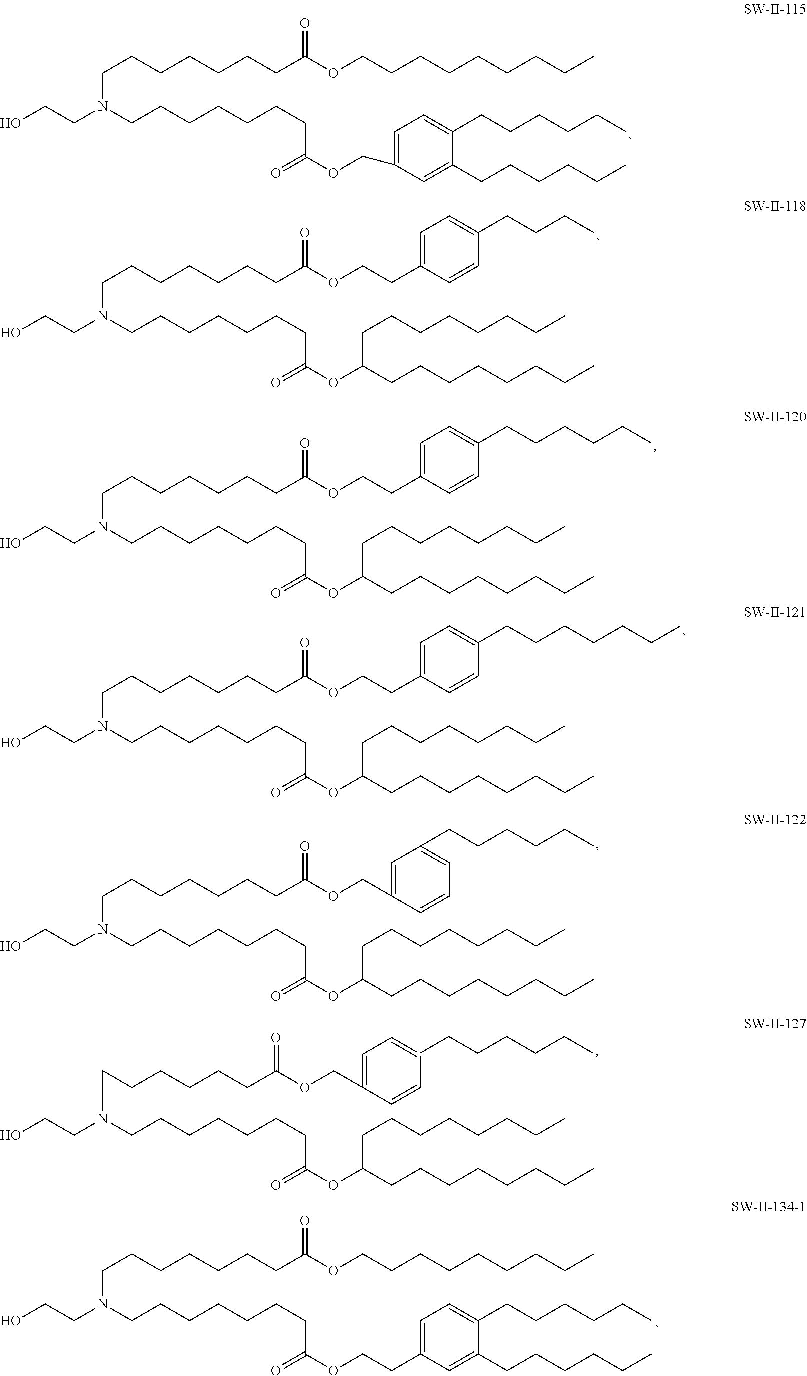

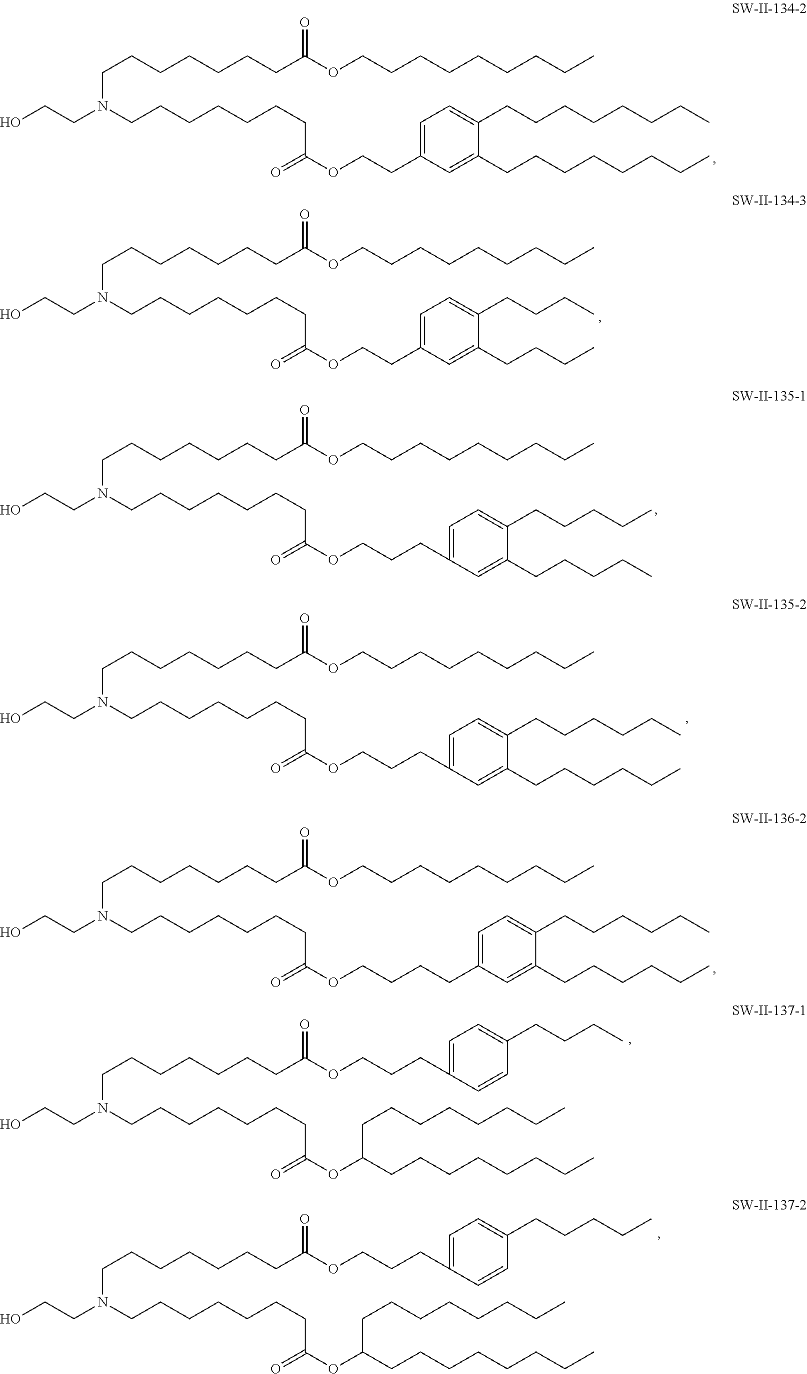

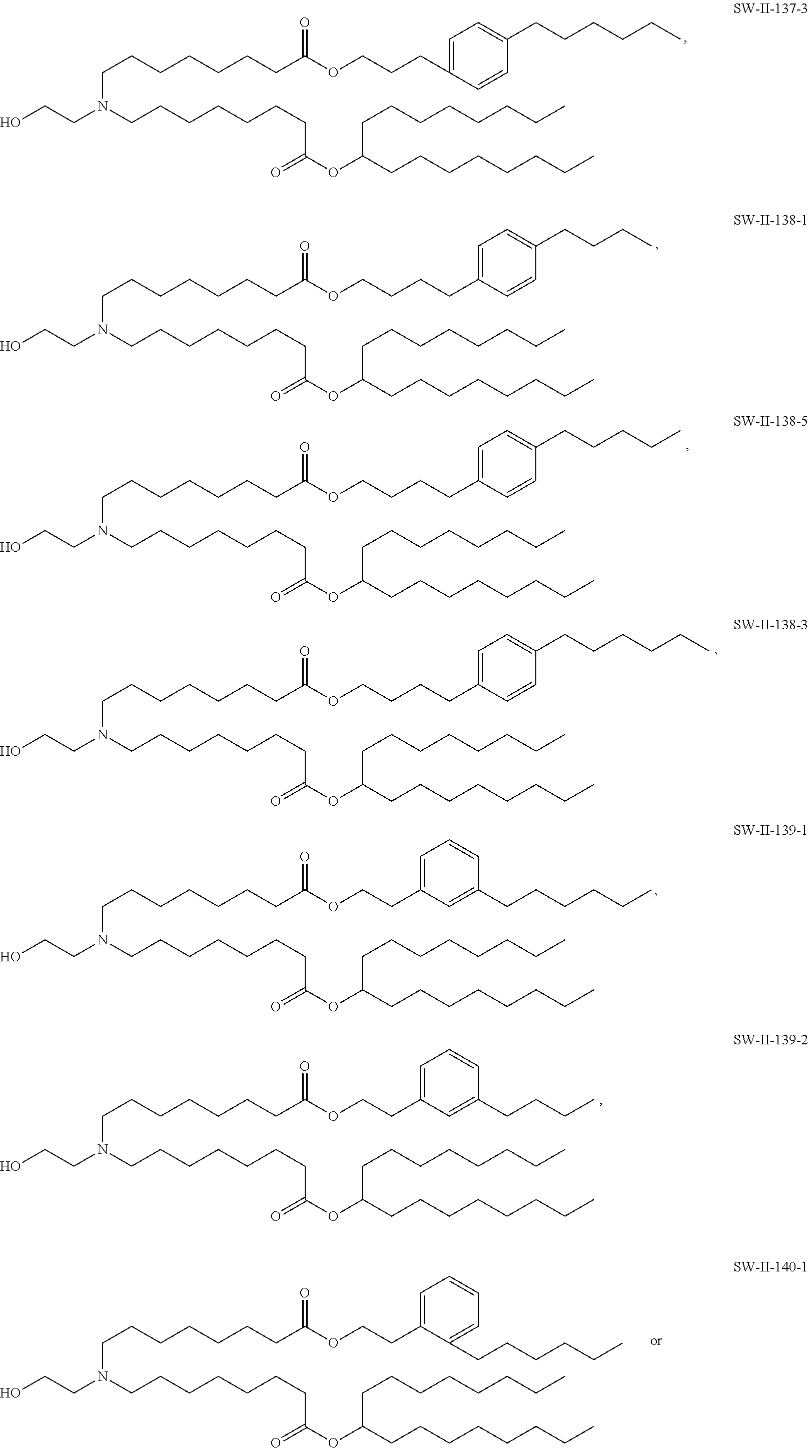

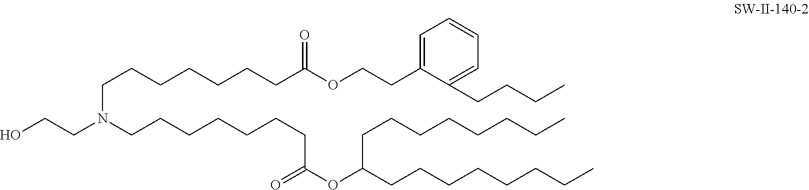 [SMILES: CCCCCCCCC(CCCCCCCC)OC(=O)CCCCCCCN(CCO)CCCCCCCC(=O)OCCc1ccccc1CCCC]

In a preferred embodiment, the cationic lipids include the following lipid compounds: SW-II-115, SW-II-121, SW-II-122, SW-II-134-3, SW-II-138-2, SW-II-139-2 or SW-II-140-2.

In a preferred embodiment, the cationic lipids comprise the following lipid compounds: M5, MC3, ALC-0315, SM-102, SW-II-115, SW-II-121, SW-II-122, SW-II-134-3, SW-II-138-2, SW-II-139-2 or SW-II-140-2.

### Phospholipids

The lipid composition of the present invention contains phospholipids, which can assist cell penetration of the lipid composition. Examples of phospholipids include, but are not limited to: 1,2-dilinoleoyl-sn-glycero-3-phosphocholine (DLPC), 1,2-dimyristoyl-sn-glycero-phosphocholine (DMPC), 1,2-Dioleoyl-sn-glycero-3-phosphocholine (DOPC), 1,2-dioleoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycerol-3-Phosphocholine (POPC), 1,2-di-O-octadecenyl-sn-glycero-3-phosphocholine (18:0Diether PC), 1-oleoyl-2-cholesteryl hemisuccinyl-sn-glycero-3-phosphocholine (OChemsPC), 1-hexadecyl-sn-glycero-3-phosphocholine (C16Lyso PC), 1,2-dilinolenoyl-sn-glycero-3-phosphate Choline, 1,2-diarachidonoyl-sn-glycero-3-phosphocholine, 1,2-bidocosahexaenoyl-sn-glycero-3-phosphocholine, 1,2-Dioleoyl-sn-glycero-3-phosphoethanolamine (DOPE), 1,2-diphytanoyl-sn-glycero-3-phosphoethanolamine (ME 16.0 PE), 1,2-distearoyl-sn-Glycerol-3-phosphoethanolamine, 1,2-dilinoleoyl-sn-glycero-3-phosphoethanolamine, 1,2-dilinoleoyl-sn-glycerol-3-phosphoethanolamine, 1,2-diarachidonene Acyl-sn-glycero-3-phosphoethanolamine, 1,2-bidocosahexaenoyl-sn-glycerol-3-phosphoethanolamine, 1,2-dioleoyl-sn-glycerol-3-phosphoethanolamine, rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoylphospholipid Acetylethanolamine (DPPE), dimyristoylphosphoethanolamine (DMPE), 1-stearoyl-2-oleoyl-stearoylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoylphosphatidylcholine, lysophosphatidylcholine, lysophosphatidylethanolamine (LPE) or combination thereof.

### Steroid

Steroids are included in the lipid compositions of the present invention and can serve as structural components of the lipid compositions.

Examples of steroids include, but are not limited to, cholesterol, coprosterol, sitosterol, ergosterol, campesterol, stigmasterol, campesterol, tomatine, ursolic acid, alpha-tocopherol, and derivatives thereof.

### Polyethylene Glycol Modified Lipids

As used herein, the term “polyethylene glycol modified lipid” or “PEG modified lipid” or “PEG lipid” refers to a molecule containing a polyethylene glycol moiety and a lipid moiety that is modified with polyethylene glycol. Alcohol-modified lipids. The PEG lipid may be selected from the non-limiting group consisting of: PEG-modified phosphatidylethanolamine, PEG-modified phosphatidic acid, PEG-modified ceramide (PEG-CER), PEG-modified dialkylamine, PEG-modified diacylglycerol (PEG-DEG), PEG-modified dialkylglycerol, or combinations thereof. For example, examples of polyethylene glycol-modified lipids include, but are not limited to: 1,2-dimyristoyl-rac-glycerol-3-methoxypolyethylene glycol (1,2-dimyristoyl-rac-glycero-3-methoxypolyethylene glycol, DMG-PEG), 1,2-Dioleoyl-rac-glycerol, methoxypolyethylene glycol (1,2-Dioleoyl-rac-glycerol, methoxypolyethylene Glycol, DOGPEG) and 1,2-Distearoyl-sn-glycero-3-phosphoethanolamine-Poly(ethylene glycol), DSPE-PEG.

In one embodiment, the polyethylene glycol modified lipid is DMG-PEG, such as DMG-PEG 2000. In one embodiment, DMG-PEG 2000 has the following structure:

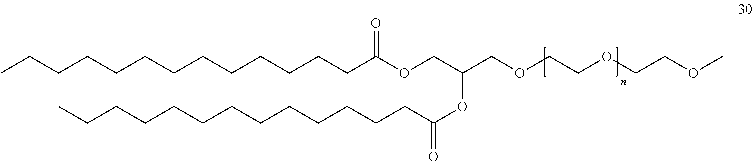

where the average value of n is 44.

### Cationic Polymer

As used herein, the term “cationic polymer” refers to any ionic polymer capable of carrying a net positive charge at a specified pH to electrostatically bind to nucleic acids. Examples of cationic polymers include, but are not limited to: poly-L-lysine, protamine, polyethylenimine (PEI), or combinations thereof. The polyethyleneimine may be linear or branched polyethyleneimine. The term “protamine” refers to an arginine-rich low molecular weight basic protein that is present in the sperm cells of various animals (especially fish) and binds to DNA instead of histones. In a preferred embodiment, the cationic polymer is protamine (eg protamine sulfate).

### Pharmaceutical Composition

The present invention also provides a pharmaceutical composition, which contains the artificial nucleic acid molecule of the present invention, the carrier of the present invention, the cell of the present invention or the lipid composition of the present invention, and a pharmaceutically acceptable carrier.

Pharmaceutically acceptable carriers may include, but are not limited to: diluents, binders and adhesives, lubricants, disintegrants, preservatives, vehicles, dispersants, glidants, sweeteners, coatings, excipients, etc. Excipients, preservatives, antioxidants (such as ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, Propyl gallate, alpha-tocopherol, citric acid, ethylenediaminetetraacetic acid (EDTA), sorbitol, tartaric acid, phosphoric acid, etc.), solubilizer, gelling agent, softener, solvent (e.g., water, alcohol, acetic acid and syrup), buffers (e.g., phosphate buffers, histidine buffers, and acetate buffers), surfactants (e.g., nonionic surfactants such as polysorbate 80, polysorbate 20, Poloxamers or polyethylene glycols), antibacterial agents, antifungal agents, isotonic agents (e.g. trehalose, sucrose, mannitol, sorbitol, lactose, glucose), absorption delaying agents, chelating agents and emulsifiers. For pharmaceutical compositions containing artificial nucleic acid molecules, vectors, cells or lipid compositions, suitable carriers may be selected from buffers (e.g. citrate buffer, acetate buffer, phosphate buffer, etc. acid buffer, histidine salt buffer), isotonic agents (such as trehalose, sucrose, mannitol, sorbitol, lactose, glucose), nonionic surfactants (such as polysorbate 80, polysorbate 20 poloxamer) or combinations thereof.

Pharmaceutical compositions provided herein may be in a variety of dosage forms, including, but not limited to, solid, semi-solid, liquid, powder, or lyophilized forms. For pharmaceutical compositions containing artificial nucleic acid molecules, carriers, cells or lipid compositions, preferred dosage forms may generally be, for example, injection solutions and lyophilized powders.

The pharmaceutical compositions provided herein may be administered to a subject by any method known in the art, such as by systemic or local administration. Routes of administration include, but are not limited to, parenteral (e.g., intravenous, intraperitoneal, intradermal, intramuscular, subcutaneous, or intracavity), topical (e.g., intratumoral), epidural, or mucosal (e.g., intranasal, oral, vaginal, rectal, sublingual or local). Preferably, the pharmaceutical composition is suitable for intravenous, intramuscular, subcutaneous, parenteral, spinal or epidermal administration (eg by injection or infusion). The method of administration may be, for example, injection or infusion.

### Treat

In yet another aspect, the invention relates to the use of the artificial nucleic acid molecule of the invention, the vector of the invention, the cell of the invention, the lipid composition of the invention or the pharmaceutical composition of the invention in the preparation of a vaccine or a medicament for gene therapy. the use of.

In yet another aspect, the invention relates to the use of the artificial nucleic acid molecule of the invention, the vector of the invention, the cell of the invention, the lipid composition of the invention or the pharmaceutical composition of the invention in the preparation of a medicament for treating or preventing diseases. the use of.

The artificial nucleic acid molecules of the invention, the vectors of the invention, the cells of the invention, the lipid compositions of the invention or the pharmaceutical compositions of the invention can be used to treat diseases, disorders or conditions. Specifically, the artificial nucleic acid molecules of the invention, the vectors of the invention, the cells of the invention, the lipid compositions of the invention or the pharmaceutical compositions of the invention can be used to treat diseases characterized by missing or abnormal protein or polypeptide activity.

Disease, disease or condition. For example, artificial nucleic acid molecules, vectors, cells, lipid compositions, or pharmaceutical compositions containing mRNA encoding a missing or aberrant polypeptide can be administered or delivered to the cell. Subsequent translation of the mRNA can produce the polypeptide, thereby reducing or eliminating problems caused by the absence or abnormal activity of the polypeptide. Because translation can occur rapidly, these methods and artificial nucleic acid molecules, vectors, cells, lipid compositions or pharmaceutical compositions are used to treat acute diseases, disorders or conditions such as sepsis, stroke and myocardial infarction.

Diseases, disorders or conditions characterized by dysfunction or abnormal protein or polypeptide activity to which the artificial nucleic acid molecules, vectors, cells, lipid compositions or pharmaceutical compositions of the present invention may be administered include, but are not limited to, rare diseases, infectious diseases (in the form of vaccines and therapeutics), cancer and proliferative diseases, genetic diseases (such as cystic fibrosis), autoimmune diseases, neurodegenerative diseases, cardiovascular and renovascular diseases, and metabolic diseases. There are a variety of diseases, disorders or conditions that can be characterized by a loss of protein activity (or a substantial reduction such that appropriate protein function cannot occur). These proteins may not be present, or they may be essentially non-functional. Specific examples of dysfunctional proteins are missense mutant variants of the cystic fibrosis transmembrane conductance regulator (CFTR) gene, which produce dysfunctional protein variants of the CFTR protein, thereby causing cystic fibrosis. The invention provides a method of treating such diseases, disorders or conditions in a subject by administering an artificial nucleic acid molecule, vector, cell, lipid composition or pharmaceutical composition of the invention, wherein the RNA may encode an antagonist or The mRNA of a polypeptide that otherwise overcomes the abnormal protein activity present in the subject's cells.

The artificial nucleic acid molecules, vectors, cells, lipid compositions, or pharmaceutical compositions of the invention may be administered to a subject in any reasonable amount and by any route of administration that is effective in achieving cure of the disease, disorder, or condition. prevention, treatment, diagnosis or for any other purpose. The specific amount administered to a given subject may vary depending on the species, age, and general condition of the subject; the purpose of administration; the specific composition; the mode of administration, and the like.

In some embodiments, the artificial nucleic acid molecules, vectors, cells, lipid compositions, or pharmaceutical compositions of the invention may be administered to a subject by any method known to those skilled in the art, such as parenterally, orally, or transmucosally. transcutaneous, intramuscular, intravenous, intradermal, subcutaneous or intraperitoneal.

### Reagent Test Kit

The present invention also provides a kit, which contains the artificial nucleic acid molecule, vector, cell, lipid composition or pharmaceutical composition of the present invention, and instructions for use. The kit may also contain suitable containers. In certain embodiments, the kit further includes a device for administering the drug. Kits generally include labels indicating the intended use and/or method of use of the contents of the kit. The term “label” includes any written or recorded material on or provided with the kit or otherwise provided with the kit.

### Beneficial Effects

The artificial nucleic acid molecules, vectors, cells, lipid compositions or pharmaceutical compositions of the present invention can exhibit excellent effects, such as but not limited to: 1) improving the translation efficiency of the included mRNA; and/or 2) included mRNA has high stability.

### Example

A further understanding of the invention may be obtained by reference to some of the specific examples given herein, which are merely illustrative of the invention and are not intended to limit the scope of the invention in any way. Obviously, various modifications and changes can be made to the present invention without departing from the essence of the invention. Therefore, these modifications and changes are also within the scope of protection claimed by this application. Proportions used herein include percentages and, unless otherwise stated, are by weight.

### Experimental Materials

The cationic lipid according to formula (I) is synthesized by microorganisms or prepared by reference, such as CN110520409A, WO2018081480A1 or U.S. Pat. No. 11,246,933B1; phospholipid (DOPE) is purchased from CordenPharma; cholesterol is purchased from Sigma-Aldrich; mPEG2000-DMG (i.e. DMG-PEG 2000) was purchased from Avanti Polar Lipids, Inc.; PBS was purchased from Invitrogen; protamine sulfate was purchased from Beijing Silian Pharmaceutical Co., Ltd.; mPEG2000-DSPE was purchased from lipoid GmbH; DSPC was purchased from Avanti Polar Lipids, Inc.

### Example 1 Synthesis of Compounds According to Formula (I)

### General Considerations

Unless otherwise stated, all solvents and reagents used were commercially available and used as received. 1H NMR spectra were obtained at 300K using a Bruker Ultrashield 300 MHz instrument at CDCl3 record. Chemical shifts are about 1H is reported in parts per million (ppm) relative to TMS (0.00). Silica gel chromatography was performed on an ISCO CombiFlash Rf+Lumen instrument using an ISCO RediSep Rf Gold column (particle size: 20-40 μm).

The procedure described below can be used to synthesize compounds SW-II-115 to SW-II-140-2.

This article uses the following abbreviations:
THF: Tetrahydrofuran
MeCN: acetonitrile
LAH: lithium aluminum hydride
DCM: dichloromethane
DMAP: 4-dimethylaminopyridine
LDA: lithium diisopropylamide
rt: room temperature
DME: 1,2-Dimethoxyethane
n-BuLi: n-butyllithium
CPME: cyclopentyl methyl ether
EDCI: N-(3-dimethylaminopropyl)-N′-ethylcarbodiimide
DIEA: N,N-diisopropylethylamine
PE: Petroleum ether
EA: Ethyl acetate

### A. Compound SW-II-115

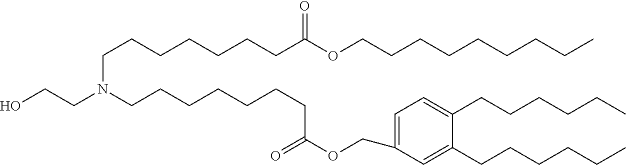 [SMILES: CCCCCCCCCOC(=O)CCCCCCCN(CCO)CCCCCCCC(=O)OCc1ccc(CCCCCC)c(CCCCCC)c1]

### 1. Synthesis of Intermediate 3

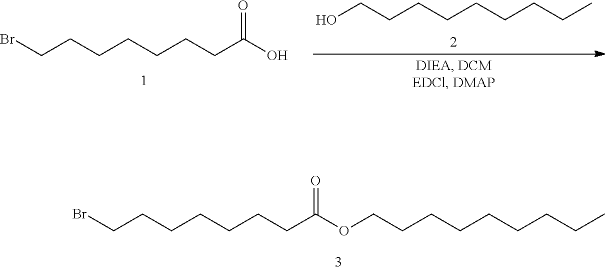

To the DCM solution (100 mL) containing compound 1 (10 g, 45 mmol, 1 eq.) and compound 2 (7.8 g, 54 mmol, 1.2 eq.), EDCI (17.3 g, 90 mmol, 2 eq.) and DMAP (2.2 g, 18 mmol, 0.4 eq.), then add DIEA (23.2 g, 180 mmol, 4 eq.). The reaction mixture was brought to room temperature in N2 Stir under protection for 16 hours. TLC (petroleum ether:ethyl acetate=30:1) showed that compound 1 was consumed and the desired product was formed. The reaction mixture was diluted with DCM (20 mL) and washed with H2O (40 mL), washed with anhydrous Na2SO4 Dry, filter and concentrate under reduced pressure. The residue was purified by silica gel column chromatography, eluting with petroleum ether:ethyl acetate (1:0-20:1), to obtain compound 3 (4.365 g, 28%) as a colorless oil.

### 2. Synthesis of Intermediate 5

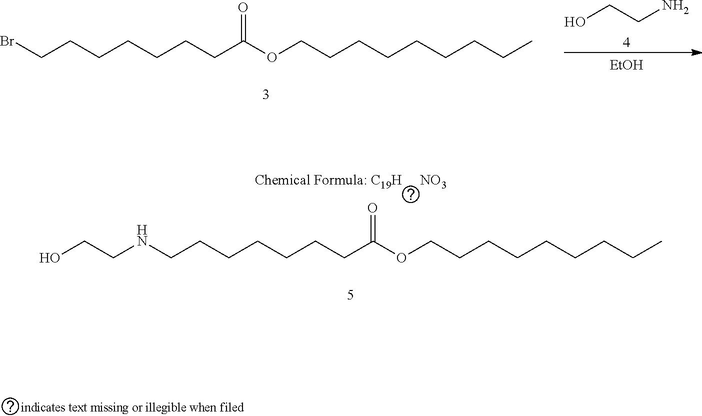

The EtOH solution of compound 3 (500 mg, 1.437 mmol, 1 eq.) and compound 4 (2.63 g, 43.103 mmol, 30 eq.) was dissolved in N₂ Stir at 60° C. for 16 hours under protection. TLC (DCM:MeOH=10:1) showed that compound 3 was consumed and TLC (DCM/MeOH=10/1) showed that a new major spot was observed. The reaction mixture was concentrated under reduced pressure. The residue was diluted with EtOAc (50 mL) and washed with H2 Wash with 0 (3×50 mL). Anhydrous Na was used for the organic layer Na2SO4 Dry, filter and concentrate under reduced pressure. The residue was purified by silica gel column chromatography, eluting with DCM/MeOH (1:0-10:1, v/v), to obtain compound 5 (264 mg, 56%) as a yellow oil. 3. Synthesis of intermediate 8

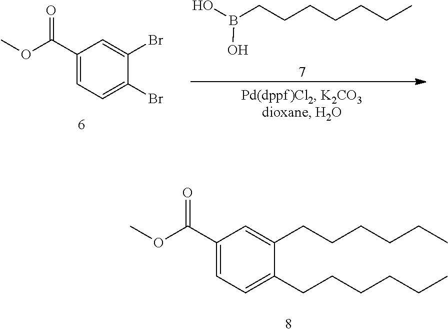

Pd(dppf)Cl was added to compound 6 (500 mg, 1.712 mmol, 1 eq.) and compound 7 (1.113 g, 8.562 mmol, 5 eq.) in dioxane/water (5 mL/0.5 mL) mixed solvent. 2(112 mg, 0.171 mmol, 0.1 eq.) and potassium carbonate (709 mg, 5.136 mmol, 3 eq.). Place the mixture in N2 Stir overnight at 100° C. TLC (PE:EA=15:1) showed the reaction was complete and a new major spot was observed. The mixture was extracted with EA and washed with water, and the organic layer was washed with anhydrous Na2SO4 Dry, filter and concentrate in vacuo. The residue was purified by silica gel column chromatography, eluting with PE:EA (1:0-10:1), to give compound 8 as a colorless oil (455 mg, 88%).

### 4. Synthesis of Intermediate 9

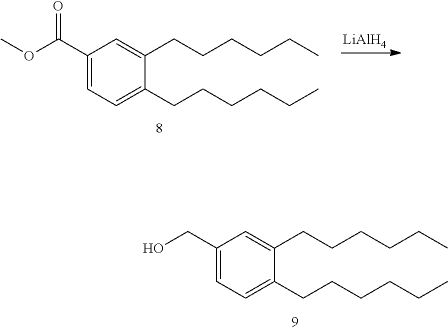 [SMILES: CCCCCCc1ccc(C(=O)OC)cc1CCCCCC ; "LiAlH4" ; CCCCCCc1ccc(CO)cc1CCCCCC]

at 0° C. and N2 Under protection, LiAlH was added to a solution of compound 8 (455 mg, 1.497 mmol, 1 eq.) in THF (5 mL). 4 (1.5 mL, 1.497 mmol, 1M in THF, 1 eq.). Bring the mixture to room temperature in N2 Stir for 2 hours. TLC (PE:EtOAc=5:1) showed the reaction was complete and a new major spot was observed. The mixture was quenched with water (1.5 mL) and treated with 2N HCl to adjust the pH between 6 and 7, extracted with EA and washed with brine. Anhydrous Na was used for the organic Na2SO4 Dry, filter and concentrate in vacuo to afford crude compound 9 (419 mg, >100%) as a colorless oil without further purification.

### 5. Synthesis of Intermediate 10

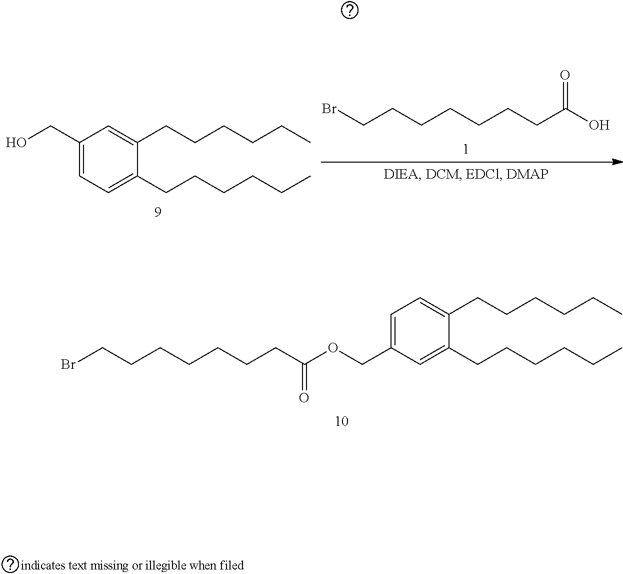

To the DCM (4 mL) solution containing compound 1 (339 mg, 1.518 mmol, 1 eq.) and compound 9 (419 mg, 1.518 mmol, 1 eq.), EDCI (583 mg, 3.036 mmol, 2 eq.) and DMAP (74 mg, 0.607 mmol, 0.4 eq.), then DIEA (783 mg, 6.072 mmol, 4 eq.) was added. The reaction mixture was brought to room temperature in N2 Stir under protection for 16 hours. TLC (petroleum ether:ethyl acetate=10:1) showed the formation of the desired product. The reaction mixture was extracted with EA and washed with water. Anhydrous Na was used for the organic Na2SO4 Dry, filter and concentrate in vacuo. The residue was purified by silica gel column chromatography, eluting with petroleum ether:ethyl acetate (1:0-10:1), to afford compound 10 (443 mg, 60.7%) as a colorless oil.

### 6. Synthesis of Final Product SW-II-115

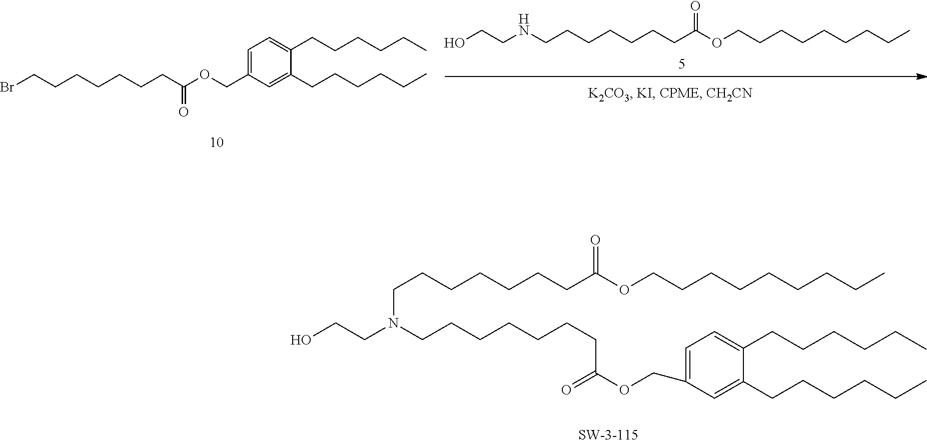

To the mixed solvent CPME/CH containing compound 10 (307 mg, 0.64 mmol, 1 eq.) and compound 5 (210 mg, 0.64 mmol, 1 eq.) 3 Add K to CN (3 mL/3 mL) 2CO3 (530 mg, 3.84 mmol, 6 eq.) and KI (212 mg, 1.28 mmol, 2 eq.). After the addition is complete, bring the mixture to N2 Stir overnight at 90° C. TLC (DCM:MeOH=10:1) showed the reaction was complete and a new major spot was observed. The mixture was extracted with EA and washed with water.

Anhydrous Na was used for the organic Na2SO4 Dry, filter and concentrate under reduced pressure. The residue was purified by silica gel column chromatography, eluting with DCM:MeOH (1:0-10:1, v/v), to obtain compound SW-II-115 (266 mg, 57%) as a yellow oil.

LCMS:Rt:1.293 min; MS m/z(ELSD):730.5[M+H]+;

HPLC: 99.472% purity, ELSD; RT=4.895 min.

¹H NMR (400 MHz, CDCl3) δ7.21-6.99 (m, 3H), 5.05 (s, 2H), 4.05 (t, J=6.8 Hz, 2H), 3.58 (t, J=5.3 Hz, 2H), 2.69-2.46 (m, 10H), 2.31 (dt, J=20.0, 7.5 Hz, 4H), 1.69-1.18 (m, 51H), 0.89 (dt, J=12.4, 6.3 Hz, 9H).

¹³C NMR (101 MHz, CDCl 3) δ173.90 (s), 173.68 (s), 140.80 (d, J=13.0 Hz), 133.31 (s), 129.25 (d, J=16.2 Hz), 128.30 (s), 125.75 (s), 77.30 (d, J=11.5 Hz), 77.04 (s), 76.72 (s), 66.22 (s), 64.43 (s), 58.12 (s), 55.72 (s), 53.90 (s), 34.32 (d, J=1.9 Hz), 32.69 (s), 32.48 (s), 31.81 (d, J=11.2 Hz), 31.25 (s), 29.59-28.91 (m), 28.66 (s), 27.17 (s), 26.64 (s), 25.94 (s), 24.91 (d, J=5.1 Hz), 22.65 (d, J=3.3 Hz), 14.10 (s).

### B. Compound SW-II-118.

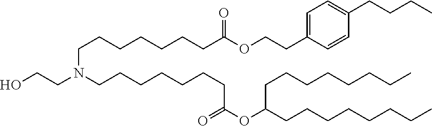 [SMILES: CCCCCCCCC(CCCCCCCC)OC(=O)CCCCCCCN(CCO)CCCCCCCC(=O)OCCc1ccc(CCCC)cc1]

1. Synthesis of intermediate 3

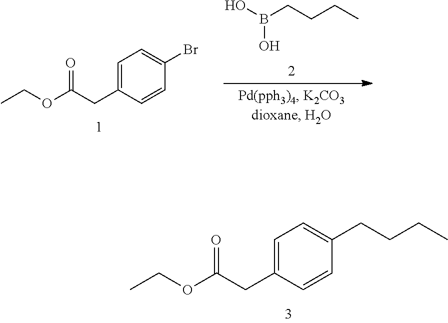

Compound 1 (1.22 g, 5.0 mmol, 1.0 eq.) and compound 2 (765 mg, 7.5 mmol, 1.5 eq.), Pd (PPh 3) 4 (Tetrakis triphenylphosphine palladium, 289 mg, 0.25 mmol, 0.05 eq.) and K2CO3 (1.38 g, 10.0 mmol, 2.0 eq.) of toluene (10 ml) and H2O (1 ml) solution at 110° C., N2 Stir protected for 1 hour. TLC (petroleum ether:ethyl acetate=19:1) showed that compound 1 was consumed and a new spot was observed. The reaction mixture was diluted with DCM (50 mL) and washed with H2O (40 mL), washed with anhydrous Na2SO4 Dry, filter and concentrate under reduced pressure.

The residue was purified by silica gel column chromatography, eluting with petroleum ether:ethyl acetate (1:0-10:1), to obtain compound 3 (0.5 g, 45%) as a colorless oil. 1H NMR (400 MHz, CDCl 3) δ7.16 (dd, J=23.5, 8.1 Hz, 4H), 4.14 (q, J=7.1 Hz, 2H), 3.57 (s, 2H), 2.64-2.48 (m, 2H), 1.66-1.51 (m, 2H), 1.35 (dd, J=15.0, 7.4 Hz, 2H), 1.25 (t, J=7.1 Hz, 3H), 0.92 (t, J=7.3 Hz, 3H).

### 2. Synthesis of Intermediate 4

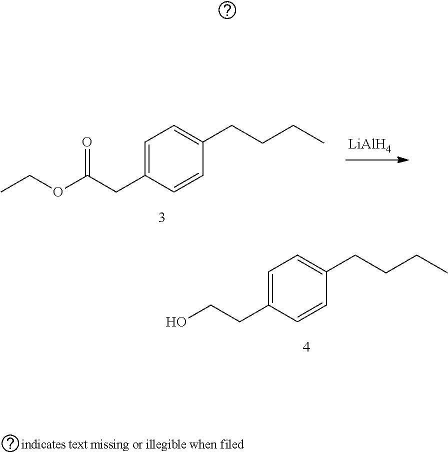

LiAlH at −78° C. 4 (193 mg, 5.09 mmol, 4.0 eq.) was added to a solution containing compound 3 (280 mg, 1.27 mmol, 1.0 eq.) in THF (10 mL), and the reaction was carried out at 10° C. for 3 hours. TLC showed that the reaction was very good, the reaction was concentrated and treated with Na2 SO4 (20 mL) was diluted and extracted with EA (30 mL×2), and the organic phase was treated with anhydrous Na2SO4 Dry, filter and concentrate under reduced pressure to obtain compound 4 (3.12 g, crude product) as a yellow oil.

### 3. Synthesis of Intermediate 6

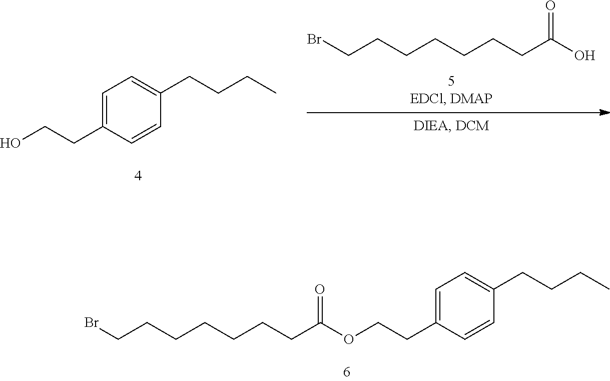 [SMILES: O=C(O)CCCCCCCBr ; CCCCc1ccc(CCO)cc1 ; CCCCc1ccc(CCOC(=O)CCCCCCCBr)cc1]

Contains compound 4 (215 mg, 1.2 mmol, 1.0 eq.), compound 5 (404 mg, 1.8 mmol, 1.5 eq.), EDCI (1.15 g, 6.0 mmol, 5.0 eq.), DMAP (732 mg, 1.8 eq.), DIEA (1.29 g, 12.0 mmol, 10.0 eq.) and DIEA (1.29 g, 12.0 mmol, 10.0 eq.) in DCM (5 mL) in N2 Under protection, stir for 16 hours at 10° C.

TLC (DCM:MeOH=10:1) showed the reaction was complete and a new major spot was observed.

The mixture was concentrated under reduced pressure, and the residue was purified by silica gel column chromatography, eluting with PE:EA (1:0-10:1, v/v) to obtain compound 6 (145 mg, 31%) as a colorless oil.

¹H NMR (400 MHz, CDCl 3) δ7.12 (s, 4H), 4.27 (t, J=7.1 Hz, 2H), 3.52 (t, J=6.7 Hz, 1H), 3.40 (t, J=6.8 Hz, 1H), 2.90 (t, J=7.1 Hz, 2H), 2.65-2.50 (m, 2H), 2.28 (t, J=7.5 Hz, 2H), 1.93-1.70 (m, 2H), 1.64-1.56 (m, 4H), 1.44-1.27 (m, 8H), 0.92 (t, J=7.3 Hz, 3H).

### 4. Synthesis of Final Product SW-II-118

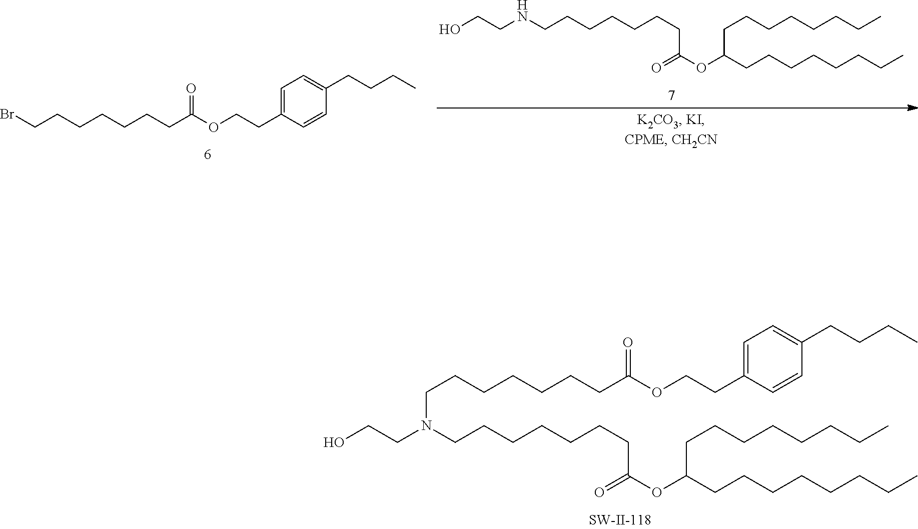 [SMILES: CCCCCCCCC(CCCCCCCC)OC(=O)CCCCCCCNCCO ; CCCCc1ccc(CCOC(=O)CCCCCCCBr)cc1 ; CCCCCCCCC(CCCCCCCC)OC(=O)CCCCCCCN(CCO)CCCCCCCC(=O)OCCc1ccc(CCCC)cc1]

Contains compound 6 (140 mg, 0.37 mmol, 1.0 eq.), compound 7 (243 mg, 0.55 mmol, 1.5 eq.), K2 CO3 A mixture of (153 mg, 1.11 mmol, 3.0 eq.) and KI (123 mg, 0.74 mmol, 2.0 eq.) was dissolved in CPME (1 mL) and CH3CN (1 mL) mixed solvent in N₂ and stirred at 90° C. for 16 hours. The reaction mixture was concentrated under reduced pressure, and the residue was diluted with EtOAc (50 mL) and washed with NaHCO₃ (30 mL) for washing. Anhydrous Na was used for the organic layer 2SO4 Dry, filter and concentrate under reduced pressure. The residue was purified by silica gel column chromatography, eluting with DCM:MeOH (1:0-10:1, v/v), to give SW-II-118 (105 mg, 61%) as a yellow oil.

LCMS:Rt:1.946 min; MS m/z(ELSD):744.4[M+H]+; HPLC: 99.64% purity, ELSD; RT=5.875 min.

¹H NMR (400 MHz, CDCl 3) δ7.11 (s, 4H), 4.91-4.79 (m, 1H), 4.26 (t, J=7.2 Hz, 2H), 3.80-3.68 (m, 2H), 2.90 (t, J=7.1 Hz, 4H), 2.81-2.67 (m, 4H), 2.62-2.52 (m, 2H), 2.28 (td, J=7.5, 2.6 Hz, 4H), 1.64-1.51 (m, 11H), 1.38-1.17 (m, 42H), 0.93-0.82 (m, 9H).

¹³C NMR (101 MHz, CDCl 3) δ173.61 (d, J=11.7 Hz), 141.11 (s), 134.90 (s), 128.74 (s), 128.51 (s), 77.40 (s), 77.08 (s), 76.77 (s), 74.17 (s), 64.90 (s), 57.48 (s), 56.24 (s), 53.98 (s), 35.25 (s), 34.66 (d, J=14.4 Hz), 34.16 (d, J=5.1 Hz), 33.67 (s), 31.86 (s), 29.52 (d, J=2.4 Hz), 29.24 (s), 29.21-28.74 (m), 26.90 (d, J=4.9 Hz), 25.42-24.92 (m), 24.92-24.88 (m), 24.74 (s), 22.67 (s), 22.37 (s), 14.04 (d, J=15.7 Hz).

### C. Compound SW-II-120

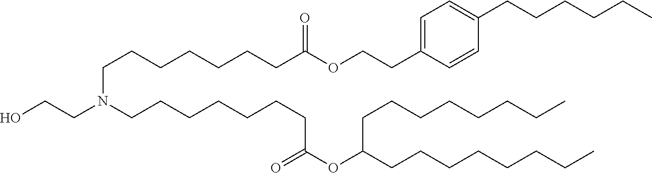 [SMILES: CCCCCCCCC(CCCCCCCC)OC(=O)CCCCCCCN(CCO)CCCCCCCC(=O)OCCc1ccc(CCCCCC)cc1]

### 1. Synthesis of Intermediate 3

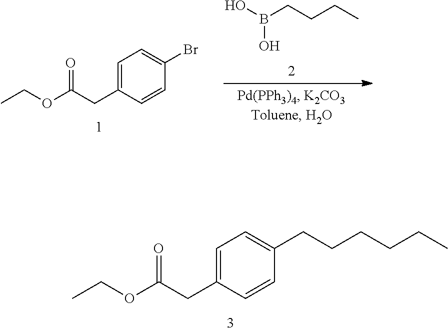

Contains compound 1 (1.22 g, 5.0 mmol, 1.0 eq.), compound 2 (1.30 mg, 10.0 mmol, 2.0 eq.), Pd (PPh 3) 4 (289 mg, 0.25 mmol, 0.05 eq.) and K2CO3 (1.38 g, 10.0 mmol, 2.0 eq.) in toluene (10 ml) and H2 In a mixed solution of O (1 ml) at 110° C., N2 Stir protected for 1 hour. TLC (petroleum ether:ethyl acetate=19:1) showed that compound 1 was consumed and a new spot was observed. The reaction mixture was diluted with DCM (50 mL) and washed with H2O (40 mL), washed with anhydrous Na2SO4 Dry, filter and concentrate under reduced pressure. The residue was purified by silica gel column chromatography, eluting with petroleum ether:ethyl acetate (1:0-10:1), to obtain compound 3 (0.78 g, 62%) as a colorless oil.

1H NMR (400 MHz, CDCl 3) δ7.19 (d, J=8.1 Hz, 2H), 7.13 (d, J=8.1 Hz, 2H), 4.14 (q, J=7.1 Hz, 2H), 3.57 (s, 2H), 2.62-2.51 (m, 2H), 1.58 (d, J=11.1 Hz, 2H), 1.35-1.21 (m, 9H), 0.88 (t, J=6.7 Hz, 3H).

### 2. Synthesis of Intermediate 4

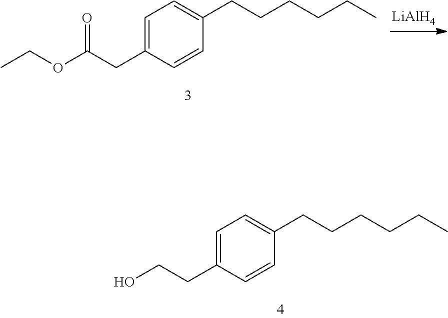

LiAlH at −78° C. 4 (477 mg, 12.56 mmol, 4.0 eq.) was added to a solution of compound 3 (780 mg, 3.14 mmol, 1.0 eq.) in THF (10 mL), and the reaction was stirred at 10° C. for 3 hours. Thin layer chromatography showed the reaction was going well. The reaction was concentrated and washed with Na2SO4 (20 mL) was diluted and extracted with EA (30 mL*2), and the organic phase was used anhydrous Na2SO4 Dry, filter and concentrate under reduced pressure to obtain compound 4 (640 mg, crude product) as colorless oil.

### 3. Synthesis of Intermediate 6

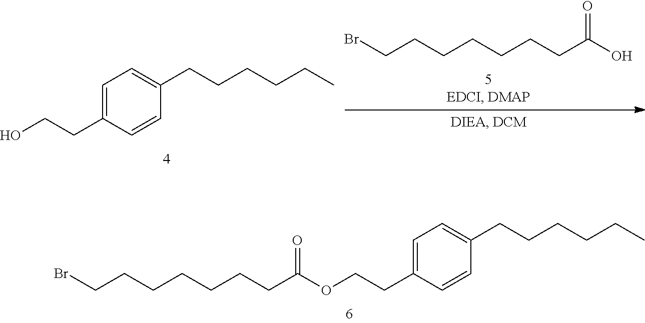 [SMILES: O=C(O)CCCCCCCBr ; CCCCCCc1ccc(CCO)cc1 ; CCCCCCc1ccc(CCOC(=O)CCCCCCCBr)cc1]

Contains compound 4 (640 mg, 3.10 mmol, 1.0 eq.), compound 5 (1.06 g, 4.70 mmol, 1.5 eq.), EDCI (2.98 g, 15.5 mmol, 5.0 eq.), DMAP (1.85 g, 15.0 eq.) and DIEA (4.0 g, 31.0 mmol, 10.0 eq.) in DCM (10 mL) in N2 Under protection, stir at 10° C. for 16 h. TLC (DCM:MeOH=10:1) showed the reaction was complete and a new major spot was observed. The mixture was concentrated under reduced pressure, and the residue was purified by silica gel column chromatography, eluting with PE:EA (1:0-10:1, v/v), to obtain compound 6 (465 mg, 36%) as a colorless oil.

### 4. Synthesis of Final Product SW-II-120

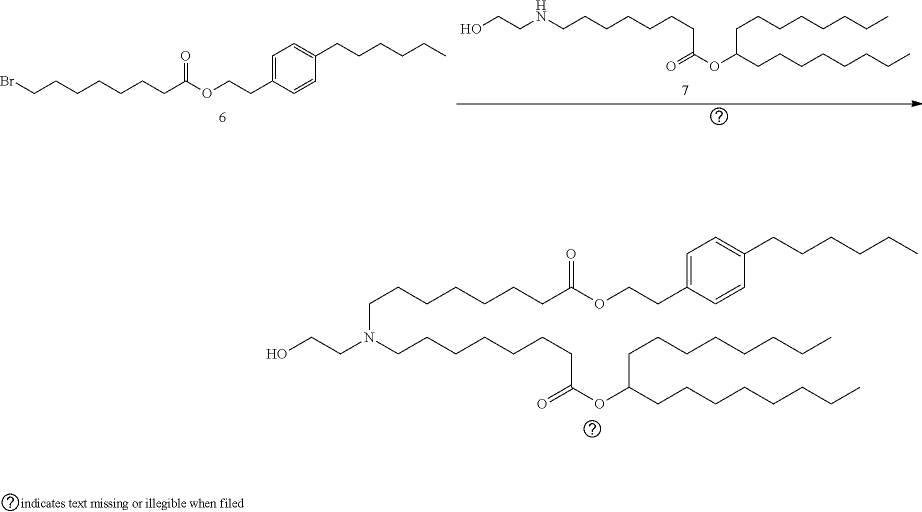 [SMILES: CCCCCCc1ccc(CCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccc(CCCCCC)cc1]

Contains compound 6 (100 mg, 0.25 mmol, 1.0 eq.), compound 7 (161 mg, 0.36 mmol, 1.5 eq.), K2CO3 A mixture of (104 mg, 0.75 mmol, 3.0 eq.) and KI (83 mg, 0.50 mmol, 2.0 eq.) was dissolved in CPME (1 mL) and CH3CN (1 mL) in N2 and stirred at 90° C. for 16 hours. The reaction mixture was concentrated under reduced pressure, the residue was diluted with EtOAc (50 mL) and washed with NaHCO3 (30 mL) for washing. Anhydrous Na was used for the organic layer 2 SO4 Dry, filter and concentrate under reduced pressure. The residue was purified by silica gel column chromatography, eluting with DCM:MeOH (1:0-10:1, v/v), to give SW-II-120 (100 mg, 52%) as a yellow oil.

LCMS:Rt:2.500 min; MS m/z(ELSD):772.4[M+H]+;

HPLC: 99.70% purity, ELSD; RT=8.675 min.

¹H NMR (400 MHz, CDCl3) δ7.07 (d, J=8.9 Hz, 4H), 4.89-4.73 (m, 1H), 4.23 (t, J=7.2 Hz, 2H), 3.83-3.65 (m, 2H), 2.87 (t, J=7.2 Hz, 4H), 2.82-2.67 (m, 4H), 2.61-2.45 (m, 2H), 2.25 (td, J=7.5, 2.5 Hz, 4H), 1.65-1.44 (m, 15H), 1.27 (dd, J=13.2, 11.3 Hz, 42H), 0.85 (t, J=6.8 Hz, 9H).

¹³C NMR (101 MHz, CDCl3) δ173.57 (d, J=11.5 Hz), 141.13 (s), 134.88 (s), 128.73 (s), 128.48 (s), 77.45 (s), 77.13 (s), 76.81 (s), 74.14 (s), 64.89 (s), 57.34 (s), 56.17 (s), 53.92 (s), 35.57 (s), 34.64 (d, J=16.1 Hz), 34.14 (d, J=3.3 Hz), 31.79 (d, J=13.4 Hz), 31.49 (s), 29.50 (d, J=2.2 Hz), 29.23 (s), 29.10-28.71 (m), 26.85 (d, J=5.0 Hz), 25.49-25.38 (m), 25.13 (d, J=35.4 Hz), 24.72 (s), 22.63 (d, J=5.8 Hz), 14.11 (s).

### D. Compound SW-II-121

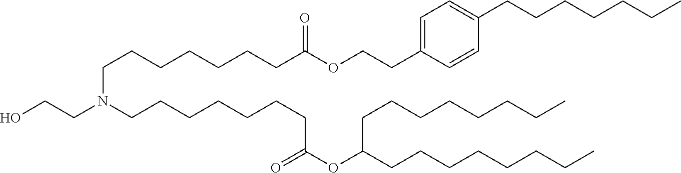 [SMILES: CCCCCCCCC(CCCCCCCC)OC(=O)CCCCCCCN(CCO)CCCCCCCC(=O)OCCc1ccc(CCCCCCC)cc1]

### 1. Synthesis of Intermediate 3

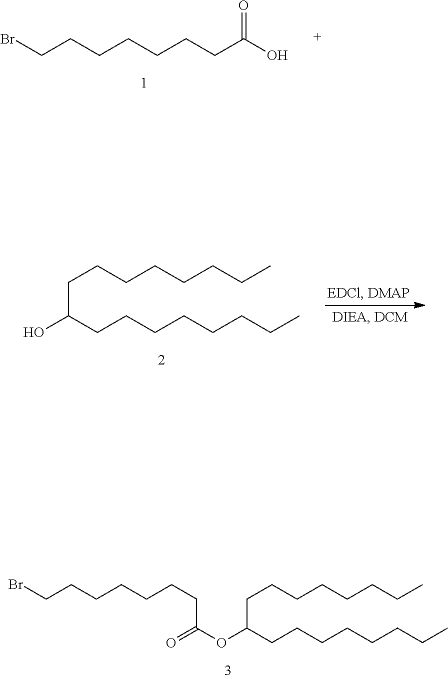 [SMILES: O=C(O)CCCCCCCBr ; CCCCCCCCC(O)CCCCCCCC ; CCCCCCCCC(CCCCCCCC)OC(=O)CCCCCCCBr]

To the DCM (20 mL) solution containing compound 1 (1.3 g, 5.86 mmol, 1.5 eq.) and compound 2 (1 g, 3.9 mmol, 1.0 eq.), EDCI (1.495 g, 7.8 mmol, 2.0 eq.) and DMAP were added (0.19 g, 1.56 mmol, 0.4 eq.) and DIEA (2.57 mL, 15.6 mmol, 4.0 eq.). The reaction mixture was brought to room temperature in N2 Stir for 16 hours. TLC (petroleum ether:ethyl acetate=19:1) showed that compound 2 was consumed and the desired product was formed. The reaction mixture was diluted with DCM (20 mL) and washed with H2O (40 mL), washed with anhydrous Na2SO4 Dry, filter and concentrate under reduced pressure. The residue was purified by silica gel column chromatography, eluting with petroleum ether:ethyl acetate (1:0-10:1), to obtain compound 3 (1.2 g, 66.9%) as a yellow oil.

1H NMR (400 MHz, CDCl 3) δ4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

2. Synthesis of intermediate 5

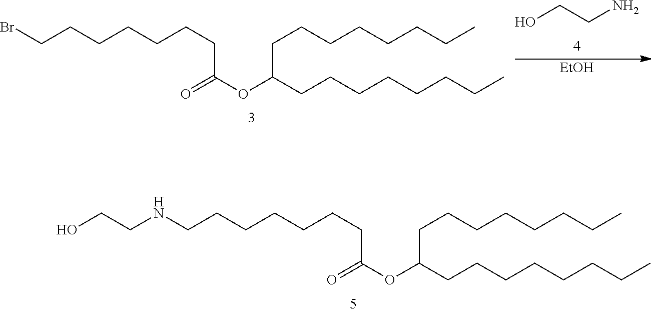

A solution of compound 3 (5.2 g, 11.30 mmol, 1.0 eq.) and compound 4 (20.6 g, 339 mmol, 30 eq.) in EtOH (5 mL) was dissolved in N2 Stir at 60° C. for 16 hours under protection. TLC (petroleum ether:ethyl acetate=19:1) showed that compound 3 was consumed and TLC (DCM/MeOH=10/1) showed that a new major spot was observed. The reaction mixture was concentrated under reduced pressure, and the residue was diluted with EtOAc (50 mL) and washed with H2 Wash with O (3×50 mL). Anhydrous Na was used for the organic layer 2SO4 After drying, filtration and concentration under reduced pressure, the residue was purified by silica gel column chromatography, eluting with DCM:MeOH (1:0-10:1, v/v) to obtain compound 5 (3 g, 60%) as a yellow oil.

### 3. Synthesis of Intermediate 8

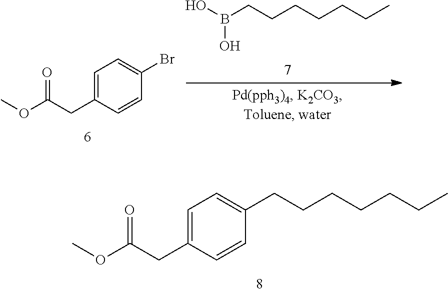

Pd (pph 3) 4 (238 mg, 0.206 mmol, 0.05 eq.), K2CO3 (1.7 g, 12.35 mmol, 3 eq). Place the mixture in N2 and stirred at 110° C. for 2 hours. TLC (PE:EA=10:1) showed the reaction was complete and a new major spot was observed. The mixture was extracted with EA and washed with water. Anhydrous Na was used for the organic layer 2SO4 Dry, filter and concentrate in vacuo. The residue was purified by silica gel column chromatography, eluting with PE:EA (1:0-10:1), to give compound 8 as a colorless oil (714 mg, 66%).

### 4. Synthesis of Intermediate 9

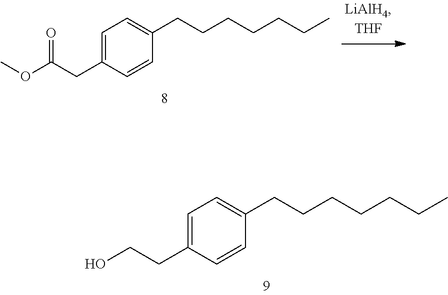 [SMILES: CCCCCCCc1ccc(CC(=O)OC)cc1 ; CCCCCCCc1ccc(CCO)cc1]

N2 To a mixture of compound 8 (714 mg, 2.725 mmol, 1 eq.) in THF (7 mL) was added LiAlH at 0° C. under protection. 4 (2.7 mL, 2.725 mmol, 1 M in THF, 1 eq.) and the mixture was stirred at room temperature for 2 hours. TLC (PE:EtOAc=10:1) showed the reaction was complete and a new major spot was observed. The mixture was quenched with water (2.7 mL) and treated with 2N HCl to adjust the pH between 6 and 7, extracted with EA and washed with brine. Anhydrous Na was used for the organic layer 2SO4 Dry, filter and concentrate in vacuo. The residue was purified by silica gel column chromatography, eluting with PE:EA (1:0-10:1), to give compound 9 as a colorless oil (103 mg, 63%).

### 5. Synthesis of Intermediate 11

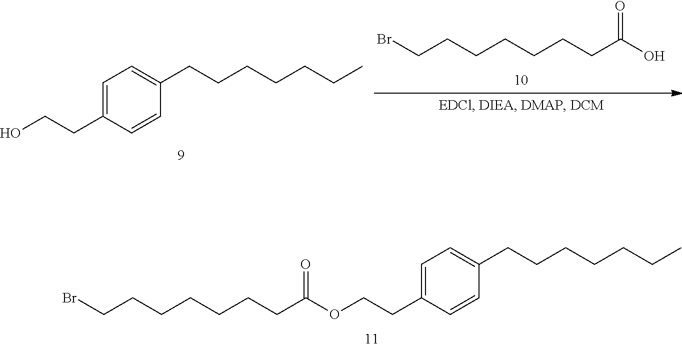

To DCM (3 mL) containing compound 9 (300 mg, 1.364 mmol, 1 eq.) and compound 10 (363 mg, 1.64 mmol, 1.2 eq.), EDCI (524 mg, 2.728 mmol, 2 eq.) and DMAP (67 mg, 0.546 mmol, 0.4 eq.), and DIEA (704 mg, 5.456 mmol, 4 eq.). The reaction mixture was brought to room temperature in N2 Stir for 16 hours. TLC (petroleum ether:ethyl acetate=10:1) showed the formation of the desired product. The reaction mixture was extracted with EA and washed with water. Anhydrous Na was used for the organic layer 2SO4 Dry, filter and concentrate in vacuo. The residue was purified by silica gel column chromatography, eluting with petroleum ether:ethyl acetate (1:0-10:1), to obtain compound 11 as a colorless oil (169 mg, 29%).

### 6. Synthesis of Final Product SW-II-121

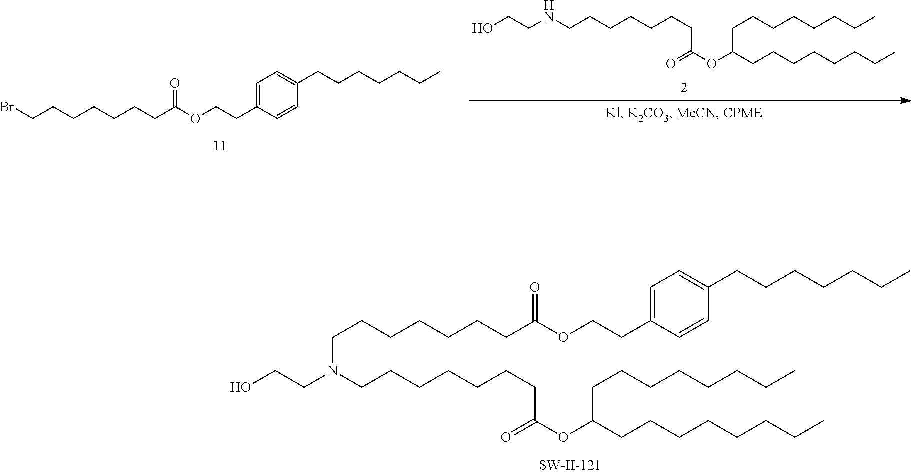 [SMILES: CCCCCCCCC(CCCCCCCC)OC(=O)CCCCCCCNCCO ; CCCCCCCc1ccc(CCOC(=O)CCCCCCCBr)cc1 ; CCCCCCCCC(CCCCCCCC)OC(=O)CCCCCCCN(CCO)CCCCCCCC(=O)OCCc1ccc(CCCCCCC)cc1]

To CPME/CH containing compound 11 (169 mg, 0.399 mmol, 1 eq.) and compound 5 (176 mg, 0.399 mmol, 1 eq.) 3 Add K to CN (2 mL/2 mL) mixed solvent 2CO3 (330 mg, 2.394 mmol, 6 eq.) and KI (132 mg, 0.798 mmol, 2 eq.). After the addition is complete, bring the mixture to N2 Stir overnight at 90° C. TLC (DCM:MeOH=10:1) showed the reaction was complete and a new major spot was observed. The mixture was extracted with EA and washed with water, and the organic layer was washed with anhydrous Na2SO4 Dry, filter and concentrate under reduced pressure. The residue was purified by silica gel column chromatography, eluting with DCM:MeOH (1:0-10:1, v/v), to obtain compound SW-II-121 (145 mg, 46%) as a yellow oil.

LCMS:Rt:1.493 min; MS m/z(ELSD):786.5[M+H]+;

HPLC: 99.869% purity, ELSD; RT=10.655 min.

¹H NMR (400 MHz, CDCl 3) δ7.11 (s, 4H), 4.92-4.80 (m, 1H), 4.26 (t, J=7.2 Hz, 2H), 3.80 (s, 2H), 2.87 (dd, J=26.6, 19.4 Hz, 7H), 2.62-2.51 (m, 2H), 2.28 (td, J=7.2, 3.6 Hz, 4H), 1.75-1.45 (m, 14H), 1.42-1.09 (m, 45H), 0.88 (t, J=6.8 Hz, 9H).

¹³C NMR (101 MHz, CDCl 3) δ173.61 (d, J=12.3 Hz), 141.20 (s), 134.90 (s), 128.75 (s), 128.51 (s), 77.35 (s), 77.03 (s), 76.72 (s), 74.21 (s), 64.93 (s), 54.15 (s), 35.59 (s), 34.66 (d, J=16.6 Hz), 34.16 (d, J=3.0 Hz), 31.85 (d, J=4.4 Hz), 31.55 (s), 29.64-29.15 (m), 29.15-28.78 (m), 26.85 (d, J=4.5 Hz), 25.33 (s), 24.95 (s), 24.72 (s), 22.68 (s), 14.12 (s)).

### E. Compound SW-JJ-122

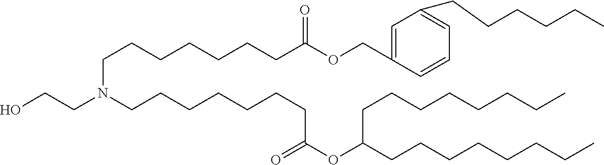 [SMILES: CCCCCCCCC(CCCCCCCC)OC(=O)CCCCCCCN(CCO)CCCCCCCC(=O)OCc1cccc(CCCCCC)c1]

### 1. Synthesis of Compound 3

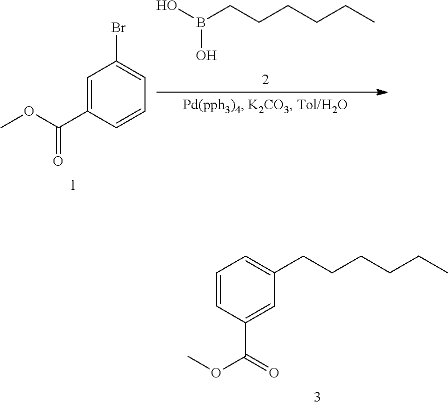

Compound 1 (lg, 4.65 mmol, 1 eq.) and compound 2 (726 mg, 5.58 mmol, 1.2 eq.) were dissolved in toluene/water (10/1, 20 mL), and then K was added to the mixture 2CO3 (1.92 g, 13.9 mmol, 3 eq.) and Pd(pph3) 4 (269 mg, 0.23 mmol, 0.05 eq). Place the reaction mixture in N2 Heat to 110° C. and stir for 2 hours. TLC (petroleum ether/ethyl acetate=19/1) showed that compound 1 was consumed and a new major spot was observed. The reaction mixture was reacted with H2 Quenched with O (80 mL) and extracted with ethyl acetate (60 mL×3), the organic layer was dried over anhydrous sodium sulfate, filtered and concentrated under reduced pressure. The residue was purified by silica gel column chromatography, eluting with petroleum ether/ethyl acetate (1/0-10/1), to obtain compound 3 (800 mg, 78%) as a yellow oil.

### 2. Synthesis of Compound 4

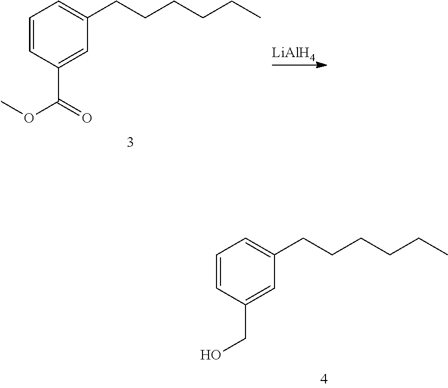

LiAlH was added to compound 3 (700 mg, 3.18 mmol, 1.0 eq.) dissolved in THF (14 mL) under nitrogen protection at 0° C. 4 (3.2 mL, 3.18 mmol, 1 eq). The reaction mixture was raised to room temperature and stirred under nitrogen protection for 2 hours. TLC (PE/EtOAc=10/1) showed the reaction was complete and new major spots were observed. The mixture was quenched with water (3.2 mL) and IM HCl (3.2 mL) respectively. Water (6 mL) was added to the mixture, and the mixture was extracted with ethyl acetate (60 mL×3). The organic layer was washed with brine (30 mL×2), dried over anhydrous sodium sulfate, filtered, and concentrated under reduced pressure. The residue was purified by silica gel column chromatography, eluting with ethyl acetate/petroleum ether=1/10, to obtain compound 4 (600 mg, 98%) as a yellow oil.

### 3. Synthesis of Compound 6

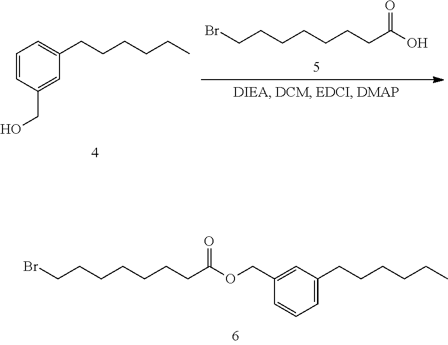 [SMILES: CCCCCCc1cccc(CO)c1 ; O=C(O)CCCCCCCBr ; CCCCCCc1cccc(COC(=O)CCCCCCCBr)c1]

Compound 4 (680 mg, 3.5 mmol, 1.0 eq.) and compound 5 (1.13 g, 5.1 mmol, 1.5 eq.) were dissolved in DCM (10 mL), and EDCI (1.20 g, 6.25 mmol, 2.0 eq.) was added to the mixture.), DMAP (166 mg, 1.36 mmol, 0.4 eq.) and DIEA (1.78 g, 13.8 mmol, 4.0 eq.). After the addition, the reaction mixture was stirred at room temperature overnight under nitrogen protection. TLC (DCM/MeOH=30/1) showed consumption of starting material and formation of a new spot. The mixture was quenched with water (70 mL) and extracted with DCM (80 mL×3). The combined organic layers were washed with brine (2×20 mL), dried over anhydrous sodium sulfate, filtered and concentrated under reduced pressure. The residue was purified by silica gel column chromatography, eluting with a solution of ethyl acetate/petroleum ether=3/97, to obtain compound 6 (680 mg, 48.5%) as a yellow oil.

### 4. Synthesis of SW-II-122

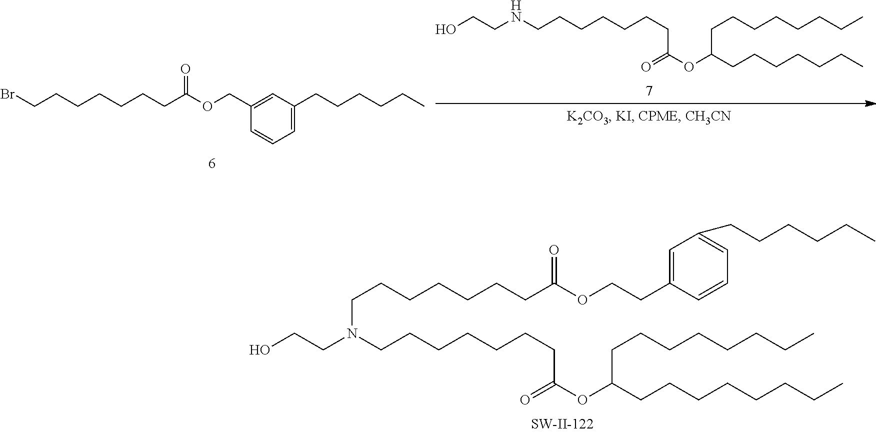 [SMILES: CCCCCCCCC(CCCCCCC)OC(=O)CCCCCCCNCCO ; CCCCCCc1cccc(COC(=O)CCCCCCCBr)c1 ; CCCCCCCCC(CCCCCCCC)OC(=O)CCCCCCCN(CCO)CCCCCCCC(=O)OCCc1cccc(CCCCCC)c1]

Compound 6 (108 mg, 0.27 mmol, 1.2 eq.) and compound 7 (100 mg, 0.23 mmol, 1 eq.) were dissolved in CPME (2 mL) and CH3CN (2 mL), to the mixture were added potassium carbonate (157 mg, 1.14 mmol, 5.0 eq) and potassium iodide (75 mg, 0.45 mmol, 2.0 eq). After the addition was completed, the reaction mixture was stirred at 90° C. for 16 hours under nitrogen protection. TLC (DCM/MeOH=10/1) showed the reaction was complete. The reaction mixture was concentrated under reduced pressure. The residue was purified by silica gel column chromatography, eluting with DCM/MeOH (1/0-10:1, v/v), to give SW-II-122 (68 mg, 40%) as a colorless oil.

LCMS:Rt:1.487 min; MS m/z(ELSD):758.5[M+H]+;

HPLC: 97.3% purity, ELSD; RT=7.622 min.

¹H NMR (400 MHz, CDCl 3) δ7.32 (d, J=26.4 Hz, 1H), 7.17 (dd, J=27.2, 21.1 Hz, 3H), 5.09 (s, 2H), 4.91-4.79 (m, 1H), 3.85 (s, 2H), 2.98 (s, 2H), 2.87 (s, 4H), 2.65-2.54 (m, 2H), 2.35 (t, J=7.6 Hz, 2H), 2.28 (t, J=7.6 Hz, 2H), 1.74-1.57 (m, 9H), 1.50 (d, J=5.6 Hz, 4H), 1.37-1.15 (m, 43H), 0.94-0.80 (m, 9H).

¹³C NMR (101 MHz, CDCl 3) δ173.55 (d, J=2.4 Hz), 143.35 (s), 135.92 (s), 128.67-128.19 (m), 125.47 (s), 77.36 (s), 77.04 (s), 76.73 (s), 74.22 (s), 66.27 (s), 57.15 (s), 56.74 (s), 54.14 (s), 35.88 (s), 34.55 (s), 34.15 (d, J=3.6 Hz), 31.79 (d, J=15.2 Hz), 31.43 (s), 29.52 (d, J=2.8 Hz), 29.25 (s), 28.92 (dd, J=14.2, 5.8 Hz), 26.77 (d, J=4.8 Hz), 25.33 (s) 24.92 (s), 24.71 (s), 24.48 (s), 22.64 (d, J=6.8 Hz), 14.12 (s).

### F Compound SW-II-127

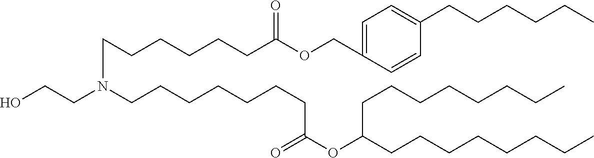 [SMILES: CCCCCCCCC(CCCCCCCC)OC(=O)CCCCCCCN(CCO)CCCCCCC(=O)OCc1ccc(CCCCCC)cc1]

### 1. Synthesis of Compound 3

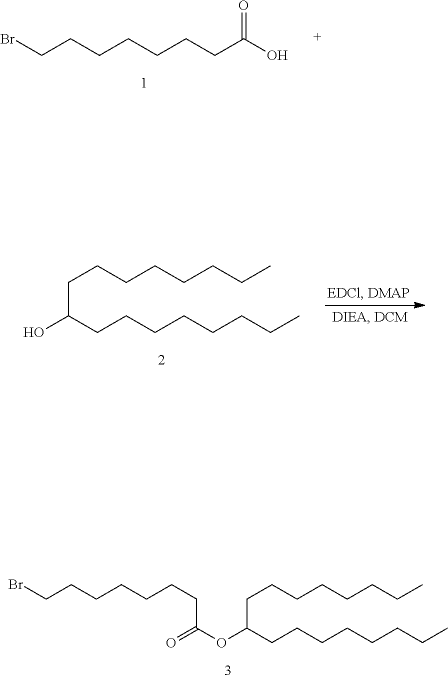 [SMILES: O=C(O)CCCCCCCBr ; CCCCCCCCC(O)CCCCCCCC ; CCCCCCCCC(CCCCCCCC)OC(=O)CCCCCCCBr]

Compound 1 (1.3 g, 5.86 mmol, 1.5 eq.) and compound 2 (lg, 3.9 mmol, 1.0 eq.) were dissolved in DCM (20 mL), and EDCI (1.495 g, 7.8 mmol, 2.0 eq.) was added to the mixture.) and DMAP (0.19 g, 1.56 mmol, 0.4 eq.), then DIEA (2.57 mL, 15.6 mmol, 4.0 eq.) was added. The reaction mixture was stirred at room temperature under nitrogen protection for 16 hours. TLC (petroleum ether/ethyl acetate=19/1) showed consumption of compound 2 and formation of the desired product. The reaction mixture was diluted with DCM (20 mL) and washed with H2O (40 mL), washed with anhydrous Na2SO4 Dry, filter and concentrate under reduced pressure. The residue was purified by silica gel column chromatography, eluting with petroleum ether/ethyl acetate (1/0-10/1), to obtain compound 3 (1.2 g, 66.9%) as a yellow oil.

1H NMR (400 MHz, CDCl 3) δ4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Compound 5

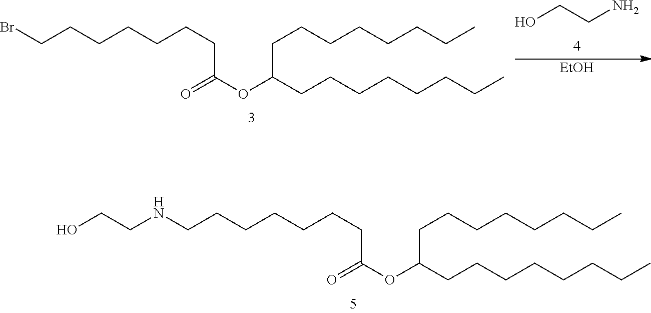

Compound 3 (5.2 g, 11.30 mmol, 1.0 eq.) and compound 4 (20.6 g, 339 mmol, 30 eq.) were added to EtOH (5 mL), and then the mixture was stirred at 60° C. for 16 hours under nitrogen protection. TLC (petroleum ether/ethyl acetate=19/1) showed that compound 3 was consumed and TLC (DCM/MeOH=10/1) showed that a new major spot was observed. The reaction mixture was concentrated under reduced pressure. The residue was diluted with EtOAc (50 mL) and washed with H2 Wash with O (3×50 mL). The organic layer was dried over anhydrous sodium sulfate, filtered and concentrated under reduced pressure. The residue was purified by silica gel column chromatography, eluting with DCM/MeOH (1/0-10:1, v/v), to obtain compound 5 as a yellow oil (3 g, 60%).

1H NMR (400 MHz, CDCl 3) δ4.95-4.75 (m, 1H), 3.74-3.58 (m, 2H), 2.87-2.74 (m, 2H), 2.69-2.56 (m, 2H), 2.36 (s, 2H), 2.28 (t, J=7.5 Hz, 2H), 1.65-1.42 (m, 8H), 1.38-1.17 (m, 30H), 0.88 (t, J=6.8 Hz, 6H).

### 3. Synthesis of Compound 8

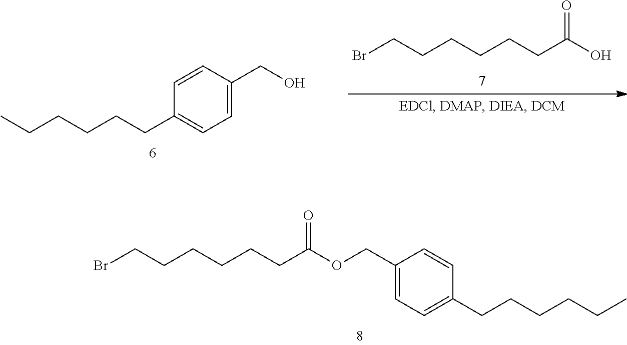 [SMILES: O=C(O)CCCCCCBr ; CCCCCCc1ccc(CO)cc1 ; CCCCCCc1ccc(COC(=O)CCCCCCBr)cc1]

Compound 7 (522 mg, 2.5 mmol, 1.2 eq.) and compound 6 (400 mg, 2.083 mmol, 1 eq.) were dissolved in DCM (4 mL), and EDCI (800 mg, 4.166 mmol, 2 eq.), DMAP (102 mg, 0.833 mmol, 0.4 eq.) and DIEA (1.075 mg, 8.332 mmol, 4 eq.). After the addition, the reaction mixture was stirred at room temperature overnight under nitrogen protection. TLC (PE:EA=10:1) showed that the starting material was consumed and a new spot formed. The reaction mixture was concentrated under reduced pressure. The residue was purified by silica gel column chromatography, eluting with PE/EA (1/0-10/1), to obtain compound 8 (454 mg, 57%) as a colorless oil.

### 4. Synthesis of SW-II-127

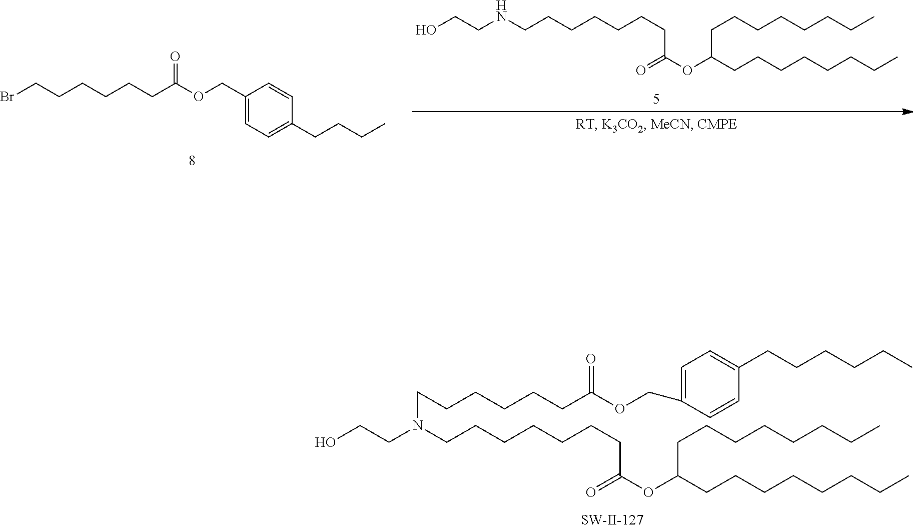 [SMILES: CCCCc1ccc(COC(=O)CCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCC(=O)OCc1ccc(CCCCCC)cc1]

Compound 8 (100 mg, 0.262 mmol, 1 eq.) and compound 5 (139 mg, 0.314 mmol, 1.2 eq.) were dissolved in CPME/CH3CN (1 mL/1 mL), and potassium carbonate (217 mg, 1.572 mmol, 6 eq.) was added to the mixture.) and potassium iodide (87 mg, 0.524 mmol, 2 eq.). After the addition, the reaction mixture was stirred at 90° C. overnight under nitrogen protection. TLC (DCM/MeOH=10/1) showed that the reaction was complete and the desired product was formed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-127 (42.49 mg, 22%) as a yellow oil.

LCMS:Rt:1.323 min; MS m/z(ELSD):744.5[M+H]+;

HPLC: 99.742% purity, ELSD; RT=7.339 min.

¹H NMR (400 MHz, CDCl 3) δ7.25 (s, 2H), 7.17 (d, J=8.0 Hz, 2H), 5.07 (s, 2H), 4.91-4.82 (m, 1H), 3.83 (s, 2H), 2.90 (d, J=44.8 Hz, 5H), 2.64-2.55 (m, 2H), 2.35 (t, J=7.4 Hz, 2H), 2.28 (t, J=7.5 Hz, 2H), 1.76-1.46 (m, 14H), 1.42-1.19 (m, 41H), 0.88 (t, J=6.8 Hz, 9H).

¹³C NMR (101 MHz, CDCl 3) δ173.50 (d, J=8.5 Hz), 133.17 (s), 128.61 (s), 128.34 (s), 77.29 (d, J=11.4 Hz), 77.03 (s), 76.71 (s), 74.23 (s), 66.19 (s), 54.20 (s), 35.71 (s), 34.56 (s), 34.10 (d, J=8.8 Hz), 31.80 (d, J=15.4 Hz), 31.43 (s), 29.53 (d, J=2.5 Hz), 29.25 (s), 28.95 (d, J=10.5 Hz), 28.63 (s), 26.71 (d, J=18.2 Hz), 25.33 (s), 24.93 (s), 24.62 (s), 22.65 (d, J=6.6 Hz), 14.13 (s).

### G. Compound SW-II-134-1

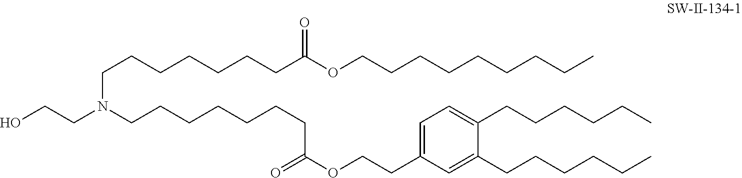 [SMILES: CCCCCCCCCOC(=O)CCCCCCCN(CCO)CCCCCCCC(=O)OCCc1ccc(CCCCCC)c(CCCCCC)c1]

### 1. Synthesis of Compound 3

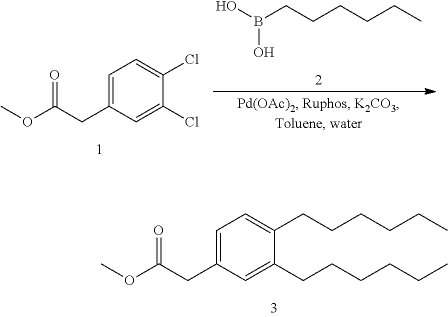

To a mixture of compound 1 (500 mg, 2.283 mmol, 1 eq.) and compound 2 (890 mg, 6.849 mmol, 3 eq.) in toluene/water (5 mL/1 mL) was added palladium acetate (51 mg, 0.228 mmol, 0.1 eq.), Ruphos (213 mg, 0.457 mmol, 0.2 eq.) and potassium carbonate (945 mg, 6.849 mmol, 3 eq.). The mixture was stirred at 110° C. overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (723 mg, 99.6%) as a colorless oil.

### 2. Synthesis of Compound 4

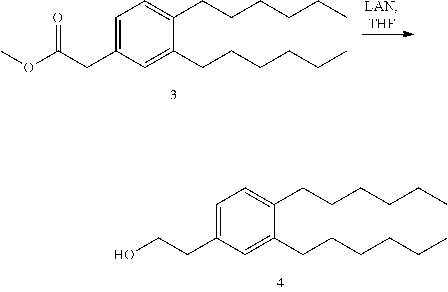

To a mixture of compound 3 (723 mg, 2.27 mmol, 1 eq.) in THF (8 mL) was added lithium aluminum hydride (2.3 mL, 2.27 mmol, 1 M, 1 eq. in THF) at 0° C. under nitrogen. The mixture was stirred at room temperature for 3 hours. TLC (PE/EA=5/1) indicated that the reaction was complete and new major spots were observed. The mixture was quenched with water (2.3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (381 mg, 58%) as a colorless oil without further purification.

### 3. Synthesis of Compound 6

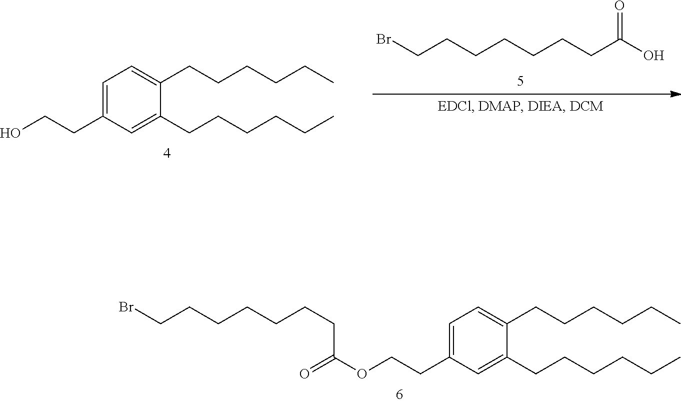 [SMILES: O=C(O)CCCCCCCBr ; CCCCCCc1ccc(CCO)cc1CCCCCC ; CCCCCCc1ccc(CCOC(=O)CCCCCCCBr)cc1CCCCCC]

To a mixture of compound 4 (381 mg, 1.3 mmol, 1 eq.) and compound 5 (352 mg, 1.6 mmol, 1.2 eq.) in DCM (4 mL) was added EDCI (499 mg, 2.6 mmol, 2 eq.) and DMAP (63 mg, 0.52 mmol, 0.4 eq.), then DIEA (671 mg, 5.2 mmol, 4 eq.) was added. The reaction mixture was stirred at room temperature under nitrogen for 16 hours. TLC (petroleum ether/ethyl acetate=20/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to give compound 6 (272 mg, 44%) as a colorless oil.

### 4. Synthesis of SW-II-134-1

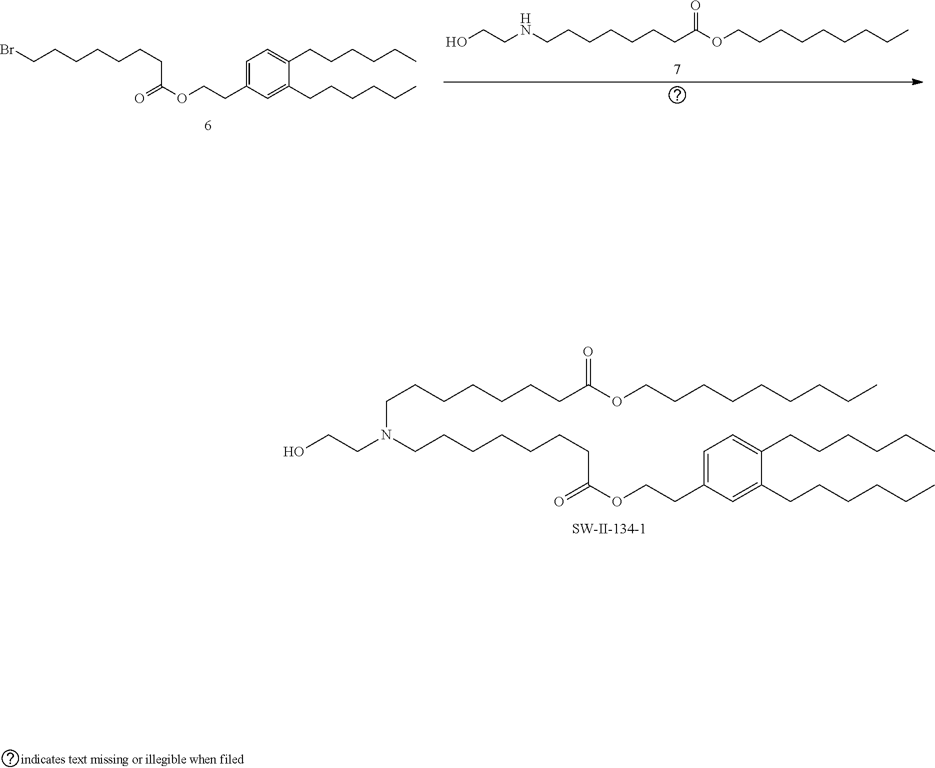 [SMILES: CCCCCCCCCOC(=O)CCCCCCCNCCO ; CCCCCCc1ccc(CCOC(=O)CCCCCCCBr)cc1CCCCCC ; CCCCCCCCCOC(=O)CCCCCCCN(CCO)CCCCCCCC(=O)OCCc1ccc(CCCCCC)c(CCCCCC)c1]

To compound 6 (150 mg, 0.303 mmol, 1 eq.) and compound 7 (110 mg, 0.333 mmol, 1.1 eq.) in CPME/CH3 Potassium carbonate (251 mg, 1.818 mmol, 6 eq.) and potassium iodide (101 mg, 0.61 mmol, 2 eq.) were added to the mixture in CN (2 mL/2 mL). After addition, the mixture was stirred at 90° C. overnight under nitrogen. TLC (DCM/MeOH=15/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-134-1 (168 mg, 75%) as a yellow oil.

LCMS:Rt:1.276 min; MS m/z(ELSD):744.4[M+H]+;

HPLC: 98.481% purity, ELSD; RT=10.724 min.

¹H NMR (400 MHz, CDCl 3) δ7.06 (d, J=7.6 Hz, 1H), 7.01-6.93 (m, 2H), 4.25 (t, J=7.3 Hz, 2H), 4.05 (t, J=6.8 Hz, 2H), 3.85-3.72 (m, 2H), 2.98-2.69 (m, 8H), 2.62-2.48 (m, 4H), 2.29 (t, J=7.5 Hz, 4H), 1.72-1.48 (m, 14H), 1.45-1.17 (m, 36H), 0.89 (dt, J=11.9, 6.0 Hz, 9H).

¹³CNMR (101 MHz, CDCl3) δ173.78 (d, J=16.7 Hz), 140.72 (s), 138.81 (s), 134.91 (s), 129.70 (s), 129.22 (s), 126.19 (s), 77.30 (d, J=11.4 Hz), 77.03 (s), 76.72 (s), 65.02 (s), 64.49 (s), 57.42 (s), 56.36 (s), 54.08 (s), 34.76 (s), 34.22 (d, J=4.2 Hz), 32.74 (s), 32.36 (s), 31.81 (d, J=9.1 Hz), 31.35 (d, J=5.3 Hz), 29.49 (d, J=2.8 Hz), 29.24 (d, J=2.2 Hz), 28.92 (s), 28.66 (s), 26.86 (s), 25.93 (s), 25.04 (s), 24.78 (d, J=6.6 Hz), 22.65 (d, J=2.6 Hz), 14.10 (s).

### H. Compound SW-II-134-2

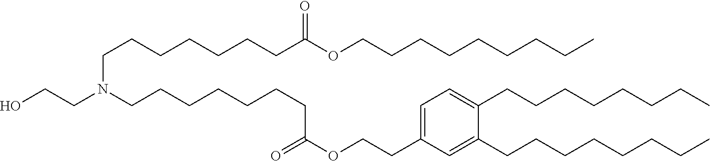 [SMILES: CCCCCCCCCOC(=O)CCCCCCCN(CCO)CCCCCCCC(=O)OCCc1ccc(CCCCCCCC)c(CCCCCCCC)c1]

### 1. Synthesis of Compound 3

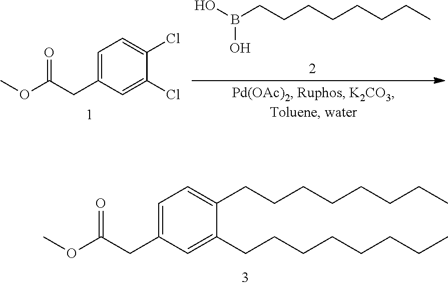 [SMILES: COC(=O)Cc1ccc(Cl)c(Cl)c1 ; CCCCCCCCB(O)O ; CCCCCCCCc1ccc(CC(=O)OC)cc1CCCCCCCC]

To a mixture of compound 1 (500 mg, 2.283 mmol, 1 eq.) and compound 2 (1.08 g, 6.849 mmol, 3 eq.) in toluene/water (5 mL/1 mL) was added palladium acetate (51 mg, 0.228 mmol, 0.1 eq.), Ruphos (213 mg, 0.457 mmol, 0.2 eq.) and potassium carbonate (945 mg, 6.849 mmol, 3 eq.). The mixture was stirred at 110° C. overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (854 mg, 100%) as a colorless oil.

### 2. Synthesis of Compound 4

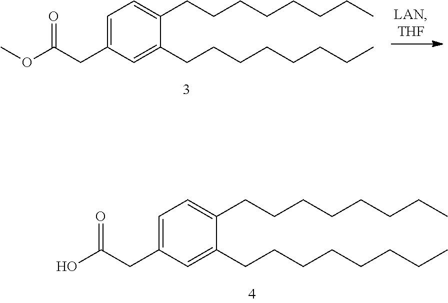 [SMILES: CCCCCCCCc1ccc(CC(=O)OC)cc1CCCCCCCC ; CCCCCCCCc1ccc(CC(=O)O)cc1CCCCCCCC]

To a mixture of compound 3 (854 mg, 2.28 mmol, 1 eq.) in THF (9 mL) was added lithium aluminum hydride (2.3 mL, 2.28 mmol, 1 M, 1 eq. in THF) at 0° C. under nitrogen. The mixture was stirred at room temperature for 3 hours. TLC (PE/EA=5/1) indicated that the reaction was complete and new major spots were observed. The mixture was quenched with water (2.3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (724 mg, 92%) as a colorless oil without further purification.

### 3. Synthesis of Compound 6

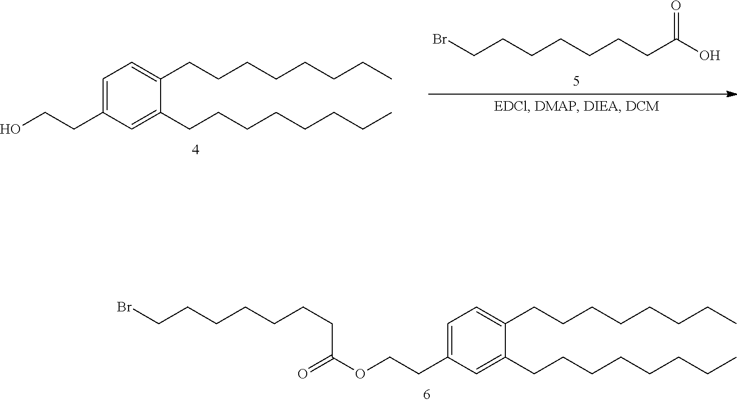 [SMILES: O=C(O)CCCCCCCBr ; CCCCCCCCc1ccc(CCO)cc1CCCCCCCC ; CCCCCCCCc1ccc(CCOC(=O)CCCCCCCBr)cc1CCCCCCCC]

To a mixture of compound 4 (724 mg, 2.09 mmol, 1 eq.) and compound 5 (560 mg, 2.51 mmol, 1.2 eq.) in DCM (8 mL) was added EDCI (803 mg, 4.18 mmol, 2 eq.) and DMAP (102 mg, 0.84 mmol, 0.4 eq.), then DIEA (1.078 g, 8.36 mmol, 4 eq.) was added. The reaction mixture was stirred at room temperature under nitrogen for 16 hours. TLC (petroleum ether/ethyl acetate=20/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to obtain compound 6 (473 mg, 41%) as a colorless oil.

### 4. Synthesis of SW-II-134-2

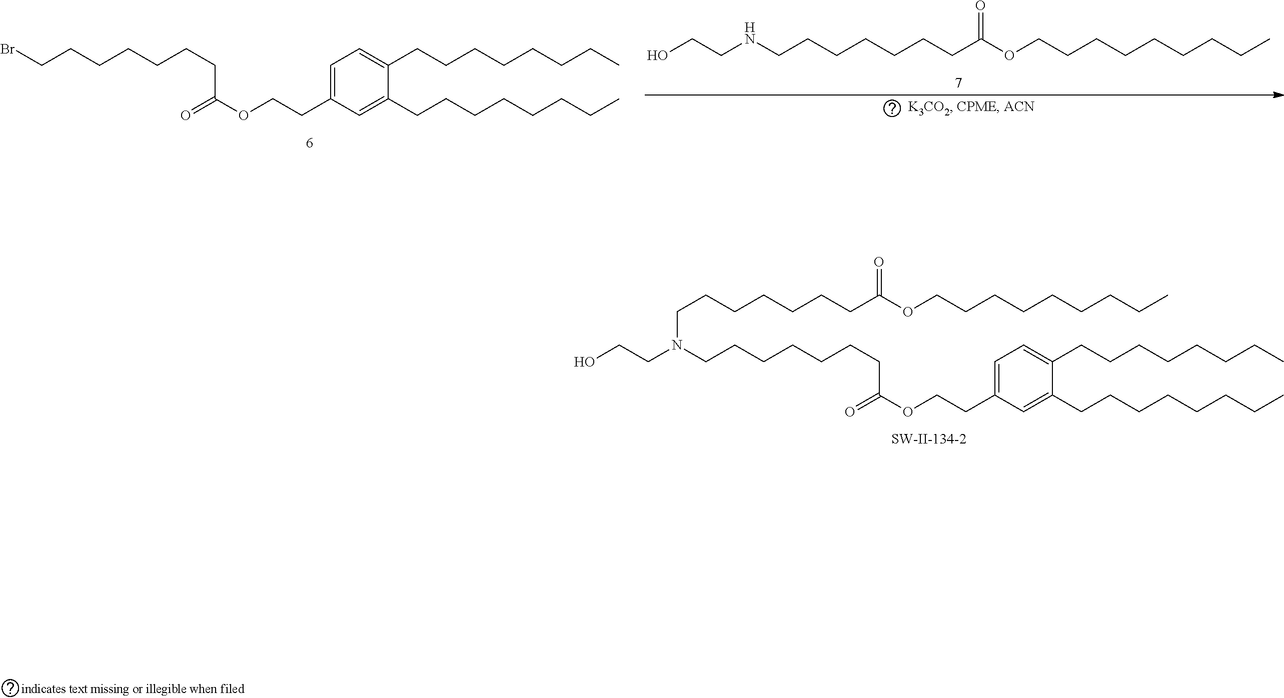

To compound 6 (150 mg, 0.27 mmol, 1 eq.) and compound 7 (108 mg, 0.33 mmol, 1.1 eq.) in CPME/CH3 Potassium carbonate (225 mg, 1.63 mmol, 6 eq.) and potassium iodide (90 mg, 0.54 mmol, 2 eq.) were added to the mixture in CN (2 mL/2 mL). After addition, the mixture was stirred at 90° C. overnight under nitrogen. TLC (DCM/MeOH=15/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-134-2 (71.77 mg, 33%) as a yellow oil.

LCMS:Rt:1.527 min; MS m/z(ELSD):800.4[M+H]+;

HPLC: 97.311% purity, ELSD; RT=9.025 min.

¹H NMR (400 MHz, CDCl 3) δ7.06 (d, J=7.6 Hz, 1H), 6.96 (d, J=9.6 Hz, 2H), 4.25 (t, J=7.3 Hz, 2H), 4.05 (t, J=6.8 Hz, 2H), 3.80-3.66 (m, 2H), 2.86 (dd, J=12.8, 5.6 Hz, 4H), 2.78-2.67 (m, 4H), 2.60-2.52 (m, 4H), 2.29 (t, J=7.5 Hz, 4H), 1.57 (dt, J=15.8, 7.3 Hz, 14H), 1.30 (d, J=20.3 Hz, 45H), 0.88 (t, J=6.7 Hz, 9H).

¹³C NMR (101 MHz, CDCl 3) δ173.82 (d, J=16.9 Hz), 140.73 (s), 138.82 (s), 134.91 (s), 129.71 (s), 129.23 (s), 126.19 (s), 77.36 (s), 77.14 (d, J=20.4 Hz), 76.72 (s), 65.03 (s), 64.49 (s), 57.57 (s), 56.13 (s), 54.02 (s), 34.76 (s), 34.25 (d, J=4.2 Hz), 32.76 (s), 32.37 (s), 31.89 (d, J=5.3 Hz), 31.40 (d, J=6.0 Hz), 29.84 (d, J=3.7 Hz), 29.63-29.14 (m), 28.97 (s), 28.65 (s), 26.93 (s), 25.66 (d, J=54.4 Hz), 24.80 (d, J=6.6 Hz), 22.68 (d, J=1.8 Hz), 14.12 (s).

### I. Compound SW-II-134-3

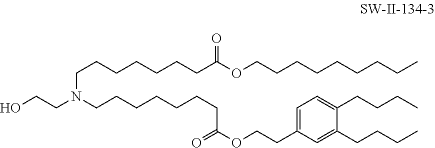 [SMILES: CCCCCCCCCOC(=O)CCCCCCCN(CCO)CCCCCCCC(=O)OCCc1ccc(CCCC)c(CCCC)c1]

### 1. Synthesis of Compound 3

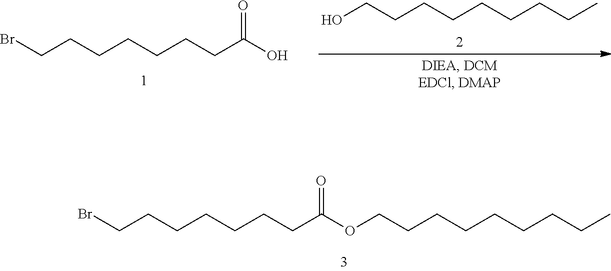

To a mixture of compound 1 (10 g, 45 mmol, 1 eq.) and compound 2 (7.8 g, 54 mmol, 1.2 eq.) in DCM (100 mL) was added EDCI (17.3 g, 90 mmol, 2 eq.) and DMAP (2.2 g, 18 mmol, 0.4 eq.), then DIEA (23.2 g, 180 mmol, 4 eq.) was added. The reaction mixture was stirred at room temperature under nitrogen for 16 hours. TLC (petroleum ether/ethyl acetate=30/1) showed that compound 1 was consumed and the desired product was formed. The reaction mixture was extracted with ethyl acetate (20 mL) and washed with water (40 mL×3), dried over anhydrous sodium sulfate, filtered and concentrated under reduced pressure. The residue was purified by silica gel column chromatography, eluting with petroleum ether/ethyl acetate (1/0-20/1), to obtain compound 3 (4.365 g, 28%) as a colorless oil.

### 2. Synthesis of Compound 5

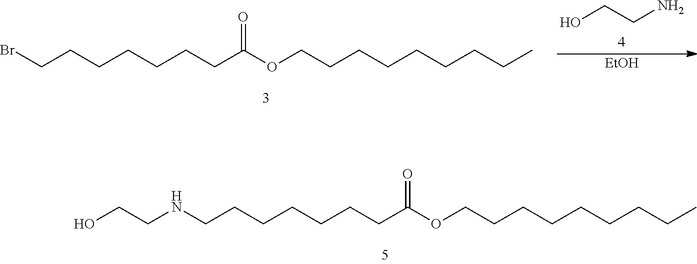

A mixture of compound 3 (5 g, 14.38 mmol, 1 eq.) and compound 4 (8.8 g, 143.7 mmol, 10 eq.) in ethanol (2 mL) was stirred at 55° C. under nitrogen for 16 hours. TLC (DCM/MeOH=10/1) showed that new major spots were observed. The reaction mixture was extracted with ethyl acetate (50 mL) and washed with water (3×50 mL). The organic layer was dried over anhydrous sodium sulfate, filtered and concentrated under reduced pressure. The residue was purified by silica gel column chromatography, eluting with DCM/MeOH (1/0-10:1, v/v), to obtain compound 5 (1.008 g, 21%) as a yellow oil.

### 3. Synthesis of Compound 8

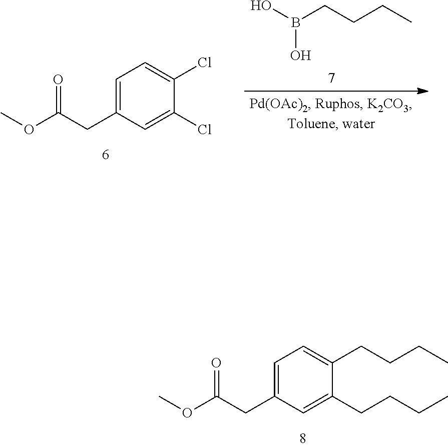

To a mixture of compound 6 (500 mg, 2.283 mmol, 1 eq.) and compound 7 (699 mg, 6.849 mmol, 3 eq.) in toluene/water (5 mL/1 mL) was added palladium acetate (51 mg, 0.228 mmol, 0.1 eq.), Ruphos (213 mg, 0.457 mmol, 0.2 eq.) and potassium carbonate (945 mg, 6.849 mmol, 3 eq.). The mixture was stirred at 110° C. overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 8 (507 mg, 85%) as a colorless oil.

### 4. Synthesis of Compound 9

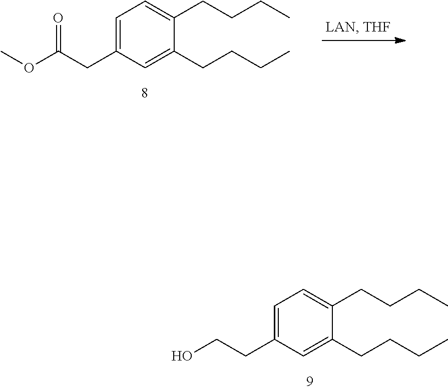 [SMILES: CCCCc1ccc(CC(=O)OC)cc1CCCC ; CCCCc1ccc(CCO)cc1CCCC]

To a mixture of compound 8 (507 mg, 1.935 mmol, 1 eq.) in THF (5 mL) was added lithium aluminum hydride (2 mL, 1.935 mmol, 1 M, 1 eq. in THF) at 0° C. under nitrogen. The mixture was stirred at room temperature for 3 hours. TLC (PE/EA=5/1) indicated that the reaction was complete and new major spots were observed. The mixture was quenched with water (2 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 9 as a colorless oil (492 mg, >100%) without further purification.

### 5. Synthesis of Compound 10

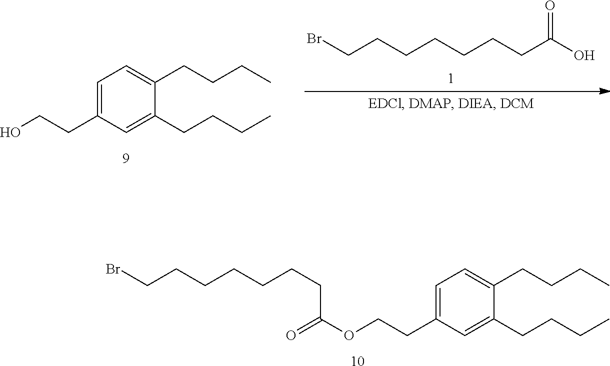

To a mixture of compound 9 (492 mg, 2.103 mmol, 1 eq.) and compound 1 (563 mg, 2.523 mmol, 1.2 eq.) in DCM (5 mL) was added EDCI (808 mg, 4.206 mmol, 2 eq.) and DMAP (103 mg, 0.84 mmol, 0.4 eq.), then DIEA (1.085 g, 8.412 mmol, 4 eq.) was added. The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-10/1), to obtain compound 10 (329 mg, 36%) as a colorless oil.

### 6. Synthesis of SW-II-134-3

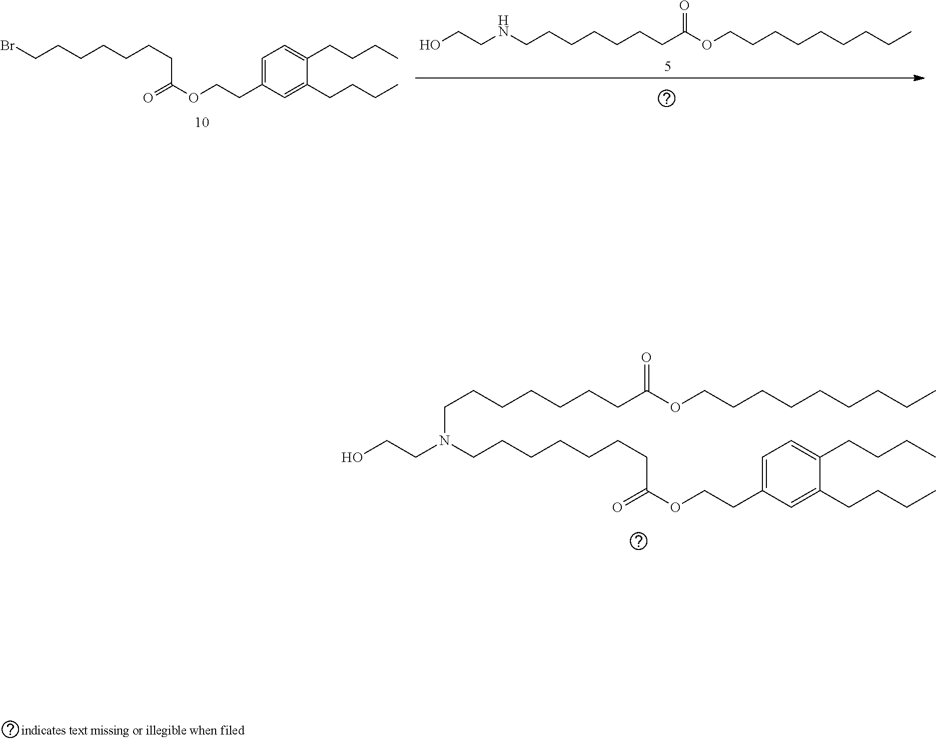

To compound 10 (150 mg, 0.34 mmol, 1 eq.) and compound 5 (134 mg, 0.41 mmol, 1.2 eq.) in CPME/CH3 To the mixture in CN (2 mL/2 mL) were added potassium carbonate (282 mg, 2.04 mmol, 6 eq.) and potassium iodide (113 mg, 0.68 mmol, 2 eq.). After addition, the mixture was stirred at 90° C. overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-134-3 (63.59 mg, 25%) as a yellow oil.

LCMS:Rt:1.247 min; MS m/z(ELSD):688.3[M+H]+;

HPLC: 95.945% purity, ELSD; RT=6.186 min.

¹H NMR (400 MHz, CDCl 3) δ7.07 (d, J=7.6 Hz, 1H), 6.97 (dd, J=9.9, 2.2 Hz, 2H), 4.26 (t, J=7.2 Hz, 2H), 4.05 (t, J=6.8 Hz, 2H), 2.88 (dd, J=14.8, 7.6 Hz, 4H), 2.78-2.74 (m, 2H), 2.67-2.54 (m, 8H), 2.29 (t, J=7.5 Hz, 4H), 1.68-1.47 (m, 15H), 1.37-1.22 (m, 27H), 0.98-0.86 (m, 9H).

¹³C NMR (101 MHz, CDCl3) δ173.86 (d, J=17.1 Hz), 140.66 (s), 138.76 (s), 134.93 (s), 129.74 (s), 129.24 (s), 126.19 (s), 77.36 (s), 77.04 (s), 76.72 (s), 65.01 (s), 64.48 (s), 57.73 (s), 55.73 (s), 53.93 (s), 34.76 (s), 34.28 (d, J=3.9 Hz), 33.54 (d, J=4.5 Hz), 32.41 (s), 31.95 (d, J=16.5 Hz), 29.49 (s), 29.15 (dd, J=21.1, 2.4 Hz), 28.66 (s), 27.04 (s), 25.95 (d, J=3.3 Hz), 24.85 (d, J=6.6 Hz), 22.98-22.58 (m), 14.08 (d, J=7.5 Hz).

### J. SW-II-135-1

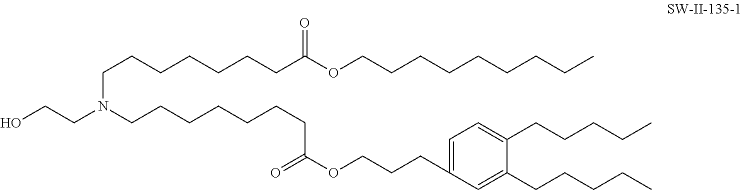 [SMILES: CCCCCCCCCOC(=O)CCCCCCCN(CCO)CCCCCCCC(=O)OCCCc1ccc(CCCCC)c(CCCCC)c1]

### 1. Synthesis of Compound 3

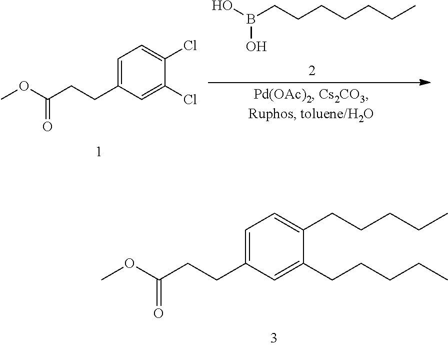

Compound 1 (500 mg, 2.16 mmol, 1.0 eq.) and compound 2 (750 mg, 6.46 mmol, 3.0 eq.) were dissolved in toluene/H2O (5 mL/1 mL), add Ruphos (201 mg, 0.43 mmol, 0.2 eq), Pd(OAc) to the mixture 2 (48.5 mg, 0.22 mmol, 0.1 eq) and Cs2CO3 (2.10 g, 6.46 mmol, 3.0 eq.). The reaction mixture was heated to reflux at 110° C. for 16 hours under nitrogen protection. TLC (petroleum ether/ethyl acetate=10/1) showed the reaction was complete and the desired product was formed. The reaction mixture was reacted with H2 Wash with O (40 mL) and extract 3 times with EA (50 mL). The obtained organic phase is washed twice with brine (20 mL) and filtered with anhydrous Na2SO4 Dry, filter and concentrate under reduced pressure. The residue was purified by silica gel column chromatography, eluting with petroleum ether/ethyl acetate (1/0-30/1), to obtain compound 3 (540 mg, 82.44%) as a yellow oil.

### 2. Synthesis of Compound 4

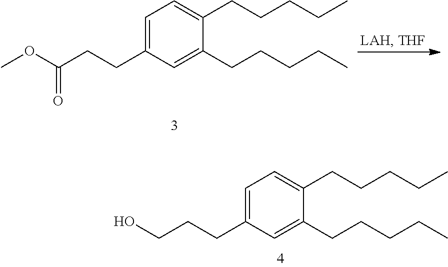 [SMILES: CCCCCc1ccc(CCC(=O)OC)cc1CCCCC ; CCCCCc1ccc(CCCO)cc1CCCCC]

LiAlH was added to compound 3 (540 mg, 1.78 mmol, 1.0 eq.) dissolved in THF (5 mL) under nitrogen protection at 0° C. 4 (3.55 mL, 3.55 mmol, 1M THF, 2 eq.). The reaction mixture was raised to room temperature and stirred under nitrogen protection for 2 hours. TLC (PE/EtOAc=10/1) showed the reaction was complete and new major spots were observed. The mixture was quenched with water (10 mL), then adjusted to pH=6-7 with 1M hydrochloric acid, and extracted three times with ethyl acetate (50 mL). The organic layer was washed with brine, dried over anhydrous sodium sulfate, filtered, and concentrated under reduced pressure. The residue was purified by silica gel column chromatography, eluting with PE/EA (1/0-10/1), to obtain compound 4 (442 mg, 90.2%) as a colorless oil.

### 3. Synthesis of Compound 6

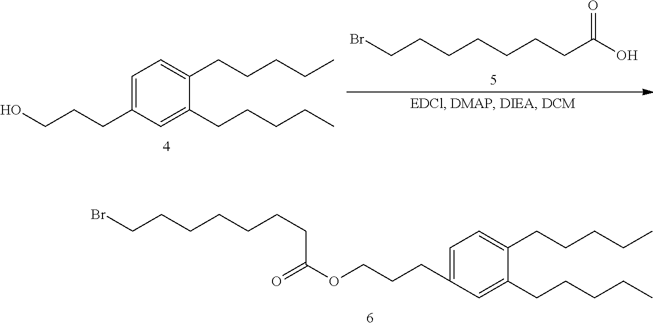

Compound 4 (442 mg, 1.60 mmol, 1.0 eq.) and compound 5 (428.5 mg, 1.92 mmol, 1.2 eq.) were dissolved in DCM (5 mL), and EDCI (612 mg, 3.2 mmol, 2.0 eq.) was added to the mixture.) and DMAP (78.2 mg, 0.64 mmol, 0.4 eq.), then DIEA (826 mg, 6.4 mmol, 4.0 eq.) was added. The reaction mixture was stirred at room temperature under nitrogen protection for 16 hours. TLC (petroleum ether/ethyl acetate=10/1) showed that compound 4 was consumed and the desired product formed. The reaction mixture was reacted with H2 Wash with O (40 mL) and extract 3 times with EA (50 mL). The obtained organic phase is washed twice with brine (20 mL) and washed with anhydrous Na2SO4 Dry, filter and concentrate under reduced pressure. The residue was purified by silica gel column chromatography, eluting with petroleum ether/ethyl acetate (1/0-10/1), to obtain compound 3 (342 mg, 44.5%) as a yellow oil.

### 4. Synthesis of SW-II-135-1

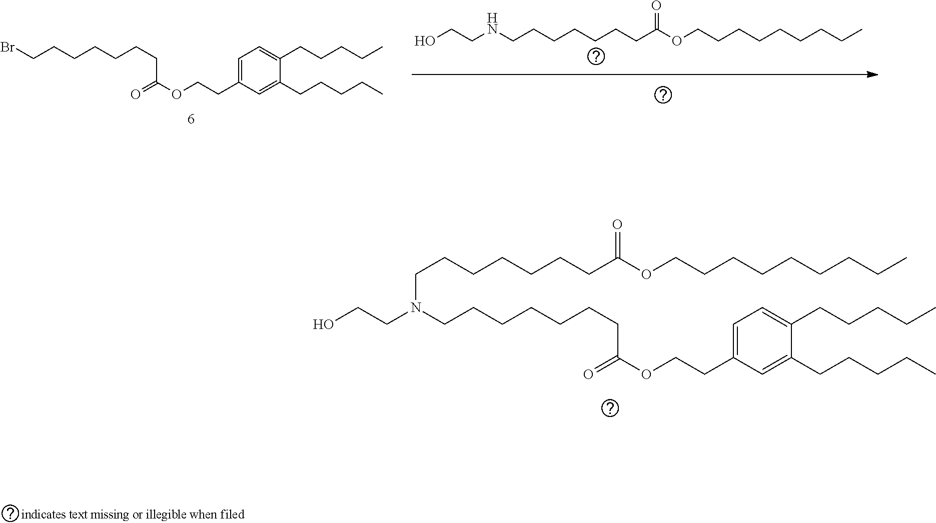 [SMILES: CCCCCCCCCOC(=O)CCCCCCCNCCO ; CCCCCc1ccc(CCOC(=O)CCCCCCCBr)cc1CCCCC ; CCCCCCCCCOC(=O)CCCCCCCN(CCO)CCCCCCCC(=O)OCCc1ccc(CCCCC)c(CCCCC)c1]

Compound 6 (175 mg, 0.365 mmol, 1.2 eq.) and compound 7 (100 mg, 0.304 mmol, 1.0 eq.) were dissolved in CPME/CH3CN (1 mL/1 mL), to the mixture were added potassium carbonate (210 mg, 1.52 mmol, 5.0 eq) and potassium iodide (101 mg, 0.61 mmol, 2.0 eq). After the addition, the reaction mixture was stirred at 90° C. overnight under nitrogen protection. TLC (DCM/MeOH=10/1) showed that the reaction was complete and the desired product was formed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-135-1 (83.89 mg, 55.6%) as a yellow oil.

LCMS:Rt:1.356 min; MS m/z(ELSD):730.5[M+H]+;

HPLC: 100% purity at ELSD; RT=12.614 min.

¹H NMR (400 MHz, CDCl3) δ6.97 (d, J=7.6 Hz, 1H), 6.91-6.74 (m, 2H), 4.76 (s, 1H), 3.99 (dt, J=13.6, 6.4 Hz, 4H), 3.72-3.58 (m, 2H), 2.85-2.73 (m, 2H), 2.72-2.61 (m, 4H), 2.59-2.41 (m, 6H), 2.22 (dd, J=13.2, 7.2 Hz, 4H), 1.93-1.79 (m, 2H), 1.62-1.41 (m, 14H), 1.23 (d, J=24.4 Hz, 32H), 0.82 (ddd, J=13.6, 8.0, 5.6 Hz, 9H).

¹³C NMR (101 MHz, CDCl3) δ172.81 (d, J=6.4 Hz), 139.55 (s), 137.38 (s), 137.14 (s), 128.16 (d, J=2.4 Hz), 124.69 (s), 76.51 (s), 76.19 (s), 75.88 (s), 63.43 (s), 62.78 (s), 56.53 (s), 54.90 (s), 52.84 (s), 33.23 (d, J=2.4 Hz), 31.73 (s), 31.28 (s), 30.91 (dd, J=20.0, 6.4 Hz), 30.10 (d, J=3.2 Hz), 29.29 (s), 28.36 (d, J=22.8 Hz), 28.23 (s), 27.97 (s), 27.64 (s), 25.92 (s), 24.92 (s), 24.34 (s), 23.84 (s), 21.62 (d, J=7.6 Hz), 13.08 (d, J=4.7 Hz).

### K. Compound SW-II-135-2

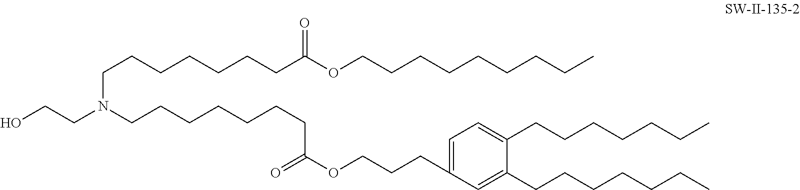 [SMILES: CCCCCCCCCOC(=O)CCCCCCCN(CCO)CCCCCCCC(=O)OCCCc1ccc(CCCCCCC)c(CCCCCCC)c1]

### 1. Synthesis of Compound 3

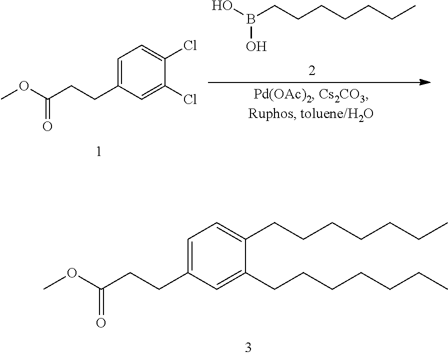

Compound 1 (500 mg, 2.16 mmol, 1.0 eq.) and compound 2 (931 mg, 6.46 mmol, 3.0 eq.) were dissolved in toluene/H2O (5 mL/1 mL), add Ruphos (201 mg, 0.43 mmol, 0.2 eq), Pd(OAc) to the mixture 2 (48.5 mg, 0.22 mmol, 0.1 eq) and Cs2CO3 (2.1 Og, 6.46 mmol, 3.0 eq.). The reaction mixture was heated to reflux at 110° C. for 16 hours under nitrogen protection. TLC (petroleum ether/ethyl acetate=10/1) showed the reaction was complete and the desired product was formed. The reaction mixture was reacted with H2 Wash with O (40 mL) and extract 3 times with EA (50 mL). The obtained organic phase is washed twice with brine (20 mL) and washed with anhydrous Na2SO4 Dry, filter and concentrate under reduced pressure. The residue was purified by silica gel column chromatography, eluting with petroleum ether/ethyl acetate (1/0-30/1), to obtain compound 3 (651 mg, 84%) as a yellow oil.

### 2. Synthesis of Compound 4

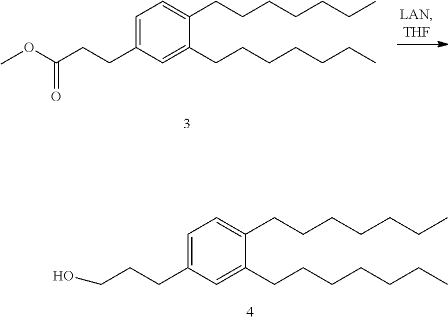 [SMILES: CCCCCCCc1ccc(CCC(=O)OC)cc1CCCCCCC ; CCCCCCCc1ccc(CCCO)cc1CCCCCCC]

LiAlH was added to compound 3 (651 mg, 1.81 mmol, 1.0 eq.) dissolved in THF (7 mL) under nitrogen protection at 0° C. 4 (3.62 mL, 3.62 mmol, 1M in THF, 2 eq.). The reaction mixture was raised to room temperature and stirred under nitrogen protection for 2 hours. TLC (PE/EtOAc=10/1) showed the reaction was complete and new major spots were observed. The mixture was quenched with water (10 mL), then adjusted to pH=6-7 with 1M hydrochloric acid, and extracted three times with ethyl acetate (50 mL). The organic layer was washed with brine, dried over anhydrous sodium sulfate, filtered, and concentrated under reduced pressure. The residue was purified by silica gel column chromatography, eluting with PE/EA (1/0-10/1), to obtain compound 4 (571 mg, 95.2%) as a colorless oil.

### 3. Synthesis of Compound 6

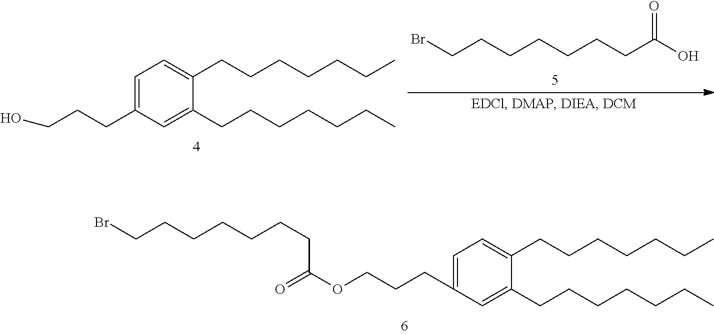

Compound 4 (571 mg, 1.72 mmol, 1.0 eq.) and compound 5 (459 mg, 2.06 mmol, 1.2 eq.) were dissolved in DCM (6 mL), and EDCI (657 mg, 3.44 mmol, 2.0 eq.) was added to the mixture. and DMAP (84 mg, 0.68 mmol, 0.4 eq.), then DIEA (887.5 mg, 6.88 mmol, 4.0 eq.) was added. The reaction mixture was stirred at room temperature under nitrogen protection for 16 hours. TLC (petroleum ether/ethyl acetate=10/1) showed that compound 4 was consumed and the desired product formed. The reaction mixture was reacted with H2 Wash with O (50 mL) and extract 3 times with EA (60 mL). The resulting organic phase is washed twice with brine (25 mL) and washed with anhydrous Na2SO4 Dry, filter and concentrate under reduced pressure. The residue was purified by silica gel column chromatography, eluting with petroleum ether/ethyl acetate (1/0-10/1), to obtain compound 3 (245 mg, 26.5%) as a yellow oil.

### 4. Synthesis of SW-II-135-2

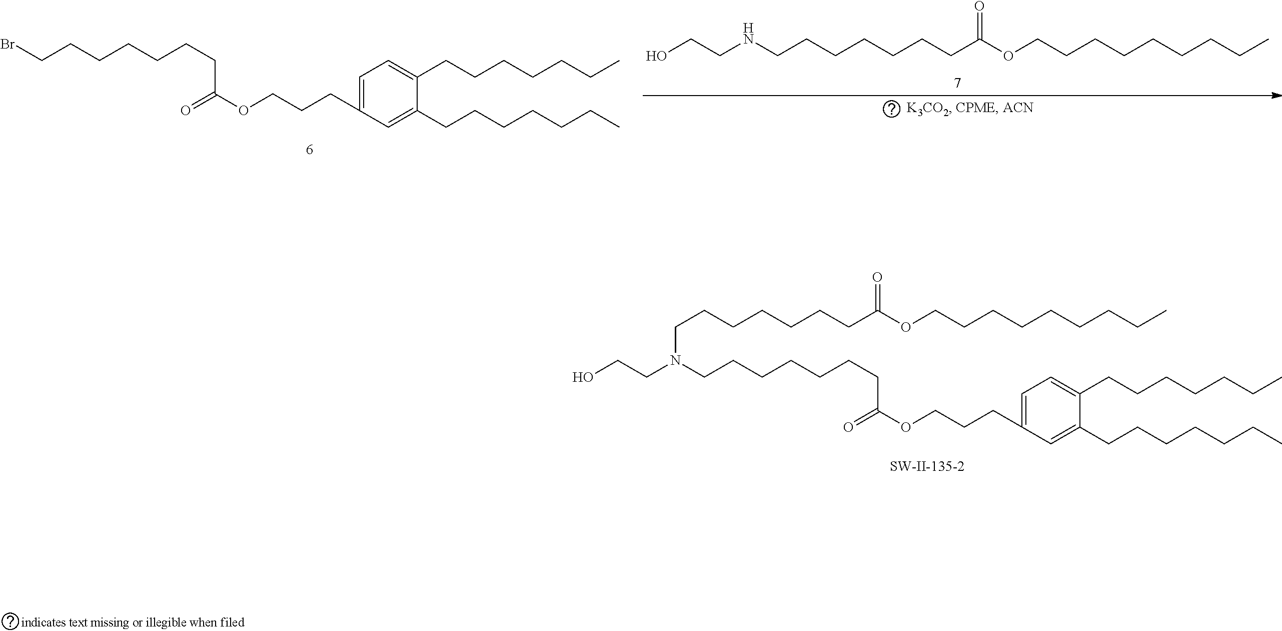

Compound 6 (245 mg, 0.456 mmol, 1.5 eq.) and compound 7 (100 mg, 0.3 mmol, 1.0 eq.) were dissolved in CPME/CH3CN (1 mL/1 mL), to the mixture were added potassium carbonate (210 mg, 1.52 mmol, 5.0 eq) and potassium iodide (101 mg, 0.61 mmol, 2.0 eq). After the addition, the reaction mixture was stirred at 90° C. overnight under nitrogen protection. TLC (DCM/MeOH=10/1) showed that the reaction was complete and the desired product was formed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-135-2 (31.41 mg, 21.9%) as a yellow oil.

LCMS:Rt:1.608 min; MS m/z(ELSD):786.4[M+H]+;

HPLC: 95.16% purity, ELSD; RT=7.919 min.

¹H NMR (400 MHz, CDCl 3) δ6.98 (d, J=7.6 Hz, 1H), 6.87 (d, J=2.4 Hz, 2H), 4.28-4.13 (m, 1H), 4.04-3.95 (m, 4H), 3.94-3.84 (m, 2H), 3.14-2.89 (m, 6H), 2.59-2.43 (m, 6H), 2.23 (dd, J=13.8, 7.2 Hz, 4H), 1.88-1.82 (m, 2H), 1.70 (s, 4H), 1.57-1.46 (m, 10H), 1.33-1.16 (m, 40H), 0.90-0.72 (m, 9H).

¹³C NMR (100 MHz, CDCl 3) δ172.82 (d, J=6.8 Hz), 139.61 (s), 137.29 (d, J=16.4 Hz), 128.15 (s), 124.67 (s), 76.41 (s), 76.09 (s), 75.77 (s), 63.50 (s), 62.87 (s), 55.49 (s), 54.92 (s), 52.98 (s), 33.16 (d, J=2.4 Hz), 31.77 (s), 31.33 (s), 30.80 (d, J=6.5H z), 30.42 (d, J=3.6 Hz), 29.29 (s), 28.99-28.66 (m), 28.47 (s), 28.23 (d, J=2.8 Hz), 28.06-27.45 (m), 25.58 (s), 24.91 (s), 23.71 (s), 22.79 (s), 21.66 (s), 13.10 (s).

### L. Compound SW-II-136-2

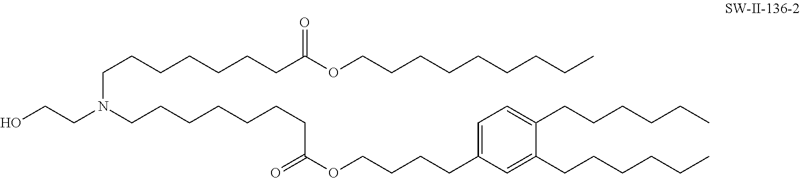 [SMILES: CCCCCCCCCOC(=O)CCCCCCCN(CCO)CCCCCCCC(=O)OCCCCc1ccc(CCCCCC)c(CCCCCC)c1]

### SW-II-136-2

### 1. Synthesis of Compound 3

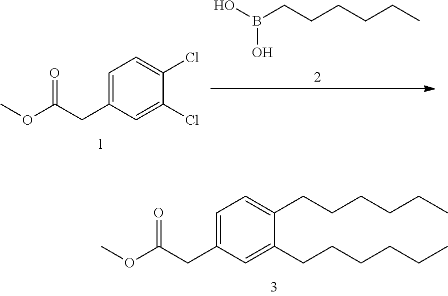

Compound 1 (3 g, 13.70 mmol, 1.0 eq.) and compound 2 (5.34 g, 41.09 mmol, 3.0 eq.) were dissolved in toluene/H2O (30 mL/3 mL), add Ruphos (1.28 g, 2.74 mmol, 0.2 eq), Pd(OAc) to the mixture 2 (308.3 mg, 1.37 mmol, 0.1 eq) and K2CO3 (5.67 g, 41.10 mmol, 3.0 eq.). The reaction mixture was heated to reflux at 110° C. for 16 hours under nitrogen protection. TLC (PE/EA=10/1) showed the reaction was complete and the desired product was formed. The reaction mixture was reacted with H2 Wash with O (90 mL) and extract 3 times with EA (110 mL). The resulting organic phase is washed twice with brine (40 mL) and washed with anhydrous Na2SO4 Dry, filter and concentrate under reduced pressure. The residue was purified by silica gel column chromatography, eluting with PE/EA (1/0-30/1), to obtain compound 3 (1.98 g, 45.5% a yellow oil.

### 2. Synthesis of Compound 4

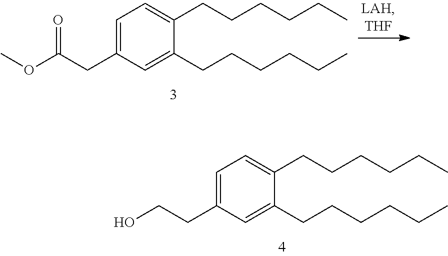 [SMILES: CCCCCCc1ccc(CC(=O)OC)cc1CCCCCC ; CCCCCCc1ccc(CCO)cc1CCCCCC]

LiAlH was added to compound 3 (1.98 g, 6.23 mmol, 1.0 eq.) dissolved in THF (20 mL) under nitrogen protection at 0° C. 4 (1M, 12.45 mL, 2.0 eq). The reaction mixture was raised to room temperature and stirred under nitrogen protection for 2 hours. TLC (PE/EtOAc=10/1) showed the reaction was complete and new major spots were observed. Mixture with H2 Quench with O (70 mL), then adjust pH=6-7 with 1M hydrochloric acid, and extract 3 times with EA (80 mL). The organic layer was washed with brine and washed with anhydrous Na2SO4 Dry, filter, and concentrate under reduced pressure. The residue was purified by silica gel column chromatography, eluting with PE/EA (1/0-10/1), to obtain compound 4 (1.28 g, 71.1%) as a colorless oil.

### 3. Synthesis of Compound 7

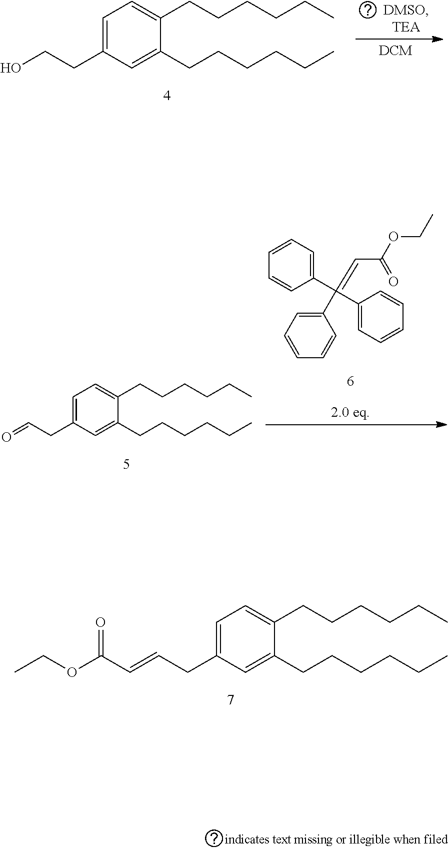

Under nitrogen protection at 0° C., DMSO (3.63 g, 51.72 mmol, 15 eq) and TEA (1.25 g, 12.4 mmol, 4.0 eq.) were added to compound 4 (900 g, 3.1 mmol, 1.0 eq.) dissolved in DCM (9 mL).) and PySO 3 (1.27 g, 7.97 mmol, 2.57 eq). The mixture was stirred at 0° C. for 30 minutes, then raised to room temperature and stirred under nitrogen protection for 90 minutes. Then compound 6 (4.74 g, 13.62 mmol, 3.0 eq.) was added to the mixture, and the reaction mixture was reacted at 25° C. for 2 hours under nitrogen protection. TLC (PE/EA=10/1) showed the reaction was complete and the desired product was formed. The reaction mixture was reacted with H2 Wash with O (60 mL) and extract with EA (70 mL) three times. The obtained organic phase is washed twice with brine (40 mL) and filtered with anhydrous Na2SO4 Dry, filter and concentrate under reduced pressure. The residue was purified by silica gel column chromatography, eluting with PE/EA (1/0-10/1), to obtain compound 7 (345 mg, 27.9%) as a yellow oil.

### 4. Synthesis of Compound 8

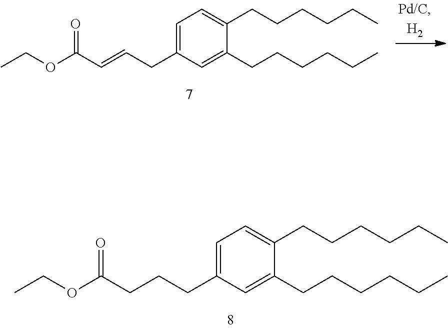

Compound 7 (340 mg, 0.95 mmol, 1.0 eq.) and Pd/C (100 mg) were added to MeOH (4 ml), and the reaction mixture was stirred under hydrogen protection at room temperature for 16 h. TLC (PE/EA=10/1) showed complete consumption of starting material and formation of desired product. The reaction mixture was filtered through celite and washed with MeOH (40 mL×2), and filtered with anhydrous Na2SO4 The filtrate was dried and concentrated under reduced pressure to obtain compound 8 (298 mg, 88.2%) as a light yellow oil.

### 5. Synthesis of Compound 9

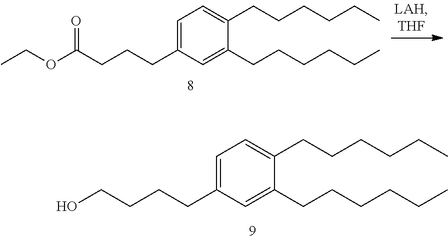 [SMILES: CCCCCCc1ccc(CCCC(=O)OCC)cc1CCCCCC ; CCCCCCc1ccc(CCCCO)cc1CCCCCC]

LiAlH was added to compound 8 (298 mg, 0.83 mmol, 1.0 eq.) dissolved in THF (3 mL) under nitrogen protection at 0° C. 4 (1M, 1.66 mL, 2.0 eq). The reaction mixture was raised to room temperature and stirred under nitrogen protection for 2 hours. TLC (PE/EtOAc=10/1) showed the reaction was complete and new major spots were observed. Mixture with H2 Quench with O (20 mL), then adjust pH=6-7 with 1M hydrochloric acid, and extract 3 times with EA (30 mL). The organic layer was washed with brine and washed with anhydrous Na2SO4 Dry, filter, and concentrate under reduced pressure. The residue was purified by silica gel column chromatography, eluting with PE/EA (1/0-10/1), to obtain compound 9 (254 mg, 98.3%) as a colorless oil.

### 6. Synthesis of Compound 11

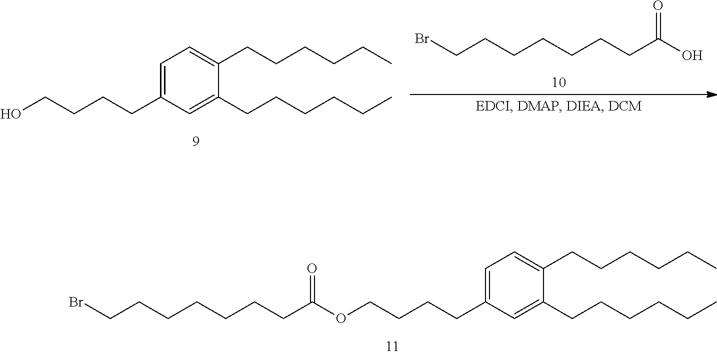

Compound 9 (254 mg, 0.80 mmol, 1.0 eq.) and compound 10 (214 mg, 0.96 mmol, 1.2 eq.) were dissolved in DCM (3 mL), and EDCI (305.6 mg, 1.6 mmol, 2.0 eq.) was added to the mixture.) and DMAP (39 mg, 0.32 mmol, 0.4 eq.), then DIEA (412.8 mg, 3.2 mmol, 4.0 eq.) was added. The reaction mixture was stirred at room temperature under nitrogen protection for 16 hours. TLC (PE/EA=10/1) showed that compound 9 was consumed and the desired product formed. The reaction mixture was adjusted to pH=4-6 with 1M hydrochloric acid, and extracted three times with EA (30 mL). The resulting organic phase was washed twice with brine (15 mL), and washed with anhydrous Na2SO4 Dry, filter and concentrate under reduced pressure. The residue was purified by silica gel column chromatography, eluting with PE/EA (1/0-7/1), to obtain compound 11 (210 mg, 50.5%) as a yellow oil.

### 7. Synthesis of SW-II-136-2

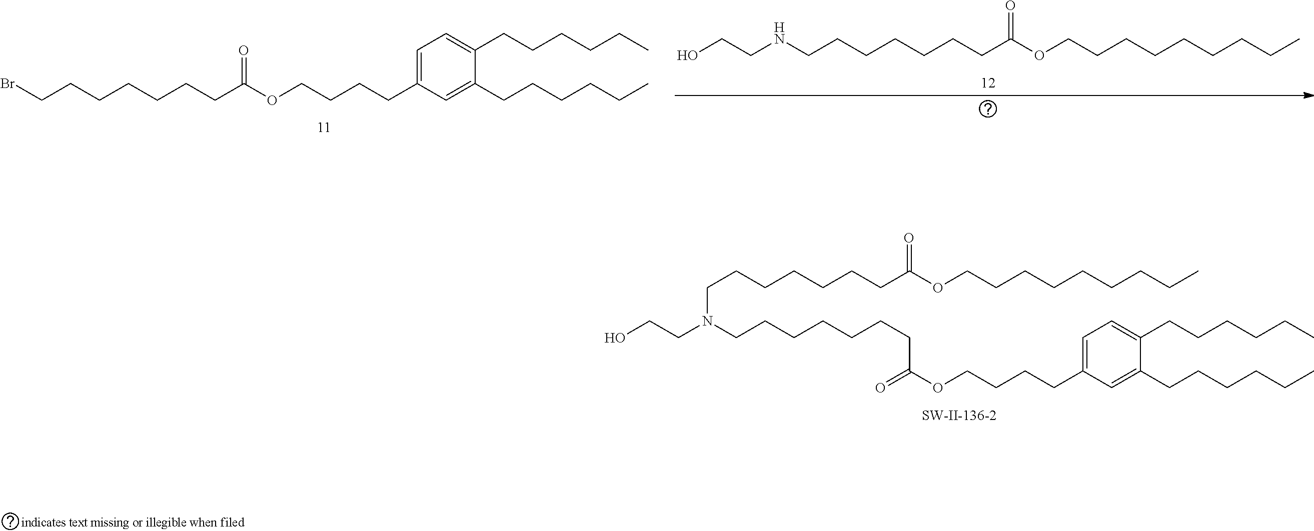

Compound 11 (200 mg, 0.38 mmol, 1.2 eq.) and compound 12 (105 mg, 0.32 mmol, 1.0 eq.) were dissolved in CPME/CH3CN (1.5 mL/1.5 mL), add K to this mixture 2CO3 (220.2 mg, 1.60 mmol, 5.0 eq) and KI (106 mg, 0.64 mmol, 2.0 eq). After the addition, the reaction mixture was stirred at 90° C. overnight under nitrogen protection. TLC (DCM/MeOH=10/1) showed that the reaction was complete and the desired product was formed. The mixture was extracted with EA and washed with water. Anhydrous Na was used for the organic layer 2SO4 Dry, filter and concentrate under reduced pressure. The residue was purified by silica gel column chromatography, eluting with DCM/MeOH (1/0-10:1, v/v), to obtain compound SW-II-136-2 (208 mg, 90.4%) as a yellow oil.

LCMS:Rt:2.146 min; MS m/z(ELSD):773.3[M+H]+;

HPLC: 99.49% purity, ELSD; RT=8.055 min.

¹H NMR (400 MHz, CDCl 3) δ7.04 (d, J=7.6 Hz, 1H), 6.92 (d, J=9.6 Hz, 2H), 4.45 (s, 1H), 4.06 (dd, J=12.0, 5.2 Hz, 4H), 3.64 (t, J=5.2 Hz, 2H), 2.72 (t, J=5.2 Hz, 2H), 2.65-2.50 (m, 10H), 2.29 (t, J=7.6 Hz, 4H), 1.69-1.48 (m, 18H), 1.41-1.24 (m, 36H), 0.95-0.78 (m, 9H).

¹³C NMR (101 MHz, CDCl 3) δ173.86 (d, J=2.8 Hz), 140.48 (s), 139.24 (s), 138.01 (s), 129.13 (d, J=14.8 Hz), 125.67 (s), 77.37 (s), 77.05 (s), 76.73 (s), 64.45 (s), 64.23 (s), 57.88 (s), 55.91 (s), 53.94 (s), 35.07 (s), 34.29 (d, J=3.2 Hz), 32.79 (s), 32.35 (s), 31.82 (d, J=8.4 Hz), 31.38 (s), 29.50 (d, J=2.4 Hz), 29.16 (dd, J=18.0, 2.0 Hz), 28.66 (s), 28.35 (s), 27.78 (s), 27.08 (s), 26.02 (d, J=17.2 Hz), 24.89 (d, J=1.6 Hz), 22.65 (s), 14.10 (s).

### M. Compound SW-JJ-137-1

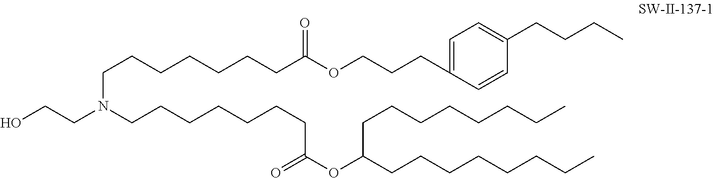 [SMILES: CCCCCCCCC(CCCCCCCC)OC(=O)CCCCCCCN(CCO)CCCCCCCC(=O)OCCCc1ccc(CCCC)cc1]

### 1. Synthesis of Compound 3

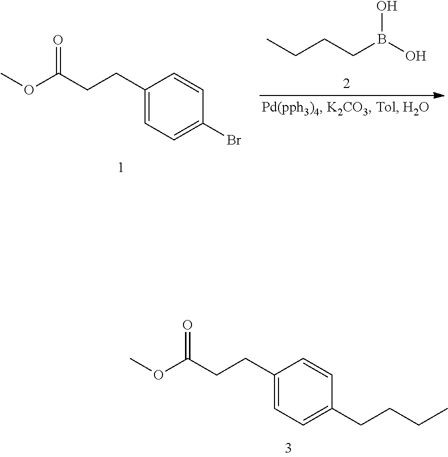

Compound 1 (500 mg, 1.95 mmol, 1.0 eq.) was dissolved in toluene (5.0 mL), then compound 2 (239 mg, 2.34 mmol, 1.2 eq.), Pd(PPh) were added 3) 4 (225 mg, 0.19 mmol, 0.1 eq), water (1 mL) and K2CO3 (808 g, 5.85 mmol, 3.0 eq.). React at 110° C. for 3 hours under nitrogen protection. TLC (PE/EA=5/1) showed that the starting material had reacted completely and the desired product was formed. Add H to the reaction 2O (70 mL), EA (80 mL×3) extraction. Combine the organic phases and wash with saturated brine (2×30 mL), anhydrous Na2SO4 Dry, filter and spin dry under reduced pressure. The residue was purified with a silica gel column and eluted with PE/EA (1/0-5:1, v/v) to obtain a colorless oily compound (320 mg, 70%).

### 2. Synthesis of Compound 4

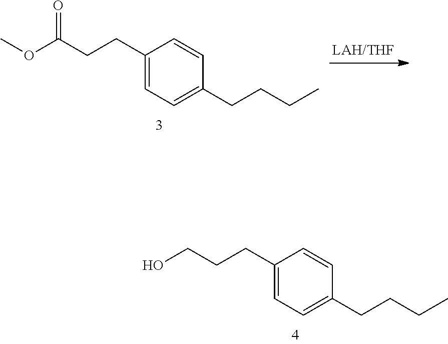 [SMILES: CCCCc1ccc(CCC(=O)OC)cc1 ; CCCCc1ccc(CCCO)cc1]

Compound 3 (300 mg, 1.28 mmol, 1.0 eq.) was dissolved in THF (4.0 mL), and LAH (97 mg, 2.56 mmol, 2.0 eq.) was added at 0° C. under nitrogen protection. Then react at room temperature for 2 hours. TLC (PE/EA=10/1) showed that the starting material reacted completely and the desired product was formed. Add HCl (1M, 4 mL) solution and H2 Quench with 0 (10 mL) and extract with EA (50 mL×3). The organic phase was washed with saturated brine (2×30 mL) and anhydrous Na2SO4 Dry, filter and spin dry under reduced pressure. The residue was purified with a silica gel column and eluted with PE/EA (1/0-10/1, v/v) to obtain compound 4 (224 mg, 84.8%) as a yellow oil.

### 3. Synthesis of Compound 6

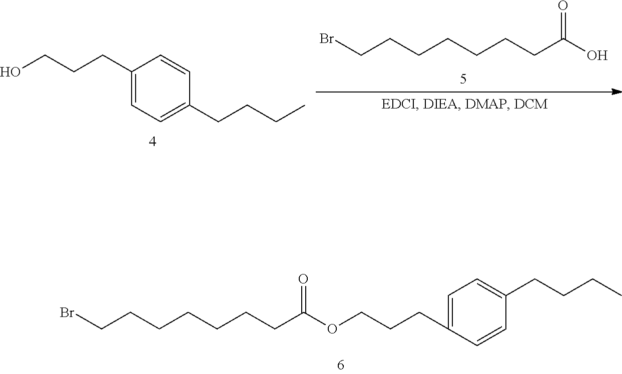 [SMILES: CCCCc1ccc(CCCO)cc1 ; O=C(O)CCCCCCCBr ; CCCCc1ccc(CCCOC(=O)CCCCCCCBr)cc1]

Compound 4 (90 mg, 0.47 mmol, 1.0 eq.) was dissolved in DCM (3.0 mL), and compound 5 (127 mg, 0.56 mmol, 1.2 eq.), EDCI (180 mg, 0.94 mmol, 2.0 eq.), DIEA (242 mg) were added, 1.88 mmol, 4.0 eq.) and DMAP (23 mg, 0.18 mmol, 0.4 eq.). Then, the reaction was carried out at room temperature overnight under nitrogen protection. TLC (PE/EA=20/1) showed that the starting material had been reacted and the desired product had been formed. Quench the reaction with HCl (1M) solution, adjust pH=4-6, and extract with EA (40 mL×3). Combine the organic phases and wash with saturated brine (2×30 mL), anhydrous Na2SO4 Dry, filter and spin dry under reduced pressure. The residue was purified using a silica gel column and eluted with PE/EA (1/0-20/1, v/v) to obtain compound 6 (90 mg, 48.6%) as a colorless oil.

### 4. Synthesis of SW-II-137-1

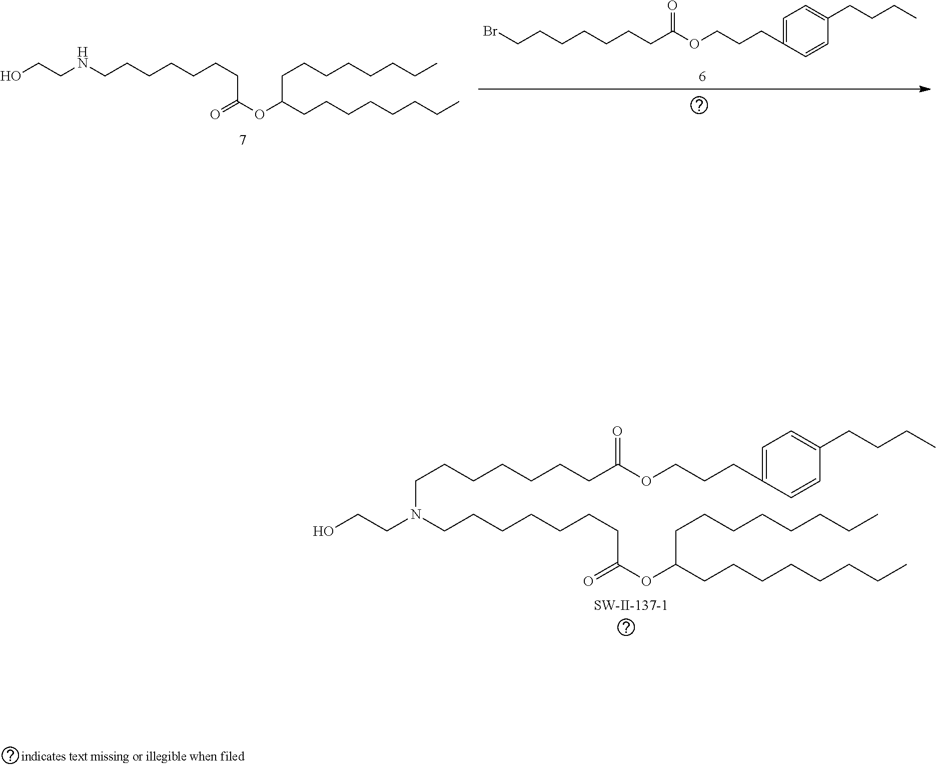 [SMILES: CCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCC)cc1]

Compound 6 (90 mg, 0.25 mmol, 1.0 eq.) was dissolved in MeCN (2 mL) and compound 7 (110 mg, 0.25 mmol, 1.0 eq.), KI (76 mg, 0.50 mmol, 2.0 eq.), CPME (2 mL) and K were added. 2CO3 (157 mg, 1.25 mmol, 5.0 eq). The reaction was carried out overnight at 90° C. under nitrogen protection. TLC (DCM/MeOH=10/1) showed complete reaction of the starting material and formation of the desired product. Quench with water (50 mL) and extract with EA (40 mL×3). Combine the organic phases and wash with saturated brine (2×30 mL), anhydrous Na2SO4 Dry, filter and spin dry under reduced pressure. The residue was purified by silica gel column and eluted with DCM/MeOH (1/0-10/1, v/v) to obtain a yellow oily compound (98 mg, 52.12%, SW-II-137-1).

LCMS:Rt:1.596 min; MS m/z(ELSD):758.4[M+H]+;

HPLC: 98.02% purity, ELSD; RT=5.993 min.

¹H NMR (400 MHz, CDCl₃) δ 7.02 (d, J=8.8 Hz, 4H), 4.92-4.71 (m, 1H), 4.01 (t, J=6.4 Hz, 2H), 3.78 (s, 1H), 3.55 (t, J=5.2 Hz, 23H) 2.76-2.40 (m, 10H), 2.21 (dd, J=15.6, 7.7 Hz, 4H), 1.95-1.80 (m, 2H), 1.49 (ddd, J=24.4, 15.8, 6.2 Hz, 15H), 1.34-1.13 (m, 37H) 0.82 (dt, J=13.6, 7.2 Hz, 9H).

¹³C NMR (101 MHz CDCl₃) δ 173.79 (s), 173.57 (s), 140.49 (s), 138.30 (s), 128.43 (s), 128.22 (s), 77.43 (s), 77.11 (s), 76.79 (s), 74.11 (s), 63.66 (s), 55.75 (s), 53.90 (s), 35.22 (s), 34.63 (s), 34.20 (d, J=11.6 Hz), 33.70 (s), 31.80 (d, J=11.2 Hz), 30.30 (s), 29.51 (d, J=2.8 Hz), 29.13 (dd, J=9.6, 6.8 Hz), 27.12 (d, J=2.8 Hz), 26.29 (s), 25.31 (s), 24.97 (d, J=15.6 Hz), 22.66 (s), 22.37 (s), 14.02 (d, J=15.2 Hz).

### N. Compound SW-II-137-2

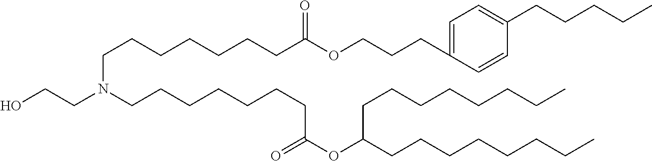 [SMILES: CCCCCCCCC(CCCCCCCC)OC(=O)CCCCCCCN(CCO)CCCCCCCC(=O)OCCCc1ccc(CCCCC)cc1]

### 1. Synthesis of Compound 3

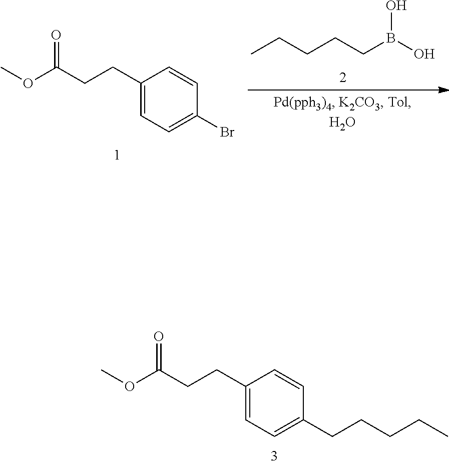

Compound 1 (500 mg, 2.06 mmol, 1.0 eq.), Compound 2 (286 mg, 2.47 mmol, 1.2 eq.), Pd(PPh 3) 4 (119 mg, 0.1 mmol, 0.1 eq) and K2CO3 (851 mg, 6.21 mmol, 3.0 eq.) was dissolved in toluene (5.0 mL), and water (0.5 mL) was added. Then, the reaction was carried out at 110° C. for 3 hours under nitrogen protection. TLC (PE/EA=5/1) showed that the starting material reacted completely and the desired compound was formed. Use H2 The reaction was quenched with O (70 mL) and extracted with EA (80 mL×3). Wash the organic phase with saturated brine (2×30 mL), anhydrous Na2SO4 Dry, filter and spin dry under reduced pressure. The residue was purified with a silica gel column and eluted with (PE/EA=5/1, v/v) to obtain compound 3 (420 mg, 87.5%) as a colorless oil.

### 2. Synthesis of Compound 4

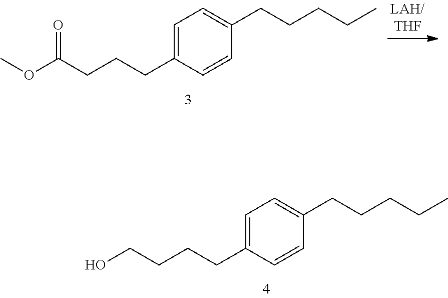 [SMILES: CCCCCc1ccc(CCCC(=O)OC)cc1 ; CCCCCc1ccc(CCCCO)cc1]

Compound 3 (420 mg, 1.78 mmol, 1.0 eq.) was dissolved in THF (3.0 mL), and LAH (1 M, 7 mL, 2.0 eq.) was added dropwise at 0° C. under nitrogen protection. Then, the reaction was carried out at room temperature for 2 hours. TLC (PE/EA=5/1) showed that the starting material was completely reacted and the desired product was formed. Use HCl (1M, 4 mL) solution and H2 Quench with O (10 mL) and extract with EA (50 mL×3). The organic phase was washed with saturated brine (2×30 mL) and anhydrous Na2SO4 Dry, filter and spin dry under reduced pressure. The residue was purified using a silica gel column and eluted with (PE/EA=5/1, v/v) to obtain compound 4 (320 mg, 94%) as a colorless oil.

### 3. Synthesis of Compound 6

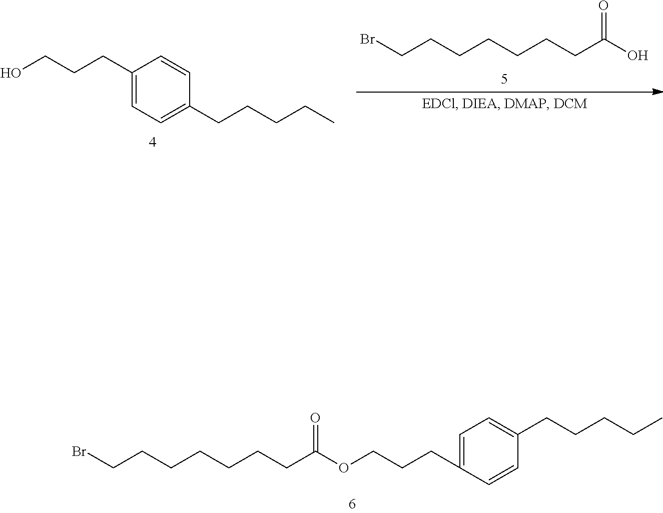 [SMILES: O=C(O)CCCCCCCBr ; CCCCCc1ccc(CCCO)cc1 ; CCCCCc1ccc(CCCOC(=O)CCCCCCCBr)cc1]

Compound 4 (320 mg, 1.55 mmol, 1.0 eq.) was dissolved in DCM (4.0 mL), and compound 5 (416 mg, 1.86 mmol, 1.2 eq.), EDCI (594 mg, 3.11 mmol, 2.0 eq.), DIEA (802 mg) were added, 6.21 mmol, 4.0 eq.) and DMAP (76 mg, 0.62 mmol, 0.4 eq.). Then, the reaction was carried out at room temperature overnight under nitrogen protection. TLC (PE/EA=20/1) showed that the starting material was completely reacted and the desired product was formed. The reaction was quenched with HCl (1M) solution and adjusted to pH=4-6, and extracted with DCM (60 mL×3). The organic phase was washed with saturated brine (2×35 mL) and anhydrous Na2SO4 Dry, filter and spin dry under reduced pressure. The residue was purified with a silica gel column and eluted with (PE/EA=5/1, v/v) to obtain compound 6 (300 mg, 47.17%) as a colorless oil.

### 4. Synthesis of SW-II-137-2

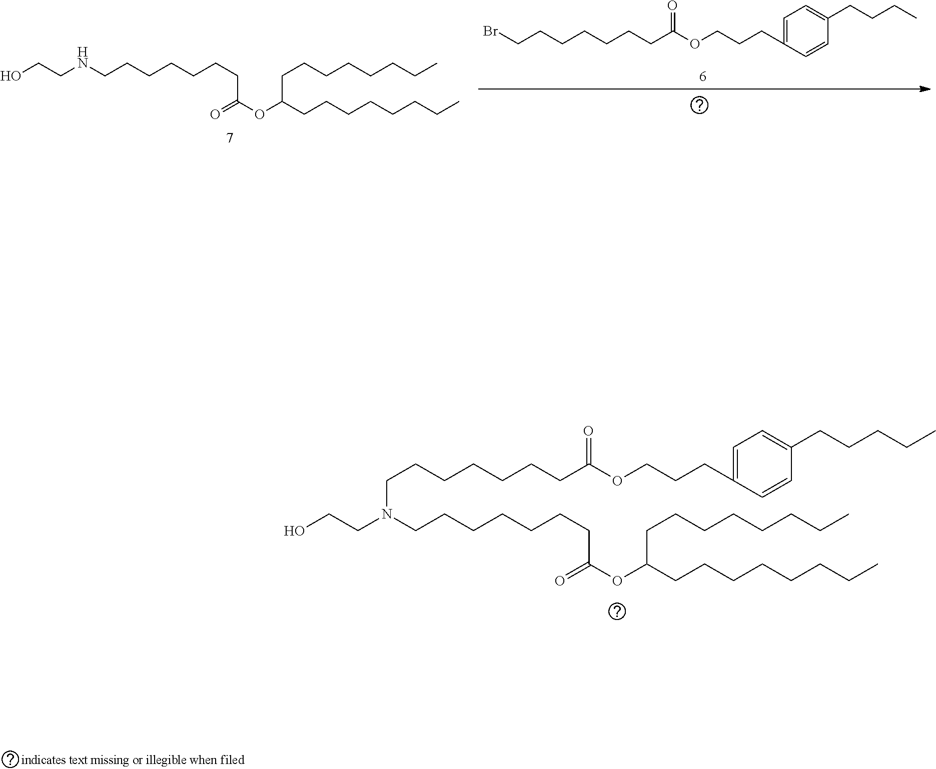 [SMILES: CCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCCC)cc1]

Compound 6 (167 mg, 0.41 mmol, 1.2 eq.), compound 7 (150 mg, 0.34 mmol, 1.0 eq.), KI (113 mg, 0.68 mmol, 2.0 eq.) and CPME (2 mL) were dissolved in MeCN (2 mL), and K was added 2CO 3 (235 mg, 1.70 mmol, 5.0 eq). The reaction was carried out overnight at 90° C. under nitrogen protection. TLC (DCM/MeOH=10/1) showed complete reaction of starting material and formation of desired product. The reaction was quenched with water (50 mL) and extracted with EA (60 ml×3). Use anhydrous Na for the organic phase 2SO4 Dry, filter and spin dry under reduced pressure. The residue was purified with a silica gel column and eluted with DCM/MeOH (1/0-10/1, v/v) to obtain a light yellow oily compound (105 mg, 40.3%, SW-II-137-2).

LCMS:Rt:1.660 min; MS m/z(ELSD):772.4[M+H]+;

HPLC: 98.38% purity, ELSD; RT=8.743 min.

¹H NMR (400 MHz, CDCl 3) δ7.10 (d, J=8.8 Hz, 4H), 5.04-4.74 (m, 1H), 4.08 (t, J=6.4 Hz, 2H), 3.58 (t, J=5.2 Hz, 2H), 2.65 (dd, J=9.6, 5.6 Hz, 4H), 2.60-2.44 (m, 6H), 2.29 (dd, J=16.4, 7.6 Hz, 4H), 2.01-1.88 (m, 2H), 1.59 (dt, J=9.2, 7.2 Hz, 6H), 1.54-1.42 (m, 8H), 1.39-1.11 (m, 41H), 0.88 (dt, J=11.8, 6.0 Hz, 9H).

¹³C NMR (101 MHz, CDCl 3) δ173.86 (s), 173.63 (s), 140.59 (s), 138.34 (s), 128.45 (s), 128.24 (s), 77.36 (s), 77.04 (s), 76.72 (s), 74.14 (s), 63.69 (s), 58.11 (s), 55.71 (s), 53.90 (s), 35.53 (s), 34.68 (s), 34.23 (d, J=14.8 Hz), 31.82 (d, J=11.6 Hz), 31.56 (s), 31.26 (s), 30.32 (s), 29.53 (d, J=2.8 Hz), 29.19 (dd, J=8.0, 4.4 Hz), 27.20 (d, J=2.4 Hz), 26.64 (s), 25.33 (s), 25.02 (d, J=15.6 Hz), 22.62 (d, J=11.6 Hz), 14.08 (d, J=8.0 Hz).

### O. Compound SW-II-137-3

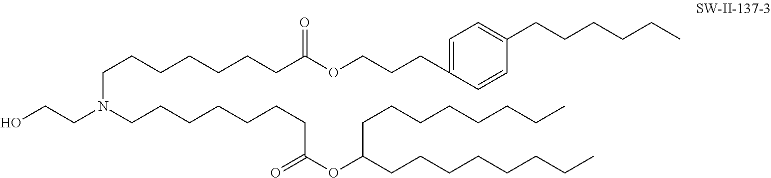 [SMILES: CCCCCCCCC(CCCCCCCC)OC(=O)CCCCCCCN(CCO)CCCCCCCC(=O)OCCCc1ccc(CCCCCC)cc1]

### 1. Synthesis of Compound 3

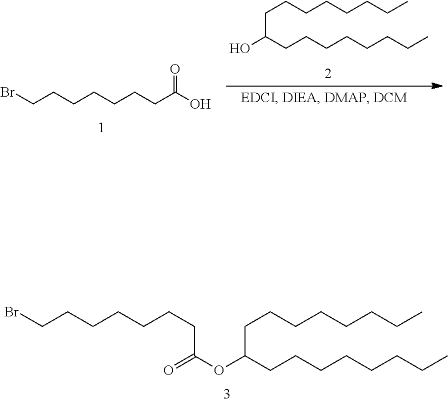 [SMILES: CCCCCCCCC(O)CCCCCCCC ; O=C(O)CCCCCCCBr ; CCCCCCCCC(CCCCCCCC)OC(=O)CCCCCCCBr]

To a mixture of compound 1 (11.8 g, 53 mmol, 1.2 eq.) and compound 2 (11.2 g, 44 mmol, 1 eq.) in DCM (110 mL) was added EDCI (16.9 g, 88 mmol, 2 eq.) and DMAP (2.1 g, 18 mmol, 0.4 eq.), then add DIEA (22.7 g, 176 mmol, 4 eq.). The reaction mixture was stirred at room temperature under nitrogen for 16 hours. TLC (petroleum ether/ethyl acetate=30/1) showed consumption of compound 1 and formation of the desired product. The reaction mixture was extracted with ethyl acetate (200 mL) and washed with water (200 mL×3), dried over anhydrous sodium sulfate, filtered and concentrated under reduced pressure. The residue was purified by silica gel column chromatography, eluting with petroleum ether/ethyl acetate (1/0-20/1), to obtain compound 3 (7.391 g, 37%) as a colorless oil.

### 2. Synthesis of Compound 5

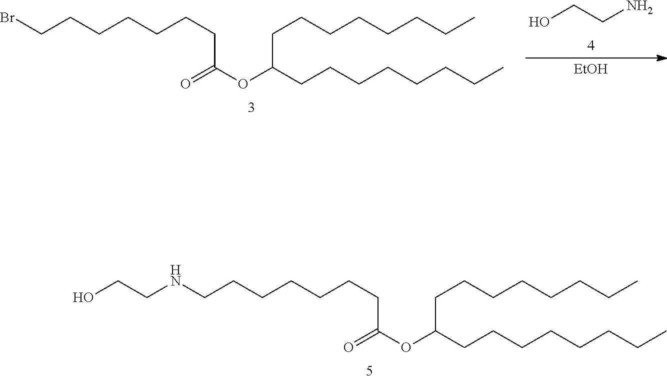

A mixture of compound 3 (7.391 mg, 16.07 mmol, 1 eq.) and compound 4 (29.4 g, 482.02 mmol, 30 eq.) in ethanol (2 mL) was stirred at 55° C. under nitrogen for 16 hours. TLC (DCM/MeOH=10/1) showed that new major spots were observed. The reaction mixture was extracted with ethyl acetate (100 mL) and washed with water (3×100 mL). The organic layer was dried over anhydrous sodium sulfate, filtered and concentrated under reduced pressure. The residue was purified by silica gel column chromatography, eluting with DCM/MeOH (1/0-10:1, v/v), to obtain compound 5 (3.695 g, 52%) as a yellow oil.

### 3. Synthesis of Compound 8

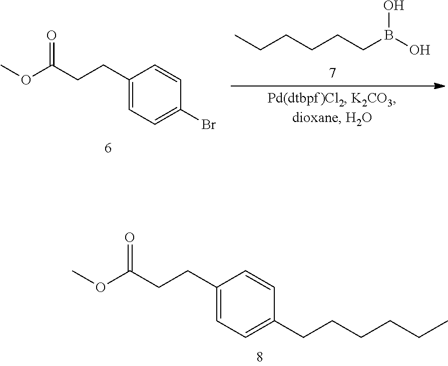

To a mixture of compound 6 (1 g, 4.12 mmol, 1 eq.) and compound 7 (803 g, 6.17 mmol, 1.5 eq.) in 1,4-dioxane/water (10 mL/1 mL) was added Pd(dtbpf)Cl 2 (269 mg, 0.41 mmol, 0.1 eq.) and potassium carbonate (1.7 g, 12.36 mmol, 3 eq.). The mixture was stirred at 100° C. overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 8 (568 mg, 56%) as a colorless oil.

### 4. Synthesis of Compound 9

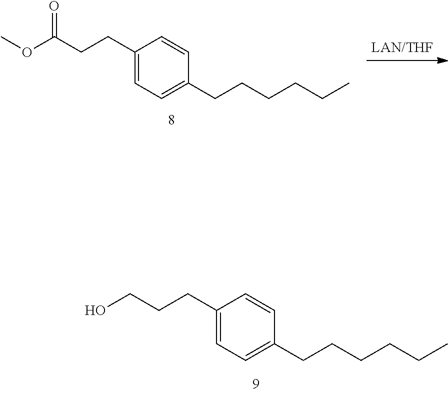 [SMILES: CCCCCCc1ccc(CCC(=O)OC)cc1 ; CCCCCCc1ccc(CCCO)cc1]

To a mixture of compound 8 (568 mg, 2.29 mmol, 1 eq.) in THF (6 mL) was added lithium aluminum hydride (2.3 mL, 2.29 mmol, 1 M, 1 eq. in THF) at 0° C. under nitrogen. The mixture was stirred at room temperature for 3 hours. TLC (PE/EA=5/1) indicated that the reaction was complete and new major spots were observed. The mixture was quenched with water (2.3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 9 as a colorless oil (541 mg, >100%) without further purification.

### 5. Synthesis of Compound 10

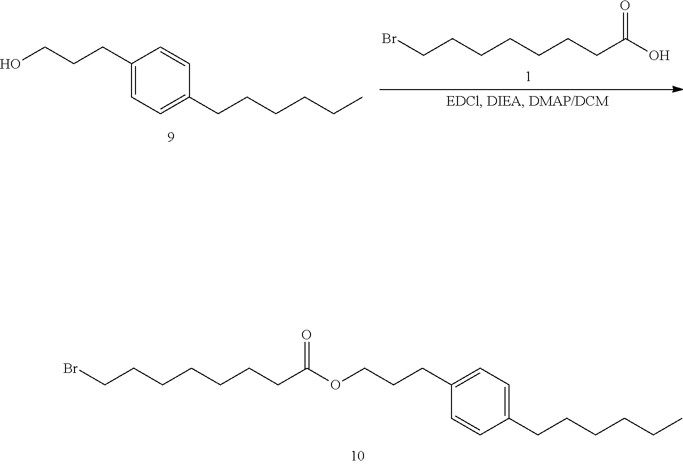 [SMILES: CCCCCCc1ccc(CCCO)cc1 ; O=C(O)CCCCCCCBr ; CCCCCCc1ccc(CCCOC(=O)CCCCCCCBr)cc1]

To a mixture of compound 9 (441 mg, 2 mmol, 1 eq.) and compound 1 (536 mg, 2.4 mmol, 1.2 eq.) in DCM (5 mL) was added EDCI (768 mg, 4 mmol, 2 eq.) and DMAP (98 mg, 0.8 mmol, 0.4 eq.), then add DIEA (1.032 g, 8 mmol, 4 eq.). The reaction mixture was stirred at room temperature under nitrogen for 16 hours. TLC (petroleum ether/ethyl acetate=10/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-10/1), to obtain compound 10 (372 mg, 44%) as a colorless oil.

### 6. Synthesis of SW-II-137-3

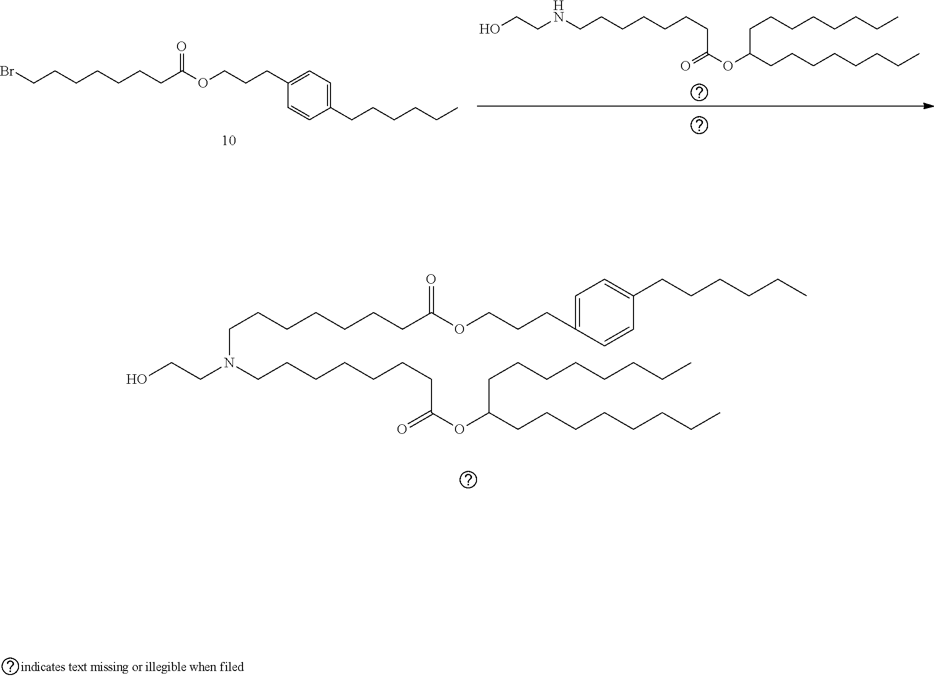 [SMILES: CCCCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCCCC)cc1]

To compound 10 (150 mg, 0.353 mmol, 1 eq.) and compound 5 (156 mg, 0.353 mmol, 1 eq.) in CPME/CH3 To the mixture in CN (2 mL/2 mL) were added potassium carbonate (244 mg, 1.765 mmol, 6 eq.) and potassium iodide (117 mg, 0.706 mmol, 2 eq.). After addition, the mixture was stirred at 90° C. overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-137-3 (56.17 mg, 20%) as a yellow oil.

LCMS:Rt:1.550 min; MS m/z(ELSD):786.4[M+H]+;

HPLC: 98.597% purity, ELSD; RT=13.153 min.

¹H NMR (400 MHz, CDCl 3) δ7.09 (s, 4H), 4.92-4.78 (m, 1H), 4.08 (t, J=6.6 Hz, 2H), 3.62 (t, J=5.2 Hz, 2H), 2.78-2.50 (m, 10H), 2.35-2.22 (m, 4H), 2.00-1.88 (m, 2H), 1.57 (ddd, J=28.9, 13.5, 4.5 Hz, 14H), 1.38-1.20 (m, 42H), 0.88 (t, J=6.8 Hz, 9H).

¹³C NMR (101 MHz, CDCl 3) δ173.83 (s), 173.60 (s), 140.60 (s), 138.33 (s), 128.44 (s), 128.24 (s), 77.36 (s), 77.04 (s), 76.72 (s), 74.15 (s), 63.69 (s), 57.95 (s), 55.83 (s), 53.95 (s), 35.56 (s), 34.65 (s), 34.21 (d, J=13.0 Hz), 31.81 (d, J=12.3 Hz), 31.54 (s), 30.32 (s), 29.52 (d, J=3.1 Hz), 29.34-28.94 (m), 27.13 (d, J=2.5 Hz), 26.31 (s), 25.33 (s), 24.99 (d, J=15.7 Hz), 22.64 (d, J=5.7 Hz), 14.11 (s).

P. Compound SW-II-138-1

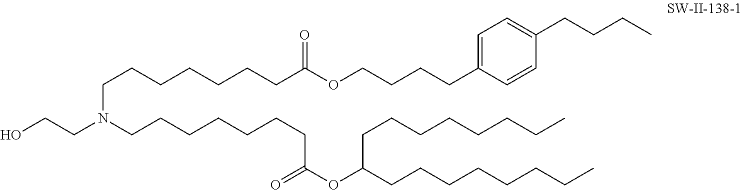 [SMILES: CCCCCCCCC(CCCCCCCC)OC(=O)CCCCCCCN(CCO)CCCCCCCC(=O)OCCCCc1ccc(CCCC)cc1]

### 1. Synthesis of Compound 2

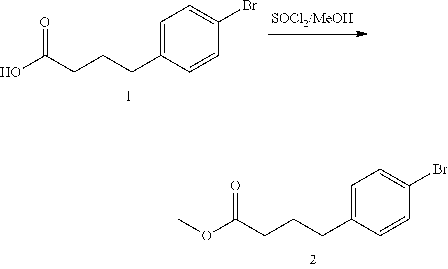

Compound 1 (4 g, 16.46 mmol, 1.0 eq.) was dissolved in MeOH (40 mL), cooled to 0° C., and SOCl was added dropwise. 2 (3.9 g, 32.92 mmol, 2.0 eq). Then react at room temperature for 1 hour. TLC (PE/EA=5/1) showed consumption of starting material and formation of desired product. The system was directly spin-dried under reduced pressure, and NaHCO was added to the residue. 3 (70 mL) solution, extracted with EA (80 mL×3). Combine the organic phases and wash with saturated brine (2×30 mL), anhydrous Na2SO4 Dry, filter and spin dry under reduced pressure. The residue was purified with a silica gel column and eluted with PE/EA (1/0-5:1, v/v) to obtain compound 2 (4.1 mg, 95%) as a yellow oil.

### 2. Synthesis of Compound 4

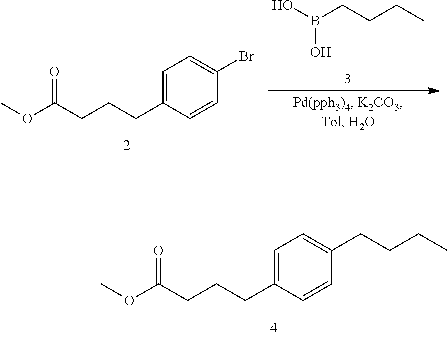

Compound 2 (500 mg, 1.95 mmol, 1.0 eq.), Compound 3 (239 mg, 2.34 mmol, 1.2 eq.), Pd (PPh 3) 4 (225 mg, 0.19 mmol, 0.1 eq) and K2CO3 (808 g, 5.85 mmol, 3.0 eq.) was dissolved in toluene (5.0 mL) and water (1 mL) was added. Then react at 110° C. for 3 hours under nitrogen protection. TLC (PE/EA=5/1) showed consumption of starting material and formation of desired product. Add H to the reaction 2 Quench with O (70 mL) and extract with EA (80 mL×3). Combine the organic phases and wash with saturated brine (2×30 mL), anhydrous Na2SO4 Dry, filter and spin dry under reduced pressure. The residue was purified using a silica gel column and eluted with PE/EA (1/0-5:1, v/v) to obtain compound 4 (320 mg, 70%) as a yellow oil.

### 3. Synthesis of Compound 5

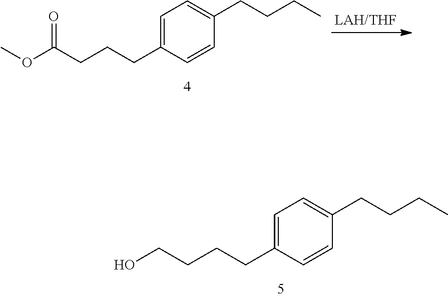 [SMILES: CCCCc1ccc(CCCC(=O)OC)cc1 ; CCCCc1ccc(CCCCO)cc1]

Compound 4 (300 mg, 1.28 mmol, 1.0 eq.) was dissolved in THF (4.0 mL), and LAH (97 mg, 2.56 mmol, 2.0 eq.) was added at 0° C. Then react at room temperature for 2 hours under nitrogen protection. TLC (PE/EA=5/1) showed consumption of starting material and formation of desired product. Add HCl (1M, 4 mL) solution and H2O (10 mL) quenched the reaction and extracted with EA (50 mL×3). The organic phase was washed with saturated brine (2×30 mL) and anhydrous Na2SO4 Dry, filter and spin dry under reduced pressure. The residue was purified with a silica gel column and eluted with PE/EA (1/0-5:1, v/v) to obtain compound 5 (224 mg, 84.8%) as a yellow oil.

### 4. Synthesis of Compound 7

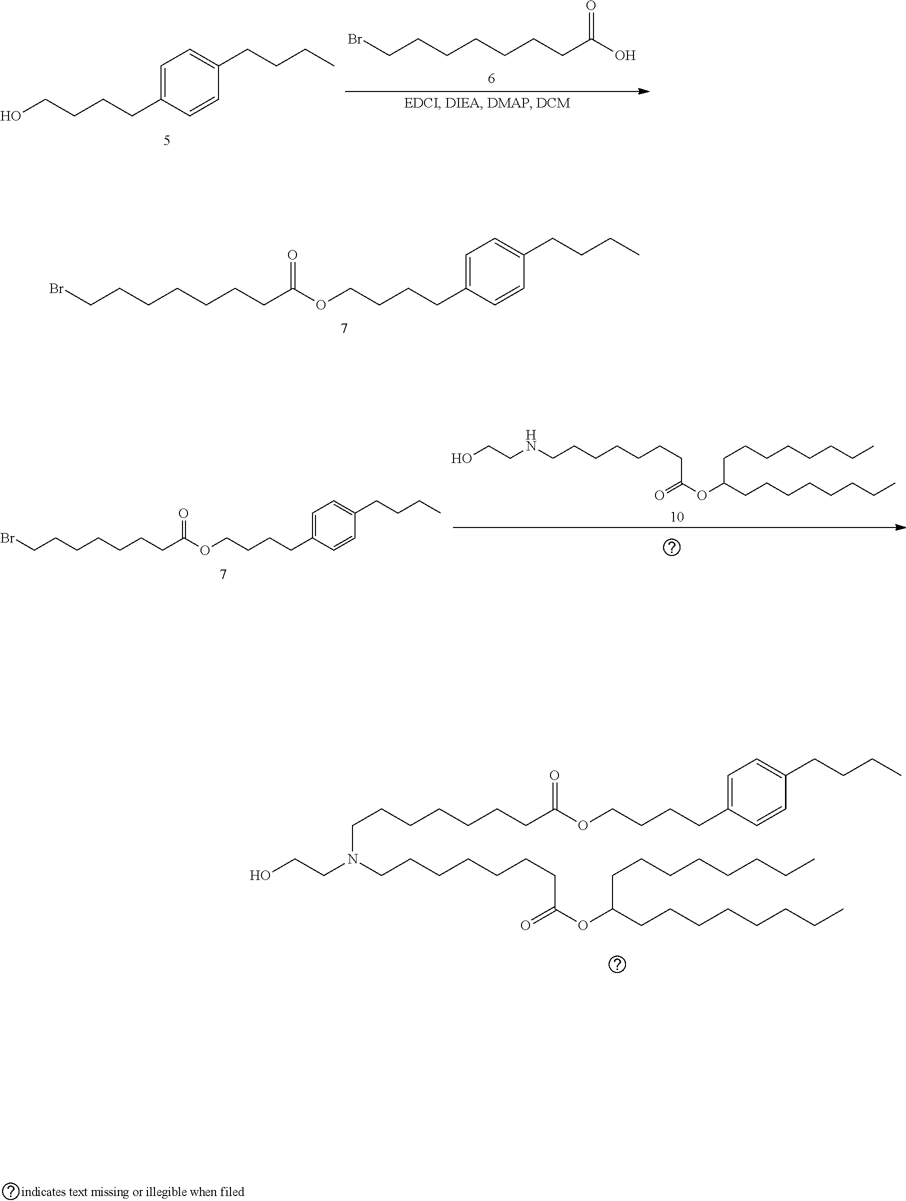

Compound 7 (224 mg, 1.09 mmol, 1.0 eq.) was dissolved in DCM (3.0 mL), and compound 6 (290 mg, 1.30 mmol, 1.2 eq.), EDCI (415 mg, 2.17 mmol, 2.0 eq.), DIEA (561 mg) were added, 4.35 mmol, 4.0 eq.) and DMAP (53 mg, 0.43 mmol, 0.4 eq.). Then, the reaction was carried out at room temperature overnight under nitrogen protection. TLC (PE/EA=30/1) showed consumption of starting material and formation of desired product. The reaction was quenched with HCl (1M) solution and adjusted to pH=4-6, and extracted with DCM (80 mL×3). The combined organic phases were washed with saturated brine (2×30 mL), anhydrous Na2SO4 Dry, filter and spin dry under reduced pressure. The residue was purified with a silica gel column and eluted with PE/EA (1/0-30:1, v/v) to obtain compound 7 (208 mg, 46.7%) as a colorless oil.

### 5. Synthesis of SW-II-138-1

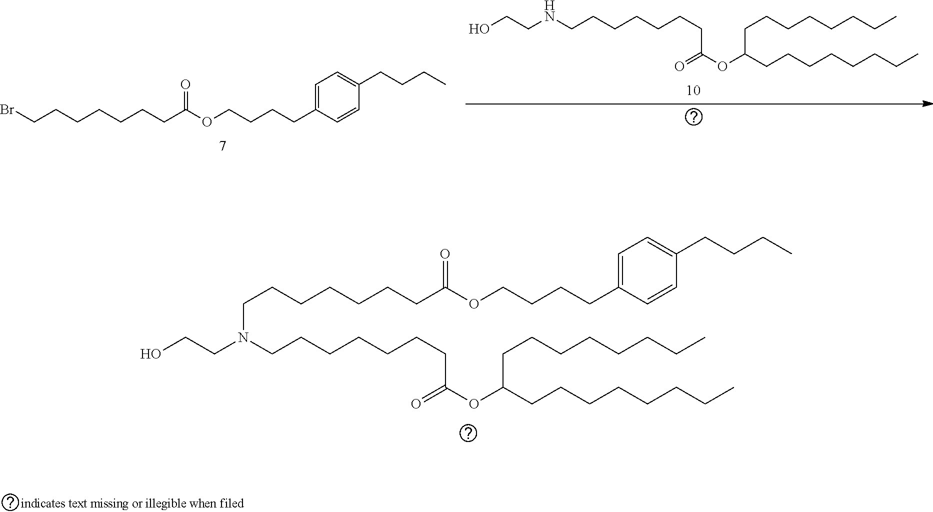 [SMILES: CCCCCCCCC(CCCCCCCC)OC(=O)CCCCCCCNCCO ; CCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCC)cc1]

Compound 10 (110 mg, 0.25 mmol, 1 eq.), compound 7 (153 mg, 0.37 mmol, 1.5 eq.), KI (83 mg, 0.50 mmol, 2.0 eq.) and CPME (2 mL) were dissolved in MeCN (2 mL) and K was added 2CO 3 (172 mg, 1.25 mmol, 5.0 eq). The reaction was carried out overnight at 90° C. under nitrogen protection. TLC (DCM/MeOH=10/1) showed consumption of starting material and formation of the desired product. The reaction was directly rotated to dryness under reduced pressure. The residue was purified by silica gel column and eluted with DCM/MeOH (1/0-10:1, v/v) to obtain a light yellow oily compound (65 mg, 32%, SW-II-138-1).

LCMS:Rt:1.684 min; MS m/z(ELSD):772.4[M+H]+;

HPLC: 96.56% purity, ELSD; RT=6.346 min.

¹H NMR (400 MHz, CDCl 3) δ7.09 (s, 4H), 4.86 (s, 1H), 4.09 (d, J=6.0 Hz, 2H), 3.97 (s, 2H), 3.07 (d, J=38.8 Hz, 6H), 2.69-2.51 (m, 4H), 2.28 (td, J=7.3, 3.6 Hz, 4H), 1.79 (s, 4H), 1.70-1.46 (m, 16H), 1.42-1.17 (m, 37H), 0.90 (dt, J=13.6, 7.2 Hz, 9H).

¹³C NMR (101 MHz, CDCl 3) δ173.80 (s), 173.53 (s), 140.32 (s), 139.13 (s), 128.28 (d, J=13.6 Hz), 77.43 (s), 77.11 (s), 76.80 (s), 74.21 (s), 64.22 (s), 56.85 (s), 55.98 (s), 53.93 (s), 35.22 (s), 35.01 (s), 34.54 (s), 34.14 (d, J=5.6 Hz), 33.71 (s), 31.85 (s), 29.50 (d, J=2.8 Hz), 29.22 (s), 29.12-28.60 (m), 28.26 (s), 27.78 (s), 26.70 (d, J=4.4 Hz), 25.31 (s), 24.82 (d, J=17.6 Hz), 24.28 (s), 22.65 (s), 22.37 (s), 14.03 (d, J=15.2 Hz).

### Q. SW-II-138-2

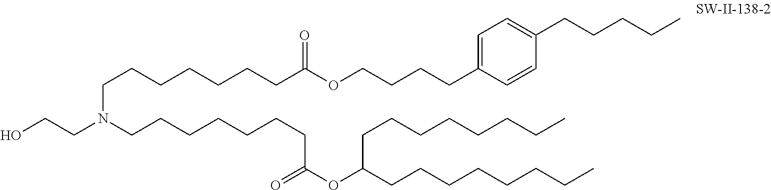 [SMILES: CCCCCCCCC(CCCCCCCC)OC(=O)CCCCCCCN(CCO)CCCCCCCC(=O)OCCCCc1ccc(CCCCC)cc1]

### 1. Synthesis of Compound 3

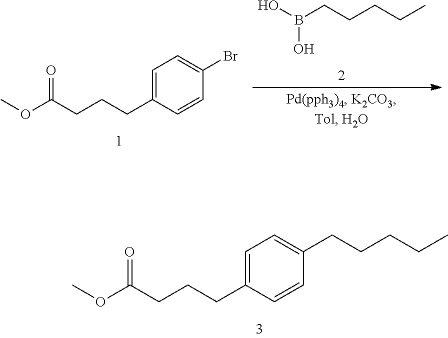

Compound 1 (500 mg, 1.95 mmol, 1.0 eq.), Compound 2 (271 mg, 2.34 mmol, 1.2 eq.), Pd (PPh 3) 4 (225 mg, 0.20 mmol, 0.1 eq) and K2CO3 (809 g, 5.86 mmol, 3.0 eq.) was dissolved in toluene (5.0 mL), water (1 mL) was added, and then reacted at 110° C. for 3 hours under nitrogen protection. TLC (PE/EA=5/1) showed consumption of starting material and formation of desired product. The reaction was quenched by adding water (70 mL) and extracted with EA (80 mL×3). Combine the organic phases and wash with saturated brine (2×30 mL), anhydrous Na2SO4 Dry, filter and spin dry under reduced pressure. The residue was purified with a silica gel column and eluted with PE/EA (1/0-30:1, v/v) to obtain compound 3 (320 mg, 70%) as a colorless oil.

### 2. Synthesis of Compound 4

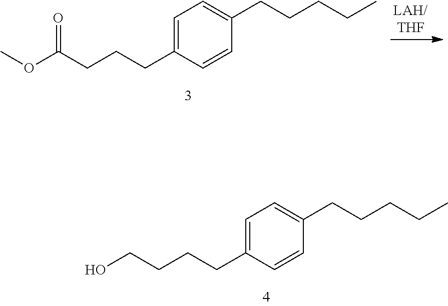 [SMILES: CCCCCc1ccc(CCCC(=O)OC)cc1 ; CCCCCc1ccc(CCCCO)cc1]

Compound 3 (320 mg, 1.29 mmol, 1.0 eq.) was dissolved in THF (3.0 mL), LAH (67 mg, 1.77 mmol, 2.0 eq.) was added at 0° C., and then reacted at room temperature under nitrogen protection for 2 hours. TLC (PE/EA=5/1) showed consumption of starting material and formation of desired product. The reaction uses HCl (1M, 2 mL) solution and H2 Quench with O (10 mL) and extract with EA (50 mL×3). The combined organic phases were washed with saturated brine (2×30 mL) and anhydrous Na2SO4 Dry, filter and spin dry under reduced pressure. The residue was purified with a silica gel column and eluted with PE/EA (1/0-30:1, v/v) to obtain compound 4 (180 mg, 64%) as a colorless oil.

### 3. Synthesis of Compound 6

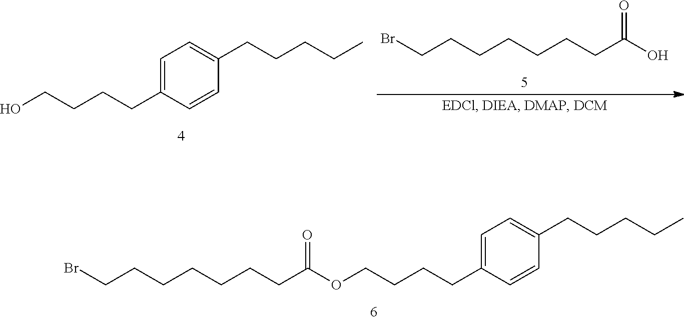

Compound 4 (180 mg, 0.82 mmol, 1.0 eq.) was dissolved in DCM (3.0 mL) and compound 5 (245 mg, 1.10 mmol, 1.2 eq.), EDCI (347 mg, 1.82 mmol, 2.0 eq.), DIEA (470 mg, 3.63 mmol, 4.0 eq.) and DMAP (45 mg, 0.36 mmol, 0.4 eq.). Then, the reaction was carried out overnight at room temperature under nitrogen protection. TLC (PE/EA=30/1) showed complete consumption of starting material and formation of the desired product. The reaction was quenched with HCl (1M) solution and adjusted to pH=5-6, and extracted with DCM (80 mL×3). The combined organic phases were washed with saturated brine (2×30 mL), anhydrous Na2SO4 Dry, filter and spin dry under reduced pressure. The residue was purified with a silica gel column and eluted with PE/EA (1/0-30:1, v/v) to obtain compound 6 (220 mg, 63.6%) as a colorless oil.

### 4. Synthesis of SW-II-138-2

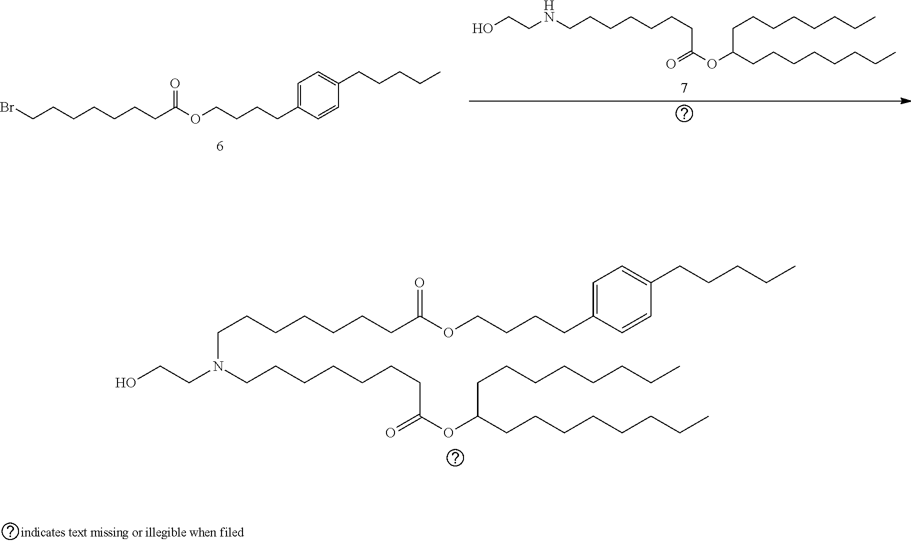 [SMILES: CCCCCCCCC(CCCCCCCC)OC(=O)CCCCCCCNCCO ; CCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCC)cc1]

Compound 6 (158 mg, 0.37 mmol, 1.5 eq.), compound 7 (110 mg, 0.25 mmol, 1.0 eq.), KI (83 mg, 0.50 mmol, 2.0 eq.) and CPME (2 mL) were dissolved in MeCN (2 mL), and K was added 2CO 3 (172 mg, 1.25 mmol, 5.0 eq). Then, the reaction was carried out overnight at 90° C. under nitrogen protection. TLC (DCM/MeOH=10/1) showed consumption of starting material and formation of desired product. The reaction was directly spin-dried under reduced pressure, and the residue was purified with a silica gel column and eluted with DCM/MeOH (1/0-10:1, v/v) to obtain the target product as a colorless oil (100 mg, 51%, SW-JJ-138-2).

LCMS:Rt:1.834 min; MS m/z(ELSD):786.4[M+H]+;

HPLC: 99.20% purity, ELSD; RT=7.990 min.

¹H NMR (400 MHz, CDCl 3) δ7.00 (s, 4H), 4.88-4.73 (m, 2H), 4.00 (t, J=5.6 Hz, 2H), 3.81-3.54 (m, 2H), 3.00-2.81 (m, 2H), 2.81-2.65 (m, 4H), 2.50 (dd, J=16.4, 8.4 Hz, 4H), 2.20 (td, J=7.6, 3.2 Hz, 4H), 1.56 (ddd, J=18.4, 10.4, 5.2 Hz, 13H), 1.43 (d, J=5.6 Hz, 4H), 1.34-1.07 (m, 40H), 0.81 (dt, J=11.2, 5.6 Hz, 9H).

¹³C NMR (101 MHz, CDCl 3) δ173.78 (s), 173.52 (s), 140.32 (s), 139.11 (s), 128.25 (d, J=11.6 Hz), 77.49 (s), 77.17 (s), 76.85 (s), 74.14 (s), 64.17 (s), 57.25 (s), 55.82 (s), 53.85 (s), 35.50 (s), 35.01 (s), 34.56 (s), 34.14 (d, J=7.2 Hz), 31.84 (s), 31.53 (s), 31.23 (s), 29.49 (d, J=2.8 Hz), 29.21 (s), 28.94 (dd, J=6.4, 4.4 Hz), 28.25 (s), 27.77 (s), 26.84 (d, J=4.4 Hz), 25.30 (s), 25.25-24.59 (m), 22.59 (d, J=11.2 Hz), 14.05 (d, J=7.6 Hz).

### R. SW-JJ-138-3

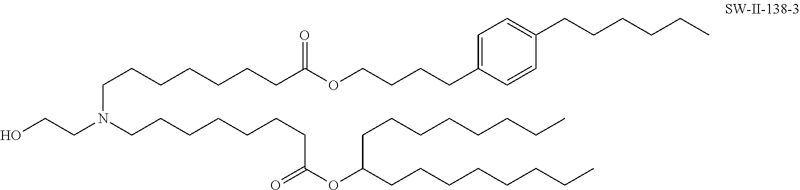 [SMILES: CCCCCCCCC(CCCCCCCC)OC(=O)CCCCCCCN(CCO)CCCCCCCC(=O)OCCCCc1ccc(CCCCCC)cc1]

### 1. Synthesis of Compound 3

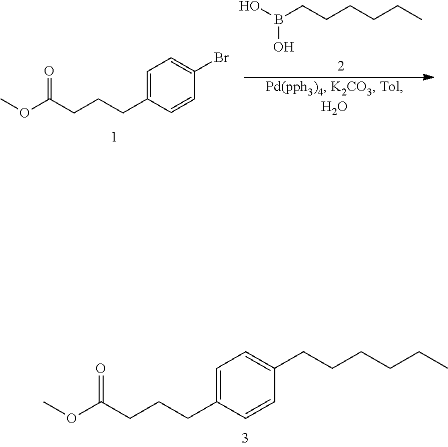

Compound 1 (500 mg, 1.95 mmol, 1.0 eq.), Compound 2 (305 mg, 2.34 mmol, 1.2 eq.), Pd (PPh 3) 4 (225 mg, 0.20 mmol, 0.1 eq) and K2CO3 (809 g, 5.86 mmol, 3.0 eq.) was dissolved in toluene (5.0 mL) and water (1 mL) was added. Then, the reaction was carried out at 110° C. for 3 hours under nitrogen protection. TLC (PE/EA=5/1) showed complete consumption of starting material and formation of the desired compound. The reaction was quenched by adding water (80 mL) and extracted with EA (80 mL×3). Combine the organic phases and wash with saturated brine (2×40 mL), anhydrous Na2SO4 Dry, filter and spin dry under reduced pressure. The residue was purified with a silica gel column and eluted with PE/EA (1/0-5:1, v/v) to obtain compound 3 (260 mg, 51.3%) as a colorless oil.

### 2. Synthesis of Compound 4

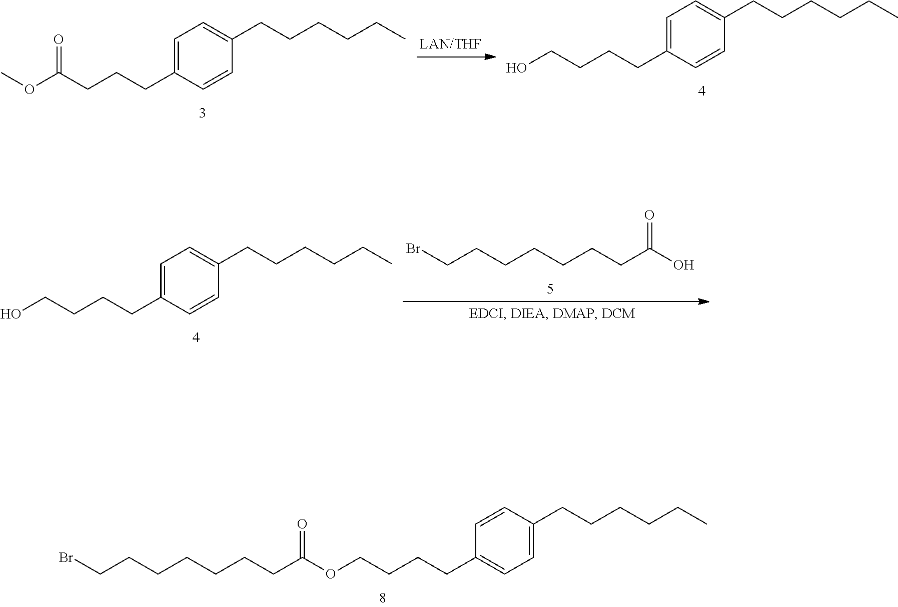

Compound 3 (260 mg, 0.99 mmol, 1.0 eq.) was dissolved in THF (4.0 mL), and LAH (75 mg, 1.98 mmol, 2.0 eq.) was added at 0° C. Then, react at room temperature for 2 hours under nitrogen protection. TLC (PE/EA=5/1) showed that the starting material was completely reacted and the desired compound was formed. The reaction uses HCl (1M, 4 mL) solution and H2 Quench with O (20 mL) and extract with EA (50 mL×3). The combined organic phases were washed with saturated brine (2×30 mL) and anhydrous Na2SO4 Dry, filter and spin dry under reduced pressure. The residue was purified with a silica gel column and eluted with PE/EA (1/0-5:1, v/v) to obtain compound 4 (230 mg, 98%) as a colorless oil.

### 3. Synthesis of Compound 6

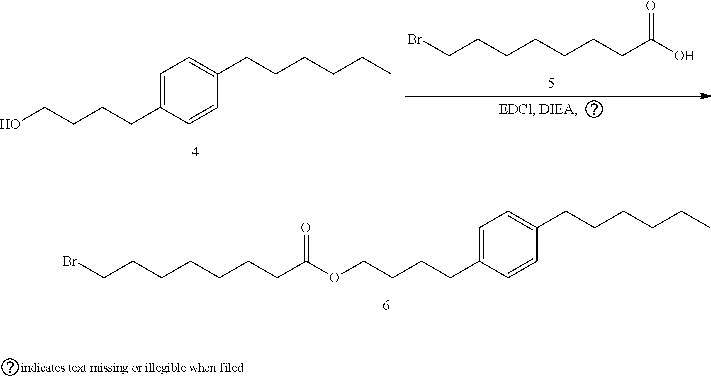

Compound 4 (240 mg, 1.03 mmol, 1.0 eq.) was dissolved in DCM (4.0 mL), and compound 5 (275 mg, 1.23 mmol, 1.2 eq.), EDCI (392 mg, 2.07 mmol, 2.0 eq.), DIEA (530 mg, 4.10 mmol, 4.0 eq.) and DMAP (50 mg, 0.41 mmol, 0.4 eq.). Then react overnight at room temperature under nitrogen protection. TLC (PE/EA=20/1) showed consumption of starting material and formation of the desired compound. The reaction was quenched with HCl (1M) and adjusted to pH=5-6, and extracted with DCM (80 mL×3). The combined organic phases were washed with saturated brine (2×30 mL), anhydrous Na2SO4 Dry, filter and spin dry under reduced pressure. The residue was purified with a silica gel column and eluted with PE/EA (1/0-20:1, v/v) to obtain compound 6 (180 mg, 40.9%) as a colorless oil.

### 4. Synthesis of SW-II-138-3

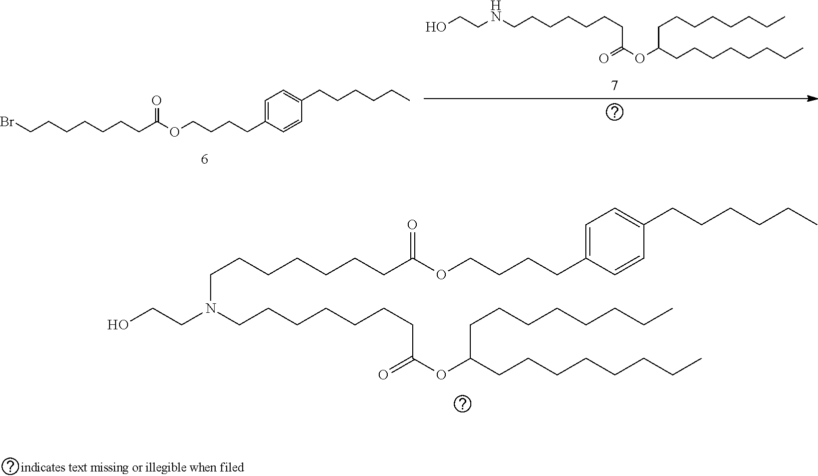 [SMILES: CCCCCCCCC(CCCCCCCC)OC(=O)CCCCCCCNCCO ; CCCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCCC)cc1]

Compound 6 (164 mg, 0.37 mmol, 1 eq.) and compound 7 (110 mg, 0.24 mmol, 1.0 eq.), KI (83 mg, 0.49 mmol, 2.0 eq.) and CPME (2 mL) were dissolved in MeCN (2 mL) and K was added 2CO 3 (172 mg, 1.24 mmol, 5.0 eq). Then, the reaction was carried out at 90° C. overnight under nitrogen protection. TLC (DCM/MeOH=10/1) showed consumption of starting material and formation of desired product. The reaction was directly rotated to dryness under reduced pressure. The residue was purified with a silica gel column and eluted with DCM/MeOH (1/0-10:1, v/v) to obtain the target product (108 mg, 52.76%, SW-II-138-3) as a colorless oil.

LCMS:Rt:2.007 min; MS m/z(ELSD):800.4[M+H]+;

HPLC: 97.95% purity, ELSD; RT=9.455 min.

¹H NMR (400 MHz, CDCl 3) δ7.08 (s, 4H), 4.86 (p, J=6.4 Hz, 1H), 4.08 (s, 2H), 3.60 (t, J=5.2 Hz, 3H), 2.76-2.42 (m, 10H), 2.28 (td, J=7.6, 2.8 Hz, 4H), 1.70-1.42 (m, 18H), 1.28 (d, J=20.0 Hz, 41H), 0.88 (t, J=6.8 Hz, 9H).

¹³C NMR (101 MHz, CDCl 3) δ173.85 (s), 173.59 (s), 140.39 (s), 139.15 (s), 128.27 (d, J=12.0 Hz), 77.38 (s), 77.07 (s), 76.75 (s), 74.13 (s), 64.17 (s), 58.06 (s), 55.75 (s), 53.92 (s), 35.57 (s), 35.03 (s), 34.66 (s), 34.22 (d, J=13.2 Hz), 31.81 (d, J=12.4 Hz), 31.54 (s), 29.52 (d, J=2.9 Hz), 29.34-28.95 (m), 28.29 (s), 27.79 (s), 27.16 (d, J=3.6 Hz), 26.50 (s), 25.32 (s), 24.99 (d, J=17.6 Hz), 22.64 (d, J=5.6 Hz), 14.10 (s).

### S. Compound SW-JJ-139-1

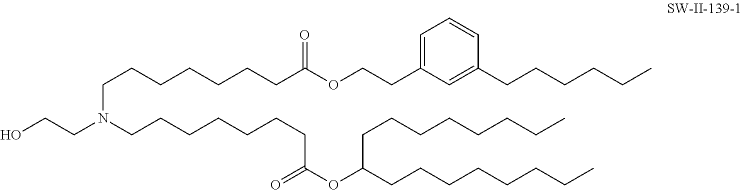 [SMILES: CCCCCCCCC(CCCCCCCC)OC(=O)CCCCCCCN(CCO)CCCCCCCC(=O)OCCc1cccc(CCCCCC)c1]

### 1. Synthesis of Compound 3

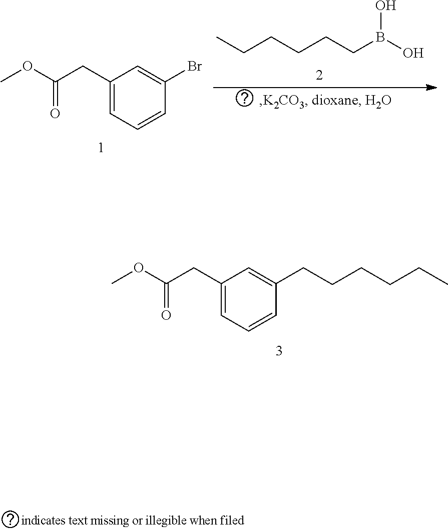

To a mixture of compound 1 (1 g, 4.37 mmol, 1 eq.) and compound 2 (852 g, 6.55 mmol, 1.5 eq.) in 1,4-dioxane/water (10 mL/1 mL) was added Pd(dtbpf)Cl 2 (286 mg, 0.437 mmol, 0.1 eq.) and potassium carbonate (1.8 g, 13.11 mmol, 3 eq.). The mixture was stirred at 100° C. overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (691 mg, 68%) as a colorless oil.

### 2. Synthesis of Compound 4

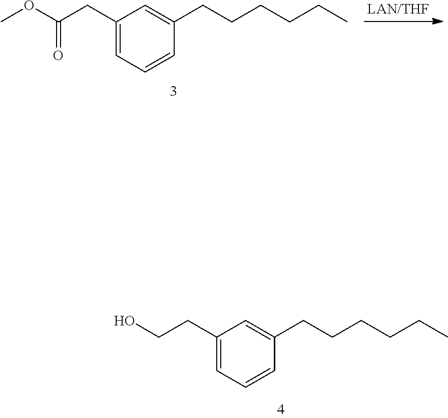 [SMILES: CCCCCCc1cccc(CC(=O)OC)c1 ; CCCCCCc1cccc(CCO)c1]

To a mixture of compound 3 (691 mg, 2.95 mmol, 1 eq.) in THF (7 mL) was added lithium aluminum hydride (3 mL, 2.95 mmol, 1 M, 1 eq. in THF) at 0° C. under nitrogen. The mixture was stirred at room temperature for 3 hours. TLC (PE/EA=5/1) indicated that the reaction was complete and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (547 mg, 90%) as a colorless oil without further purification.

### 3. Synthesis of Compound 6

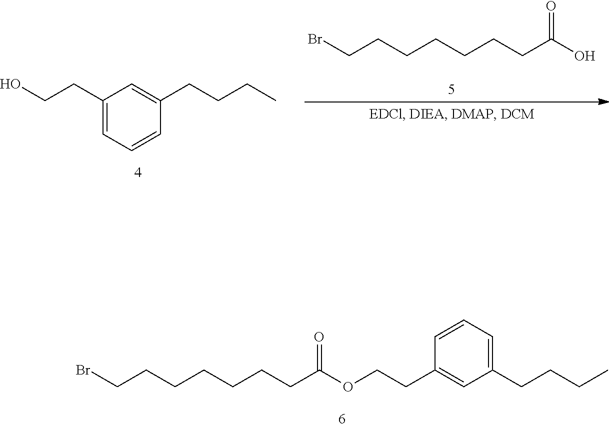 [SMILES: O=C(O)CCCCCCCBr ; CCCCc1cccc(CCO)c1 ; CCCCc1cccc(CCOC(=O)CCCCCCCBr)c1]

To a mixture of compound 4 (447 mg, 2.17 mmol, 1 eq.) and compound 5 (581 mg, 2.6 mmol, 1.2 eq.) in DCM (5 mL) was added EDCI (833 mg, 4.34 mmol, 2 eq.) and DMAP (106 mg, 0.87 mmol, 0.4 eq.), then DIEA (1.12 g, 8.68 mmol, 4 eq.) was added. The reaction mixture was stirred at room temperature under nitrogen for 16 hours. TLC (petroleum ether/ethyl acetate=15/11) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to obtain compound 6 (455 mg, 51%) as a colorless oil.

### 4. Synthesis of SW-II-139-1

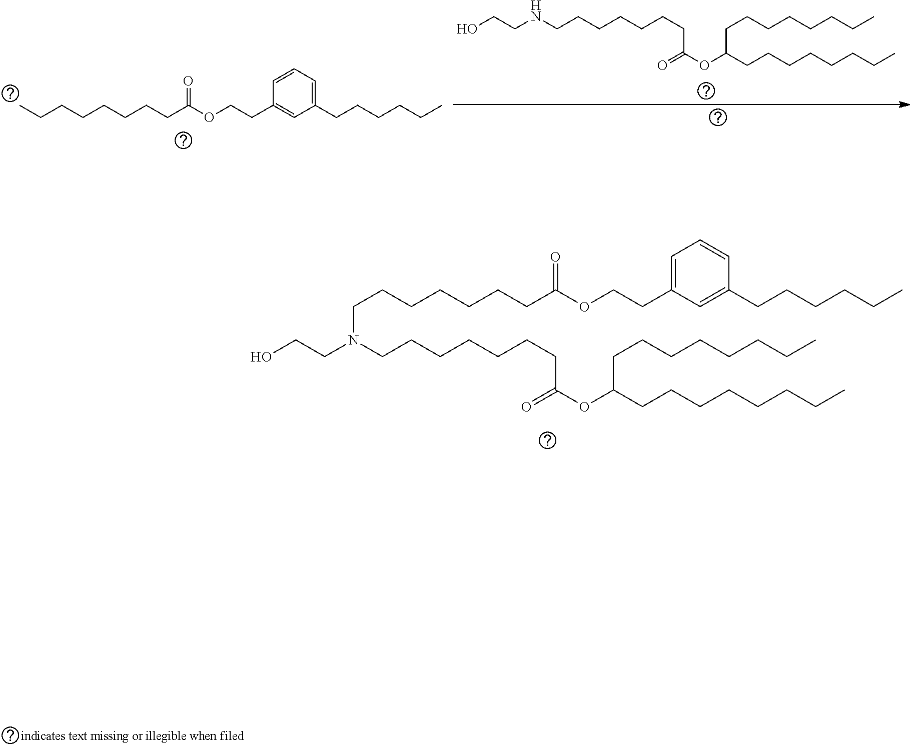 [SMILES: CCCCCCCCC(CCCCCCCC)OC(=O)CCCCCCCNCCO ; CCCCCCCCC(=O)OCCc1cccc(CCCCCC)c1 ; CCCCCCCCC(CCCCCCCC)OC(=O)CCCCCCCN(CCO)CCCCCCCC(=O)OCCc1cccc(CCCCCC)c1]

To compound 6 (150 mg, 0.365 mmol, 1 eq.) and compound 7 (161 mg, 0.365 mmol, 1 eq.) in CPME/CH3 To the mixture in CN (2 mL/2 mL) were added potassium carbonate (252 mg, 1.825 mmol, 6 eq.) and potassium iodide (121 mg, 0.73 mmol, 2 eq.). After addition, the mixture was stirred at 90° C. overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-139-1 (54.53 mg, 19%) as a yellow oil.

LCMS:Rt:1.521 min; MS m/z(ELSD):772.4[M+H]+;

HPLC: 99.637% purity, ELSD; RT=12.347 min.

¹H NMR (400 MHz, CDCl 3) δ7.20 (t, J=7.7 Hz, 1H), 7.03 (t, J=6.8 Hz, 3H), 4.94-4.78 (m, 1H), 4.27 (t, J=7.2 Hz, 2H), 3.65 (t, J=5.1 Hz, 2H), 2.90 (t, J=7.2 Hz, 2H), 2.73 (t, J=4.9 Hz, 2H), 2.67-2.41 (m, 6H), 2.28 (td, J=7.5, 2.7 Hz, 4H), 1.67-1.45 (m, 14H), 1.41-1.19 (m, 42H), 0.88 (dd, J=7.9, 5.7 Hz, 9H).

¹³C NMR (101 MHz, CDCl 3) δ173.65 (d, J=11.3 Hz), 143.17 (s), 137.67 (s), 129.04 (s), 128.34 (s), 126.61 (s), 126.11 (s), 77.30 (d, J=11.6 Hz), 77.04 (s), 76.72 (s), 74.16 (s), 64.85 (s), 57.88 (s), 55.93 (s), 53.97 (s), 35.94 (s), 35.13 (s), 34.64 (s), 34.20 (d, J=10.5 Hz), 31.80 (d, J=13.7 Hz), 31.50 (s), 29.52 (d, J=2.9 Hz), 29.34-28.92 (m), 27.08 (d, J=3.9 Hz), 26.10 (s), 25.33 (s), 25.05 (s), 24.82 (s), 22.64 (d, J=6.5 Hz), 14.11 (s).

### T. Compound SW-II-139-2

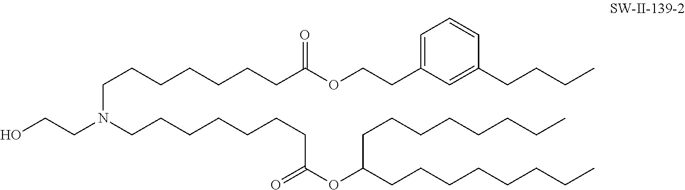 [SMILES: CCCCCCCCC(CCCCCCCC)OC(=O)CCCCCCCN(CCO)CCCCCCCC(=O)OCCc1cccc(CCCC)c1]

### 1. Synthesis of Compound 3

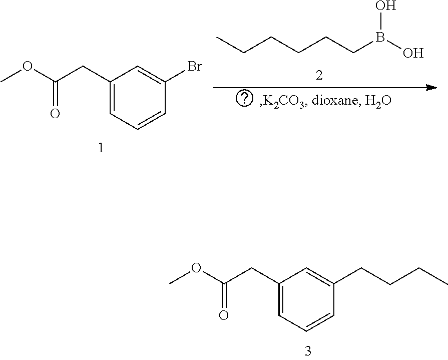

To a mixture of compound 1 (1 g, 4.37 mmol, 1 eq.) and compound 2 (668 g, 6.55 mmol, 1.5 eq.) in 1,4-dioxane/water (10 mL/1 mL) was added Pd(dtbpf)Cl 2 (286 mg, 0.437 mmol, 0.1 eq.) and potassium carbonate (1.8 g, 13.11 mmol, 3 eq.). The mixture was stirred at 100° C. overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (605 mg, 67%) as a colorless oil.

### 2. Synthesis of Compound 4

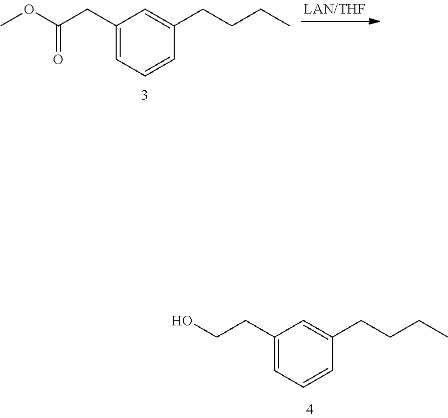

To a mixture of compound 3 (605 mg, 2.94 mmol, 1 eq.) in THF (7 mL) was added lithium aluminum hydride (3 mL, 2.94 mmol, 1 M, 1 eq. in THF) at 0° C. under nitrogen. The mixture was stirred at room temperature for 3 hours. TLC (PE/EA=5/1) indicated that the reaction was complete and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (534 mg, >100%) as a colorless oil without further purification.

### 3. Synthesis of Compound 6

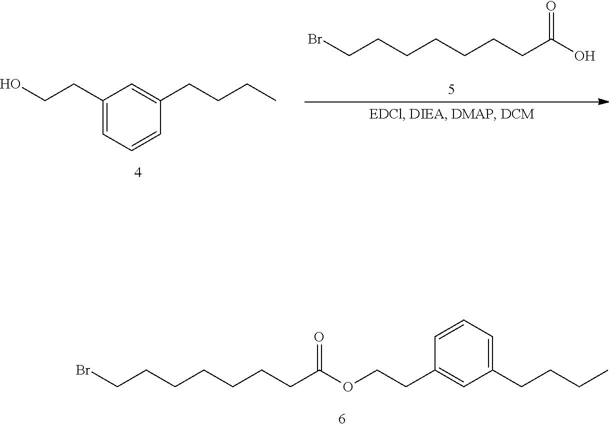 [SMILES: O=C(O)CCCCCCCBr ; CCCCc1cccc(CCO)c1 ; CCCCc1cccc(CCOC(=O)CCCCCCCBr)c1]

To a mixture of compound 4 (434 mg, 2.44 mmol, 1 eq.) and compound 5 (652 mg, 2.93 mmol, 1.2 eq.) in DCM (5 mL) was added EDCI (937 mg, 4.88 mmol, 2 eq.) and DMAP (119 mg, 0.976 mmol, 0.4 eq.), then DIEA (1.259 g, 9.76 mmol, 4 eq.) was added. The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to obtain compound 6 (355 mg, 38%) as a colorless oil.

### 4. Synthesis of SW-II-139-2

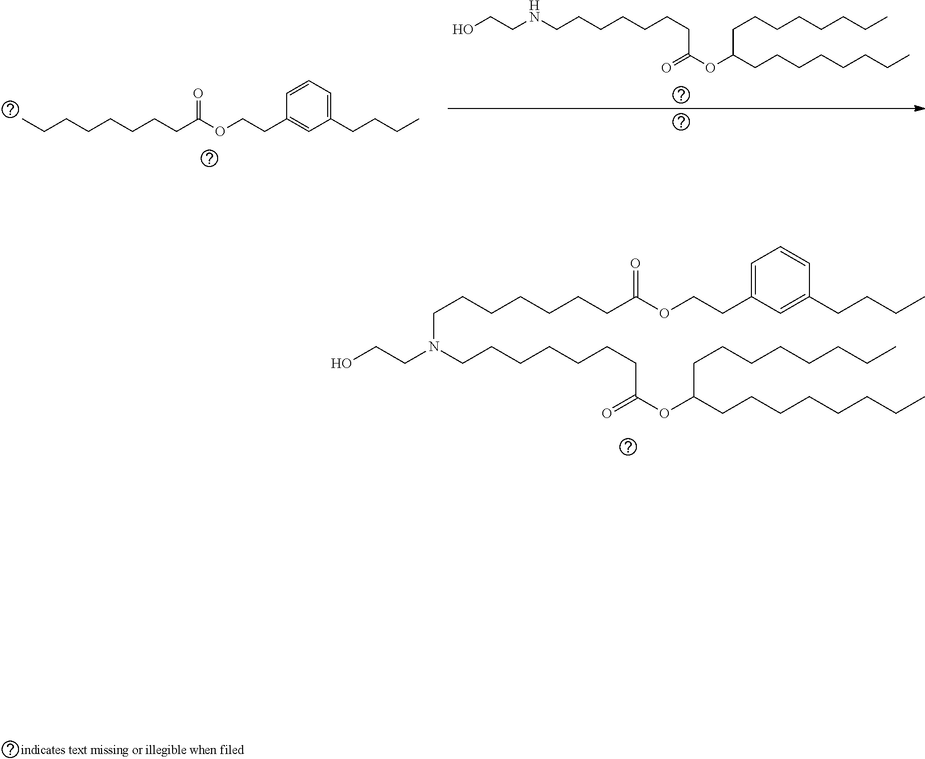 [SMILES: CCCCCCCCC(CCCCCCCC)OC(=O)CCCCCCCNCCO ; CCCCCCCCC(=O)OCCc1cccc(CCCC)c1 ; CCCCCCCCC(CCCCCCCC)OC(=O)CCCCCCCN(CCO)CCCCCCCC(=O)OCCc1cccc(CCCC)c1]

To compound 6 (122 mg, 0.319 mmol, 1 eq.) and compound 7 (140 mg, 0.319 mmol, 1 eq.) in CPME/CH3 To the mixture in CN (2 mL/2 mL) were added potassium carbonate (220 mg, 1.595 mmol, 5 eq.) and potassium iodide (106 mg, 0.638 mmol, 2 eq.). After addition, the mixture was stirred at 90° C. overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-139-2 (45.48 mg, 19%) as a yellow oil.

LCMS:Rt:1.346 min; MS m/z(ELSD):744.3[M+H]+;

HPLC: 97.994% purity, ELSD; RT=11.235 min.

1H NMR (400 MHz, CDCl 3) δ7.20 (t, J=7.8 Hz, 1H), 7.03 (t, J=7.6 Hz, 3H), 4.91-4.81 (m, 1H), 4.27 (t, J=7.2 Hz, 2H), 3.89-3.75 (m, 2H), 2.99-2.79 (m, 7H), 2.64-2.48 (m, 2H), 2.28 (td, J=7.5, 3.1 Hz, 4H), 1.74-1.08 (m, 53H), 0.90 (dt, J=13.6, 7.2 Hz, 9H).

13C NMR (101 MHz, CDCl 3) δ173.60 (d, J=11.7 Hz), 143.13 (s), 137.65 (s), 129.06 (s), 128.34 (s), 126.64 (s), 126.11 (s), 77.30 (d, J=11.4 Hz), 77.04 (s), 76.72 (s), 74.22 (s), 64.88 (s), 57.28 (s), 56.55 (s), 54.11 (s), 35.60 (s), 35.12 (s), 34.56 (s), 34.15 (d, J=4.0 Hz), 33.68 (s), 31.86 (s), 29.52 (d, J=2.8 Hz), 29.24 (s), 28.91 (dd, J=7.0, 4.2 Hz), 26.81 (d, J=3.9 Hz), 25.33 (s), 25.12-24.98 (m), 24.83 (d, J=22.2 Hz), 22.67 (s), 22.40 (s), 14.04 (d, J=14.4 Hz).

### U. Compound SW-II-140-1

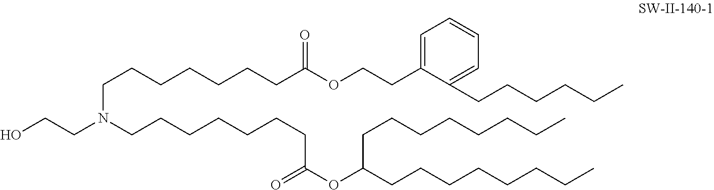 [SMILES: CCCCCCCCC(CCCCCCCC)OC(=O)CCCCCCCN(CCO)CCCCCCCC(=O)OCCc1ccccc1CCCCCC]

### 1. Synthesis of Compound 3

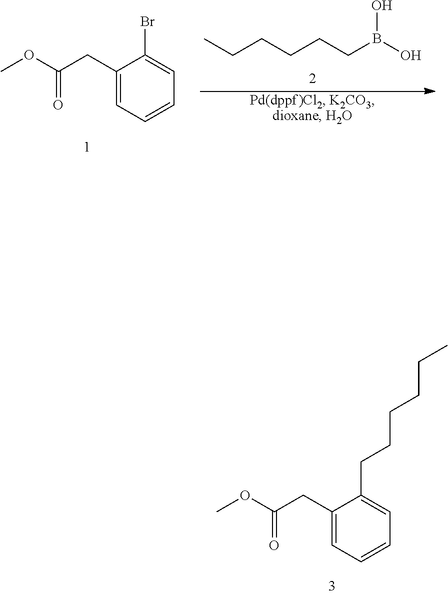

To a mixture of compound 1 (1 g, 4.37 mmol, 1 eq.) and compound 2 (852 g, 6.55 mmol, 1.5 eq.) in 1,4-dioxane/water (10 mL/1 mL) was added Pd(dppf)Cl 2 (286 mg, 0.437 mmol, 0.1 eq.) and potassium carbonate (1.8 g, 13.11 mmol, 3 eq.). The mixture was stirred at 100° C. overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (748 mg, 73%) as a colorless oil.

### 2. Synthesis of Compound 4

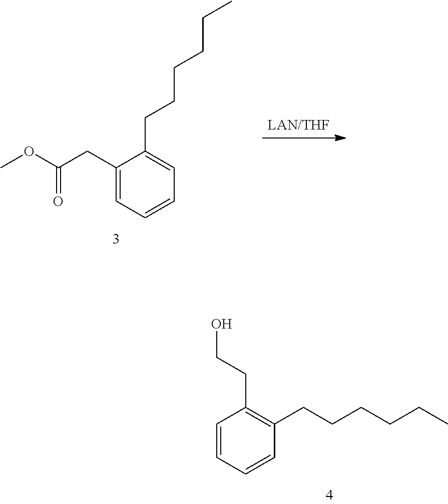 [SMILES: CCCCCCc1ccccc1CC(=O)OC ; CCCCCCc1ccccc1CCO]

To a mixture of compound 3 (748 mg, 3.2 mmol, 1 eq.) in THF (8 mL) was added lithium aluminum hydride (3.2 mL, 3.2 mmol, 1 M, 1 eq. in THF) at 0° C. under nitrogen. The mixture was stirred at room temperature for 3 hours. TLC (PE/EA=5/1) indicated that the reaction was complete and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (493 mg, 75%) as a colorless oil without further purification.

### 3. Synthesis of Compound 6

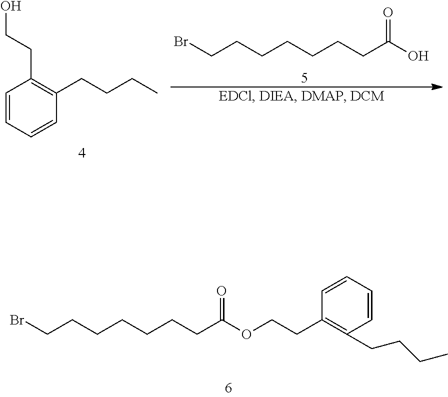

To a mixture of compound 4 (393 mg, 1.91 mmol, 1 eq.) and compound 5 (511 mg, 2.29 mmol, 1.2 eq.) in DCM (5 mL) was added EDCI (733 mg, 3.82 mmol, 2 eq.) and DMAP (93 mg, 0.76 mmol, 0.4 eq.), then DIEA (986 mg, 7.64 mmol, 4 eq.) was added. The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to give compound 6 (327 mg, 42%) as a colorless oil.

### 4. Synthesis of SW-II-140-1

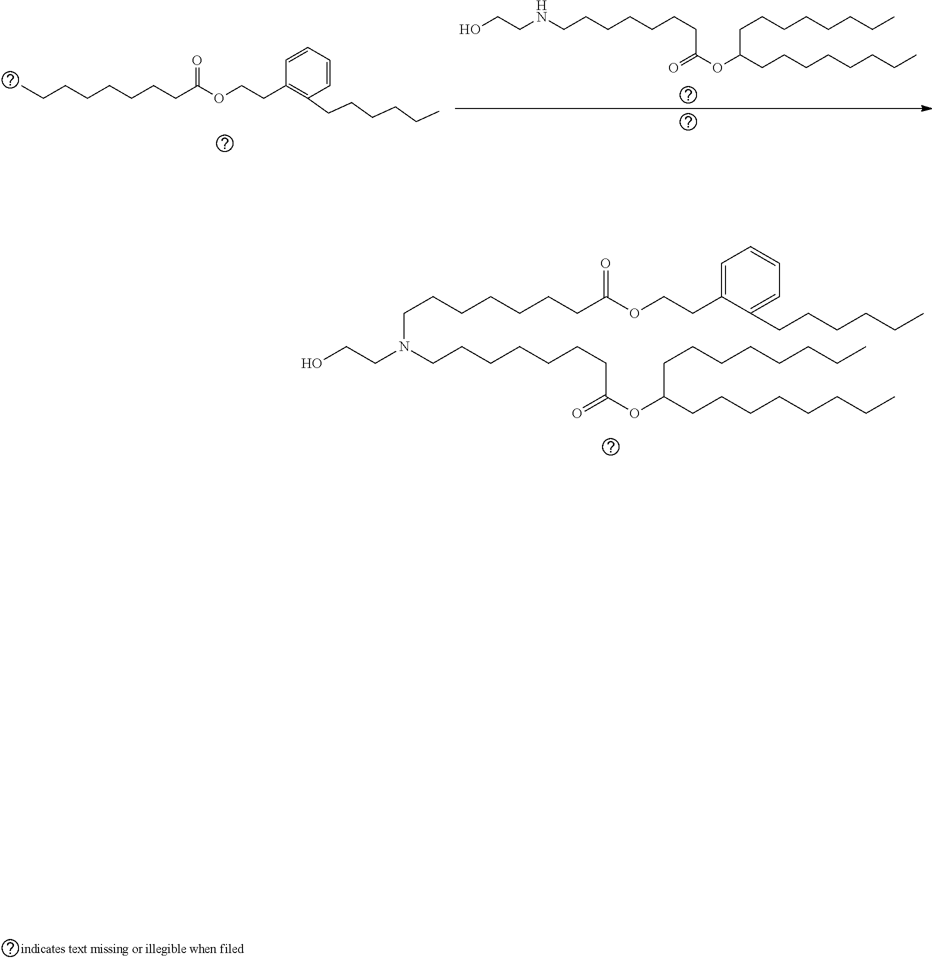 [SMILES: CCCCCCCCC(=O)OCCc1ccccc1CCCCCC ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccccc1CCCCCC]

To compound 6 (150 mg, 0.365 mmol, 1 eq.) and compound 7 (161 mg, 0.365 mmol, 1 eq.) in CPME/CH3 Potassium carbonate (302 mg, 2.19 mmol, 6 eq.) and potassium iodide (121 mg, 0.73 mmol, 2 eq.) were added to the mixture in CN (2 mL/2 mL). After addition, the mixture was stirred at 90° C. overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-140-1 (180 mg, 64%) as a yellow oil.

LCMS:Rt:1.568 min; MS m/z(ELSD):772.4[M+H]+;

HPLC: 98.053% purity, ELSD; RT=8.702 min.

1H NMR (400 MHz, CDCl 3) δ7.23-7.05 (m, 4H), 4.95-4.79 (m, 1H), 4.25 (t, J=7.4 Hz, 2H), 3.62 (t, J=4.8 Hz, 2H), 2.96 (dd, J=15.4, 8.0 Hz, 2H), 2.74-2.49 (m, 8H), 2.28 (dd, J=14.2, 7.2 Hz, 4H), 1.67-1.44 (m, 14H), 1.41-1.20 (m, 42H), 0.90 (dt, J=13.2, 7.1 Hz, 9H).

13C NMR (101 MHz, CDCl 3) δ173.68 (d, J=10.2 Hz), 141.26 (s), 135.23 (s), 129.73 (s), 129.37 (s), 126.72 (s), 125.92 (s), 77.35 (s), 77.03 (s), 76.71 (s), 74.17 (s), 64.52 (s), 57.99 (s), 55.87 (s), 53.94 (s), 34.66 (s), 34.21 (d, J=11.5 Hz), 32.75 (s), 31.83 (d, J=9.8 Hz), 31.32 (s), 29.65-28.88 (m), 27.15 (d, J=3.7 Hz), 26.35 (s), 25.33 (s), 25.07 (s), 24.83 (s), 22.66 (d, J=3.4 Hz), 14.12 (s).

### V. SW-II-140-2

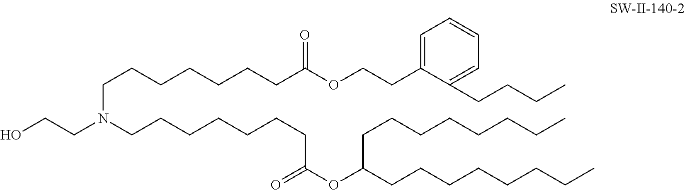 [SMILES: CCCCCCCCC(CCCCCCCC)OC(=O)CCCCCCCN(CCO)CCCCCCCC(=O)OCCc1ccccc1CCCC]

### 1. Synthesis of Compound 3

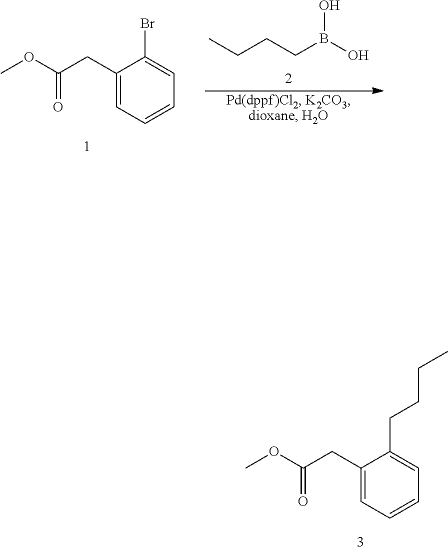

To a mixture of compound 1 (1 g, 4.37 mmol, 1 eq.) and compound 2 (668 g, 6.55 mmol, 1.5 eq.) in 1,4-dioxane/water (10 mL/1 mL) was added Pd(dppf)Cl 2 (286 mg, 0.437 mmol, 0.1 eq.) and potassium carbonate (1.8 g, 13.11 mmol, 3 eq.). The mixture was stirred at 100° C. overnight under nitrogen. TLC (PE/EA=20/1) showed the reaction was complete and new major spots were observed. The mixture was extracted with ethyl acetate and washed with water. The organic layer was dried over anhydrous sodium sulfate, filtered and concentrated in vacuo. The residue was purified by silica gel column chromatography, eluting with PE/EA (1/0-20/1), to obtain compound 3 (406 mg, 45%) as a colorless oil.

### 2. Synthesis of Compound 4

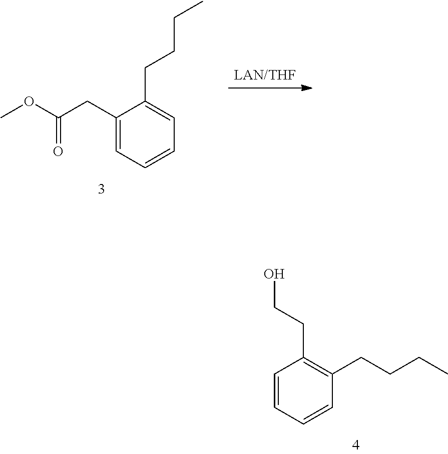

To a mixture of compound 3 (406 mg, 1.97 mmol, 1 eq.) in THF (5 mL) was added lithium aluminum hydride (2 mL, 1.97 mmol, 1 M, 1 eq. in THF) at 0° C. under nitrogen. The mixture was stirred at room temperature for 3 hours. TLC (PE/EA=5/1) indicated that the reaction was complete and new major spots were observed. The mixture was quenched with water (2 mL) and treated with 2N hydrochloric acid to adjust the pH between 6 and 7, extracted with ethyl acetate and washed with brine. The organic layer was dried over anhydrous sodium sulfate, filtered and concentrated in vacuo to afford compound 4 (341 mg, 97%) as a colorless oil without further purification.

### 3. Synthesis of Compound 6

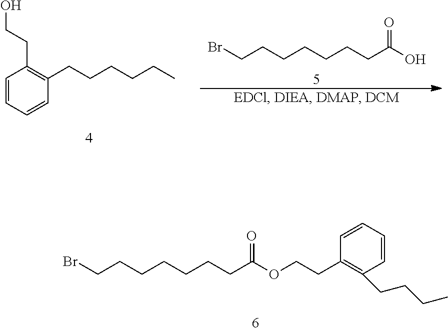

To a mixture of compound 4 (241 mg, 1.35 mmol, 1 eq.) and compound 5 (361 mg, 1.62 mmol, 1.2 eq.) in DCM (3 mL) was added EDCI (518 mg, 2.7 mmol, 2 eq.) and DMAP (66 mg, 0.54 mmol, 0.4 eq.), then DIEA (697 mg, 5.4 mmol, 4 eq.) was added. The reaction mixture was stirred at room temperature under nitrogen for 16 hours. TLC (petroleum ether/ethyl acetate=15/1) showed the formation of the desired product. The reaction mixture was extracted with ethyl acetate and washed with water. The organic layer was dried over anhydrous sodium sulfate, filtered and concentrated in vacuo. The residue was purified by silica gel column chromatography, eluting with petroleum ether/ethyl acetate (1/0-20/1), to obtain compound 6 (185 mg, 32%) as a colorless oil.

### 4. Synthesis of SW-II-140-2

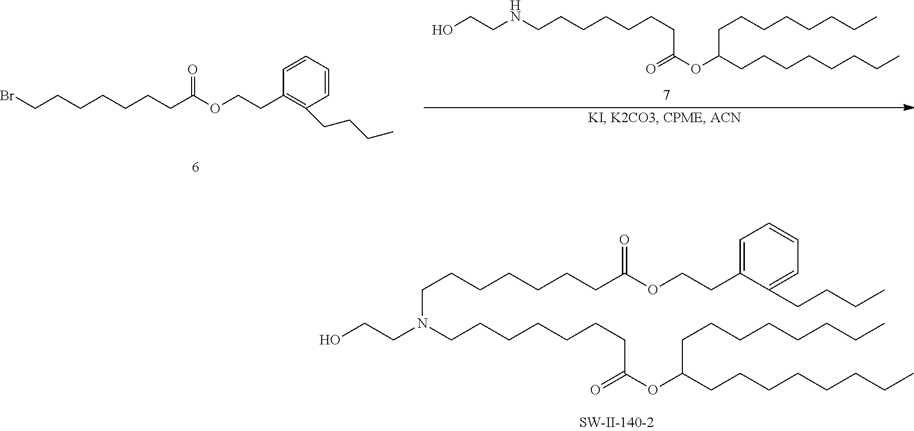 [SMILES: CCCCc1ccccc1CCOC(=O)CCCCCCCBr ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCc1ccccc1CCCC]

To compound 6 (185 mg, 0.483 mmol, 1 eq.) and compound 7 (213 mg, 0.483 mmol, 1 eq.) in CPME/CH3 To the mixture in CN (2 mL/2 mL) were added potassium carbonate (400 mg, 2.898 mmol, 6 eq.) and potassium iodide (160 mg, 0.966 mmol, 2 eq.). After addition, the mixture was stirred at 90° C. overnight under nitrogen. TLC (DCM/MeOH=10/1) showed the reaction was complete and new major spots were observed. The mixture was extracted with ethyl acetate and washed with water. The organic layer was dried over anhydrous sodium sulfate, filtered and concentrated under reduced pressure. The residue was purified by silica gel column chromatography, eluting with DCM/MeOH (1/0-10:1, v/v), to obtain compound SW-II-140-2 (161 mg, 45%) as a yellow oil.

LCMS:Rt:1.696 min; MS m/z(ELSD):744.3[M+H]+;

HPLC: 94.658% purity, ELSD; RT=5.938 min.

1H NMR (400 MHz, CDCl 3) δ7.22-7.03 (m, 4H), 4.94-4.78 (m, 1H), 4.25 (t, J=7.3 Hz, 2H), 3.70-3.54 (m, 2H), 2.96 (t, J=7.4 Hz, 2H), 2.77-2.41 (m, 8H), 2.28 (dd, J=14.3, 7.1 Hz, 4H), 1.65-1.18 (m, 52H), 0.91 (dt, J=13.3, 7.1 Hz, 9H).

13C NMR (101 MHz, CDCl 3) δ173.67 (d, J=10.8 Hz), 141.22 (s), 135.23 (s), 129.73 (s), 129.39 (s), 126.72 (s), 125.92 (s), 77.36 (s), 77.04 (s), 76.72 (s), 74.17 (s), 64.52 (s), 57.92 (s), 55.92 (s), 53.96 (s), 34.66 (s), 34.21 (d, J=11.2 Hz), 33.51 (s), 32.44 (s), 31.83 (d, J=9.3 Hz), 29.53 (d, J=2.9 Hz), 29.14 (dd, J=11.3, 8.5 Hz), 27.12 (d, J=4.1 Hz), 26.23 (s), 25.33 (s), 25.06 (s), 24.82 (s), 22.73 (d, J=9.9 Hz), 14.08 (d, J=8.8 Hz).

### Example 2. Gene Cloning of Plasmids Containing Different 5′-UTR Elements and Internal Control Plasmids

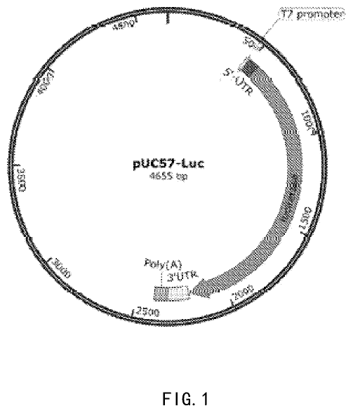

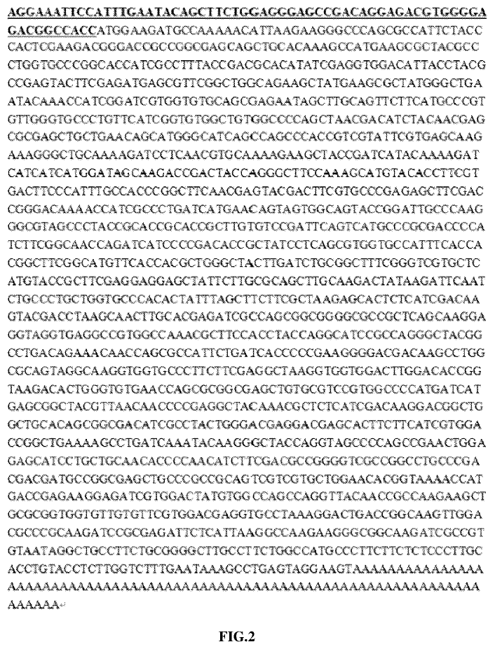

The plasmids containing different 5′-UTR elements are: the different 5′-UTR elements (sequences shown in SEQ ID NO: 1-69) are cloned into the reporter gene Firefly Luciferase by genetic means of homologous recombination.) before the upstream start codon, that is, the 5′-UTR region as shown in FIG. 1, and the rest are the same. The plasmid containing only the Kozak sequence before the upstream start codon of firefly luciferase was used as the reference construct. FIG. 2 shows an exemplary tested artificial nucleic acid molecule (SEQ ID NO: 143, in which the exemplary 5′-UTR element is SEQ ID NO: 1), and the underlined area is the tested 5′-UTR element. All elements of the sequence shown in FIG. 2 are identical to the reference artificial nucleic acid molecule (SEQ ID NO: 144) except for the underlined elements. Therefore, the only difference between SEQ ID NO:143 and SEQ ID NO:144 is that before the start codon, SEQ ID NO:144 only contains the Kozak sequence (GCCACC), while SEQ ID NO:143 contains the different 5 tested ′-UTR element.

The internal reference control plasmid is as follows: the reporter gene Firefly Luciferase (Firefly Luciferase) is replaced by the Renilla luciferase gene (Rluc), and other sequences of the plasmid are the same.

Plasmids containing different 5′-UTR elements and internal reference control plasmids were fully synthesized by Shanghai Sangon Bioengineering Company.

Utilize a pair of primers (upstream primer: 5′TTGGACCCTCCGTACAGAAGCTAATACG 3′; and downstream poly(T) long primer: 5′TTTTTTTTTTTTTTTTTTTTTTTTTTTTTTTTTTTTTTTTTTTTTTTTTTTTTTTTTTTTTTAC TTCCTACTCAGGCTTTATTCAAAGACCA 3′) and a high-fidelity DNA polymerase-based PCR amplification kit (Norway Biotechnology Co., Ltd. Company) performs PCR amplification to obtain DNA template.

### Example 3. Preparation of mRNA

Using the purified PCR product (Takara purification kit) prepared in Example 2 as a template, T7 RNA polymerase is used to perform a co-transcriptional capping reaction, and in vitro transcription of RNA is performed to generate Cap1 mRNA. During in vitro transcription, 1-methyl-pseudouridine-triphosphate was added instead of uridine triphosphate (UTP). Therefore, the modification ratio of 1-methyl-pseudouracil in the in vitro-transcribed Cap1 mRNA was 100%. After transcription, DNaseI (Thermo Fisher Scientific Co., Ltd.) was used to digest the DNA template to reduce the risk of residual DNA template. The mRNA was purified using DynabeadsMyone (Thermo Fisher Scientific, Inc.). Purified mRNA was dissolved in 1 mM sodium citrate buffer (pH 6.5+/−0.1), sterile filtered, and stored frozen at −80° C. until use.

### Example 4. Cellular Expression Verification of the Effect of Candidate 5′-UTR Elements on Reporter Genes

HEK-293 cells in good growth status were seeded into a 96-well cell culture plate at a seeding density of 3.5×10 4 cells/well, and then placed in a 37° C. cell culture incubator for 18-24 hours. Using Lipofectamine Messenger MAX reagent (Thermo Fisher), the mRNA of the plasmid containing the reporter gene firefly luciferase in Example 3 and the internal reference control plasmid containing the reporter gene sea cucumber luciferase were simultaneously transfected at an mRNA mass ratio of 2:1 per well. mRNA (total 100 ng mRNA) was injected into HEK293 cells in a 96-well plate, and three duplicate wells were set for each sample. Place the cell plate transfected with mRNA at 37° C., 5% CO2 Culture in the cell culture incubator for 24 hours.

Dual-luciferase reporter gene detection kit (Novizan, DL101-01) was used to detect the transfected cell samples. First perform pretreatment and add 100 μL/well of cell lysate into the 96-well cell plate. Then, carefully draw 14 μL of the lysed cell supernatant and transfer it to a 96-well all-black microplate. Then add 70 μL of firefly luciferase substrate that has been balanced to room temperature into the microplate. Mix quickly and use the enzyme immediately. Standard instrument (BioTek), shake the plate for 10 seconds, and detect the relative light unit (RLU) value of firefly luciferase at a wavelength of 560 nm; after the detection is completed, continue to add 70 μL of freshly prepared Renilla luciferase to the above reaction solution. The substrate working solution was mixed quickly and immediately used a microplate reader (BioTek) to detect the relative light unit (RLU) value of Renilla luciferase at a wavelength of 480 nm. The actual report for each well is the ratio of firefly luciferase RLU (Fluc) to Renilla luciferase RLU (Rluc) in each well (Fluc/Rluc). During data analysis, the Fluc/Rluc of the artificial nucleic acid molecule containing only the Kozak sequence was used as the reference nucleic acid molecule. The Fluc/Rluc ratio of the artificial nucleic acid molecule containing the different 5′-UTR elements tested relative to the reference was the final value. relative expression amount. Based on the results of three independent repeated experiments, the effects of different 5′-UTR elements on reporter gene expression were analyzed, a statistical histogram was drawn, and statistical analysis was performed.

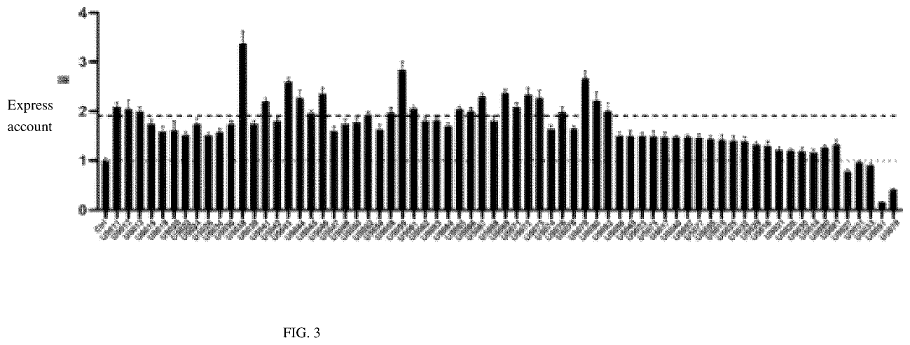

The experimental results are shown in FIG. 3 and Table 2. The translation efficiency of most artificial nucleic acid molecules containing the tested 5′-UTR elements is higher than that of the reference nucleic acid molecules. Among them, the numbers are U5061, U5065, U5066, U5067, U5069, U5071, U5072, U5073, U5076, U5079, U5080, U5082, U5011, U5012, U5013, U5038, U5041, U5043, U5044, U5045. U5046, U5052, U5058 The relative expression amount of the artificial nucleic acid molecule of the 5′-UTR element of U5059 (SEQ ID NO: 1-24) is greater than 1.9, indicating higher translation efficiency.

TABLE 2
Cell expression verification results

| No. of 5′-UTR | SEQ ID NO | Expression amount |
|---|---|---|
| U5061 | 1 | 2.05 |
| U5065 | 2 | 2.05 |
| U5066 | 3 | 2 |
| U5067 | 4 | 2.29 |
| U5069 | 5 | 2.37 |
| U5071 | 6 | 2.07 |
| U5072 | 7 | 2.33 |
| U5073 | 8 | 2.27 |
| U5076 | 9 | 1.98 |
| U5079 | 10 | 2.66 |
| U5080 | 11 | 2.22 |
| U5082 | 12 | 2 |
| U5011 | 13 | 2.08 |
| U5012 | 14 | 2.04 |
| U5013 | 15 | 1.99 |
| U5038 | 16 | 3.38 |
| U5041 | 17 | 2.2 |
| U5043 | 18 | 2.59 |
| U5044 | 19 | 2.28 |
| U5045 | 20 | 1.96 |
| U5046 | 21 | 2.35 |
| U5052 | 22 | 1.92 |
| U5058 | 23 | 1.97 |
| U5059 | 24 | 2.83 |
| U5014 | 25 | 1.17 |
| U5015 | 26 | 1.74 |
| U5016 | 27 | 1.3 |
| U5017 | 28 | 1.48 |
| U5018 | 29 | 1.42 |
| U5019 | 30 | 1.58 |
| U5020 | 31 | 1.61 |
| U5021 | 32 | 1.22 |
| U5022 | 33 | 1.52 |
| U5023 | 34 | 1.40 |
| U5024 | 35 | 1.74 |
| U5025 | 36 | 1.4 |
| U5026 | 37 | 1.51 |
| U5027 | 38 | 0.78 |
| U5028 | 39 | 1.2 |
| U5029 | 40 | 1.33 |
| U5030 | 41 | 1.2 |
| U5031 | 42 | 0.96 |
| U5032 | 43 | 1.39 |
| U5033 | 44 | 0.9 |
| U5034 | 45 | 1.58 |
| U5036 | 46 | 1.74 |
| U5039 | 47 | 1.74 |
| U5040 | 48 | 1.48 |
| U5042 | 49 | 1.8 |
| U5047 | 50 | 1.6 |
| U5048 | 51 | 1.74 |
| U5049 | 52 | 1.5 |
| U5050 | 53 | 1.78 |
| U5051 | 54 | 0.16 |
| U5054 | 55 | 1.63 |
| U5055 | 56 | 1.43 |
| U5056 | 57 | 1.5 |
| U5057 | 58 | 1.47 |
| U5060 | 59 | 1.27 |
| U5062 | 60 | 1.79 |
| U5063 | 61 | 1.82 |
| U5064 | 62 | 1.7 |
| U5068 | 63 | 1.8 |
| U5070 | 64 | 0.41 |
| U5074 | 65 | 1.64 |
| U5075 | 66 | 1.49 |
| U5077 | 67 | 1.47 |
| U5078 | 68 | 1.64 |
| U5081 | 69 | 1.32 |
| Kozak | 70 | 1 |

### Example 5. Preparation of LPP Formulation

### 5.1. Experimental Materials

Cationic lipid SW-II-140-2 was synthesized by Sri Lankan microorganisms; auxiliary phospholipid (DOPE) was purchased from CordenPharma; cholesterol was purchased from Sigma-Aldrich; mPEG2000-DMG (ie DMG-PEG 2000) was purchased from Avanti Polar Lipids, Inc.; PBS was purchased from Invitrogen; protamine sulfate was purchased from Beijing Silian Pharmaceutical Co., Ltd.

5.2. Preparation of Lipid Polyplex (LPP) of mRNA

Preparation of nucleic acid aqueous solution: Dilute each mRNA prepared as in Example 3 with 10 mM citric acid-sodium citrate buffer (pH 4.0) to a 0.2 mg/mL mRNA solution.

Preparation of lipid solution: Dissolve SW-II-140-2: DOPE:cholesterol:mPEG2000-DMG in ethanol solution at a molar ratio of 40:15:43.5:1.5 to prepare a lipid solution of 10 mg/mL.

Preparation of protamine sulfate solution: Dissolve protamine sulfate in nuclease-free water to prepare a protamine sulfate solution with a working concentration of 0.25 mg/mL.

Preparation of core nanoparticle solution: Using microfluidic technology (Maiana (Shanghai) Technology Co., Ltd., model: Inano D), the protamine sulfate solution and the nucleic acid solution were mixed under the following conditions to obtain the solution obtained from fish Nuclear nanoparticle solution formed by protamine and artificial nucleic acid molecules: volume=4.0 mL; flow rate ratio=5 (mRNA): 1 (protamine solution), total flow rate=12 mL/min, start waste=0.35 mL, end waste=0.1 mL, room temperature.

Preparation of LPP: Mix the core nanoparticle solution and the lipid solution twice under the following conditions: volume=4.0 mL, flow rate ratio=3 (lipid solution): 1 (core nanoparticle solution), total flow rate=12 mL/min, front waste=0.35 mL, rear waste=0.1 mL, room temperature, obtain LPP solution.

Centrifugal ultrafiltration: Centrifuge the LPP solution to remove ethanol through ultrafiltration (centrifugal force 3000 g, centrifugation time 60 minutes, temperature 4° C.) to obtain 0.1 mg/ml LPP preparations containing different artificial nucleic acid molecules.

It will be apparent to those skilled in the art that many modifications and variations of the present invention can be made without departing from its spirit and scope. The specific embodiments described herein are provided by way of example only and are not meant to be limiting in any way.

The true scope and spirit of the invention is indicated by the appended claims, and the specification and examples are exemplary only.

### Sequence List

| SEQ ID NO: | [symbol] | [symbol] |
|---|---|---|
| 1 | U5061 DNA | AGGAAATTCCATTTGAATACAGCTTCTGGAGGGAGCCGACAGGAGACGTGGGGAGACGGCCACC |
| 2 | U5065 DNA | AGGTGCGACTTACCGCGCAATCTAACCCAGCAGTCTGCATTGATTAACGCCACC |
| 3 | U5066 DNA | AGGCACTTTTATTAGTGTTGTCCAGTCATTACACTCGGATACCTTGTTGCCACC |
| 4 | U5067 DNA | AGAACGGAACAACCTGTTGTCATTGGATCATACCGTATCACTGACCTAGCCACC |
| 5 | U5069 DNA | AGAATATCTCGCTATTCTACACGTGATCTCACGCAGTGTATCCCTCCGCCACC |
| 6 | U5071 DNA | AGTGTTCCAAGCTTCGTCTCAAGTTGAGTTGTTTTAGATTGCTGAGTTTGATCACAGAAAAGGGTAATTTCGG |
| | | TCCGCACTGCCACC |
| 7 | U5072 DNA | AGGGGCTATCTACCTTCAGGCCTTGTCTTGTAGAACAGTTAGTAAGGACCTACTATTCAAGAACTAACTAAGG |
| | | CGCGTCCAGCCACC |
| 8 | U5073 DNA | AGACGATCATAGACTAGGACGTGTCGTCGGTCATTGGAATATAAAGGAACTTGCCGTCATAGTATCTTTAAGG |
| | | TCACGCGTGCCACC |
| 9 | U5076 DNA | AGCGAAATTTATTAAAAAAACGTTCTGAATCGGCCAGGAGAGGCCGACAAAAACCGCCACC |
| 10 | U5079 DNA | AGGCAAAATACATCATAGTGCAGCCTATAGATAAATCGGACAGGATATTATCGGAGACAGAAACCGCCACC |
| 11 | U5089 DNA | AGCAGGCAAGTACATCCTTCGTGTCCTAGGTGATAGTAATTGGTATCGAGGTATATCAGAAGACAGGCAATTT |
| | | CATCTGGCGGTGCACACCCTACCAGCAACGCCACC |
| 12 | U5082 DNA | AGAATCAAATCATCTTGACGGTTCAGAGTCGGGCAACGAGCAGTTTCCCGCTTGTGTCCTATCACGTTCGCAG |
| | | AGGGGGCATCGAGGATCTTAACTGCACTCGCCACC |
| 13 | U5011 DNA | AGACTCTTCTGGTCCCCACAGACTCAGAGAGAACCCACC |
| 14 | U5012 DNA | AGCGCCCGCTGCAGCCGCCGGCTCTGAATCCTTCTGGCTTCCGCCTCAGAGGAGTTCTTAGCCTGTCCCGAAC |
| | | CGTAACCCCGGCGAGCAGGCCACC |
| 15 | U5013 DNA | AGAGAGCGCCGGACCACCCTCTGTCCGCCCCGAGGAGTTTGCCGCCTGCCGGAGCACCTGCGCACAGCCCACC |
| 16 | U5038 DNA | AGCATTCCAACCTTCCAGCCTGCGACCTGCGGAGAAAAAAAATTACTTATTTTCTTGCCCCATACATACCTTG |
| | | AGGCGAGCAAAAAAATTAAATTTTAACC |
| 17 | U5041 DNA | AGCACTCTGCGCGCCCGCTCTTCTGCTGCTGTTTGTCTACTTCCTCCTGCTTCCCCGCCGCCGCCGCCGCCAT |
| | | CGCCACC |
| 18 | U5043 DNA | AGGGGAGATCTTCGTCACCCTTTTTCACTTCCTCAGTTTTCGCGGACCACTTCAAGGACTAAAAGCCACC |
| 19 | U5044 DNA | AGCTCCTCCCGAAGTGCTCCTCTTCTACAGCTGTTCCGCAGTCGCCGCCGCCGCCACC |
| 20 | U5045 DNA | AGGCTCTTCCAGACTCTGCGACCTAACTGACTCTTGGGTTCAAGGCTAGGGCCACC |
| 21 | U5046 DNA | AGCGCAGCCTGCTGTCCGCCTGCCTTTTCGTCTCTAGCCGCGTGAAGTCAGCGCCACC |
| 22 | U5052 DNA | AGCTCAAAGAAAAACCAAACGTAACAAGGGCGAATTCGTTGGTAAAGCCACC |
| 23 | U5058 DNA | AGGGTCCCACAGTCTGGTCCTGCTTCACCTTCCCCTGACCTGAGTAGTCGCCGCCACC |
| 24 | U5059 DNA | AGCTCAGTGAGGCAACCCTAATTAACCTGATTTTTTGCTGATAATCACTCTCAGCCACC |
| 25 | U5614 DNA | AGAGTCCCGAGCCTAAAGCCTCGCCTGCCTGCCCAGTGCCAGGGCCACC |
| 26 | U5615 DNA | AGCTCAGTCCTGGTCCCCCAGTCCCAAGCCTCACCTGCCTGCCCAGCGCCAGGGCCACC |
| 27 | U5616 DNA | AGAAAAGAAAGAAGCCAGAACATACCACCAGCCCTTTGAGTATACAACATAACAAACGTCATTACTTTACAAC |
| | | TTGATTAGGAGACAGTACAGAGATCTGAAGGGCCACC |
| 28 | U5017 DNA | AGAGAACACGACACAACGAACTTTATATTTTACCACTTCCTTGAATAGTTGCAGGAGAAATAACAAGGCATTG |
| | | AAGAGCCACC |
| 29 | U5018 DNA | AGGAGACGCCATCCACGCTGTTTTGACCTCCATAGAAGACACCGGGACCGATCCAGCCTCCGCGGCCGGGAAC |
| | | GGTGCATTGGAACGCGGATTCCCCGTGCCAAGAGTGACTCACCGTCCTTGACACGGCCACC |
| 30 | U5019 DNA | AGAACGGTGCATTGGAACGCGGATTCCCCGTGCCAAGAGTGACTCACCGTCCTTGACACGGCCACC |
| 31 | U5020 DNA | AGGGAGACGCCATCCACCCTGTTTTGACCTCCATAGAAGACACCGGGACCG |
| 32 | U5021 DNA | AGATAAACGCTCAACTTTGGCC |
| 33 | U5022 DNA | AGATAAACGCTCAACTTTGGCCGCCACC |
| 34 | U5023 DNA | AGATAAACGCTCAACTTTGGCCGCCAAG |
| 35 | U5024 DNA | AGATTCCCCGCCTCCTCCTGTCCCGCAGTTCGGATAAACGCTCAACTTTGGCC |
| 36 | U5025 DNA | AGGGAGACCATATTGAAGAGACAGAGTGATATATAAAACTGCTAA |
| 37 | U5026 DNA | AGGGAGACCATATTGAAGAGACAGAGTGATATATAAAACTGCTAAGCCACC |
| 38 | U5027 DNA | AGGGAGACAAAACTGCTAA |
| 39 | U5028 DNA | AGGGAGACAAAACTGCTAAGCCAAG |
| 40 | U5029 DNA | AGGGAGACTGCCACC |
| 41 | U5030 DNA | AGGGAGACTGCCAAG |
| 42 | U5031 DNA | AGGGAAGTGCAGCCCCAGGTTCCCGAGCGGCAGGTTGGGTGCGGACC |
| 43 | U5032 DNA | AGGGAACTCCTCCCCATCCTCTCCCTCTGTCCCTCTGTCCCTCTGACCCTGCACTGTCCCAGCACC |
| 44 | U5033 DNA | AGGGACTCCCGGGCTGCAGCAGGGCCCCAGCGGCACC |
| 45 | U5034 DNA | AGGGAAATCCTTTCTTTCAGCTGGAGTGCTCCTCAGGAGCCAGCCCCACCCTTAGAAAAG |
| 46 | U5036 DNA | AGCACTCGCGCTGCCATCACTCTTCCGCCGTCTTCGCCGCCATCCTCGGCGCGACTCGCTTCTTTCGGTTCTA |
| | | CCAGGTAGAGTCCGCCGCCATCCTCCACC |
| 47 | U5039 DNA | AGCCGGTGGCGGCAGGATACAGCGGCTTCTGCGCGACTTATAAGAGCTCCTTGTGCGGCGCCATTTTAAGCCT |
| | | CTCGGTCTGTGGCAGCAGCGTTGGCCCG |
| 48 | U5040 DNA | AGCTACAGAAACGAAAGAAAAAGTCTGTATAAGCCAAAGGTGTTCGGGAAGAAAATAACCCCATTGCCTTGAG |
| | | TTTGTAGGTGCCACTACTACTCTGGAAAA |
| 49 | U5042 DNA | AGTTGCCGCTGTCGTCCGCAGAGCCAGTTCCTAGCGCAGAGCCGCGCCCGCCGCCACC |
| 50 | U5047 DNA | AGCGCGTTATTGTTCTGCCGGGCGGACACGTGACGCGAAGCTTACCGCCGAGACCGCGTCCGCCCCGCGAGCA |
| | | CAGAGCCTCGCCTTTGCCGATCCGCCGCCCGTCCACACCCGCCGCCAGCTCACCGCCACC |
| 51 | U5048 DNA | AGACCGCCGAGACCGCGTCCGCCCCGCGAGCACAGAGCCTCGCCTTTGCCGATCCGCCGCCCGTCCACACCCG |
| | | CCGCCAGCTCACCGCCACC |
| 52 | U5049 DNA | AGTATAAAACCCGGCGGCGCAACGCGCAGCCACTGTCGAGTCGCGTCCACCCGCGAGCACAGCTTCTTTGCAG |
| | | CTCCTTCGTTGCCGGTCCACACCCGCCACCAGTTCGCCCCGCCACC |
| 53 | U5050 DNA | AGCGCGTTATTGTTCTGCCGGGCGGACACGTGACGCGAAGCTTTATAAAACCCGGCGGCGCAACGCGCAGCCA |
| | | CTGTCGAGTCGCGTCCACCCGCGAGCACAGCTTCTTTGCAGCTCCTTCGTTGCCGGTCCACACCCGCCACCAG |
| | | TTCGCCCCGCCACC |
| 54 | U5051 DNA | AGTTGGGGGCGACACTCCACCATAGATCACTCCCCTGTGAGGAACTACTGTCTTCACGCAGAAAGCGTCTAGC |
| | | CATGGCGTTAGTATGAGTGTCGTGCAGCCTCCAGGACCCCCCCTCCCGGGAGAGCCATAGTGGTCTGCGGAAC |
| | | CGGTGAGTACACCGGAATTGCCAGGACGACCGGGTCCTTTCTTGGATTAACCCGCTCAATGCCTGGAGATTTG |
| | | GGCGTGCCCCCGCGAGACTGCTAGCCGAGTAGTGTTGGGTCGCGAAAGGCCTTGTGGTACTGCCTGATAGGGT |
| | | GCTTGCGAGTGCCCCGGGAGGTCTCGTAGACCGTGCATCATGAGCACAAATCCTAAACCTCAAAGAAAAACCA |
| | | AACGTAACAAGGGCGAATTCGTTGGTAAAGCCACC |
| 55 | U5054 DNA | AGTCAACGTAGTTCTAACAGTTTTTTAATTAGAGAGCAGATCTCTGGCCACC |
| 56 | U5055 DNA | AGATCTTTGGGTGGTGGAGTGCAAAGGAGGCGACCTGCAACAGAGGAGTCCCGGTCACCAGCCACC |
| 57 | U5056 DNA | AGAAACCCCAGCTGTTGGGGCCAGGACACCCAGTGAGCCCATACTTGCTCTTTTTGTCTTCTTCAGACTGCGC |
| | | CGCCACC |
| 58 | U5059 DNA | AGCAAAAGGAATTCTTCTCGGGAGGCAGTGCTGGGTCCTTTCCACCGCCACC |
| 59 | U5060 DNA | AGGAAATTGGATTTGGCTGCAGCTTCTGGAGGGAGCCGACAGGAGACGTGGGGAGACGGCCACC |
| 60 | U5062 DNA | AGGAAATTCCATTTGGCTGCAGCTTCTGGAGGGAGCCGACAGGAGACGTGGGGAGAGTGCCACC |
| 61 | U5063 DNA | AGGAAATTCCATTTGGCTGCAGCTTAAAGAGGGAGCCGACAGGAGACGTGGGGAGACGGCCACC |
| 62 | U5064 DNA | AGGAAATTCCATTTGAATTCAGCTTCTGGAGGGAGCCGACAGGAGACGTGGGGAGACGGCATCC |
| 63 | U5068 DNA | AGGGTCTATTCAAAGACTGCGGACGCGATCAAGTGCTCTGTTCCATCTGCCACC |
| 64 | U5070 DNA | AGTCGGTTGTCTAATCAAGATCATATCTTGGGACCGGGCTATACTTCATCAAAAACTGTCCATATAGTCGCGG |
| | | CGGGGAAAGCCACC |
| 65 | U5074 DNA | AGCGAACTTACAAAAAAAAGCATCTTATCGAAACCGCCGCTCGGAGACGAGCGCACAGCCACC |
| 66 | U5075 DNA | AGCGAAAAAAAATTAAAATCCAGCTTCTGTAAAGAGCCGACAGGAGAGGTGGCGAGACAAAAACCGCCACC |
| 67 | U5077 DNA | AGGTAGTAAAAAAGCTGATTGAGTTGTTAGGAGAGGTGGCGAGAGTTTAGGGGAGGGCCACC |
| 68 | U5078 DNA | AGGCACGGTAACCGAAAGCGTTCGAACAGGGGCGCCAATACATTAAAATTGCCACC |
| 69 | U5081 DNA | AGTTAGAAAGAGTACTCATAAACGATAATAAAGTGAACCTATTAGGCCCGTCTTGCGGAGTTGAGGAGCTTGC |
| | | ATCTCTGCTGAGGTTAAAACTAATATCTTGCCACC |
| 70 | Kozak | GCCACC |
| 71 | [symbol] | AGGAAATTCCATTTGGCTGCAGCTTCTGGAGGGAGCCGACAGGAGACGTGGGGAGACGGCCACC |
| 72 | 3′-UTR | GCTGCCTTCTGCGGGGCTTGCCTTCTGGCCATGCCCTTCTTCTCTCCCTTGCACCTGTACCTCTTGGTCTTTG |
| | | AATAAAGCCTGAGTAGGAAGT |
| 73 | poly(A) | AAAAAAAAAAAAAAAAAAAAAAAAAAAAAAAAAAAAAAAAAAAAAAAAAAAAAAAAAAAAAAAAAAAAAAAAA |
| | | AA |
| 74 | U5061 RNA | AGGAAAYUUCCAUUUGAAUACAGCUUCUGGAGGGAGCCGACAGGAGACGUGGGGAGACGGCCACC |
| 75 | U5065 RNA | AGGUGCGACUUACCGCGCAAUCUAACCCAGCAGUCUGCAUUGAUUAACGCCACC |
| 76 | U5066 RNA | AGGCACUUUUAUUAGUGUUGUCCAGUCAUUACACUCGGAUACCUUGUUGCCACC |
| 77 | U5067 RNA | AGAACGGAACAACCUGUUGUCAUUGGAUCAUACCGUAUCACUGACCUAGCCACC |
| 78 | U5069 RNA | AGAAUAUCUCGCUAUUCUACACGUGAUCUCACGCAGUGUAUCCCUCCGCCACC |
| 79 | U5071 RNA | AGUGUUCCAAGCUUCGUCUCAAGUUGAGUUGUUUUAGAUUGCUGAGUUUGAUCACAGAAAAGGGUAAUUUCGG |
| | | UCCGCACUGCCACC |
| 80 | U5072 RNA | AGGGGCUAUCUACCUUCAGGCCUUGUCUUGUAGAACAGUUAGUAAGGACCUACUAUUCAAGAACUAACUAAGG |
| | | CGCGUCCAGCCACC |
| 81 | U5073 RNA | AGACGAUCAUAGACUAGGACUGUGUCGUCGGUCAUUGGAAUAUAAAGGAACUUGCCGUCAUAGCAUCUUUAAG |
| | | GUCACGCGUGCCACC |
| 82 | U5076 RNA | AGCGAAAUUUAUUAAAAAAACGUUCUGAAUCGGCCAGGAGAGGCCGACAAAAACCGCCACC |
| 83 | U5079 RNA | AGGCAAAAUACAUCAUAGUGCAGCCUAUAGAUAAAUCGGACAGGAUAUUAUCGGAGACAGAAACCGCCACC |
| 84 | U5080 RNA | AGCAGGCAAGUACAUCCUUCGUGUCCUAGGUGAUAGUAAUUUGUAUCGAGGUAUAUCAGAAGACAGGCAAUUU |
| | | CAUCUGGCGGUGCACACCCUACCAGCAACGCCACC |
| 85 | U5082 RNA | AGAAUCAAAUCAUCUUGACGGUUCAGAGUCGGGCAACGAGCAGUUUCCCGCUUGUGUCCUAUCACGUUCGCAG |
| | | AGGGGGCAUCGAGGAUCUUAACUGCACUCGCCACC |
| 86 | U5011 RNA | AGACUCUUCUGGUCCCCACAGACUCAGAGAGAACCCACC |
| 87 | U5012 RNA | AGCGCCCGCUGCAGCCGCCGGCUCUGAAUCCUUCUGGCUUCCGCCUCAGAGGAGUUCUUAGCCUGUCCCGAAC |
| | | CGUAACCCCGGCGAGCAGGCCACC |
| 88 | U5013 RNA | AGAGAGCGCCGGACCACCCUCUGUCCGCCCCGAGGAGUUUGCCGCCUGCCGGAGCACCUGCGCACAGCCCACC |
| 89 | U5038 RNA | AGCAUUCCAACCUUCCAGCCUGCGACCUGCGGAGAAAAAAAAUUACUUAUUUUCUUGCCCCAUACAUACCUUG |
| | | AGGCGAGCAAAAAAAUUAAAUUUUAACC |
| 90 | U5041 RNA | AGCACUCUGCGCGCCCGCUCUUCUGCUGCUGUUUGUCUACUUCCUCCUUGCUUCCCCGCCGCCGCCGCCGCCA |
| | | UCGCCACC |
| 91 | U5043 RNA | AGGGGAGAUCUUCGUCACCCUUUUUCACUUCCUCAGUUUUCGCGGACCACUUCAAGGACUAAAAGCCACC |
| 92 | U5044 RNA | AGCUCCUCCCGAAGUGCUCCUCUUCUACAGCUGUUCCGCAGUCGCCGCCGCCGCCACC |
| 93 | U5045 RNA | AGGCUCUUCCAGACUCUGCGACCUAACUGACUCUUGGGUUCAAGGCUAGGGCCACC |
| 94 | U5046 RNA | AGCGCAGCCUGCUGUCCGCCUGCCUUUUCGUCUCUAGCCGCGUGAAGUCAGCGCCACC |
| 95 | U5052 RNA | AGCUCAAAGAAAAACCAAACGUAACAAGGGCGAAUUCGUUGGUAAAGCCACC |
| 96 | U5058 RNA | AGGGUCCCACAGUCUGGUCCUGCUUCACCUUCCCCUGACCUGAGUAGUCGCCGCCACC |
| 97 | U5059 RNA | AGCUCAGUUGAGCAACCCUAAUUAACCUGAUUUUUUGCUGAUAAUCACUCUCAGCCACC |
| 98 | U5014 RNA | AGAGUCCCGAGCCUAAAGCCUCGCCUGCCUGCCCAGUGCCAGGGCCACC |
| 99 | U5015 RNA | AGCUCAGUCCUGGUCCCCCAGUCCCAAGCCUCACCUGCCUGCCCAGCGCCAGGGCCACC |
| 100 | U5016 RNA | AGAAAAGAAAGAAGCCAGAACAUACCACCAGCCCUUUGAGUAUACAACAUAACAAACGUCAUUACUUUACAAC |
| | | UUGAUUAGGAGACAGUACAGAGAUCUGAAGGGCCACC |
| 101 | U5017 RNA | AGAGAACACGACACAACGAACUUUAUAUUUUACCACUUCCUUGAAUAGUUGCAGGAGAAAUAACAAGGCAUUG |
| | | AAGAGCCACC |
| 102 | U5018 RNA | AGGAGACGCCAUCCACGCUGUUUUGACCUCCAUAGAAGACACCGGGACCGAUCCAGCCUCCGCGGCCGGGAAC |
| | | GGUGCAUUGGAACGCGGAUUCCCCGUGCCAAGAGUGACUCACCGUCCUUGACACGGCCACC |
| 103 | U5019 RNA | AGAACGGUGCAUUGGAACGCGGAUUCCCCGUGCCAAGAGUGACUCACCGUCCUUGACACGGCCACC |
| 104 | U5020 RNA | AGGGAGACGCCAUCCACGCUGUUUUGACCUCCAUAGAAGACACCGGGACCG |
| 105 | U5021 RNA | AGAUAAACGCUCAACUUUGGCC |
| 106 | U5022 RNA | AGAUAAACGCUCAACUUUGGCCGCCACC |
| 107 | U5023 RNA | AGAUAAACGCUCAACUUUGGCCGCCAAG |
| 108 | U5024 RNA | AGAUUCCCCGCCUCCUCCUGUCCCGCAGUCGGAUAAACGCUCAACUUUGGCC |
| 109 | U5025 RNA | AGGGAGACCAUAUUGAAGAGACAGAGUGAUAUAUAAAACUGCUU |
| 110 | U5026 RNA | AGGGAGACCAUAUUGAAGAGACAGAGUGAUAUAUAAAACUGCUAAGCCACC |
| 111 | U5027 RNA | AGGGAGACAAAACUGCUAA |
| 112 | U5028 RNA | AGGGAGACAAAACUGCUAAGCCAAG |
| 113 | U5029 RNA | AGGGAGACUGCCACC |
| 114 | U5030 RNA | AGGGAGACUGCCAAG |
| 115 | U5031 RNA | AGGGAAGUGCAGCCCCAGGUUCCCGAGCGGCAGGUUGGGUGCGGACC |
| 116 | U5032 RNA | AGGGAACUCCUCCCCAUCCUCUCCCUCUGUCCCUCUGUCCCUCUGACCCUGCACUGUCCCAGCACC |
| 117 | U5033 RNA | AGGGACUCCCGGGCUGGCAGCAGGGCCCCAGCGGCACC |
| 118 | U5034 RNA | AGGGAAAUCCUUUCUUUCAGCUGGAGUGCUCCUCAGGAGCCAGCCCCACCCUUAGAAAAG |
| 119 | U5036 RNA | AGCACUCGCGCUGCCAUCACUCUUCCGCCGUCUUCGCCGCCAUCCUCGGCGCGACUCGCUUCUUUCGGUUCUA |
| | | CCAGGUAGAGUCCGCCGCCAUCCUCCACC |
| 120 | U5039 RNA | AGCCGGUGGCGGCAGGAUACAGCGGCUUCUGCGCGACUUAUAAGAGCUCCUUGUGCGGCGCCAUUUUAAGCCU |
| | | CUCGGUCUGUGGCAGCAGCGUUGGCCCG |
| 121 | U5040 RNA | AGCUACAGAAACGAAAGAAAAAGUCUGUAUAAGCCAAAGGUGUUCGGGAAGAAAAUAACCCCAUUGCCUUGAG |
| | | UUUGUAGGUGCCACUACUACUCUGGAAAA |
| 122 | U5042 RNA | AGUUGCCGCUGUCGUCCGCAGAGCCAGUUCCUAGCGCAGAGCCGCGCCCGCCGCCACC |
| 123 | U5047 RNA | AGCGCGUUAUUGUUCUGCCGGGCGGACACGUGACGCGAAGCUUACCGCCGAGACCGCGUCCGCCCCGCGAGCA |
| | | CAGAGCCUCGCCUUUGCCGAUCCGCCGCCCGUCCACACCCGCCGCCAGCUCACCGCCACC |
| 124 | U5048 RNA | AGACCGCCGAGACCGCGUCCGCCCCGCGAGCACAGAGCCUCGCCUUUGCCGAUCCGCCGCCCGUCCACACCCG |
| | | CCGCCAGCUCACCGCCACC |
| 125 | U5049 RNA | AGUAUAAAACCCGGCGGCGCAACGCGCAGCCACUGUCGAGUCGCGUCCACCCGCGAGCACAGCUUCUUUGCAG |
| | | CUCCUUCGUUGCCGGUCCACACCCGCCACCAGUUCGCCCCGCCACC |
| 126 | U5050 RNA | AGCGCGUUAUUGUUCUGCCGGGCGGACACGUGACGCGAAGCUUUAUAAAACCCGGCGGCGCAACGCGCAGCGA |
| | | CUGUCGAGUCGCGUCCACCCGCGAGCACAGCUUCUUUGCAGCUCCUUCGUUGCCGGUCCACACCCGCCACCAG |
| | | UUCGCCCCGCCACC |
| 127 | U5051 RNA | AGUUGGGGGCGACACUCCACCAUAGAUCACUCCCCUGUGAGGAACUACUGUCUUCACGCAGAAAGCGUCUAGC |
| | | CAUGGCGUUAGUAUGAGUGUCGUGCAGCCUCCAGGACCCCCCCGGGAGAGCCAUAGUGGUCUGCGGAACCGGU |
| | | GAGUACACCGGAAUUGCCAGGACGACCGGGUCCUUUCUUGGAUUAACCCGCUCAAUGCCUGGAGAUUUGGGCG |
| | | UGCCCCCGCGAGACUGCUAGCCGAGUAGUGUUGGGUCGCGAAAGGCCUUGUGGUACUGCCUGAUAGGGUGCUU |
| | | GCGAGUGCCCCGGGAGGUCUCGUAGACCGUGCAUCAUGAGCACAAAUCCUAAACCUCAAAGAAAAACCAAACG |
| | | UAACAAGGGCGAAUUCGUUGGUAAAGCCACC |
| 128 | U5054 RNA | AGUCAACGUAGUUCUAACAGUUUUUUAAUUAGAGAGCAGAUCUCUGGCCACC |
| 129 | U5055 RNA | AGAUCUUUGGGUGGUGGAGUGCAAAGGAGGCGACCUGCAACAGAGGAGUCCCGGUCACCAGCCACC |
| 130 | U5056 RNA | AGAAACCCCAGCUGUUGGGGCCAGGACACCCAGUGAGCCCAUACUUGCUCUUUUGUCUUCUUCAGACUGCGCC |
| | | GCCACC |
| 131 | U5057 RNA | AGCAAAAGGAAUUCUUCUCGGGAGGCAGUGCUGGGUCCUUUCCACCGCCACC |
| 132 | U5060 RNA | AGGAAAUUGGAUUUGGCUGCAGCUUCUGGAGGGAGCCGACAGGAGACGUGGGGAGACGGCCACC |
| 133 | U5062 RNA | AGGAAAUUCCAUUUGGCUGCAGCUUCUGGAGGGAGCCGACAGGAGACGUGGGGAGAGUGCCACC |
| 134 | U5063 RNA | AGGAAAUUCCAUUUGGCUGCAGCUUAAAGAGGGAGCCGACAGGAGACGUGGGGAGACGGCCACC |
| 135 | U5064 RNA | AGGAAAUUCCAUUUGAAUUCAGCUUCUGGAGGGAGCCGACAGGAGACGUGGGGAGACGGCAUCC |
| 136 | U5068 RNA | AGGGUCUAUUCAAAGACUGCGGACGCGAUCAAGUGCUCUGUUCCAUCUGCCACC |
| 137 | U5070 RNA | AGUCGGUUGUCUAAUCAAGAUCAUAUCUUGGGACCGGGCUAUACUUCAUCAAAAACUGUCCAUAUAGUCGCGG |
| | | CGGGGAAAGCCACC |
| 138 | U5074 RNA | AGCGAACUUACAAAAAAAAGCAUCUUAUCGAAACCGCCGCUCGGAGACGAGCGCACAGCCACC |
| 139 | U5075 RNA | AGCGAAAAAAAAUUAAAAUCCAGCUUCUGUAAAGAGCCGACAGGAGAGGUGGCGAGACAAAAACCGCCACC |
| 140 | U5077 RNA | AGGUAGUAAAAAAGCUGAUUGAGUUGUUAGGAGAGGUGGCGAGAGUUUAGGGGAGGGCCACC |
| 141 | U5078 RNA | AGGCACGGUAACCGAAAGCGUUCGAACAGGGGCGCCAAUACAUUAAAAUUGCCACC |
| 142 | U5081 RNA | AGUUAGAAAGAGUACUCAUAAACGAUAAUAAAGUGAACCUAUUAGGCCCGUCUUGCGGAGUUGAGGAGCUUGC |
| | | AUCUCUGCUGAGGUUAAAACUAAUAUCUUGCCACC |
| 143 | [symbol] | AGGAAATTCCATTTGAATACAGCTTCTGGAGGGAGCCGACAGGAGACGTGGGGAGACGGCCACCATGGAAGAT |
| | | GCCAAAAACATTAAGAAGGGCCCAGCGCCATTCTACCCACTCGAAGACGGGACCGCCGGCGAGCAGCTGCACA |
| | | AAGCCATGAAGCGCTACGCCCTGGTGCCCGGCACCATCGCCTTTACCGACGCACATATCGAGGTGGACATTAC |
| | | CTACGCCGAGTACTTCGAGATGAGCGTTCGGCTGGCAGAAGCTATGAAGCGCTATGGGCTGAATACAAACCAT |
| | | CGGATCGTGGTGTGCAGCGAGAATAGCTTGCAGTTCTTCATGCCCGTGTTGGGTGCCCTGTTCATCGGTGTGG |
| | | CTGTGGCCCCAGCTAACGACATCTACAACGAGCGCGAGCTGCTGAACAGCATGGGCATCAGCCAGCCCACCGT |
| | | CGTATTCGTGAGCAAGAAAGGGCTGCAAAAGATCCTCAACGTGCAAAAGAAGCTACCGATCATACAAAAGATC |
| | | ATCATCATGGATAGCAAGACCGACTACCAGGGCTTCCAAAGCATGTACACCTTCGTGACTTCCCATTTGCCAC |
| | | CCGGCTTCAACGAGTACGACTTCGTGCCCGAGAGCTTCGACCGGGACAAAACCATCGCCCTGATCATGAACAG |
| | | TAGTGGCAGTACCGGATTGCCCAAGGGCGTAGCCCTACCGCACCGCACCGCTTGTGTCCGATTCAGTCATGCC |
| | | CGCGACCCCATCTTCGGCAACCAGATCATCCCCGACACCGCTATCCTCAGCGTGGTGCCATTTCACCACGGCT |
| | | TCGGCATGTTCACCACGCTGGGCTACTTGATCTGCGGCTTTCGGGTCGTGCTCATGTACCGCTTCGAGGAGGA |
| | | GCTATTCTTGCGCAGCTTGCAAGACTATAAGATTCAATCTGCCCTGCTGGTGCCCACACTATTTAGCTTCTTC |
| | | GCTAAGAGCACTCTCATCGACAAGTACGACCTAAGCAACTTGCACGAGATCGCCAGCGGCGGGGCGCCGCTCA |
| | | GCAAGGAGGTAGGTGAGGCCGTGGCCAAACGCTTCCACCTACCAGGCATCCGCCAGGGCTACGGCCTGACAGA |
| | | AACAACCAGCGCCATTCTGATCACCCCCGAAGGGGACGACAAGCCTGGCGCAGTAGGCAAGGTGGTGCCCTTC |
| | | TTCGAGGCTAAGGTGGTGGACTTGGACACCGGTAAGACACTGGGTGTGAACCAGCGCGGCGAGCTGTGCGTCC |
| | | GTGGCCCCATGATCATGAGCGGCTACGTTAACAACCCCGAGGCTACAAACGCTCTCATCGACAAGGACGGCTG |
| | | GCTGCACAGCGGCGACATCGCCTACTGGGACGAGGACGAGCACTTCTTCATCGTGGACCGGCTGAAAAGCCTG |
| | | ATCAAATACAAGGGCTACCAGGTAGCCCCCAGCCGAACTGGAGAGCATCCTGCTGCAACACCCCAACATCTTC |
| | | GACGCCGGGGTCGCCGGCCTGCCCGACGACGATGCCGGCGAGCTGACCCGCCGCAGTCGTCGTGCTGGAACAC |
| | | GGTAAAACCATGACCGAGAAGGAGATCGTGGACTATGTGGCCAGCCAGGTTACAACCGCCAAGAAGCTGCGCG |
| | | GTGGTGTTGTGTTCGTGGACGAGGTGCCTAAAGGACTGACCGGCAAGTTGGACGCCCGCAAGATCCGCGAGAT |
| | | TCTCATTAAGGCCAAGAAGGGCGGCAAGATCGCCGTGTAATAGGCTGCCTTCTGCGGGGCTTGCCTTCTGGCC |
| | | ATGCCCTTCTTCTCTCCCTTGCACCTGTACCTCTTGGTCTTTGAATAAAGCCTGAGTAGGAAGTAAAAAAAAA |
| | | AAAAAAAAAAAAAAAAAAAAAAAAAAAAAAAAAAAAAAAAAAAAAAAAAAAAAAAAAAAAAAAAAA |
| 144 | [symbol] | GCCACCATGGAAGATGCCAAAACATTAAGAAGGGCCCAGCGCCATTCTACCCACTCGAAGACGGGACCGCCGG |
| | | CGAGCAGCTGCACAAGCCATGAAGCGCTACGCCCTGGTGCCCGGCACCATCGCCTTTACCGACGCACATATCG |
| | | AGGTGGACATTACCTACGCCGAGTACTTCGAGATGAGCGTTCGGCTGGCAGAAGCTATGAAGCGCTATGGGCT |
| | | GAATACAAACCATCGGATCGTGGTGTGCAGCGAGAATAGCTTGCAGTTCTTCATGCCCGTGTTGGGTGCCCTG |
| | | TTCATCGGTGTGGCTGTGGCCCCAGCTAACGACATCTACAACGAGCGCGAGCTGCTGAACAGCATGGGCATCA |
| | | GCCAGCCCACCGTCGTATTCGTGAGCAAGAAAGGGCTGCAAAAGATCCTCAACGTGCAAAAGAAGCTACCGAT |
| | | CATACAAAAGATCATCATCATGGATAGCAAGACCGACTACCAGGGCTTCCAAAGCATGTACACCTTCGTGACT |
| | | TCCCATTTGCCACCCGGCTTCAACGAGTACGACTTCGTGCCCGAGAGCTTCGACCGGGACAAAACCATCGCCC |
| | | TGATCATGAACAGTAGTGGCAGTACCGGATTGCCCAAGGGCGTAGCCCTACCGCACCGCACCGCTTGTGTCCG |
| | | ATTCAGTCATGCCCGCGACCCCATCTTCGGCAACCAGATCATCCCCGACACCGCTATCCTCAGCGTGGTGCCA |
| | | TTTCACCACGGCTTCGGCATGTTCACCACGCTGGGCTACTTGATCTGCGGCTTTCGGGTCGTGCTCATGTACC |
| | | GCTTCGAGGAGGAGCTATTCTTGCGCAGCTTGCAAGACTATAAGATTCAATCTGCCCTGCTGGTGCCACACTA |
| | | TTTAGCTTCTTCGCTAAGAGCACTCTCATCGACAAGTACGACCTAAGCAACTTGCACGAGATCGCCAGCGGCG |
| | | GGGGCGCCGCTCAGCAAGGAGGTAGGTGAGGCCGTGGCCAAACGCTTCCACCTACCAGGCATCCGCCAGGGCT |
| | | ACGGCCTGACAGAAACAACCAGCGCCATTCTGATCACCCCCGAAGGGGACGACAAGCCTGGCGCAGTAGGCAA |
| | | GGTGGTGCCCTTCTTCGAGGCTAAGGTGGTGGACTTGGACACCGGTAAGACACTGGGTGTGAACCAGCGCGGC |
| | | GAGCTGTGCGTCCGTGGCCCCATGATCATGAGCGGCTACGTTAACAACCCCGAGGCTACAAACGCTCTCATCG |
| | | ACAAGGACGGCTGGCTGCACAGCGGCGACATCGCCTACTGGGACGAGGACGAGCACTTCTTCATCGTGGACCG |
| | | GCTGAAAAGCCTGATCAAATACAAGGGCTACCAGGTAGCCCCAGCCGAACTGGAGAGCATCCTGCTGCAACAC |
| | | CCAACATCTTCGACGCCGGGGTCGCCGGCCTGCCCGACGACGATGCCGGCGAGCTGCCCGCCGCAGTCGTCGT |
| | | GCTGGAACACGGTAAAACCATGACCGAGAAGGAGATCGTGGACTATGTGGCCAGCCAGGTTACAACCGCCAAG |
| | | AAGCTGCGCGGTGGTGTTGTGTTCGTGGACGAGGTGCCTAAAGGACTGACCGGCAAGTTGGACGCCCGCAAGA |
| | | TCCGCGAGATTCTCATTAAGGCCAAGAAGGGCGGCAAGATCGCCGTGTAATAGGCTGCCTTCTGCGGGGCTTG |
| | | CCTTCTGGCCATGCCCTTCTTCTCTCCCTTGCACCTGTACCTCTTGGTCTTTGAATAAAGCCTGAGTAGGAAG |
| | | TAAAAAAAAAAAAAAAAAAAAAAAAAAAAAAAAAAAAAAAAAAAAAAAAAAAAAAAAAAAAAAAAAAAAAAAA |
| | | AAA |

[symbol]

indicates data missing or illegible when filed

1-51. (canceled)

52. An artificial nucleic acid molecule comprising:
a. at least one open reading frame (ORF); and
b. at least one 5′-untranslated region element (5′-UTR element), the 5′-UTR element comprising a nucleic acid sequence selected from the following:
(i) wherein the 5′-UTR element contains 1, 2, 3 or more mutations compared to SEQ ID NO: 71; or
(ii) wherein the 5′-UTR element comprises the nucleic acid sequence of SEQ ID NO: 2, 3, 4, 5, 6, 7, 8, 9, 10, 11 or 12;
or (iii) wherein said 5′-UTR element comprises a nucleic acid sequence derived from a 5′-UTR of a transcript of the following genes: alpha-1-globin, ARC, TUBB1, TUBB4B, ANXA5, A1CF, TUBA1C, TUBB3 and HCV, and is truncated;
or the corresponding RNA sequence of the above nucleic acid sequence.

53. The artificial nucleic acid molecule according to claim 52, wherein the 5′-UTR element, compared with SEQ ID NO:71, comprises one or more mutations selected from the group consisting of: C9G, C10G, G16A, C17A, G19A, G19T, C26G, T27A, G28C, C57G, G58T, C61A and A62C.

54. The artificial nucleic acid molecule according to claim 53, wherein the 5′-UTR element compared to SEQ ID NO: 71 includes G16A, C17A and G19A.

55. The artificial nucleic acid molecule according to claim 52, wherein the 5′-UTR element comprises the nucleic acid sequence of SEQ ID NO: 1.

56. The artificial nucleic acid molecule according to claim 52, wherein the nucleic acid sequence is derived from the nucleic acid sequence of the 5′-UTR of the transcript of the following human genes: alpha-1-globin, ARC, TUBB1, TUBB4B and ANXA5; or derived from the following the nucleic acid sequence of the 5′-UTR of the transcript of the mouse gene: A1CF, TUBB1, TUBA1C, TUBB4B and TUBB3; or the nucleic acid sequence of the 5′-UTR of the transcript of the rat gene ARC; or derived from the viral gene HCV Nucleic acid sequence of the 5′-UTR of the transcript.

57. The artificial nucleic acid molecule according to claim 56, wherein the 5′-UTR element exhibits a length of 3-500 nucleotides, preferably 5-150 nucleotides, more preferably 50-110 nucleotides.

58. The artificial nucleic acid molecule according to claim 52, wherein the 5′-UTR element comprises the nucleic acid sequence of SEQ ID NO: 13, 14, 15, 16, 17, 18, 19, 20, 21, 22, 23 or 24.

59. The artificial nucleic acid molecule according to claim 52, wherein the 5′-UTR element comprises a Kozak sequence.

60. The artificial nucleic acid molecule according to claim 59, wherein the Kozak sequence comprises the nucleic acid sequence of SEQ ID NO:70.

61. The artificial nucleic acid molecule according to claim 52, further comprising at least one 3′-untranslated region element (3′-UTR element).

62. The artificial nucleic acid molecule according to claim 61, wherein the 3′-UTR element comprises the nucleic acid sequence of SEQ ID NO:72.

63. The artificial nucleic acid molecule according to claim 52, which further comprises a 5′ cap structure, a polycytidylic acid sequence, a polyadenylic acid sequence and/or a histone stem loop; preferably, the artificial nucleic acid the molecule comprises a poly(A) sequence; preferably, the poly(A) sequence comprises the nucleic acid sequence of SEQ ID NO:73.

64. The artificial nucleic acid molecule according to claim 52, wherein the ORF is codon optimized.

65. Artificial nucleic acid molecule according to claim 52, wherein the nucleic acid is RNA, preferably mRNA.

66. The artificial nucleic acid molecule according to claim 65, wherein the 5′-UTR element comprises nucleic acid sequence selected from SEQ ID NO: 74, 75, 76, 77, 78, 79, 80, 81, 82, 83, 84, 85, 86, 87, 88, 89, 90, 91, 92, 93, 94, 95, 96 or 97.

67. A vector comprising the artificial nucleic acid molecule according to claim 52.

68. The vector according to claim 67, which is a DNA vector.

69. The vector according to claim 67, which is a plasmid vector or a viral vector, preferably a plasmid vector.

70. The vector according to claim 67, which is a circular molecule.

71. A cell comprising the artificial nucleic acid molecule or a vector comprising the artificial nucleic acid molecule of claim 52.
